(19) United States
(12) Patent Application Publication
(10) Pub. No.: US 2012/0108501 A1
(43) Pub. Date: May 3, 2012

(54) Protease Inhibitors

(75) Inventor: Jennifer Riggs-Sauthier, Huntsville, AL (US)

(73) Assignee: NEKTAR THERAPEUTICS, SAN FRANCISCO, CA (US)

(21) Appl. No.: 13/375,689

(22) PCT Filed: Jun. 11, 2010
(86) PCT No.: PCT/US10/38416

§ 371 Date: Jan. 19, 2012

Related U.S. Application Data
(60) Provisional application No. 61/186,768, filed on Jun. 12, 2009.

(51) Int. Cl. A61K 31/4418; A61P 31/18; A61K 38/05; A61K 31/706; C07D 213/56; C07H 15/26

(52) U.S. Cl. 514/4.1; 546/332; 536/55; 514/357; 514/23

(57) ABSTRACT

The invention relates to (among other things) protease inhibitors containing both a water-soluble, non-peptidic oligomer and a lipophilic moiety-containing residue. A compound of the invention, when administered by any of a number of administration routes, exhibits advantages over protease inhibitor compounds lacking the water-soluble, non-peptidic oligomer and a lipophilic moiety-containing residue.

### CROSS REFERENCE TO RELATED APPLICATION

This application claims the benefit of priority under 35 U.S.C. §119(e) to U.S. Provisional Patent Application No. 61/186,768, filed Jun. 12, 2009, the disclosure of which is incorporated herein by reference in its entirety.

### FIELD OF THE INVENTION

This invention provides (among other things) methods for administering an antiviral protease inhibitor with increased therapeutic index and/or increased potency. The methods and active agents described herein relate to and/or have applications in (among others) the fields of pharmacotherapy, physiology, organic chemistry and polymer chemistry.

### BACKGROUND OF THE INVENTION

Since the first cases of acquired immunodeficiency syndrome (AIDS) were reported in 1981, infection with human immunodeficiency virus (HIV) has grown to pandemic proportions, resulting in an estimated 65 million infections and 25 million deaths. See Aug. 11, 2006, MMWR 55(31):841-844 (Center for Disease Control and Prevention). Protease inhibitors represent an important class of compounds used to treat individuals infected with HIV, although these compounds can also treat individuals suffering from other viral infections (e.g., Hepatitis C).

With respect to HIV, protease inhibitors act to inhibit the HIV viral proteases that are necessary for the proteolytic cleavage of the gag and gag/pol fusion polypeptides necessary for the generation of infective viral particles. Thus, by inhibiting this proteolytic cleavage, protease inhibitors diminish the ability of larger HIV-fusion polypeptide precursors to generate the mature form of proteins necessary for effective viral replication. McQuade et al. (1990) Science 247(4941):454-456.

Protease inhibitor-based therapy is acknowledged as an initial treatment for patients presenting symptomatic HIV disease and in non-symptomatic patients after the CD4 cell count is below 350/μL but before a level of 200/μL. Hammer et al. (2006) JAMA 296(7):827-843. In such cases, a protease inhibitor-based regimen will include a protease inhibitor (typically boosted with ritonavir) along with a combination of two nucleoside (or nucleotide) reverse transcriptase inhibitors. Id.

These conventional HIV protease inhibitors, as well as other protease inhibitors, have relatively low potency and/or relatively low (or narrow) therapeutic index.

HIV and other protease inhibitors having a relatively high potency and/or relatively high (or wide) therapeutic index would represent an improvement over conventional HIV protease inhibitors.

The present invention seeks to address this and other needs in the art.

### SUMMARY OF THE INVENTION

In one or more embodiments, a compound is provided, the compound comprising a residue of a protease inhibitor covalently attached, either directly or through one or more atoms to a water-soluble, non-peptidic oligomer, and further either directly or through one or more atoms, via a releasable linkage to a lipophilic moiety-containing residue.

In one or more embodiments, a compound is provided, the compound comprising a residue of a protease inhibitor covalently attached, either directly or through one or more atoms, to a water-soluble, non-peptidic oligomer, and further either directly or through one or more atoms, via a releasable linkage to a lipophilic moiety-containing residue, wherein the protease inhibitor is encompassed by Formula I.

In one or more embodiments, a compound is provided, the compound comprising a residue of a protease inhibitor covalently attached, either directly or through one or more atoms, to a water-soluble, non-peptidic oligomer, and further either directly or through one or more atoms, via a releasable linkage to a lipophilic moiety-containing residue, wherein the protease inhibitor is encompassed by Formula II.

In one or more embodiments, a compound is provided, the compound comprising a residue of a protease inhibitor covalently attached, either directly or through one or more atoms, to a water-soluble, non-peptidic oligomer, and further either directly or through one or more atoms, via a releasable linkage to a lipophilic moiety-containing residue, wherein the protease inhibitor is encompassed by Formula III.

In one or more embodiments, a compound is provided, the compound comprising a residue of a protease inhibitor covalently attached, either directly or through one or more atoms, to a water-soluble, non-peptidic oligomer, and further either directly or through one or more atoms, via a releasable linkage to a lipophilic moiety-containing residue, wherein the protease inhibitor is encompassed by Formula IV.

In one or more embodiments, a compound is provided, the compound comprising a residue of a protease inhibitor covalently attached, either directly or through one or more atoms, to a water-soluble, non-peptidic oligomer, and further either directly or through one or more atoms, via a releasable linkage to a lipophilic moiety-containing residue, wherein the protease inhibitor is encompassed by Formula V.

In one or more embodiments, a compound is provided, the compound comprising a residue of a protease inhibitor covalently attached, either directly or through one or more atoms, to a water-soluble, non-peptidic oligomer, and further either directly or through one or more atoms, via a releasable linkage to a lipophilic moiety-containing residue, wherein the protease inhibitor is encompassed by Formula VI.

In one or more embodiments, a compound is provided, the compound comprising a residue of a protease inhibitor covalently attached, either directly or through one or more atoms, to a water-soluble, non-peptidic oligomer, and further either directly or through one or more atoms, via a releasable linkage to a lipophilic moiety-containing residue, wherein the protease inhibitor is encompassed by Formula VII.

In one or more embodiments, a compound is provided, the compound comprising a residue of a protease inhibitor covalently attached, either directly or through one or more atoms, to a water-soluble, non-peptidic oligomer, and further either directly or through one or more atoms, via a releasable linkage to a lipophilic moiety-containing residue, wherein the protease inhibitor is encompassed by Formula VIII.

In one or more embodiments, a composition is provided, the composition comprising a residue of a protease inhibitor covalently attached, either directly or through one or more atoms, to a water-soluble, non-peptidic oligomer, and further either directly or through one or more atoms, via a releasable linkage to a lipophilic moiety-containing residue, wherein the protease inhibitor is encompassed by one of Formulae I to VIII, and, optionally, a pharmaceutically acceptable excipient.

In one or more embodiments of the invention, a dosage form is provided, the dosage form comprising a compound is provided, the compound comprising a residue of a protease inhibitor covalently attached, either directly or through one or more atoms, to a water-soluble, non-peptidic oligomer, and further either directly or through one or more atoms, via a releasable linkage to a lipophilic moiety-containing residue, wherein the protease inhibitor is encompassed by Formula I.

In one or more embodiments, a compound is provided, the compound having the following structure:

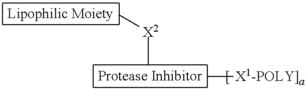

wherein:

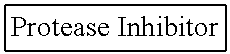

is an integer having a value of one to three, inclusive;

X¹, in each occurrence, is a spacer moiety;

X² is a releasable-linkage containing spacer moiety;

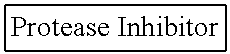

is a lipophilic moiety-containing residue; and

POLY, in each occurrence, is a water-soluble, non-peptidic oligomer.

In one or more embodiments of the invention, a method is provided, the method comprising, in any order, covalently attaching a water-soluble, non-peptidic oligomer to a small molecule protease inhibitor and also covalently attaching a linker moiety to the protease inhibitor.

In one or more embodiments of the invention, a method is provided, the method comprising administering a protease inhibitor conjugate of the invention to an individual in need thereof.

Additional embodiments of the present conjugates, compositions, methods, and the like will be apparent from the following description, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### DETAILED DESCRIPTION OF THE INVENTION

As used in this specification, the singular forms “a,” “an,” and “the” include plural referents unless the context clearly dictates otherwise.

In describing and claiming the present invention, the following terminology will be used in accordance with the definitions described below.

“Water soluble, non-peptidic oligomer” indicates an oligomer that is at least 35% (by weight) soluble, preferably greater than 70% (by weight), and more preferably greater than 95% (by weight) soluble, in water at room temperature. Typically, an unfiltered aqueous preparation of a “water-soluble” oligomer transmits at least 75%, more preferably at least 95%, of the amount of light transmitted by the same solution after filtering. It is most preferred, however, that the water-soluble oligomer is at least 95% (by weight) soluble in water or completely soluble in water. With respect to being “non-peptidic,” an oligomer is non-peptidic when it has less than 35% (by weight) of peptidic residues.

The terms “monomer,” “monomeric subunit” and “monomeric unit” are used interchangeably herein and refer to one of the basic structural units of a polymer or oligomer. In the case of a homo-oligomer, a single repeating structural unit forms the oligomer. In the case of a co-oligomer, two or more structural units are repeated—either in a pattern or randomly—to form the oligomer. Preferred oligomers used in connection with present the invention are homo-oligomers. The water-soluble, non-peptidic oligomer comprises one or more monomers serially attached to form a chain of monomers. The oligomer can be formed from a single monomer type (i.e., is homo-oligomeric) or two or three monomer types (i.e., is co-oligomeric).

An “oligomer” is a molecule possessing from about 1 to about 30 monomers. Specific oligomers for use in the invention include those having a variety of geometries such as linear, branched, or forked, to be described in greater detail below.

“PEG” or “polyethylene glycol,” as used herein, is meant to encompass any water-soluble poly(ethylene oxide). Unless otherwise indicated, a “PEG oligomer” or an oligoethylene glycol is one in which substantially all (preferably all) monomeric subunits are ethylene oxide subunits, though, the oligomer may contain distinct end capping moieties or functional groups, e.g., for conjugation. PEG oligomers for use in the present invention will comprise one of the two following structures: “—(CH₂CH₂O)ₙ—” or “—(CH₂CH₂O)ₙ₋₁CH₂CH₂—,” depending upon whether or not the terminal oxygen(s) has been displaced, e.g., during a synthetic transformation. As stated above, for the PEG oligomers, the variable (n) ranges from about 1 to 30, and the terminal groups and architecture of the overall PEG can vary. When PEG further comprises a functional group, A, for linking to, e.g., a small molecule drug, the functional group when covalently attached to a PEG oligomer does not result in formation of (i) an oxygen-oxygen bond (—O—O—, a peroxide linkage), or (ii) a nitrogen-oxygen bond (N—O, O—N).

The terms “end-capped” or “terminally capped” are interchangeably used herein to refer to a terminal or endpoint of a polymer having an end-capping moiety. Typically, although not necessarily, the end-capping moiety comprises a hydroxy or C₁₋₂₀ alkoxy group. Thus, examples of end-capping moieties include alkoxy (e.g., methoxy, ethoxy and benzyloxy), as well as aryl, heteroaryl, cyclo, heterocyclo, and the like. In addition, saturated, unsaturated, substituted and unsubstituted forms of each of the foregoing are envisioned. Moreover, the end-capping group can also be a silane. The end-capping group can also advantageously comprise a detectable label. When the polymer has an end-capping group comprising a detectable label, the amount or location of the polymer and/or the moiety (e.g., active agent) of interest to which the polymer is coupled, can be determined by using a suitable detector. Such labels include, without limitation, fluorescers, chemiluminescers, moieties used in enzyme labeling, colorimetric moieties (e.g., dyes), metal ions, radioactive moieties, and the like. Suitable detectors include photometers, films, spectrometers, and the like. In addition, the end-capping group may contain a targeting moiety.

The term “targeting moiety” is used herein to refer to a molecular structure that helps the conjugates of the invention to localize to a targeting area, e.g., help enter a cell, or bind a receptor. Preferably, the targeting moiety comprises of vitamin, antibody, antigen, receptor, DNA, RNA, sialyl Lewis X antigen, hyaluronic acid, sugars, cell specific lectins, steroid or steroid derivative, RGD peptide, ligand for a cell surface receptor, serum component, or combinatorial molecule directed against various intra- or extracellular receptors. The targeting moiety may also comprise a lipid or a phospholipid. Exemplary phospholipids include, without limitation, phosphatidylcholines, phospatidylserine, phospatidylinositol, phospatidylglycerol, and phospatidylethanolamine. These lipids may be in the form of micelles or liposomes and the like. The targeting moiety may further comprise a detectable label or alternately a detectable label may serve as a targeting moiety. When the conjugate has a targeting group comprising a detectable label, the amount and/or distribution/location of the polymer and/or the moiety (e.g., active agent) to which the polymer is coupled can be determined by using a suitable detector. Such labels include, without limitation, fluorescers, chemiluminescers, moieties used in enzyme labeling, colorimetric (e.g., dyes), metal ions, radioactive moieties, gold particles, quantum dots, and the like.

“Branched,” in reference to the geometry or overall structure of an oligomer, refers to an oligomer having two or more polymers “arms” extending from a branch point.

“Forked,” in reference to the geometry or overall structure of an oligomer, refers to an oligomer having two or more functional groups (typically through one or more atoms) extending from a branch point.

A “branch point” refers to a bifurcation point comprising one or more atoms at which an oligomer branches or forks from a linear structure into one or more additional arms.

The term “reactive” or “activated” refers to a functional group that reacts readily or at a practical rate under conventional conditions of organic synthesis. This is in contrast to those groups that either do not react or require strong catalysts or impractical reaction conditions in order to react (i.e., a “nonreactive” or “inert” group).

“Not readily reactive,” with reference to a functional group present on a molecule in a reaction mixture, indicates that the group remains largely intact under conditions that are effective to produce a desired reaction in the reaction mixture.

A “protecting group” is a moiety that prevents or blocks reaction of a particular chemically reactive functional group in a molecule under certain reaction conditions. The protecting group may vary depending upon the type of chemically reactive group being protected as well as the reaction conditions to be employed and the presence of additional reactive or protecting groups in the molecule. Functional groups which may be protected include, by way of example, carboxylic acid groups, amino groups, hydroxyl groups, thiol groups, carbonyl groups and the like. Representative protecting groups for carboxylic acids include esters (such as a p-methoxybenzyl ester), amides and hydrazides; for amino groups, carbamates (such as tert-butoxycarbonyl) and amides; for hydroxyl groups, ethers and esters; for thiol groups, thioethers and thioesters; for carbonyl groups, acetals and ketals; and the like. Such protecting groups are well-known to those skilled in the art and are described, for example, in T. W. Greene and G. M. Wuts, Protecting Groups in Organic Synthesis, Third Edition, Wiley, New York, 1999, and references cited therein.

A functional group in “protected form” refers to a functional group bearing a protecting group. As used herein, the term “functional group” or any synonym thereof encompasses protected forms thereof.

A “physiologically cleavable” or “hydrolyzable” or “degradable” bond is a relatively labile bond that reacts with water (i.e., is hydrolyzed) under physiological conditions. The tendency of a bond to hydrolyze in water may depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides, oligonucleotides, thioesters, and carbonates.

An “enzymatically degradable linkage” means a linkage that is subject to degradation by one or more enzymes.

A “stable” linkage or bond refers to a chemical bond that is substantially stable in water, that is to say, does not undergo hydrolysis under physiological conditions to any appreciable extent over an extended period of time. Examples of hydrolytically stable linkages include but are not limited to the following: carbon-carbon bonds (e.g., in aliphatic chains), ethers, amides, urethanes, amines, and the like. Generally, a stable linkage is one that exhibits a rate of hydrolysis of less than about 1-2% per day under physiological conditions. Hydrolysis rates of representative chemical bonds can be found in most standard chemistry textbooks.

“Substantially” or “essentially” means nearly totally or completely, for instance, 95% or greater, more preferably 97% or greater, still more preferably 98% or greater, even more preferably 99% or greater, yet still more preferably 99.9% or greater, with 99.99% or greater being most preferred of some given quantity.

“Monodisperse” refers to an oligomer composition wherein substantially all of the oligomers in the composition have a well-defined, single molecular weight and defined number of monomers, as determined by chromatography or mass spectrometry. Monodisperse oligomer compositions are in one sense pure, that is, substantially having a single and definable number (as a whole number) of monomers rather than a large distribution. A monodisperse oligomer composition possesses a MW/Mn value of 1.0005 or less, and more preferably, a MW/Mn value of 1.0000. By extension, a composition comprised of monodisperse conjugates means that substantially all oligomers of all conjugates in the composition have a single and definable number (as a whole number) of monomers rather than a large distribution and would possess a MW/Mn value of 1.0005, and more preferably, a MW/Mn value of 1.0000 if the oligomer were not attached to the therapeutic moiety. A composition comprised of monodisperse conjugates may, however, include one or more nonconjugate substances such as solvents, reagents, excipients, and so forth.

“Bimodal,” in reference to an oligomer composition, refers to an oligomer composition wherein substantially all oligomers in the composition have one of two definable and different numbers (as whole numbers) of monomers rather than a large distribution, and whose distribution of molecular weights, when plotted as a number fraction versus molecular weight, appears as two separate identifiable peaks. Preferably, for a bimodal oligomer composition as described herein, each peak is generally symmetric about its mean, although the size of the two peaks may differ. Ideally, the polydispersity index of each peak in the bimodal distribution, Mw/Mn, is 1.01 or less, more preferably 1.001 or less, and even more preferably 1.0005 or less, and most preferably a MW/Mn value of 1.0000. By extension, a composition comprised of bimodal conjugates means that substantially all oligomers of all conjugates in the composition have one of two definable and different numbers (as whole numbers) of monomers rather than a large distribution and would possess a MW/Mn value of 1.01 or less, more preferably 1.001 or less and even more preferably 1.0005 or less, and most preferably a MW/Mn value of 1.0000 if the oligomer were not attached to the therapeutic moiety. A composition comprised of bimodal conjugates may, however, include one or more nonconjugate substances such as solvents, reagents, excipients, and so forth.

A “protease inhibitor” is broadly used herein to refer to an organic, inorganic, or organometallic compound having a molecular weight of less than about 1000 Daltons and having some degree of activity as a protease inhibitor therapeutic. Protease inhibitor activity of a compound may be measured by assays known in the art and also as described herein.

A “biological membrane” is any membrane made of cells or tissues that serves as a barrier to at least some foreign entities or otherwise undesirable materials. As used herein a “biological membrane” includes those membranes that are associated with physiological protective barriers including, for example: the blood-brain barrier (BBB); the blood-cerebrospinal fluid barrier; the blood-placental barrier; the blood-milk barrier; the blood-testes barrier; and mucosal barriers including the vaginal mucosa, urethral mucosa, anal mucosa, buccal mucosa, sublingual mucosa, and rectal mucosa. Unless the context clearly dictates otherwise, the term “biological membrane” does not include those membranes associated with the middle gastro-intestinal tract (e.g., stomach and small intestines).

A “biological membrane crossing rate,” provides a measure of a compound's ability to cross a biological membrane, such as the blood-brain barrier (“BBB”). A variety of methods may be used to assess transport of a molecule across any given biological membrane. Methods to assess the biological membrane crossing rate associated with any given biological barrier (e.g., the blood-cerebrospinal fluid barrier, the blood-placental barrier, the blood-milk barrier, the intestinal barrier, and so forth), are known, described herein and/or in the relevant literature, and/or may be determined by one of ordinary skill in the art.

A “reduced rate of metabolism” refers to a measurable reduction in the rate of metabolism of a water-soluble oligomer-small molecule drug conjugate as compared to the rate of metabolism of the small molecule drug not attached to the water-soluble oligomer (i.e., the small molecule drug itself) or a reference standard material. In the special case of “reduced first pass rate of metabolism,” the same “reduced rate of metabolism” is required except that the small molecule drug (or reference standard material) and the corresponding conjugate are administered orally. Orally administered drugs are absorbed from the gastro-intestinal tract into the portal circulation and may pass through the liver prior to reaching the systemic circulation. Because the liver is the primary site of drug metabolism or biotransformation, a substantial amount of drug may be metabolized before it ever reaches the systemic circulation. The degree of first pass metabolism, and thus, any reduction thereof, may be measured by a number of different approaches. For instance, animal blood samples may be collected at timed intervals and the plasma or serum analyzed by liquid chromatography/mass spectrometry for metabolite levels. Other techniques for measuring a “reduced rate of metabolism” associated with the first pass metabolism and other metabolic processes are known, described herein and/or in the relevant literature, and/or may be determined by one of ordinary skill in the art. Preferably, a conjugate of the invention may provide a reduced rate of metabolism reduction satisfying at least one of the following values: at least about 30%; at least about 40%; at least about 50%; at least about 60%; at least about 70%; at least about 80%; and at least about 90%. A compound (such as a small molecule drug or conjugate thereof) that is “orally bioavailable” is one that preferably possesses a bioavailability when administered orally of greater than 25%, and preferably greater than 70%, where a compound's bioavailability is the fraction of administered drug that reaches the systemic circulation in unmetabolized form.

“Alkyl” refers to a hydrocarbon chain, ranging from about 1 to 20 atoms in length. Such hydrocarbon chains are preferably but not necessarily saturated and may be branched or straight chain. Exemplary alkyl groups include methyl, ethyl, propyl, butyl, pentyl, 2-methylbutyl, 2-ethylpropyl, 3-methylpentyl, and the like. As used herein, “alkyl” includes cycloalkyl when three or more carbon atoms are referenced. An “alkenyl” group is an alkyl of 2 to 20 carbon atoms with at least one carbon-carbon double bond.

The terms “substituted alkyl” or “substituted C_{q-r} alkyl” where q and r are integers identifying the range of carbon atoms contained in the alkyl group, denotes the above alkyl groups that are substituted by one, two or three halo (e.g., F, Cl, Br, I), trifluoromethyl, hydroxy, C₁₋₇ alkyl (e.g., methyl, ethyl, n-propyl, isopropyl, butyl, t-butyl, and so forth), C₁₋₇ alkoxy, C₁₋₇ acyloxy, C₃₋₇ heterocyclic, amino, phenoxy, nitro, carboxy, acyl, cyano. The substituted alkyl groups may be substituted once, twice or three times with the same or with different substituents.

“Lower alkyl” refers to an alkyl group containing from 1 to 6 carbon atoms, and may be straight chain or branched, as exemplified by methyl, ethyl, n-butyl, i-butyl, t-butyl. “Lower alkenyl” refers to a lower alkyl group of 2 to 6 carbon atoms having at least one carbon-carbon double bond.

“Non-interfering substituents” are those groups that, when present in a molecule, are typically non-reactive with other functional groups contained within the molecule.

“Alkoxy” refers to an —O—R group, wherein R is alkyl or substituted alkyl, preferably C₁-C₂₀ alkyl (e.g., methoxy, ethoxy, propyloxy, etc.), preferably C₁-C₇.

“Pharmaceutically acceptable excipient” or “pharmaceutically acceptable carrier” refers to component that may be included in the compositions of the invention causes no significant adverse toxicological effects to a patient.

The term “aryl” means an aromatic group having up to 14 carbon atoms. Aryl groups include phenyl, naphthyl, biphenyl, phenanthrenyl, naphthalenyl, and the like. “Substituted phenyl” and “substituted aryl” denote a phenyl group and aryl group, respectively, substituted with one, two, three, four or five (e.g. 1-2, 1-3 or 1-4 substituents) chosen from halo (F, Cl, Br, I), hydroxy, cyano, nitro, alkyl (e.g., C₁₋₆ alkyl), alkoxy (e.g., C₁₋₆ alkoxy), benzyloxy, carboxy, aryl, and so forth.

Chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g., CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding multivalent moiety, arylene). All atoms are understood to have their normal number of valences for bond formation (i.e., 1 for H, 4 for carbon, 3 for N, 2 for O, and 2, 4, or 6 for S, depending on the oxidation state of the S).

“Pharmacologically effective amount,” “physiologically effective amount,” and “therapeutically effective amount” are used interchangeably herein to mean the amount of a water-soluble oligomer-small molecule drug conjugate present in a composition that is needed to provide a desired level of active agent and/or conjugate in the bloodstream or in the target tissue. The precise amount may depend upon numerous factors, e.g., the particular active agent, the components and physical characteristics of the composition, intended patient population, patient considerations, and may readily be determined by one skilled in the art, based upon the information provided herein and available in the relevant literature.

A “difunctional” oligomer is an oligomer having two functional groups contained therein, typically at its termini. When the functional groups are the same, the oligomer is said to be homodifunctional. When the functional groups are different, the oligomer is said to be heterodifunctional.

A basic reactant or an acidic reactant described herein include neutral, charged, and any corresponding salt forms thereof.

The term “patient,” refers to a living organism suffering from or prone to a condition that can be prevented or treated by administration of a conjugate as described herein, and includes both humans and animals.

“Optional” or “optionally” means that the subsequently described circumstance may but need not necessarily occur, so that the description includes instances where the circumstance occurs and instances where it does not.

As indicated above, the present invention is directed to (among other things) a compound comprising a protease inhibitor residue covalently attached via a stable or degradable linkage to a water-soluble, non-peptidic oligomer.

HIV proteases such as atazanavir may have an amphiphilic pocket close to the protease binding site. Current protease inhibitors bind to the binding site in a manner that does not engage the amphiphilic pocket specifically. Without wishing to be found by theory, conjugation of a flexible water-soluble oligomer to the protease inhibitor enables (relevant bonding patterns that lead to) higher affinity interaction between the protease inhibitor and the HIV protease. This is believed to lead to higher potency. Without wishing to be bound by theory, it is further believed that adding and/or balancing the amphiphilicity of PEG with various linkers enables (relevant bonding patterns that lead to) higher affinity interaction between the protease inhibitor and the HIV protease. This is believed to lead to higher potency.

The invention provides conjugates having the following structure

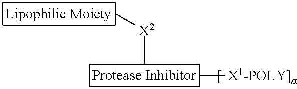

wherein:

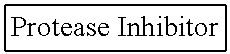

is a residue of a small molecule protease inhibitor;

(a) is an integer having a value of one to three, inclusive;

X¹, in each occurrence, is a spacer moiety;

X² is a releasable linkage;

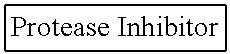

is a lipophilic-containin moiety; and

POLY, in each occurrence, is a water-soluble, non-peptidic oligomer.

Known compounds that act as small molecule protease inhibitors include those selected from the following classes: azahexane derivatives; amino acid derivatives; non-peptidic derivatives; pyranone compounds; pentan-1-amine derivatives; hexan-2-ylcarbamate derivatives; sulfonamide derivatives; and tri-substituted phenyl derivatives. Other small molecule protease inhibitors not necessarily belonging to any of the foregoing classes can also be used.

With respect to azahexane derivatives that are small molecule protease inhibitors, preferred azahexane derivatives have the following formula:

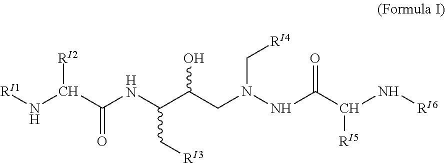

wherein:

R¹¹ is lower alkoxycarbonyl;

R¹² is secondary or tertiary lower alkyl or lower alkylthio-lower alkyl;

R¹³ is phenyl that is unsubstituted or substituted by one or more lower alkoxy radicals, or C4-8 cycloalkyl;

R¹⁴ is phenyl or cyclohexyl, each substituted in the 4-position by unsaturated heterocyclyl that is bonded by way of a ring carbon atom, has from 5 to 8 ring atoms, contains from 1 to 4 hetero atoms selected from the group nitrogen, oxygen, sulfur, sulfinyl (—SO—), and sulfonyl (—SO₂—) and is unsubstituted or substituted by lower alkyl or by phenyl-lower alkyl;

R¹⁵ is secondary or tertiary lower alkyl or lower alkylthio-lower alkyl; and

R¹⁶ is lower alkoxycarbonyl, and salts thereof.

A particularly preferred azahexane derivative is a compound of the following formula:

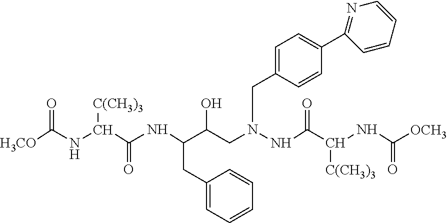

which is also known as atazanavir. Atazanavir and other azahexane derivatives, as well as methods for their synthesis, are described in U.S. Pat. No. 5,849,911.

With respect to amino acid derivatives that are small molecule protease inhibitors, preferred amino acid derivatives have the following formula:

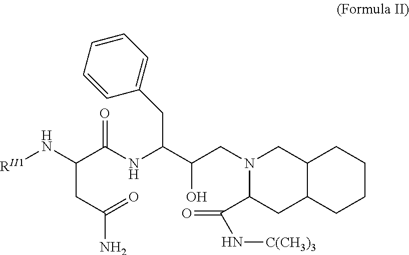

wherein:

R^{III} is benzyloxycarbonyl or 2-quinolylcarbonyl, and pharmaceutically acceptable acid addition salts thereof. A particularly preferred amino acid derivative is a compound of Formula II wherein R^{III} is 2-quinolylcarbonyl, also known as saquinavir. Such amino acid derivatives, as well as methods for their synthesis, are described in U.S. Pat. No. 5,196,438.

With respect to non-peptidic derivatives that are small molecule protease inhibitors, preferred non-peptidic derivatives have the following structure:

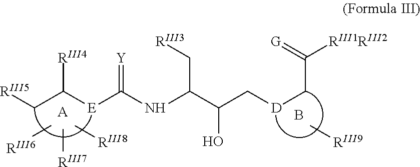

wherein:

R^{III1} and R^{III2} are independently selected from hydrogen, and substituted and unsubstituted alkyl and aryl, and R^{III1} and R^{III2} may form a ring with G;

R^{III3} is selected from mercapto and substituted and unsubstituted alkoxyl, aryloxyl, thioether, amino, alkyl, cycloalkyl, saturated and partially saturated heterocycle, and aryl;

R^{III4}, R^{III5}, R^{III6}, R^{III7}, and R^{III8} are independently selected from hydrogen, hydroxyl, mercapto, nitro, halo, —O-J, wherein J is a substituted or unsubstituted hydrolyzable group, and substituted and unsubstituted alkoxyl, aryloxyl, thioether, acyl, sulfinyl, sulfonyl, amino, alkyl, cycloalkyl, saturated and partially saturated heterocycle and aryl, and further wherein any of R^{III4}, R^{III5}, R^{III6}, R^{III7}, and R^{III8} may be a member of a spiro ring and any two of R^{III4}, R^{III5}, R^{III6}, R^{III7}, and R^{III8} may together be members of a ring;

Y and G are independently selected from oxygen, —NH, —N-alkyl, sulfur, selenium, and two hydrogen atoms,

D is a carbon or nitrogen;

E is a carbon or nitrogen;

R^{III9} is selected from hydrogen, halo, hydroxyl, mercapto, and substituted and unsubstituted alkoxyl, aryloxyl, thioether, amino, alkyl, and aryl, wherein R^{III9} may form part of a ring;

A is a carbocycle or heterocycle, which is optionally further substituted, and

B is a carbocycle or heterocycle, which is optionally further substituted, or

a pharmaceutically acceptable salt thereof.

A particularly preferred non-peptidic derivative that is a small molecule protease inhibitor is a compound of the following formula:

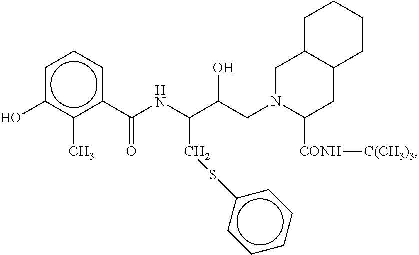

which is also known as nelfinavir. Nelfinavir and other non-peptidic derivatives, as well as methods for their synthesis, are described in U.S. Pat. No. 5,484,926 and WO 95/09843.

With respect to pyranone compounds that are small molecule protease inhibitors, preferred pyranone compounds have the following structure:

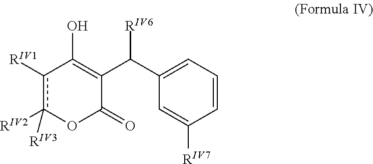

wherein:

R^{IV4} is H; R^{IV2} is C₃₋₅ alkyl, phenyl-(CH₂)₂—, heterocycyl-SO₂NH—(CH₂)₂—, cyclopropyl-(CH₂)₂—, F-phenyl-(CH₂)₂—, heterocycyl-SO₂NH-phenyl-, or F₃C—(CH₂)₂—; or R^{IV1} and R^{IV2} taken together are a double bond;

R^{IV3} is R^{IV4}—(CH₂)_{n′}—CH(R^{IV5})—, H₃C—[O(CH₂)₂]₂—CH₂—, C₃₋₅ alkyl, phenyl-(CH₂)₂—, heterocycyl-SO₂NH—(CH₂)₂—, (HOCH₂)₃C—NH—C(O)—NH—(CH₂)₃—, (H₂C)(H₂N)CH—(CH₂)₂—C(O)—NH—(CH₂)₃—, piperazin-1-yl-C(O)—NH—(CH₂)₃—, HO₃S(CH₂)₂—N(CH₃)—C(O)—(CH₂)₆—C(O)—NH—(CH₂)₃—, cyclopropyl-(CH₂)₂—, F-phenyl-(CH₂)₂—, heterocycyl-SO₂NH-phenyl-, or F₃—(CH₂)₂—; n′ is 0, 1 or 2; R^{IV4} is phenyl, heterocycyl, cyclopropyl, H₃C—[O(CH₂)₂]₂—, heterocycyl-SO₂NH—, Br—, N₃—, or HO₃S(CH₂)₂—N(CH₃)—C(O)—(CH₂)₆—C(O)—NH—; R^{IV5} is —CH₂—CH₃, or —CH₂-cyclopropyl;

R^{IV6} is cyclopropyl, CH₃—CH₂—, or t-butyl;

R^{IV7} is —NR^{IV8}SO₂-heterocycyl, NR^{IV8}SO₂-phenyl, optionally substituted with R^{IV9}, or —CH₂—SO₂-phenyl, optionally substituted with R^{IV9}, or —CH₂—SO₂-heterocycyl; R^{IV8} is H, or —CH₃; R^{IV9} is —CN, —F, —OH, or —NO₂; wherein heterocycyl is a 5-, 6- or 7-membered saturated or unsaturated ring containing from one to three heteroatoms selected from the group consisting of nitrogen, oxygen and sulfur; and including any bicyclic group in which any of the above heterocyclic rings is fused to a benzene ring or another heterocycle, optionally substituted with —CH₃, —CN, —OH, —C(O)OC₂H₅, —CF₃, —NH₂, or —C(O)—NH₂; or a pharmaceutically acceptable salt thereof.

A particularly preferred pyranone compound that is a small molecule protease inhibitor is a compound of the following formula:

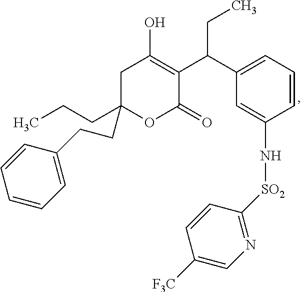

which is also known as tipranavir. Tipranavir and other non-peptidic derivatives, as well as methods for their synthesis, are described in U.S. Pat. Nos. 6,147,095, 6,231,887, and 5,484,926.

With respect to pentan-1-amine derivatives that are small molecule protease inhibitors, preferred pentan-1-amine derivatives have the following structure:

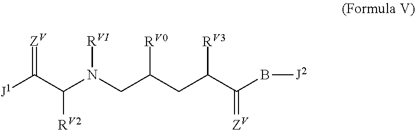

wherein:

R^{V0} is —OH or —NH₂;

Z^{V}, in each instance, is independently O, S, or NH;

R^{V1} and R^{V2} are independently hydrogen or optionally substituted C₁₋₄ alkyl, aryl, heterocycle, carbocyclic, —NH—SO₂C₁₋₃ alkyl, —O-aryl, —S-aryl, —NH-aryl, —O—C(O)-aryl, —S—C(O)-aryl, and —NH—C(O)-aryl, or R^{V1} and R^{V2} are joined together the form a monocyclic or bicyclic ring system;

R^{V3} is hydrogen, C₁₋₄ alkyl, benzyl (substituted or unsubtituted);

J¹ and J² are independently —OH, —NH₂, or optionally substituted C₁₋₆ alkyl, aryl, heterocycle, and carbocyclic, and

B is absent or selected from the group consisting of —NH—CH(CH₃)₂—C(O)—, —NH—CH(CH₃)₂—C(S)—, —NH—CH(CH₃)₂—C(NH)—, —NH—CH(CH₃)(CH₂CH₃)—C(O)—, —NH—CH(CH₃)(CH₂CH₃)—C(S)—, —NH—CH(CH₃)(CH₂CH₃)—C(NH)—, —NH—CH(phenyl)-C(O)—, —NH—CH(phenyl)-C(S)—, and —NH—CH(phenyl)-C(NH)—,

and pharmaceutically acceptable salts thereof.

A particularly preferred pentan-1-amine derivative that is a small molecule protease inhibitor is a compound of the following formula:

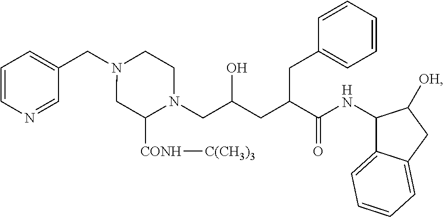

which is also known as indinavir. Indinavir and other pentan-1-amine derivatives, as well as methods for their synthesis, are described in U.S. Pat. No. 5,413,999 and European Patent Application No. EP 541 168.

With respect to hexan-2-ylcarbamate derivatives that are small molecule protease inhibitors, preferred hexane derivatives have the following structure:

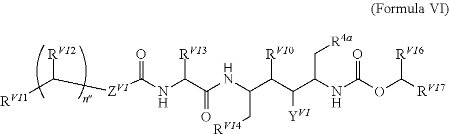

wherein:

R^{VI1} is monosubstituted thiazolyl, monosubstituted oxazolyl, monosubstituted isoxazolyl or monosubstituted isothiazolyl wherein the substituent is selected from (i) lower alkyl, (ii) lower alkenyl, (iii) cycloalkyl, (iv) cycloalkylalkyl, (v) cycloalkenyl, (vi) cycloalkenylalkyl, (vii) heterocyclic wherein the heterocyclic is selected from aziridinyl, azetidinyl, pyrrolidinyl, piperidinyl, piperazinyl, morpholinyl, thiomorpholinyl, thiazolyl, oxazolyl, isoxazolyl, isothiazolyl, pyridinyl, pyrimidinyl, pyridazinyl and pyrazinyl and wherein the heterocyclic is unsubstituted or substituted with a substituent selected from halo, loweralkyl, hydroxy, alkoxy and thioalkoxy, (viii) (heterocyclic)alkyl wherein heterocyclic is defined as above, (ix) alkoxyalkyl, (x) thioalkoxyalkyl, (xi) alkylamino, (xii) dialkylamino, (xiii) phenyl wherein the phenyl ring is unsubstituted or substituted with a substituent selected from halo, lower alkyl, hydroxy, alkoxy and thioalkoxy, (xiv) phenylalkyl wherein the phenyl ring is unsubstituted or substituted as defined above, (xv) dialkylaminoalkyl, (xvi) alkoxy and (xvii) thioalkoxy;

n″ is 1, 2 or 3;

R^{VI2} (is hydrogen or lower alkyl;

R^{VI3} is lower alkyl;

R^{VI4} and R^{4a} are independently selected from phenyl, thiazolyl and oxazolyl wherein the phenyl, thiazolyl or oxazolyl ring is unsubstituted or substituted with a substituent selected from (i) halo, (ii) loweralkyl, (iii) hydroxy, (iv) alkoxy and (v) thioalkoxy;

R^{VI6} is hydrogen or lower alkyl;

R^{VI7} is thiazolyl, oxazolyl, isoxazolyl or isothiazolyl wherein the thiazolyl, oxazolyl, isoxazolyl or isothiazolyl ring is unsubstituted or substituted with lower alkyl;

R^{VI0} is hydrogen and Y^{VI} is —OH or X^{VI} is —OH and Y^{VI} is hydrogen, with the proviso that X^{VI} is hydrogen and Y^{VI} is —OH when Z^{VI} is —N(R^{VI8})— and R^{VI7} is unsubstituted and with the proviso that X^{VI} is hydrogen and Y^{VI} is —OH when R^{VI3} is methyl and R^{VI7} is unsubstituted; and

Z^{VI} is absent, —O—, —S—, —CH²— or —N(R^{VI8})— wherein R^{VI8} is lower alkyl, cycloalkyl, —OH or —NHR^{8a} wherein R^{8a} is hydrogen, lower alkyl or an amine-protecting group;

and pharmaceutically acceptable salts, esters or prodrug thereof.

A particularly preferred hexan-2-ylcarbamate derivative that is a small molecule protease inhibitor is a compound of the following formula:

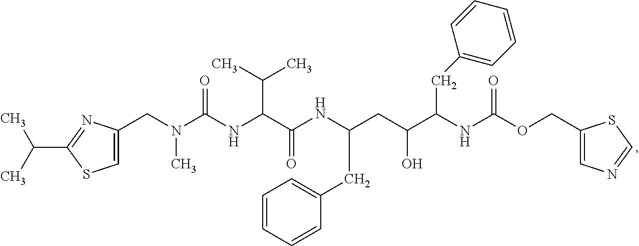

which is also known as ritonavir.

Another particularly preferred hexan-2-ylcarbamate derivative that is a small molecule protease inhibitor is a compound of the following formula:

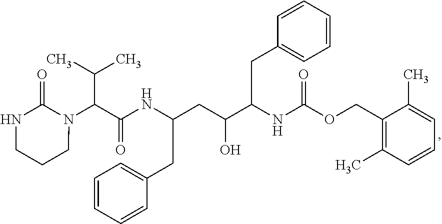

which is also known as lopinavir. Ritonavir, lopinavir and other hexan-2-ylcarbamate derivatives, as well as methods for their synthesis, are described in U.S. Pat. No. 5,541,206 and WO 94/14436.

With respect to sulfonamide derivatives that are small molecule protease inhibitors, preferred sulfonamide derivatives have the following structure:

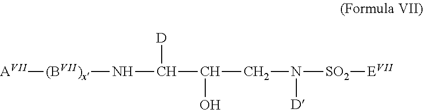

wherein:

A^{VI1} is selected from the group consisting of H, Het, —R^{VII1}-Het, —R^{VII1}—C₁₋₆ alkyl, which may be optionally substituted with one or more groups selected from the group consisting of hydroxy, C₁₋₄ alkoxy, Het, —O-Het, —NR^{VII2}—C(O)—N(R^{VII2})(R^{VII2}) and —C(O)—N(R^{VII2})(R^{VII2}); and —R^{VII1}—C₂₋₆ alkenyl, which may be optionally substituted with one or more groups selected from the group consisting of hydroxy, C₁₋₄ alkoxy, Het, —O-Het, —NR^{VII2}—C(O)N(R^{VII2})(R^{VII2}) and —C(O)—N(R^{VII2})(R^{VII2});

each R^{VII1} is independently selected from the group consisting of —C(O)—, —SO₂—, —C(O)C(O)—, —O—C(O)—, —SO₂, —S(O)₂—C(O)— and —NR^{VII2}—C(O)— and —NR^{VII2}—C(O)—C(O)—;

each Het is independently selected from the group consisting of C₃₋₇ cycloalkyl; C₅₋₇ cycloalkenyl; C₆₋₁₀ aryl; and 5-7 membered saturated or unsaturated heterocycle, containing one or more heteroatoms selected from N, N(R^{VII2}), O, S and S(O)_{n′″}, wherein said heterocycle may optionally be benzofused; and wherein any member of said Het may be optionally substituted with one or more substituents selected from the group consisting of oxo, —OR^{VII2}, —R^{VII2}, —N(R^{VII2}), —R^{VII2}—OH, —CN, CO₂R^{VII2}, —C(O)N(R^{VII2})(R^{VII2}), SO₂—N(R^{VII2})(R^{VII2}), —N(R^{VII2})—C(O)—R^{VII2}, —C(O)—R^{VII2}, —S(O)_{n′″}—R^{VII2}, —OCF₃, —S(O)_{n′″}—Ar, methylenedioxy, —N(R^{VII2})—SO₂(R^{VII2}), halo, —CF₃, —NO₂, Ar and —O—Ar;

each R^{VII2} is independently selected from the group consisting of H and C₁₋₃ alkyl optionally substituted with Ar;

B^{VI1}, when present, is —N(R^{VII2})—C(R^{VII3})(R^{VII3})—C(O)—;

x′ is 0 or 1;

each R^{VII3} is independently selected from the group consisting of H, Het, C₁₋₆ alkyl, C₂₋₆ alkenyl, C₃₋₆ cycloalkyl and C₅₋₆ cycloalkenyl, wherein any member of said R^{VII3}, except H, may be optionally substituted with one or more substituents selected from the group consisting of —OR^{VII2}, —C(O)—NH—R^{VII2}, —S(O)_{n′″}—N(R^{VII2})(R^{VII2}), Het, —CN, —SR^{VII2}, —CO₂R^{VII2}, NR^{VII2}—C(O)—R^{VII2};

each n′″ is independently 1 or 2;

D and D′ are independently selected from the group consisting of Ar; C₁₋₄ alkyl, which may be optionally substituted with one or more groups selected from C₃₋₆ cycloalkyl, —OR^{VII2}, —R^{VII3}, —O—Ar and Ar; C₂₋₄ alkenyl, which may be optionally substituted with one or more groups selected from the group consisting of C₃₋₆ cycloalkyl, —OR^{VII2}, —R^{VII3}, —O—Ar and Ar; C₃₋₆ cycloalkyl, which may be optionally substituted with or fused with Ar; and C₅₋₆ cycloalkenyl, which may be optionally substituted with or fused with Ar;

each Ar is independently selected from the group consisting of phenyl; 3-6 membered carbocyclic ring and 5-6 membered heterocyclic ring containing one or more heteroatoms selected from O, N, S, S(O)_{n′″} and N(R^{VII2}), wherein said carbocyclic or heterocyclic ring may be saturated or unsaturated and optionally substituted with one or more groups selected from the group consisting of oxo, —OR^{VII2}, —R^{VII2}, —N(R^{VII2})(R^{VII2}), —N(R^{VII2})—C(O)R^{VII2}, —R^{VII2}—OH, —CN, —CO₂R^{VII2}, —C(O)—N(R^{VII2})(R^{VII2}), halo and —CF₃;

E is selected from the group consisting of Het; O-Het; Het-Het; —O—R^{VII3}; —NR^{VII2}R^{VII3}; C₁₋₆ alkyl, which may be optionally substituted with one or more groups selected from the group consisting of R^{VII4} and Het; C₂₋₆ alkenyl, which may be optionally substituted with one or more groups selected from the group consisting of R^{VII4} and Het; C₃₋₆ saturated carbocycle, which may optionally be substituted with one or more groups selected from the group consisting of R^{VII4} and Het; and C₅₋₆ unsaturated carbocycle, which may optionally be substituted with one or more groups selected from the group consisting of R^{VII4} and Het; and

each R^{VII4} is independently selected from the group consisting of —OR^{VII2}, —C(O)—NHR^{VII2}, SO₂—NHR^{VII2}, halo, —NR^{VII2}—C(O)—R^{VII3} and —CN, and

and pharmaceutically acceptable salts, esters or prodrug thereof.

A particularly preferred sulfonamide derivative that is a small molecule protease inhibitor is a compound of the following formula:

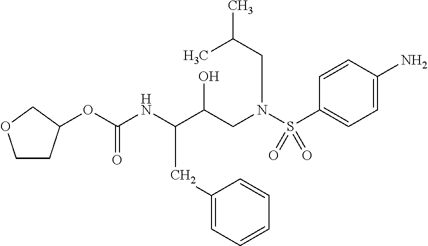

which is also known as amprenavir. Another particularly preferred sulfonamide derivative that is a small molecule protease inhibitor is a compound of the following formula:

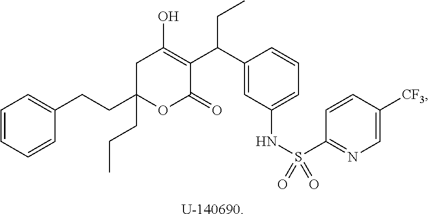

Amprenavir, U-140690 and other sulfonamide derivatives, as well as methods for their synthesis, are described in U.S. Pat. Nos. 5,732,490 and 5,585,397, WO 93/23368, and WO 95/30670.

A particularly preferred prodrug form of a sulfonamide derivative is the phosphonooxy-based prodrug of the following formula:

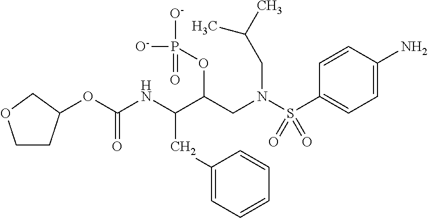

which is known as fosamprenavir and pharmaceutically acceptable salts thereof. Fosamprenavir and other sulfonamide derivatives, as well as methods for their synthesis, are described in U.S. Pat. Nos. 6,514,953 and 6,436,989.

With respect to tri-substituted phenyl derivatives that are small molecule protease inhibitors, preferred tri-substituted phenyl derivatives have the following structure:

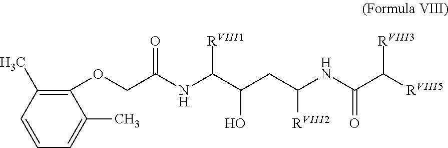

wherein:

R^{VIII1} is benzyl;

R^{VIII2} is benzyl or lower alkyl;

R^{VIII3} is lower alkyl; and

R^{VIII5} is

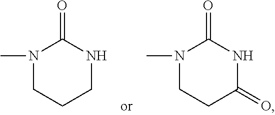

and pharmaceutically acceptable salts thereof. These and other small molecule protease inhibitors, as well as methods for their synthesis, are described in WO 97/21685.

As previously indicated, the small molecule protease inhibitor may not necessarily be categorized within one of the aforementioned classes. Such small molecule protease inhibitors, however, can still be conjugated to a water-soluble, non-peptidic oligomer as described herein. Nonlimiting additional small molecule protease inhibitors include the compounds:

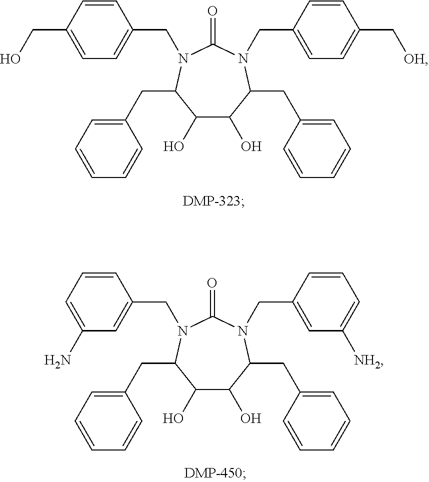

and
related compounds, disclosed in WO 93/07128.

Still other small molecule protease inhibitors include:

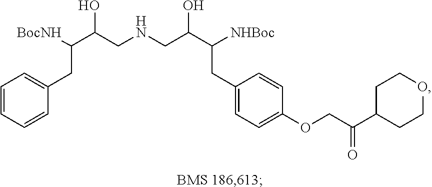

and other others described in European Patent Application No. EP 580 402.

Still other small molecule protease inhibitors include:

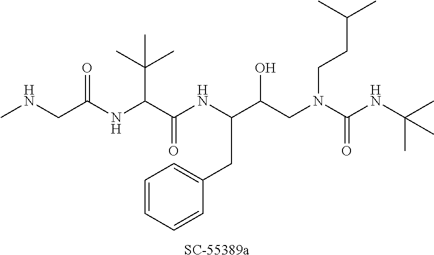

and other others described in WO 95/06061.

Still other small molecule protease inhibitors include:

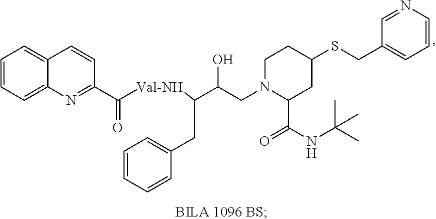

and others described in EP 560268.

In some embodiments, it is preferred that the small molecule protease inhibitor is selected from the group selected from the group consisting of amprenavir, atazanavir, fosamprenavir, indinavir, lopinavir, saquinavir, nelfinavir, ritonavir, tipranovir and darunavir.

Assays for determining whether a given compound (regardless of whether the compound includes a water-soluble, non-peptidic oligomer or not) can act as a protease inhibitor are known and/or may be prepared by one of ordinary skill in the art and are further described infra.

Each of these (and other) protease inhibitor moieties can be covalently attached (either directly or through one or more atoms) to a water-soluble, non-peptidic oligomer and to a lipophilic moiety-containing residue.

Exemplary molecular weights of small molecule drugs representing the protease inhibitor “pharmacophore” include molecular weights of: less than about 950; less than about 900; less than about 850; less than about 800; less than about 750; less than about 700; less than about 650; less than about 600; less than about 550; less than about 500; less than about 450; less than about 400; less than about 350; and less than about 300 Daltons.

The small molecule drug used in the invention, if chiral, may be obtained from a racemic mixture, or an optically active form, for example, a single optically active enantiomer, or any combination or ratio of enantiomers (i.e., scalemic mixture). In addition, the small molecule drug may possess one or more geometric isomers. With respect to geometric isomers, a composition can comprise a single geometric isomer or a mixture of two or more geometric isomers. A small molecule drug for use in the present invention can be in its customary active form, or may possess some degree of modification. For example, a small molecule drug may have a targeting agent, tag, or transporter attached thereto, prior to or after covalent attachment of an oligomer. Alternatively, the small molecule drug may possess a lipophilic moiety attached thereto, such as a phospholipid (e.g., distearoylphosphatidylethanolamine or “DSPE,” dipalmitoylphosphatidylethanolamine or “DPPE,” and so forth) or a small fatty acid. In some instances, however, it is preferred that the small molecule drug moiety does not include attachment to a lipophilic moiety.

The protease inhibitor moiety for coupling to a water-soluble, non-peptidic oligomer possesses a free hydroxyl, carboxyl, thio, amino group, or the like (i.e., “handle”) suitable for covalent attachment to the oligomer. In addition, the protease inhibitor moiety may be modified by introduction of a reactive group, preferably by conversion of one of its existing functional groups to a functional group suitable for formation of a stable or releasable covalent linkage between the oligomer and the drug. With respect to the reversible attachment of the lipophilic moiety-containing residue, a preferred functional group on the protease inhibitor is a hydroxyl group.

The water-soluble, non-peptidic oligomer (e.g., “POLY” in various structures provided herein) can have any of a number of different geometries. For example, the water-soluble, non-peptidic oligomer can be linear, branched, or forked. Most typically, the water-soluble, non-peptidic oligomer is linear or is branched, for example, having one branch point. Although much of the discussion herein is focused upon poly(ethylene oxide) as an illustrative oligomer, the discussion and structures presented herein can be readily extended to encompass any water-soluble, non-peptidic oligomers described above.

The molecular weight of the water-soluble, non-peptidic oligomer, excluding the linker portion, is generally relatively low. Exemplary values of the molecular weight of the water-soluble polymer include: below about 1500; below about 1450; below about 1400; below about 1350; below about 1300; below about 1250; below about 1200; below about 1150; below about 1100; below about 1050; below about 1000; below about 950; below about 900; below about 850; below about 800; below about 750; below about 700; below about 650; below about 600; below about 550; below about 500; below about 450; below about 400; below about 350; below about 300; below about 250; below about 200; and below about 100 Daltons.

Exemplary ranges of molecular weights of the water-soluble, non-peptidic oligomer (excluding the linker) include: from about 100 to about 1400 Daltons; from about 100 to about 1200 Daltons; from about 100 to about 800 Daltons; from about 100 to about 500 Daltons; from about 100 to about 400 Daltons; from about 200 to about 500 Daltons; from about 200 to about 400 Daltons; from about 75 to 1000 Daltons; and from about 75 to about 750 Daltons.

Preferably, the number of monomers in the water-soluble, non-peptidic oligomer falls within one or more of the following ranges: between about 1 and about 30 (inclusive); between about 1 and about 25; between about 1 and about 20; between about 1 and about 15; between about 1 and about 12; between about 1 and about 10. In certain instances, the number of monomers in series in the oligomer (and the corresponding conjugate) is one of 1, 2, 3, 4, 5, 6, 7, or 8. In additional embodiments, the oligomer (and the corresponding conjugate) contains 9, 10, 11, 12, 13, 14, 15, 16, 17, 18, 19, or 20 monomers. In yet further embodiments, the oligomer (and the corresponding conjugate) possesses 21, 22, 23, 24, 25, 26, 27, 28, 29 or 30 monomers in series. Thus, for example, when the water-soluble, non-peptidic polymer includes CH₃—(OCH₂CH₂)ₙ—, “n” is an integer that can be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, and can fall within one or more of the following ranges: between about 1 and about 25; between about 1 and about 20; between about 1 and about 15; between about 1 and about 12; between about 1 and about 10.

When the water-soluble, non-peptidic oligomer has 1, 2, 3, 4, 5, 6, 7, 8, 9, or 10 monomers, these values correspond to a methoxy end-capped oligo(ethylene oxide) having a molecular weights of about 75, 119, 163, 207, 251, 295, 339, 383, 427, and 471 Daltons, respectively. When the oligomer has 11, 12, 13, 14, or 15 monomers, these values correspond to methoxy end-capped oligo(ethylene oxide) having molecular weights corresponding to about 515, 559, 603, 647, and 691 Daltons, respectively.

When the water-soluble, non-peptidic oligomer is attached to the protease inhibitor (in contrast to the step-wise addition of one or more monomers to effectively “grow” the oligomer onto the protease inhibitor), it is preferred that the composition containing an activated form of the water-soluble, non-peptidic oligomer be monodisperse. In those instances, however, where a bimodal composition is employed, the composition will possess a bimodal distribution centering around any two of the above numbers of monomers. For instance, a bimodal oligomer may have any one of the following exemplary combinations of monomer subunits: 1-2, 1-3, 1-4, 1-5, 1-6, 1-7, 1-8, 1-9, 1-10, and so forth; 2-3, 2-4, 2-5, 2-6, 2-7, 2-8, 2-9, 2-10, and so forth; 3-4, 3-5, 3-6, 3-7, 3-8, 3-9, 3-10, and so forth; 4-5, 4-6, 4-7, 4-8, 4-9, 4-10, and so forth; 5-6, 5-7, 5-8, 5-9, 5-10, and so forth; 6-7, 6-8, 6-9, 6-10, and so forth; 7-8, 7-9, 7-10, and so forth; and 8-9, 8-10, and so forth.

In some instances, the composition containing an activated form of the water-soluble, non-peptidic oligomer will be trimodal or even tetramodal, possessing a range of monomers units as previously described. Oligomer compositions possessing a well-defined mixture of oligomers (i.e., being bimodal, trimodal, tetramodal, and so forth) can be prepared by mixing purified monodisperse oligomers to obtain a desired profile of oligomers (a mixture of two oligomers differing only in the number of monomers is bimodal; a mixture of three oligomers differing only in the number of monomers is trimodal; a mixture of four oligomers differing only in the number of monomers is tetramodal), or alternatively, can be obtained from column chromatography of a polydisperse oligomer by recovering the “center cut”, to obtain a mixture of oligomers in a desired and defined molecular weight range.

It is preferred that the water-soluble, non-peptidic oligomer is obtained from a composition that is preferably unimolecular or monodisperse. That is, the oligomers in the composition possess the same discrete molecular weight value rather than a distribution of molecular weights. Some monodisperse oligomers can be purchased from commercial sources such as those available from Sigma-Aldrich, or alternatively, can be prepared directly from commercially available starting materials such as Sigma-Aldrich. Water-soluble, non-peptidic oligomers can be prepared as described in Chen Y., Baker, G. L., J. Org. Chem., 6870-6873 (1999), WO 02/098949, and U.S. Patent Application Publication 2005/0136031.

The various components comprising the compounds of the invention are attached via a “spacer moiety.” For example, a spacer moiety that may optionally contain a degradable linkage connects the water-soluble, non-peptidic polymer to the protease inhibitor. In addition, a spacer moiety that includes a degradable linkage connects the lipophilic moiety-containing residue to the protease inhibitor.

Each spacer moiety may be a single bond, a single atom, such as an oxygen atom or a sulfur atom, two atoms, or a number of atoms. A spacer moiety is typically but is not necessarily linear in nature. The spacer moieties, “X¹” and “X² (commonly referred to as X),” are hydrolytically stable or releasable, and is preferably also enzymatically stable or releasable. Preferably, the spacer moiety “X” is one having a chain length of less than about 12 atoms, and preferably less than about 10 atoms, and even more preferably less than about 8 atoms and even more preferably less than about 5 atoms, whereby length is meant the number of atoms in a single chain, not counting substituents. For instance, a urea linkage such as this, R_{oligomer}—NH—(C═O)—NH—R′_{drug}, is considered to have a chain length of 3 atoms (—NH—C(O)—NH—). In selected embodiments, the linkage does not comprise further spacer groups.

In some instances, the spacer moiety “X¹” comprises an ether, amide, urethane, amine, thioether, urea, or a carbon-carbon bond. Functional groups such as those discussed below, and illustrated in the examples, are typically used for forming the linkages. The spacer moiety may less preferably also comprise (or be adjacent to or flanked by) other atoms, as described further below.

More specifically, in selected embodiments, a spacer moiety of the invention, X, may be any of the following: “—” (i.e., a covalent bond, that may be stable or degradable, between the protease inhibitor residue and the water-soluble, non-peptidic oligomer or the lipophilic moiety-containing residue), —O—, —NH—, —S—, —C(O)—, —C(O)O—, —OC(O)—, —CH₂—C(O)O—, —CH₂—OC(O)—, —C(O)O—CH₂—, —OC(O)—CH₂—, C(O)—NH, NH—C(O)—NH, O—C(O)—NH, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂, —CH₂—CH₂—NH—C(O)—CH₂—CH₂, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—CH₂—C(O)—NH—CH₂—CH₂—NH—, —CH₂—CH, —CH₂—C(O)—NH—CH₂—CH₂—NH—C(O)—, —CH₂—CH₂—CH₂—C(O)—NH—CH₂—CH₂—NH—C(O)—CH₂—, phosphate (and its derivatives), carbonate (and its derivatives), bivalent cycloalkyl group, —N(R⁶)—, R⁶ is H or an organic radical selected from the group consisting of alkyl, substituted alkyl, alkenyl, substituted alkenyl, alkynyl, substituted alkynyl, aryl and substituted aryl. Additional spacer moieties include, acylamino, acyl, aryloxy, alkylene bridge containing between 1 and 5 inclusive carbon atoms, alkylamino, dialkylamino having about 2 to 4 inclusive carbon atoms, piperidino, pyrrolidino, N-(lower alkyl)-2-piperidyl, morpholino, 1-piperizinyl, 4-(lower alkyl)-1-piperizinyl, 4-(hydroxyl-lower alkyl)-1-piperizinyl, 4-(methoxy-lower alkyl)-1-piperizinyl, and guanidine. In some instances, a portion or a functional group of the drug compound may be modified or removed altogether to facilitate attachment of the oligomer.

For purposes of the present invention, however, a group of atoms is not considered a spacer when it is immediately adjacent to an oligomer segment, and the group of atoms is the same as a monomer of the oligomer such that the group would represent a mere extension of the oligomer chain.

The linkages, “X” between the water-soluble, non-peptidic oligomer and the small molecule protease inhibitor, and also the linkage between the small molecule protease inhibitor and the lipophilic moiety-containing residue is formed by reaction of a functional group on a terminus of the oligomer (or nascent oligomer when it is desired to “grow” the oligomer onto the protease inhibitor) with a corresponding functional group within the protease inhibitor. Illustrative reactions are described briefly below. For example, an amino group on an oligomer or lipophilic moiety-containing residue may be reacted with a carboxylic acid or an activated carboxylic acid derivative on the small molecule, or vice versa, to produce an amide linkage. Alternatively, reaction of an amine on an oligomer or lipophilic moiety-containing residue with an activated carbonate (e.g. succinimidyl or benzotriazolyl carbonate) on the drug, or vice versa, forms a carbamate linkage. Reaction of an amine on an oligomer or lipophilic moiety-containing residue with an isocyanate (R—N═C═O) on a drug, or vice versa, forms a urea linkage (R—NH—(C═O)—NH—R′). Further, reaction of an alcohol (alkoxide) group on an oligomer or lipophilic moiety-containing residue with an alkyl halide, or halide group within a drug, or vice versa, forms an ether linkage. In yet another coupling approach, a small molecule having an aldehyde function is coupled to an oligomer or lipophilic moiety-containing residue amino group by reductive amination, resulting in formation of a secondary amine linkage between the oligomer and the small molecule.

Exemplary lipophilic-containing moieties include those selected from the group consisting of alkyl (e.g., C₁₋₂₀ alkyl), naturally occurring amino acids, non-naturally occurring amino acids, lipids, carbohydrates, lipids, phosphoholipids, vitamins, cofactors. For example, the lipophilic moiety can be selected from the group consisting of are acetyl, ethyl, propionate, octonoyl, butyl, valine, isoleucine, t-leucine, long chain fatty acids, and diacetone-glucose.

The termini of the water-soluble, non-peptidic oligomer not bearing a functional group may be capped to render it unreactive. When the oligomer includes a further functional group at a terminus other than that intended for formation of a conjugate, that group is either selected such that it is unreactive under the conditions of formation of the linkage “X,” or it is protected during the formation of the linkage “X.”

As stated above, the water-soluble, non-peptidic oligomer includes at least one functional group prior to conjugation. The functional group comprises an electrophilic or nucleophilic group for covalent attachment to a small molecule, depending upon the reactive group contained within or introduced into the small molecule. Examples of nucleophilic groups that may be present in either the oligomer or the small molecule include hydroxyl, amine, hydrazine (—NHNH₂), hydrazide (—C(O)NHNH₂), and thiol. Preferred nucleophiles include amine, hydrazine, hydrazide, and thiol, particularly amine. Most small molecule drugs for covalent attachment to an oligomer will possess a free hydroxyl, amino, thio, aldehyde, ketone, or carboxyl group.

Examples of electrophilic functional groups that may be present in either the oligomer or the small molecule include carboxylic acid, carboxylic ester, particularly imide esters, orthoester, carbonate, isocyanate, isothiocyanate, aldehyde, ketone, thione, alkenyl, acrylate, methacrylate, acrylamide, sulfone, maleimide, disulfide, iodo, epoxy, sulfonate, thiosulfonate, silane, alkoxysilane, and halosilane. More specific examples of these groups include succinimidyl ester or carbonate, imidazoyl ester or carbonate, benzotriazole ester or carbonate, vinyl sulfone, chloroethylsulfone, vinylpyridine, pyridyl disulfide, iodoacetamide, glyoxal, dione, mesylate, tosylate, and tresylate (2,2,2-trifluoroethanesulfonate).

Also included are sulfur analogs of several of these groups, such as thione, thione hydrate, thioketal, 2-thiazolidine thione, etc., as well as hydrates or protected derivatives of any of the above moieties (e.g. aldehyde hydrate, hemiacetal, acetal, ketone hydrate, hemiketal, ketal, thioketal, thioacetal).

An “activated derivative” of a carboxylic acid refers to a carboxylic acid derivative that reacts readily with nucleophiles, generally much more readily than the underivatized carboxylic acid. Activated carboxylic acids include, for example, acid halides (such as acid chlorides), anhydrides, carbonates, and esters. Such esters include imide esters, of the general form —(CO)O—N[(CO)—]₂; for example, N-hydroxysuccinimidyl (NHS) esters or N-hydroxyphthalimidyl esters. Also preferred are imidazolyl esters and benzotriazole esters. Particularly preferred are activated propionic acid or butanoic acid esters, as described in co-owned U.S. Pat. No. 5,672,662. These include groups of the form —(CH₂)₂₋₃C(═O)O-Q, where Q is preferably selected from N-succinimide, N-sulfosuccinimide, N-phthalimide, N-glutarimide, N-tetrahydrophthalimide, N-norbornene-2,3-dicarboximide, benzotriazole, 7-azabenzotriazole, and imidazole.

Other preferred electrophilic groups include succinimidyl carbonate, maleimide, benzotriazole carbonate, glycidyl ether, imidazoyl carbonate, p-nitrophenyl carbonate, acrylate, tresylate, aldehyde, and orthopyridyl disulfide.

These electrophilic groups are subject to reaction with nucleophiles, e.g., hydroxy, thio, or amino groups, to produce various bond types. Preferred for the present invention are reactions which favor formation of a hydrolytically stable linkage. For example, carboxylic acids and activated derivatives thereof, which include orthoesters, succinimidyl esters, imidazolyl esters, and benzotriazole esters, react with the above types of nucleophiles to form esters, thioesters, and amides, respectively, of which amides are the most hydrolytically stable. Carbonates, including succinimidyl, imidazolyl, and benzotriazole carbonates, react with amino groups to form carbamates. Isocyanates (R—N═C═O) react with hydroxyl or amino groups to form, respectively, carbamate (RNH—C(O)—OR′) or urea (RNH—C(O)—NHR′) linkages. Aldehydes, ketones, glyoxals, diones and their hydrates or alcohol adducts (i.e., aldehyde hydrate, hemiacetal, acetal, ketone hydrate, hemiketal, and ketal) are preferably reacted with amines, followed by reduction of the resulting imine, if desired, to provide an amine linkage (reductive amination).

Several of the electrophilic functional groups include electrophilic double bonds to which nucleophilic groups, such as thiols, can be added, to form, for example, thioether bonds. These groups include maleimides, vinyl sulfones, vinyl pyridine, acrylates, methacrylates, and acrylamides. Other groups comprise leaving groups that can be displaced by a nucleophile; these include chloroethyl sulfone, pyridyl disulfides (which include a cleavable S—S bond), iodoacetamide, mesylate, tosylate, thiosulfonate, and tresylate. Epoxides react by ring opening by a nucleophile, to form, for example, an ether or amine bond. Reactions involving complementary reactive groups such as those noted above on the oligomer and the small molecule are utilized to prepare the conjugates of the invention.

In some instances the protease inhibitor may not have a functional group suited for conjugation. In this instance, it is possible to modify (or “functionalize”) the “original” protease inhibitor so that it does have a functional group suited for conjugation. For example, if the protease inhibitor has an amide group, but an amine group is desired, it is possible to modify the amide group to an amine group by way of a Hofmann rearrangement, Curtius rearrangement (once the amide is converted to an azide) or Lossen rearrangement (once amide is concerted to hydroxamide followed by treatment with tolyene-2-sulfonyl chloride/base).

It is possible to prepare a conjugate of small molecule protease inhibitor bearing a carboxyl group wherein the carboxyl group-bearing small molecule protease inhibitor is coupled to an amino-terminated oligomeric ethylene glycol, to provide a conjugate having an amide group covalently linking the small molecule protease inhibitor to the oligomer. This can be performed, for example, by combining the carboxyl group-bearing small molecule protease inhibitor with the amino-terminated oligomeric ethylene glycol in the presence of a coupling reagent, (such as dicyclohexylcarbodiimide or “DCC”) in an anhydrous organic solvent.

Further, it is possible to prepare a conjugate of a small molecule protease inhibitor bearing a hydroxyl group wherein the hydroxyl group-bearing small molecule protease inhibitor is coupled to an oligomeric ethylene glycol halide to result in an ether (—O—) linked small molecule conjugate. This can be performed, for example, by using sodium hydride to deprotonate the hydroxyl group followed by reaction with a halide-terminated oligomeric ethylene glycol.

Further, it is possible to prepare a conjugate of a small molecule protease inhibitor moiety bearing a hydroxyl group wherein the hydroxyl group-bearing small molecule protease inhibitor moiety is coupled to an oligomeric ethylene glycol or lipophilic moiety-containing residue bearing an haloformate group [e.g., CH₃(OCH₂CH₂)ₙOC(O)-halo, where halo is chloro, bromo, iodo] to result in a carbonate [—O—C(O)—O—] linked small molecule conjugate. This can be performed, for example, by combining a protease inhibitor moiety and an oligomeric ethylene glycol or lipophilic moiety-containing residue bearing a haloformate group in the presence of a nucleophilic catalyst (such as 4-dimethylaminopyridine or “DMAP”) to thereby result in the corresponding carbonate-linked conjugate.

In another example, it is possible to prepare a conjugate of a small molecule protease inhibitor bearing a ketone group by first reducing the ketone group to form the corresponding hydroxyl group. Thereafter, the small molecule protease inhibitor now bearing a hydroxyl group can be coupled as described herein.

In still another instance, it is possible to prepare a conjugate of a small molecule protease inhibitor bearing an amine group. In one approach, the amine group-bearing small molecule protease inhibitor and an aldehyde-bearing oligomer or lipophilic moiety-containing residue are dissolved in a suitable buffer after which a suitable reducing agent (e.g., NaCNBH₃) is added. Following reduction, the result is an amine linkage formed between the amine group of the amine group-containing small molecule protease inhibitor and the carbonyl carbon of the aldehyde-bearing oligomer.

In another approach for preparing a conjugate of a small molecule protease inhibitor bearing an amine group, a carboxylic acid-bearing oligomer or lipophilic moiety-containing residue and the amine group-bearing small molecule protease inhibitor are combined, in the presence of a coupling reagent (e.g., DCC). The result is an amide linkage formed between the amine group of the amine group-containing small molecule protease inhibitor and the carbonyl of the carboxylic acid-bearing oligomer.

One exemplary approach for preparing the compounds of the invention, a protease inhibitor already containing a water-soluble, non-peptidic oligomer attached thereto is used in conjugation reaction to attach via a degradable linkage to a lipophilic moiety-containing residue. Protease inhibitors containing a water-soluble, non-peptidic oligomer attached thereto are described herein and in, for example, WO 2008/112289.

Exemplary compounds of the invention of Formula I include those having the following structures (L is the Linker moiety):

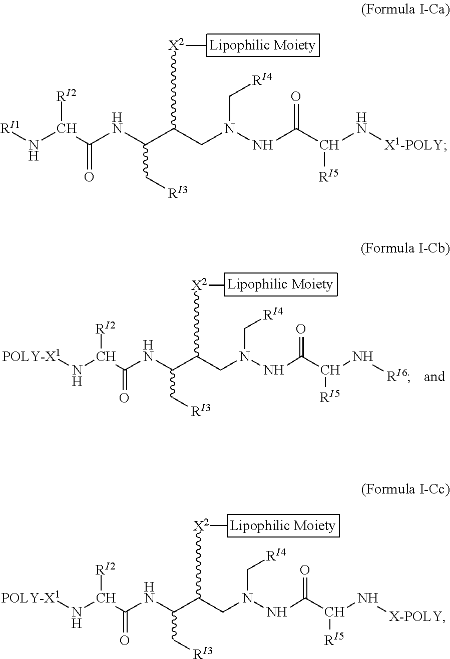

wherein for each of Formula I-Ca, Formula I-Cb and Formula I-Cc: X is a spacer moiety (releasable or stable); X¹ is a spacer moiety (releaseable or stable); X² is a releasable linkage-containing spacer moiety; POLY is a water-soluble, non-peptidic oligomer;

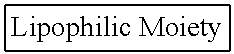

is a lipophilic moiety-containing residue and each of R¹¹, R¹², R¹³, R¹⁴, R¹⁵ and R¹⁶ is as defined with respect to Formula I.

Exemplary conjugates of small molecule protease inhibitors of Formula II include those having the following structures:

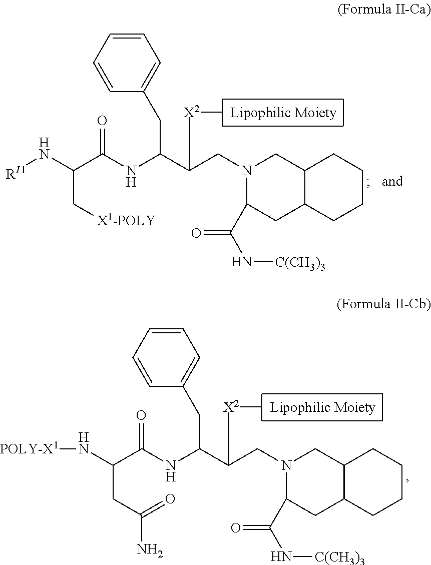

wherein, in each instance in which it appears: X¹ is a spacer moiety (releasable or stable); X² is a releasable linkage-containing spacer moiety; POLY is a water-soluble, non-peptidic oligomer;

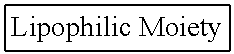

is a lipophilic moiety-containing residue; and R^{II1} is benzyloxycarbonyl or 2-quinolylcarbonyl.

Exemplary conjugates of the small molecule protease inhibitors of Formula III include those having the following structures:

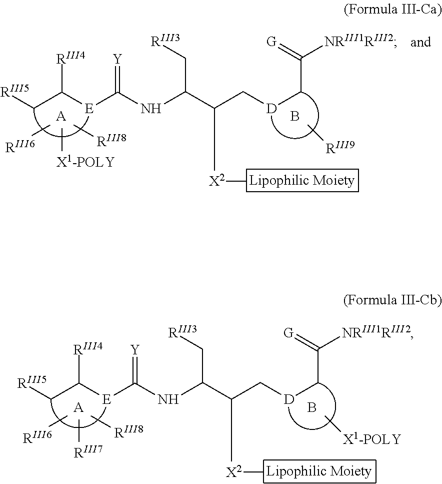

wherein, in each instance in where it appears: X¹ is a spacer moiety (stable or releasable); POLY is a water-soluble, non-peptidic oligomer; X² is a releasable linkage-containing spacer moiety;

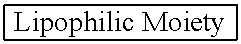

is a lipophilic moiety-containing residue; and each of R^{III1}, R^{III2}, R^{III3}, R^{III4}, R^{III5}, R^{III6}, R^{III7}, R^{III8}, Y, G, D, E, R^{III9}, A and B is as defined with respect to Formula III.

Exemplary conjugates of the small molecule protease inhibitors of Formula IV include those having the following structure:

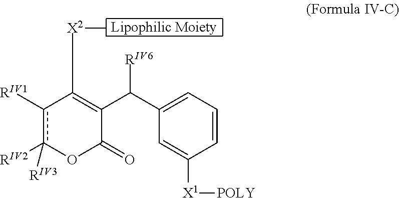

wherein: X¹ is a spacer moiety (stable or releasable); POLY is a water-soluble, non-peptidic oligomer; X² is a releasable linkage-containing spacer moiety;

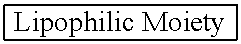

is a lipophilic moiety-containing residue; and R^{IV1}, R^{IV2}, R^{IV3} and R^{IV6} is as defined with respect to Formula IV.

Exemplary conjugates of the small molecule protease inhibitors of Formula V include those having the following structure:

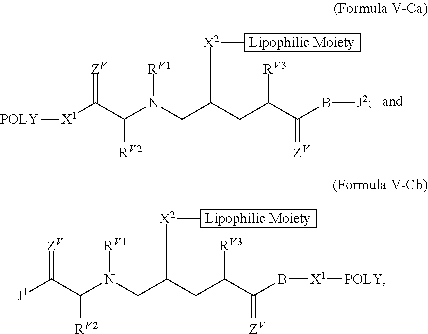

wherein, in each instance where it appears: X¹ is a spacer moiety (stable or releasable); POLY is a water-soluble, non-peptidic oligomer; X² is a releasable linkage-containing spacer moiety;

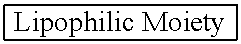

is a lipophilic moiety-containing residue; and each of Z^{V}, R^{V1}, R^{V2}, R^{V3}, J¹, J² and B is as defined with respect to Formula V.

Exemplary conjugates of the small molecule protease inhibitors of Formula VI include those having the following structure:

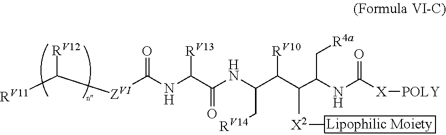

wherein: X¹ is a spacer moiety (stable or releasable; POLY is a water-soluble, non-peptidic oligomer; X² is a releasable linkage-containing spacer moiety;

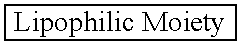

is a lipophilic moiety-containing residue; R^{VI0} is H; and each of R^{VI1}; n″, R^{VI2}, R^{VI3}, R^{VI4}, R^{4a} and Z^{V1} is as defined with respect to Formula VI.

Exemplary conjugates of the small molecule protease inhibitors of Formula VII include those having the following structure:

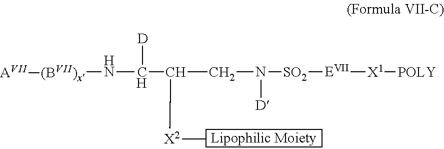

wherein: X¹ is a spacer moiety (stable or releasable); POLY is a water-soluble, non-peptidic oligomer; X² is a releasable linkage-containing spacer moiety;

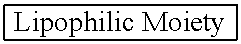

is a lipophilic moiety-containing residue; and each of A^{VI1}, B^{VI1}, x′, D, D′ and E^{VI1} is as defined with respect to Formula VII.

Exemplary conjugates of the small molecule protease inhibitors of Formula VIII include those having the following structures:

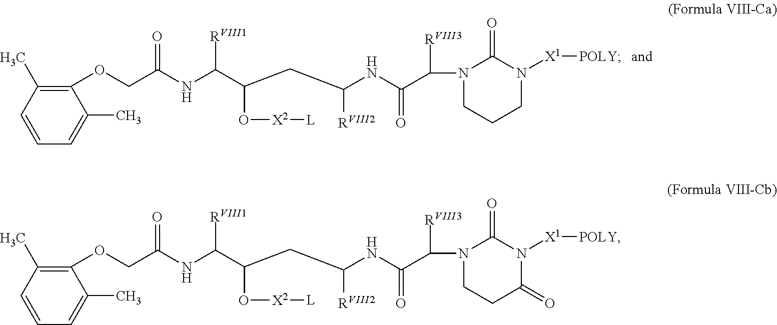

wherein: X¹ is a stable or releasable linkage; POLY is a water-soluble, non-peptidic oligomer; and each of
R^{VIII1}, R^{VIII2} and R^{VII3} is as defined with respect to Formula VIII.

While it is believed that the full scope of the conjugates disclosed herein behave as described, an optimally sized oligomer can be identified as follows.

First, an oligomer obtained from a monodisperse or bimodal water soluble oligomer is conjugated to the small molecule drug. Preferably, the drug is orally bioavailable, and on its own, exhibits a non-negligible blood-brain barrier crossing rate. Next, the ability of the conjugate to cross the blood-brain barrier is determined using an appropriate model and compared to that of the unmodified parent drug. If the results are favorable, that is to say, if, for example, the rate of crossing is significantly reduced, then the bioactivity of conjugate is further evaluated. Preferably, the compounds according to the invention maintain a significant degree of bioactivity relative to the parent drug, i.e., greater than about 30% of the bioactivity of the parent drug, or even more preferably, greater than about 50% of the bioactivity of the parent drug.

The above steps are repeated one or more times using oligomers of the same monomer type but having a different number of subunits and the results compared.

For each conjugate whose ability to cross the blood-brain barrier is reduced in comparison to the non-conjugated small molecule drug, its oral bioavailability is then assessed. Based upon these results, that is to say, based upon the comparison of conjugates of oligomers of varying size to a given small molecule at a given position or location within the small molecule, it is possible to determine the size of the oligomer most effective in providing a conjugate having an optimal balance between reduction in biological membrane crossing, oral bioavailability, and bioactivity. The small size of the oligomers makes such screenings feasible and allows one to effectively tailor the properties of the resulting conjugate. By making small, incremental changes in oligomer size and utilizing an experimental design approach, one can effectively identify a conjugate having a favorable balance of reduction in biological membrane crossing rate, bioactivity, and oral bioavailability. In some instances, attachment of an oligomer as described herein is effective to actually increase oral bioavailability of the drug.

Similarly, it is possible to identity an optimal size for the lipophilic moiety-containing residue by testing different sizes of lipophilic moiety-containing residues.

Assays for HIV Activity

To determine whether the small molecule protease inhibitor, or the conjugate of a small molecule protease inhibitor and a water-soluble non-peptidic polymer, or the conjugate of a small molecule protease inhibitor and a water-soluble non-peptidic polymer and a linker, has anti-HIV activity, it is possible to test such compounds. Anti-HIV activity can be tested as described in the Experimental. In addition, Anti-HIV activity can be tested in a human T-cell line by, for example, the method disclosed in Kempf et al. (1991) Antimicrob. Agents Chemother. 35(11):2209-2214, HIV-1_{3B} stock (10^{4.7} 50% tissue culture infection doses per ml) can be diluted 100-fold and incubated with MT-4 cells at 4×10⁵ cells per ml for one hour at 37° C. (multiplicity of infection, 0.001 50% tissue culture infective dose per cell). The resulting culture is then washed twice, resuspended to 10⁵ cells per ml of medium, seeded in a volume of 1% dimethyl sulfoxide solution of compound in a series of half-log-unit dilutions in medium in triplicate. The virus control culture can be treated in an identical manner, except that no compound is added to the medium. The cell control is incubated in the absence of compound or virus. Optical density (OD) is then measured at day 5 by using 3-(4,5-dimethylthiazol-2-yl)-2,5-diphenyltetrazolium bromide (MTT) in a colorimetric assay. See Pauwels et al. (1988) J. Virol Methods 20:309-321. Virus and control OD values are averaged over six determinations. Percent inhibition of HIV cytopathic effect (CPE) is calculated by the following formula: [(average OD−virus control OD/(cell control OD−virus control OD)]×100. Cytotoxicity is determined by the incubation in duplicate with serial dilutions of compound in the absence of virus. Percent cytotoxicity is determined according to the following formula: (average OD/cell control OD)×100. The EC₅₀ represents the concentration of compound that gave 50% inhibition of the cytopathic effect. The CCIC₅₀ is the concentration of compound which gives a 50% cytotoxic effect. It is noted that when conjugation of the water-soluble, non-peptidic oligomer occurs at the hydroxyl group located at 26 position of saquinavir, no anti-HIV activity is measured. See Table 1, Example 3. While not wishing to be bound by theory, it appears that the availability of this hydroxyl group is required for activity (a “binding hydroxyl group”). As a consequence, it is preferred in some embodiments that the conjugate lacks attachment of the water-soluble, non-peptidic oligomer at a binding hydroxyl group. A “binding hydroxyl group” for any given protease inhibitor can be determined by one of ordinary skill in the art by, for example, experimental testing and/or by comparing the structure of the protease inhibitor of interest with the structure of saquinavir and determining which hydroxyl group in the protease inhibitor corresponds to the “binding hydroxyl group” at position 26 in saquinavir. In one or more embodiments, however, it is preferred that the “binding hydroxyl group” serves as the attachment point for a degradably attached lipophilic moiety-containing residue.

The present invention also includes pharmaceutical preparations comprising an HIV protease inhibitor (whether “potent” or not) in combination with a pharmaceutical excipient. Generally, the conjugate itself will be in a solid form (e.g., a precipitate), which can be combined with a suitable pharmaceutical excipient that can be in either solid or liquid form.

Exemplary excipients include, without limitation, those selected from the group consisting of carbohydrates, inorganic salts, antimicrobial agents, antioxidants, surfactants, buffers, acids, bases, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The preparation may also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the preparation as well. Antioxidants are used to prevent oxidation, thereby preventing the deterioration of the conjugate or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant may be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations.

Acids or bases may be present as an excipient in the preparation. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the conjugate in the composition will vary depending on a number of factors, but will optimally be a therapeutically effective dose when the composition is stored in a unit dose container. A therapeutically effective dose can be determined experimentally by repeated administration of increasing amounts of the conjugate in order to determine which amount produces a clinically desired endpoint.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5%-98% by weight, more preferably from about 15-95% by weight of the excipient, with concentrations less than 30% by weight most preferred.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

The pharmaceutical compositions can take any number of forms and the invention is not limited in this regard. Exemplary preparations are most preferably in a form suitable for oral administration such as a tablet, caplet, capsule, gel cap, troche, dispersion, suspension, solution, elixir, syrup, lozenge, transdermal patch, spray, suppository, and powder.

Oral dosage forms are preferred for those conjugates that are orally active, and include tablets, caplets, capsules, gel caps, suspensions, solutions, elixirs, and syrups, and can also comprise a plurality of granules, beads, powders or pellets that are optionally encapsulated. Such dosage forms are prepared using conventional methods known to those in the field of pharmaceutical formulation and described in the pertinent texts.

Tablets and caplets, for example, can be manufactured using standard tablet processing procedures and equipment. Direct compression and granulation techniques are preferred when preparing tablets or caplets containing the conjugates described herein. In addition to the conjugate, the tablets and caplets will generally contain inactive, pharmaceutically acceptable carrier materials such as binders, lubricants, disintegrants, fillers, stabilizers, surfactants, coloring agents, and the like. Binders are used to impart cohesive qualities to a tablet, and thus ensure that the tablet remains intact. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate, calcium stearate, and stearic acid. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard drug decomposition reactions that include, by way of example, oxidative reactions.

Capsules are also preferred oral dosage forms, in which case the conjugate-containing composition can be encapsulated in the form of a liquid or gel (e.g., in the case of a gel cap) or solid (including particulates such as granules, beads, powders or pellets). Suitable capsules include hard and soft capsules, and are generally made of gelatin, starch, or a cellulosic material. Two-piece hard gelatin capsules are preferably sealed, such as with gelatin bands or the like.

Included are parenteral formulations in the substantially dry form (typically as a lyophilizate or precipitate, which can be in the form of a powder or cake), as well as formulations prepared for injection, which are typically liquid and requires the step of reconstituting the dry form of parenteral formulation. Examples of suitable diluents for reconstituting solid compositions prior to injection include bacteriostatic water for injection, dextrose 5% in water, phosphate-buffered saline, Ringer's solution, saline, sterile water, deionized water, and combinations thereof.

In some cases, compositions intended for parenteral administration can take the form of nonaqueous solutions, suspensions, or emulsions, each typically being sterile. Examples of nonaqueous solvents or vehicles are propylene glycol, polyethylene glycol, vegetable oils, such as olive oil and corn oil, gelatin, and injectable organic esters such as ethyl oleate.

The parenteral formulations described herein can also contain adjuvants such as preserving, wetting, emulsifying, and dispersing agents. The formulations are rendered sterile by incorporation of a sterilizing agent, filtration through a bacteria-retaining filter, irradiation, or heat.

The conjugate can also be administered through the skin using conventional transdermal patch or other transdermal delivery system, wherein the conjugate is contained within a laminated structure that serves as a drug delivery device to be affixed to the skin. In such a structure, the conjugate is contained in a layer, or “reservoir,” underlying an upper backing layer. The laminated structure can contain a single reservoir, or it can contain multiple reservoirs.

The conjugate can also be formulated into a suppository for rectal administration. With respect to suppositories, the conjugate is mixed with a suppository base material which is (e.g., an excipient that remains solid at room temperature but softens, melts or dissolves at body temperature) such as coca butter (theobroma oil), polyethylene glycols, glycerinated gelatin, fatty acids, and combinations thereof. Suppositories can be prepared by, for example, performing the following steps (not necessarily in the order presented): melting the suppository base material to form a melt; incorporating the conjugate (either before or after melting of the suppository base material); pouring the melt into a mold; cooling the melt (e.g., placing the melt-containing mold in a room temperature environment) to thereby form suppositories; and removing the suppositories from the mold.

The invention also provides a method for administering a conjugate as provided herein to a patient suffering from a condition that is responsive to treatment with the conjugate. As previously mentioned, the method comprises an administering a potent HIV protease inhibitor. The mode of administration can be oral, but other modes of administration are also contemplated, such as pulmonary, nasal, buccal, rectal, sublingual, transdermal, and parenteral. As used herein, the term “parenteral” includes subcutaneous, intravenous, intra-arterial, intraperitoneal, intracardiac, intrathecal, and intramuscular injection, as well as infusion injections.

In instances where parenteral administration is utilized, it may be necessary to employ somewhat bigger oligomers than those described previously, with molecular weights ranging from about 500 to 30K Daltons (e.g., having molecular weights of about 500, 1000, 2000, 2500, 3000, 5000, 7500, 10000, 15000, 20000, 25000, 30000 or even more).

The method of administering may be used to treat any condition that can be remedied or prevented by administration of the particular conjugate. Those of ordinary skill in the art appreciate which conditions a specific conjugate can effectively treat. The actual dose to be administered will vary depend upon the age, weight, and general condition of the subject as well as the severity of the condition being treated, the judgment of the health care professional, and conjugate being administered. Therapeutically effective amounts are known to those skilled in the art and/or are described in the pertinent reference texts and literature and/or can be determined experimentally. Generally, a therapeutically effective amount is an amount within one or more of the following ranges: from 0.001 mg/day to 10000 mg/day; from 0.01 mg/day to 7500 mg/day; from 0.10 mg/day to 5000 mg/day; from 1 mg/day to 4000 mg/day; and from 10 mg/day to 2000 mg/day.

The unit dosage of any given potent HIV protease inhibitor (again, preferably provided as part of a pharmaceutical preparation) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Once the clinical endpoint has been achieved, dosing of the composition is halted.

All articles, books, patents, patent publications and other publications referenced herein are incorporated by reference in their entireties. In the event of an inconsistency between the teachings of this specification and the art incorporated by reference, the meaning of the teachings in this specification shall prevail.

### EXPERIMENTAL

It is to be understood that while the invention has been described in conjunction with certain preferred and specific embodiments, the foregoing description as well as the examples that follow are intended to illustrate and not limit the scope of the invention. Other aspects, advantages and modifications within the scope of the invention will be apparent to those skilled in the art to which the invention pertains.

All chemical reagents referred to in the appended examples are commercially available unless otherwise indicated. The preparation of PEG-mers is described in, for example, U.S. Patent Application Publication No. 2005/0136031.

The nomenclature used in the Experimental that follows corresponds to the following chemical structures.

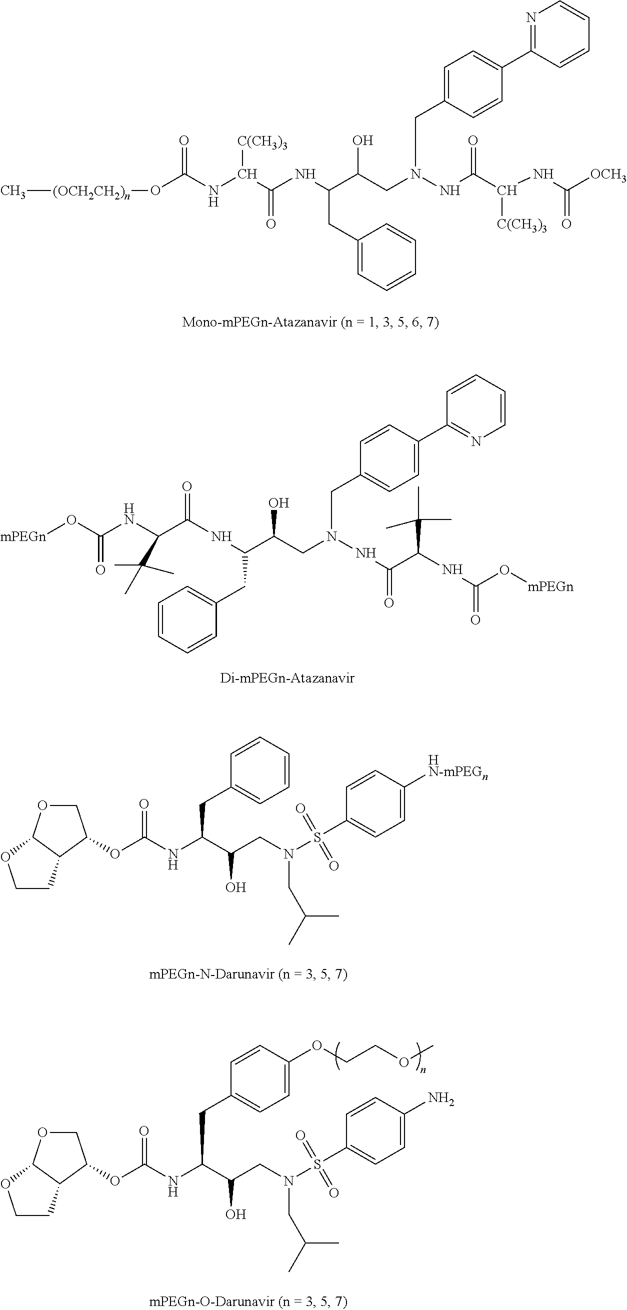

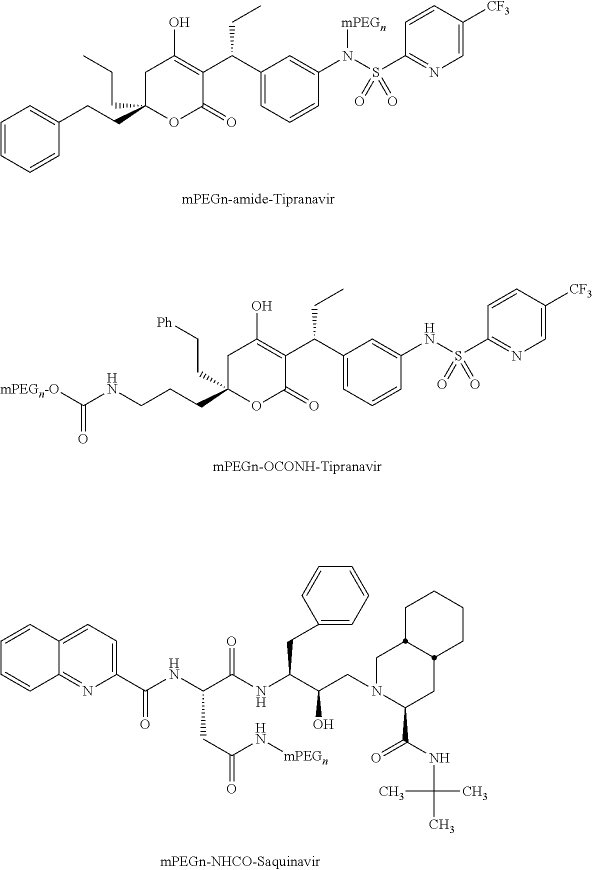

The synthesis of these structures is provided in PCT preparation of these structures is provided herein and/or in PCT/US2008/003354 (WO2008/112289).

With respect to mono-mPEG3-Atazanavir, for example, the following synthesis was followed.

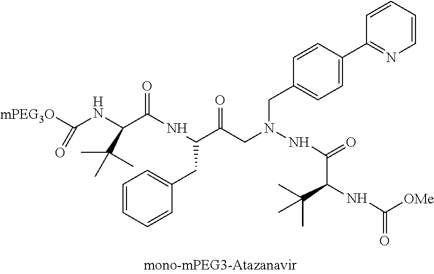

L-tert-Leucine, methyl chloroformate, tert-butyl carbazate, N-(3-Dimethylaminopropyl)-N′-ethylcarbodiimide (EDC), 1-hydroxybenzotriazole (HOBT), 4-methylmorpholine (NMM), ethyl acetate, 4 M HCl in dioxane, 37% HCl (aqueous), Z-L-Phe chloromethyl ketone, NaHCO₃, Sodium Iodide, acetonitrile, isopropyl alcohol, triethylamine, sodium cyanoborohydride, p-toluenesulfonic acid, tetrahydrofuran, DSC, DCM, 10% Pd—C, methanol, ethanol, TPTU, DIPEA, LTBA, diethyl ether.

Methods: All reactions with air- or moisture-sensitive reactants and solvents were carried out under nitrogen atmosphere. In general, reagents and solvents were used as purchased without further purification. Analytical thin-layer chromatography was performed on silica F₂₅₄ glass plates (Biotage). Components were visualized by UV light of 254 nm or by spraying with phosphomolybdic acid, or ninhydrin. Flash chromatography was performed on a Biotage SP4 system. ¹H NMR spectra: Bruker 300 MHz; chemical shifts of signals are expressed in parts per million (ppm) and are referenced to the deuterated solvents used. MS spectra: rapid resolution Zorbax C18 column; 4.6×50 mm; 1.8 μm. HPLC method had the following parameters: column, Betasil C18, 5-μm (100×2.1 mm); flow, 0.5 mL/min; gradient, 0-23 min, 20% acetonitrile/0.1% TFA in water/0.1% TFA to 100% acetonitrile/0.1% TFA; detection, 230 nm. t_{R} refers to the retention time.

Abbreviations: TPTU, O-(1,2-Dihydro-2-oxo-1-pyridyl)-N,N,N′,N′-tetramethyluroniumtetrafluoroborate; DIPEA, N,N′-Diisopropylethylamine; DSC, N,N′-Disuccinimidyl carbonate.

The general synthetic scheme for preparing an aryl hydrazine (9) used in the preparation of mono-mPEG3-Atazanavir is provided below.

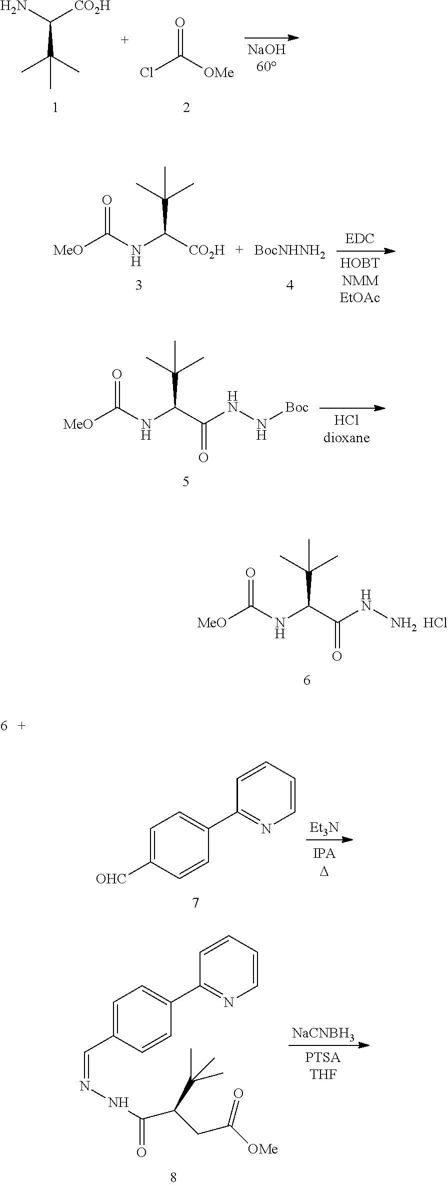

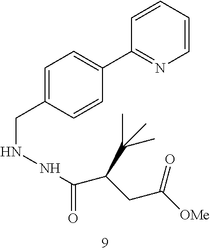

### 2-Methoxycarbonylamino-3,3-dimethyl-butyric acid (3)

Into a 250 mL flask was placed L-tert-Leucine (5.0 gm, 38 mmol), 2N NaOH (66 mL), and methyl chloroformate (5.86 mL, 76 mmol, 2.0 equivalents). The reaction mixture was heated to 60° C., turning light-yellow. After approximately 20 hours, the heat was removed and the mixture cooled to room temperature, and then to 0° C. The reaction mixture was quenched at 0° C. with 2 N HCl (40 mL) to pH 1. The acidified mixture was transferred to a separatory funnel and extracted with ethyl acetate (3×50 mL). The combined organic layers were washed with water (2×150 mL), and saturated NaCl (150 mL), and then dried over Na₂SO₄. The organic layer was filtered and concentrated under reduced pressure to give a clear oil. The oil was azeotroped with toluene (3×50 mL), and then dried under high vacuum to give 6.4 gm (89%) of 3 as a white solid. ¹H NMR (DMSO) δ 12.51 (bs, 1H), 7.28 (d, 1H), 3.80 (d, 1H), 3.53 (s, 3H), 0.93 (s, 9H); MS (M)⁺=190; HPLC t_{R} 2.8 minutes.

### N′-(2-Methoxycarbonylamino-3,3-dimethyl-butyryl)-hydrazinecarboxylic acid tert-butyl ester (5)

Methoxycarbonyl-L-tert-Leucine (3) (1.37 gm, 7.24 mmol) was dissolved in anhydrous ethyl acetate (21 mL). To the clear solution was added N-(3-dimethylaminopropyl)-N′-ethylcarbodiimide (EDC) (1.12 gm, 5.82 mmol, 1.1 equivalents). The suspension was stirred under nitrogen at room temperature. After ten minutes added HOBT (1.08 gm, 7.97 mmol, 1.1 equivalents), followed by 4-methyl-morpholine (1.35 mL, 12.32 mmol, 1.7 equivalents). After another 30 minutes, added t-butyl carbazate (1.05 gm, 7.97 mmol, 1.1 equivalents) and the light-yellow suspension continued stirring at room temperature. After 20 hours, the reaction mixture was diluted with ethyl acetate (50 mL) and transferred to a separatory funnel. The aqueous layer was partitioned with saturated NaHCO₃. The aqueous layer was extracted with ethyl acetate (3×50 mL). The combined organic layers were washed with saturated NaCl and dried over Na₂SO₄. After filtering and concentrating under reduced pressure, the residue was purified by Biotage chromatography (0 to 3% methanol/dichloromethane gradient) to give 2.05 gm (94%) of 5 as a white foam solid. ¹H NMR (MeOD) δ 3.91 (bs, 1H), 3.62 (s, 3H), 1.42 (s, 9H), 0.98 (s, 9H); MS (M)⁺=304; HPLC t_{R} 5.5 minutes.

### (1-Hydrazinocarbonyl-2,2-dimethyl-propyl)-carbamic acid methyl ester HCl (6)

Intermediate (5) (12.7 gm, 41 mmol) was dissolved in 1,4-dioxane (100 mL), followed by slow addition of 4.0 M HCl in dioxane (25 mL). The light-yellow mixture was stirred under nitrogen at room temperature. After 18 hours the cloudy mixture was concentrated under reduced pressure. The residue was azeotroped with toluene (3×30 mL), and then dried under high vacuum to give 10.9 gm of a white solid (quantitative). ¹H NMR (DMSO) δ 11.23 (s, 1H), 7.41 (d, 1H), 7.22 (m, 1H), 3.98 (d, 1H), 0.92 (s, 9H); MS (M)⁺=204; HPLC t_{R} 0.67 minutes.

### [2,2-Dimethyl-1-(4-pyridin-2-yl-benzylidene-hydrazinocarbonyl)-propyl]-carbamic acid methyl ester (8)

Methoxycarbonyl-L-tert-Leucine hydrazine (6) (1.35 gm, 6.65 mmol) was taken up in i-PrOH (60 mL) and then added pyridyl benzaldehyde (7) (1.22 gm, 6.65 mmol). The yellow reaction mixture was heated to reflux (85° C.). After approximately two hours, TLC and HPLC showed the reaction was complete. The heat was removed and the thick yellow mixture was cooled to 0° C. The solvent was removed under reduced pressure. The yellow residue was taken up in DCM (250 mL) and partitioned with water. The aqueous layer was extracted with DCM (4×50 mL). The combined organics were washed with saturated NaHCO₃, water, 0.05 M HCl, and saturated NaCl (approximately 300 mL each). The organic layers were dried (Na₂SO₄), filtered and concentrated under reduced pressure to give a yellow solid. Purification by Biotage® chromatography (0 to 3% methanol/DCM gradient) gave 1.34 gm (55%) of 8 as a white solid. By NMR it is approximately a 1:1 mixture of cis-trans isomers. ¹H NMR (CDCl₃) δ 10.30 (s, 1H), 9.80 (s, 1H), 8.64 (d, 1H), 8.60 (d, 1H), 8.11 (s, 1H), 8.04 (d, 2H), 7.98 (m, 2H), 7.85 (m, 6H), 7.83 (m, 2H), 7.22 (m, 2H), 5.92 (d, 1H), 5.56 (d, 1H), 5.32 (d, 1H), 4.10 (d, 1H), 3.64 (d, 6H), 1.02 (d, 18H); MS (M)⁺=369; HPLC t_{R} 2.9 minutes.

### {2,2-Dimethyl-1-[N′-(4-pyridin-2-yl-benzyl)-hydrazinocarbonyl]-propyl}-carbamic acid methyl ester (9)

Hydrazone (8) (1.10 gm, 2.98 mmol) was dissolved in anhydrous THF (30 mL). Then added solid NaCNBH₃ (0.40 gm, 5.97 mmol, 2.0 equivalents) all at once, followed by slow addition via syringe of PTSA (p-toluene sulfonic acid) (1.13 gm, 5.97 mmol, 2.0 equivalents) in THF (15 mL). There was bubbling observed during the PTSA addition. The cloudy mixture was heated to reflux (70° C.). After approximately 40 h, the cloudy reaction mixture was concentrated under reduced pressure and the white residue partitioned with DCM (30 mL) and water (50 mL). The aqueous layer was extracted with DCM (3×40 mL). The combined organic layers were washed with water, and saturated NaCl. The organic layers were dried (Na₂SO₄), filtered and concentrated under reduced pressure to give a white foam solid. Purification by Biotage® chromatography (0 to 3% methanol/DCM gradient) gave 0.34 gm (67%) of 9 as a white solid. ¹H NMR (MeOD) δ 8.55 (s, 1H), 7.85 (m, 4H), 7.50 (d, 2H), 7.32 (m, 1H), 4.05 (s, 2H), 3.62 (s, 3H), 0.91 (s, 9H); MS (M)⁺=371; HPLC t_{R} 1.8 minutes.

The general synthetic scheme for preparing Cbz-Azaketo Isotere (11) used in the preparation of mono-mPEG3-Atazanavir is provided below.

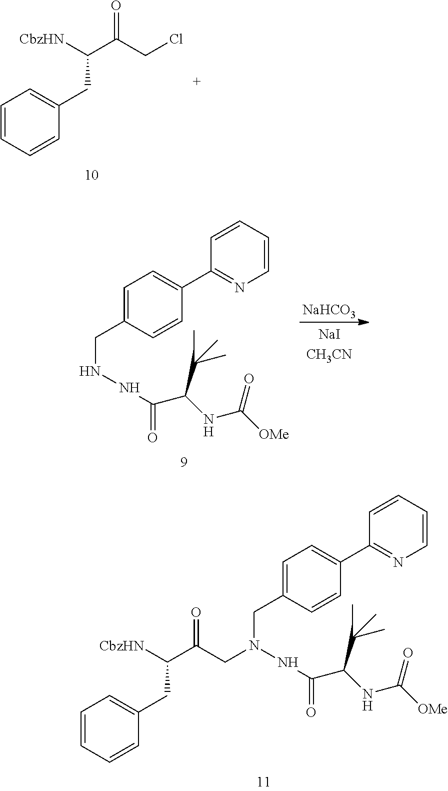

### {1-Benzyl-3-[N′(2-methoxycarbonylamino-3,3-dimethyl-butyryl)-N-(4-pyridin-2-yl-benzyl)-hydrazino]-2-oxo-propyl}-carbamic acid benzyl ester (11)

Z-L-Phe chloromethyl ketone (0.67 gm, 2.01 mmol) was taken up in anhydrous acetonitrile (30 mL). Then added NaI (0.33 gm, 2.21 mmol, 1.1 equivalents), followed by NaHCO₃ (0.33 gm, 4.02 mmol, 2.0 equivalents). The solution was stirred for ten minutes at room temperature. Hydrazine (9) (0.82 gm, 2.21 mmol, 1.1 equivalents) in acetonitrile (20 mL) was then added via syringe. The cloudy yellow reaction mixture was heated to 60° C. After approximately 18 hours, the cloudy yellow mixture was cooled to room temperature. The solvent was removed under reduced pressure. The yellow residue was partitioned with DCM (30 mL) and water (90 mL). The aqueous layer was extracted with DCM (3×30 mL). The combined organic layers were washed with water, and saturated NaCl. The organic layers were dried (Na₂SO₄), filtered and concentrated under reduced pressure to give a white foam solid. Purification by Biotage® chromatography (0 to 3% methanol/DCM gradient; 15 CV) gave 1.14 gm (85%) of 11 as a white solid. TLC R_{f} (5% methanol/dichloromethane)=0.26; ¹H NMR (CDCl₃) δ 8.69 (d, 1H), 7.92 (d, 2H), 7.72 (m, 2H), 7.44 (d, 2H), 7.26 (m, 10H), 7.11 (d, 2H), 4.70 (d, 1H), 4.12 (dd, 2H), 3.75 (dd, 2H), 3.62 (m, 1H), 3.57 (s, 3H), 2.99 (m, 1H), 2.87 (m, 1H), 1.45 (s, 1H), 1.03 (m, 2H), 0.81 (s, 9H). (s, 2H), 3.62 (s, 3H); MS (M)⁺=666; HPLC t_{R} 9.5 minutes.

The general synthetic scheme for preparing Cbz-Aza Isotere (12) used in the preparation of mono-mPEG3-Atazanavir is provided below.

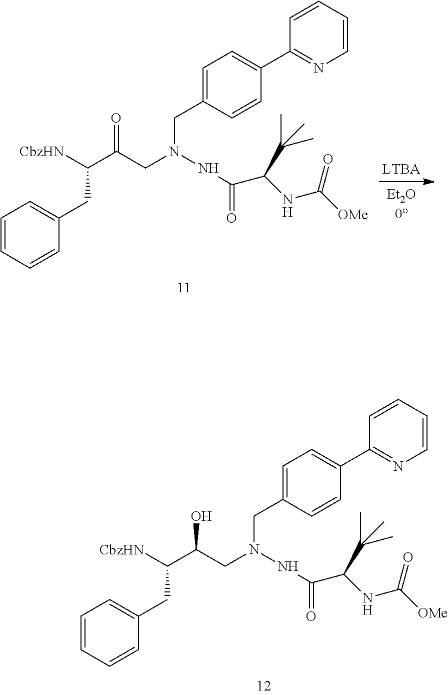

### {1-Benzyl-2-hydroxy-3-[N′-(2-methoxycarbonylamino-3,3-dimethyl-butyryl)-N-(4-pyridin-2-yl-benzyl)-hydrazino]-propyl}-carbamic acid benzyl ester (12)

The Cbz-azaketone (11) (0.84 gm, 1.26 mmol) was taken up in diethyl ether (15 mL) and cooled to 0° C. To the white suspension was added LTBA (Lithium tri-tert-butoxy-aluminum hydride) (0.80 gm, 3.15 mmol, 2.5 equivalents), at 0° C. The light-yellow suspension was stirred under nitrogen at 0° C. After one hour at 0° C., the cloudy yellow mixture was stored overnight at −20° C. The reaction mixture was quenched with water (0.9 mL), at 0° C. The solvent was removed under reduced pressure. The residue was partitioned with DCM (30 mL) and water (90 mL). The aqueous layer was extracted with DCM (3×30 mL). The combined organic layers were washed with water, and saturated NaCl. The organic layers were dried (Na₂SO₄), filtered and concentrated under reduced pressure to give a white foam solid. Purification by Biotage® chromatography (0 to 3% methanol/diethyl ether gradient; 20 CV) gave 0.45 gm (54%) of 12 as a white solid. TLC R_{f} (5% methanol/diethyl ether)=0.50; ¹H NMR (CDCl₃) δ 8.64 (d, 1H), 7.88 (d, 2H), 7.74 (m, 1H), 7.65 (m, 1H), 7.35 (m, 2H), 7.20 (m, 14H), 5.20 (dd, 2H), 4.95 (m, 2H), 4.62 (d, 1H), 4.02 (d, 1H), 3.82 (d, 1H), 3.70 (m, 1H), 3.55 (m, 2H), 3.45 (m, 2H), 3.42 (s, 1H), 2.86 (m, 2H), 2.78 (m, 1H), 2.54 (d, 1H), 1.35 (s, 1H), 1.15 (m, 1H), 0.75 (m, 2H), 0.64 (s, 6H); MS (M)⁺=668; HPLC t_{R} 9.2 minutes.

The general synthetic scheme for preparing Amino-azaisotere (13) used in the preparation of mono-mPEG3-Atazanavir is provided below.

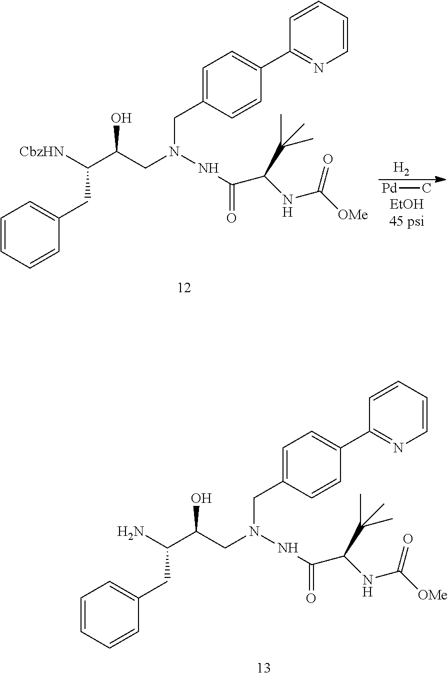

### {1-[N′-(3-Amino-2-hydroxy-4-phenyl-butyl)-N′-(4-pyridin-2-yl-benzyl)-hydrazinocarbonyl]-2,2-dimethyl-propyl}-carbamic acid methyl ester (13)

The Cbz-aza-isostere (12) (0.34 gm, 0.50 mmol) was taken up in absolute ethanol (150 mL) and charged with 10% Pd—C (0.10 gm). The mixture was subjected to hydrogenolysis, at 45 psi. After 18 hours the catalyst was filtered through celite. The cake was washed with ethanol (35 mL), and the filtrate was concentrated under reduced pressure to give 0.19 gm (70%) of 13 as a clear oil. TLC R_{f} (5% methanol/dichloro-methane)=0.02; MS (M)⁺=534; HPLC t_{R} 3.9 min. The material was used in the next step without further purification.

The general synthetic scheme for preparing PEG-tert-Leucine reagent (16) and mono-mPEG₃-Atazanavir Conjugate (17) used in the preparation of mono-mPEG3-Atazanavir is provided below.

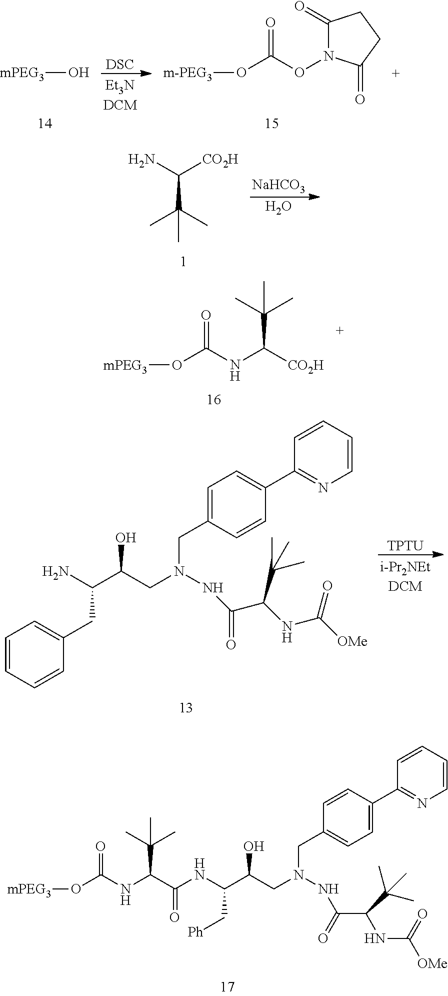

### m-PEG-₃-SC-carbonate (15)

Into a 100 mL flask was placed mPEG₃-OH (14) (2.0 g, 12.1 mmol) and anhydrous dichloromethane (25 mL). The clear solution was cooled to 0° C., and then triethylamine (1.86 mL, 13.4 mmol, 1.1 equivalents) was added slowly. The solution was stirred for 15 minutes at 0° C., and then was added to a second flask containing a suspension of DSC (3.1 g, 12.1 mmol) in dichloromethane (20 mL). The reaction mixture was allowed to equilibrate to room temperature. After approximately 18 hours, the light-yellow reaction mixture was diluted with dichloromethane (60 mL), transferred to a separatory funnel, and partitioned with deionized water (100 mL). The aqueous layer was extracted with dichloromethane (4×80 mL). The combined organics were washed with water, saturated sodium bicarbonate, and saturated sodium chloride. The dried organic layer was filtered, concentrated under reduced pressure and dried overnight under high vacuum, to give 2.79 g (75%) of mPEG₃-SC-carbonate as a light yellow oil. ¹H NMR (CDCl₃) δ 4.40 (m, 2H), 3.80 (m, 2H), 3.70 (bs, 6H), 3.60 (m, 2H), 3.35 (s, 3H), 2.80 (s, 4H); LC/MS=306 (M+1).

### mPEG-₃-L-tert-Leucine (16)

Into a 125 mL flask was placed L-tert-Leucine (1) (0.43 g, 3.27 mmol) and deionized water (12 mL). The solution was stirred for 30 min until clear, followed by the addition of solid sodium bicarbonate (1.27 g, 15.0 mmol, 4.6 equivalents). The cloudy solution was stirred at room temperature, under nitrogen. In a second flask the mPEG₃-SC-carbonate (15) (1.24 g, 4.09 mmol, 1.25 equiv.) was taken up in deionized water (12 mL) and this solution was added all at once to the basic L-tert-Leucine solution. The cloudy light-yellow reaction mixture was stirred at room temperature, under nitrogen. After approximately 20 h, the clear mixture was cooled to 0° C., and carefully acidified with 2 N HCl to pH 1 (20 mL). The acidic mixture was transferred to a separatory funnel and partitioned with dichloromethane (50 mL) and additional water (50 mL). The aqueous layer was extracted with dichloromethane (4×50 mL). The combined organic layers were washed with water and saturated sodium chloride, and dried over sodium sulfate. The dried organic layer was filtered, concentrated under reduced pressure and dried under high vacuum overnight, to give 0.83 g (79%) of mPEG₃-L-tert-Leucine (16) as a pale yellow oil. ¹H NMR (CDCl₃) δ 5.45 (d, 1H), 4.26-4.35 (m, 2H), 4.14 (m, 1H), 3.70 (bs, 17H), 3.65 (m, 2H), 3.32 (s, 3H), 0.96 (s, 9H); LC/MS=322 (M+1).

### {1-[N′-[2-Hydroxy-3-(2-{2-[2-(2-methoxy-ethoxy)-ethoxy]-ethoxycarbonylamino}-3,3-dimethyl-butyrylamino)-4-phenyl-butyl]-N′(4-pyridin-2-yl-benzyl)-hydrazinocarbonyl]-2,2-dimethyl-propyl}-carbamic acid methyl ester (17)

The mPEG₃-tert-Leucine reagent (16) (0.34 gm, 1.06 mmol, 3.0 equivalents) was taken up in anhydrous dichloromethane (3.0 mL) and cooled to 0° C. TPTU (0.31 gm, 1.06 mmol, 3.0 equivalents) was added all at once, and the solution was stirred under nitrogen at 0° C. In a separate flask, the amino aza-isostere (13) (0.19 gm, 0.35 mmol) was taken up in anhydrous dichloromethane (3.0 mL) and diisopropylethylamine (0.37 mL, 2.13 mmol, 6.0 equivalents). This solution was added via syringe to the m-PEG₃-tert-Leucine solution, at 0° C. The ice bath was removed and the reaction mixture was allowed to equilibrate to room temperature. After approximately 18 hours at room temperature, the reaction mixture was diluted with dichloromethane (10 mL) and transferred to a separatory funnel, where it was partitioned with water (30 mL). The aqueous layer was extracted with dichloromethane (3×10 mL). The combined organics were washed with water, and saturated NaCl (20 mL each). The organic layer was dried (Na₂SO₄), filtered, and concentrated under reduced pressure, to give a clear oil. Purification by Biotage® chromatography (0 to 3% methanol/dichloromethane gradient; 20 CV) gave 0.21 gm (72%) of 17 as a white solid. TLC R_{f} (8% methanol/dichloromethane)=0.32; ¹H NMR (CDCl₃) δ 8.59 (d, 1H), 7.83 (d, 2H), 7.64 (m, 2H), 7.36 (m, 3H), 7.13 (m, 7H), 6.64 (m, 1H), 5.45 (d, 2H), 4.95 (m, 1H), 4.11 (m, 2H), 3.98 (m, 2H), 3.90 (m, 1H), 3.74 (m, 1H), 3.58 (m, 10H), 3.48 (m, 2H), 3.29 (s, 3H), 2.88 (m, 2H), 2.57 (m, 3H), 0.75 (s, 9H); MS (M)⁺=668; HPLC t_{R} 7.7 min.

The synthesis of the bis-aryl hydrazine (9) is described above and represents an approach for preparing an intermediate useful for the preparing the atazanavir “core.” The synthesis began with reaction of the chiral amino acid, L-tert-Leucine (1), with methyl chloroformate (2), to give methoxycarbonyl-L-tert-Leucine (3). In addition to serving as an amino protecting group, the methoxycarbonyl-L-tert-Leucine moiety also establishes the correct stereochemistry of the t-butyl group. Reaction of (3) with tert-butylcarbazate gave the methoxycarbonyl-L-tert-Leucine-Boc protected hydrazine (5). Deprotection of the Boc group proceeded in quantitative yield to give the hydrazine hydrochloride salt (6). Reaction of the hydrazine salt (6) with the bis-aryl aldehyde (7) under reflux conditions gave bis-aryl hydrazone (8). Chemical reduction of the hydrazone (8), using sodium cyanoborohydride, provided the key building block bis-aryl hydrazine (9). With (9) in hand, an SN2 reaction with another intermediate, Cbz-chloromethyl ketone (10), gave Cbz-azaketone (11). Introduction of the required (S)-hydroxyl group was done via a diastereoselective reduction of (11) using the bulky reducing agent LTBA (lithium tri-tert-butoxyaluminum hydride) to give Cbz-aza-isostere (12). Removal of the Cbz protecting group was done under hydrogenolysis conditions (H₂, 10% Pd—C, 45 psi) to give the amino aza-isostere (13). The other intermediate for the mono-PEG₃-Atazanavir conjugate was the specialized PEG reagent containing the required stereochemistry for the Atazanavir conjugate. The synthesis began with m-PEG₃-OH (14) reacted with N,N′-disuccinimidyl carbonate (DSC), to give m-PEG₃-DSC (15). Reaction with L-tert-Leucine gave the desired m-PEG3-L-tert-Leucine reagent (16). Finally, under coupling conditions, the amino-aza-isostere (13) was reacted with m-PEG₃-L-tert-Leucine (16) to provide the mono-mPEG3-Atazanavir conjugate.

Using an approach similar to the one used to prepare mono-mPEG3-Atazanavir, Mono-mPEGn-Atazanavir conjugates of different PEG sizes (e.g., n=1, 5, 6 and 7) were prepared.

With respect to mPEGn-OCONH-Tripranavir conjugates, for example, the following synthesis was followed.

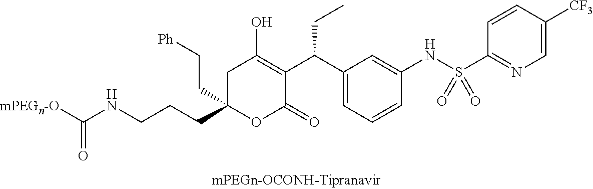

N-Carbethoxyphthalimide, 4-Aminobutyraldehyde diethyl acetal, Cupper(I) bromide-dimethylsulfide complex (CuBr.DMS), Phenethylmagnesium chloride (1.0 M in THF), Boron trifluoride diethyl etherate (BF₃.Et₂O), Dimethylsulfoxide (DMSO), Trifluoroacetic acid, Dicyclohexylcarbodiimide (DCC), Butyllithium (1.6 M), Pyridinium chlorochromate (PCC), Magnesium bromide diethyl etherate (MgBr₂.OEt₂), Potassium bis(trimethylsilyl)-amide (KHMDS, 0.5 M), Acetyl chloride, Titanium (IV) isopropoxide, Titanium chloride (TiCl₄), Potassium tert-butoxide (KOBu^{t}), 4-(Dimethylamine)-pyridine (DMAP), Hydrozine (NH₂NH₂), 2-Methoxyethanol, Tri(ethyleneglycol)monomethylether (95%), 4-Nitrophenyl chloroformate, Sodium hydroxide, N,N-diisopropylethylamine (DIPEA), Palladium 10% wt % on activated carbon, Methylamine, Triethylamine, Anhydrous methanol, and Pyridine were purchased from Sigma-Aldrich (St Louis, Mo.). mPEG₅-OH were obtained from India Sai CRO. 5-Trifluoromethyl-2-pyridinesulfonyl chloride was purchased from Toronto Research Chemicals, Inc. (North York, ON, Canada). DCM was distilled from Cal-h. Tetrahydrofuran (THF), ether, Ethyl acetate, and other organic solvents were used as they purchased.

A general synthetic scheme for preparing a ketone intermediate useful for preparing mPEGn-OCONH-Tripranavir conjugates is provided below.

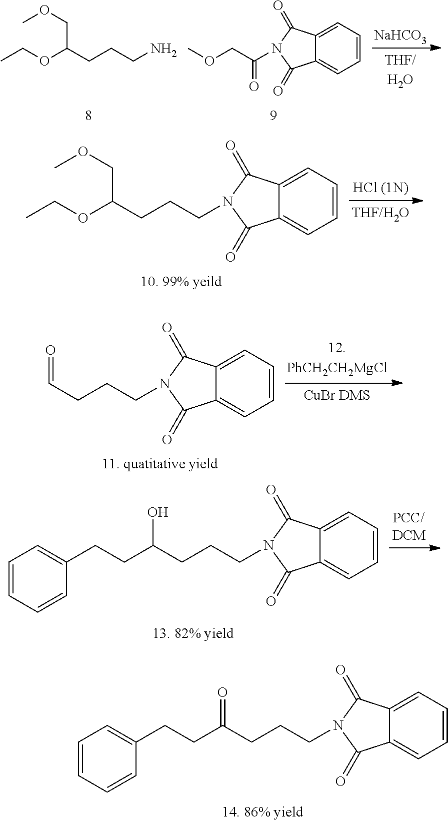

Phthalimide Protection:

Deionized water (50 mL) and THF (50 mL) were added in a 250-mL flask. NaHCO₃ (5.67 g, 67.5 mmol) and 4-aminobutyraldehyde diethyl acetal (8) (12.0 mL, 67.5 mmol) were suspended in this solution. N-carbethoxyphthalimide (9) (15.54 g, 70.9 mmol) was then added and the solution began become clear within 15 minutes. The reaction was kept at ambient temperature for two hours and was diluted with EtOAc (500 mL) and water (20 mL) to dissolve the salt precipitation. The organic phase was separated and washed with brine (100 mL×2). It was then dried over Na₂SO₄, filtered, and concentrated to a colorless crude product. The residue was loaded on Biotage column (40 M×2, 5-27% EtOAc in Hex in 16 CV) and a colorless product (10) was collected (95-100% yield). The product solidified after overnight high vacuo drying. R_{f}=0.43 (Hex:EtOAc=3:1), LC-MS (ESI, MH⁺) 292.1. ¹H NMR (300 MHz, CDCl₃) δ 1.19 (6H, t, J=7.2 Hz), 1.65-1.80 (4H, m), 3.46-3.54 (2H, m), 3.58-3.74 (4H, m), 4.51 (1H, t, J=5.7 Hz), 7.70-7.74 (2H, m), 7.83-7.86 (2H, m).

Acidic Acetal Hydrolysis:

In a 250-mL flask, the above phthalimide was dissolved in THF (56 mL) at ambient temperature. Hydrochloric acid (1N, 18 mL) was added and the hydrolysis was monitored by TLC. It was completed in 5-6 hrs and stopped by carefully addition of NaHCO₃ saturated solution. The solution was diluted with EtOAc (300 mL) and the organic phase was washed with brine and dried over Na₂SO₄. After filtration, it was concentrated and the product was solidified during overnight high vacuo drying. R_{f}=0.22 (Hex:EtOAc=3:1), RP-HPLC (betasil C18, 0.5 mL/min, 30-100% ACN in 10 min) 4.65 min, LC-MS (ESI, MH⁺) 218.1. ¹H NMR (300 MHz, CDCl₃) δ 2.02 (2H, p, J=6.9 Hz), 2.54 (2H, dt, J=0.9, 7.2 Hz), 3.75 (2H, t, J=6.6 Hz), 7.71-7.74 (2H, m), 7.84-7.87 (2H, m), 9.78 (1H, t, J=0.9 Hz).

Grignard-Cupper Alkylation:

In a 500-mL flask, Cupper(I) bromide DMS (7.2 g, 35.1 mmol) was dissolved in THF (43 mL) and the solution was cooled to −35° C. Phenylethyl magnesium chloride (1M, 35.1 mL, 35.1 mmol) was added dropwise in ten minutes. The Mg-cupper reagent was kept at −30 to −10° C. over 20 minutes before it was cooled down to −78° C. and above aldehdye (2.54 g, 11.7 mmol) in THF (20 mL) was added dropwise during 15 minutes. BF₃.Et₂O (5.88 mL, 46.8 mL) was also added dropwise during six minutes. The reaction temperature was kept below −65° C. over 30 minutes and then warm up to 6° C. over 2.5 hours. It was stopped by adding NH₄OH to pH=9. The solution was then diluted with NH₄Cl (100 mL) and Et₂O (250 mL). The separated ether phase was washed with NaHCO₃ and brine and dried over Na₂SO₄. After filtration, it was concentrated on vacuo and sample residue was purified on Biotage (40 M, 20-44% EtOAc in Hex) in 16 CV. A slight yellow color product (13) was solidified (3.30 g, 87% yield) after overnight vacuo. R_{f}=0.16 (Hex:EtOAc=1:1), RP-HPLC (betasil C18, 0.5 mL/min, 30-100% ACN in 10 min) 6.06 min, LC-MS (ESI, MH⁺) 324.1. ¹H NMR (300 MHz, CDCl₃) δ 1.46-1.54 (2H, m), 1.72-1.88 (4H, m), 2.61-2.84 (2H, m), 3.63-3.69 (1H, m), 3.73 (2H, t, J=7.2 Hz), 7.15-7.29 (5H, m), 7.68-7.73 (2H, m), 7.81-7.87 (2H, m).

Moffatt Oxidation:

The above secondary alcohol (13) (3.07 g, 9.50 mmol) was dissolved in DCM (125 mL) at ambient temperature. DMSO (6.75, 95 mmol), pyridine (1.54 mL, 19.0 mL), TFA (1.55 mL, 20.9 mL) were added in the order. Finally DCC (7.84 g, 38.0 mmol) was added and the reaction was kept at ambient temperature for overnight. The reaction was diluted with NH₄Cl and extracted with DCM (20 mL×2). The combine organic phase was washed with brine and dried over Na₂SO₄. The DCU together with drying agent was filterated and the residue was dried over with Silica gel (20 g). The silical gel was loaded on Biotage column and purified (6-40% EtOAc/Hex over 16 CV). A colorless solidified product (14) was collected (1.50 g, 75% yield) after overnight drying. R_{f}=0.33 (Hex:EtOAc=3:1), RP-HPLC (betasil C18, 0.5 mL/min, 30-100% ACN in 10 min) 7.52 minutes, LC-MS (ESI, MH⁺) 322.1. ¹H NMR (300 MHz, CDCl₃) δ 1.96 (2H, p, J=6.9 Hz), 2.45 (2H, t, J=7.2 Hz), 2.73 (2H, t, J=6.9 Hz), 2.88 (2H, t, J=7.2 Hz), 3.70 (2H, t, J=6.6 Hz), 7.15-7.28 (5H, m), 7.70-7.73 (2H, m), 7.83-7.86 (2H, m).

PCC Oxidation:

In a 500-mL flask, the above secondary alcohol (13) (12.86 g, 39.8 mmol) was dissolved in DCM. PCC (8.6 g, 39.8 mmol) was added and the reaction was kept at room temperature. In three hours, the additional PCC (about 8%) was added based on the TLC of remaining of starting material. The reaction was kept over 40 hours and the product mixture was filtrated though celite/silica gel layer and washed with DCM. The combined DCM solution was concentrated and the product residue was loaded on a Biotage column (40M×2, 6-40% EtOAc/Hex in 16 CV). A colorless solid product (14) (10.72 g, 86%) was collected after high vacuo drying.

An approach for conducting basic C—C conjugation and ti-catalyzed C—C conjugation useful in preparing mPEGn-OCONH-Tripranavir conjugates is provided below.

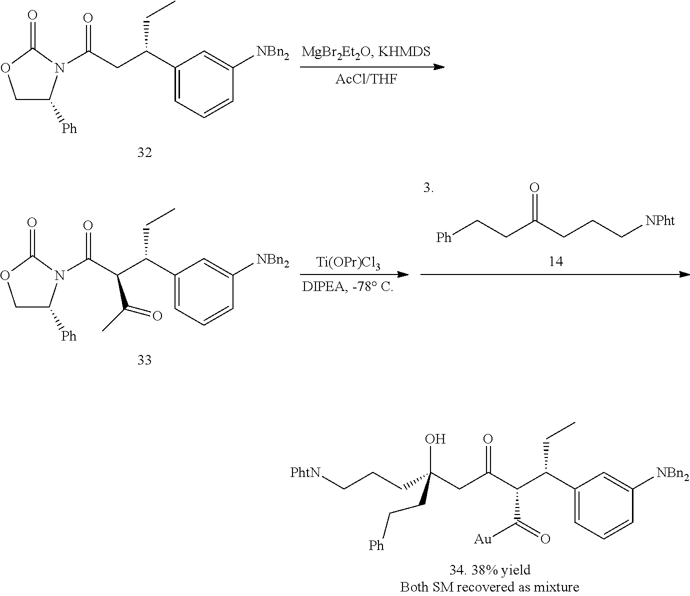

Base-Catalyzed Acetylation:

In a 250-mL flask, the substrate (32) (7.28 g, 14.05 mmol) and MgBr₂.OEt₂ (4.0 g, 15.5 mmol) were added. The flask was protected in dry N₂ and THF (68 mL) was added. The solution was cooled down to −78° C. in acetone/dry ice bath before KHMDS (0.5 M, 42.1 mL, 21.08 mmol) was dropwise added in ten minutes. The above mixture was kept at −78° C. for 30 minutes before acetyl chloride (1.50 mL, 21.08 mmol) was added in five minutes. The reaction mixture was warm up gradually during the overnight reaction. It was quenched by NH₄Cl (200 mL) and extracted with EtOAc (100 mL+50 mL×2). The combined organic phase was washed with brine and dry over Na₂SO₄. After filtration, it was concentrated and purified on the Biotage (40M×2, 6-16% EtOAc/Hex in 16 CV). The collected product gives a colorless foam product 33 (6.66 g, 85% yield) after vacuo drying. R_{f}=0.31 (Hex:EtOAc=3:1), RP-HPLC (betasil C18, 0.5 mL/min, 60-100% ACN in 10 minutes) 6.58 minutes; LC-MS (ESI, MH⁺) 561.3. ¹H NMR (300 MHz, CDCl₃) δ 0.61 (3H, t, J=7.2 Hz), 1.63 (3H, s), 3.07 (1H, dt, J=3.3, 10.8 Hz), 4.22 (1H, dd, J=3.9, 8.7 Hz), 4.61 (4H, s), 4.67 (1H, t, J=9.0 Hz), 4.98 (1H, d, J=10.5 Hz), 5.42 (1H, dd, J=3.6, 8.7 Hz), 6.54-6.64 (3H, m), 7.09 (1H, t, J=8.1 Hz), 7.21-7.39 (15H, m).

Ti-Catalyzed C—C Conjugation:

In a N₂-protected 250-mL flask, distilled DCM (50 mL) was added following by addition of Ti(OPr)₄ (982 μL, 3.35 mmol) and TiCl₄ (1.03 mL, 9.41 mmol) in order. The mixture was cooled down to −78° C. in acetone/dry-ice bath and a mixture of substrate (33) (5.86 g, 10.5 mmol) in DCM (16 mL) was dropwise added in ten minutes. The reaction was kept at this temperature for 5 min before DIPEA (2.37 mL, 13.6 mmol) was added slowly in 5 min. The reaction was warm up to 0° C. and kept in 30 minutes. It was recooled down to −78° C. and a mixture of ketone (14) (from PCC oxidation, 3.36 g, 10.5 mmol) in DCM (10 mL) was added. The reaction mixture was warm up to 0° C. and kept in ice-water bath for another two hours. It was quenched by NH₄Cl (200 mL) and diluted with DCM (100 mL). The aqueous phase was extracted with DCM (50 mL×2) and the combined organic phase was washed with brine (150 mL). The orgnic phase was then dried over Na₂SO₄ and concentrated under the vacuo. The crude product mixture was purified on Biotage (40S×2, 12-42% EtOAc in Hex over 16 CV). The product (34) was solidified (3.48 g, 38% yield) after high vacuo. The starting material mixture also has been recovery (5.43 g, 47%). Since this product is a diasteromer mixture, the ¹H NMR cannot be read and recorded. R_{f}=0.13 (Hex:EtOAc=3:1), RP-HPLC (betasil C18, 0.5 mL/min, 60-100% ACN in 10 minutes) 923 minutes; LC-MS (ESI, MH⁺) 882.5.

An approach for the final steps for preparing mPEGn-OCONH-Tripranavir conjugates is provided below.

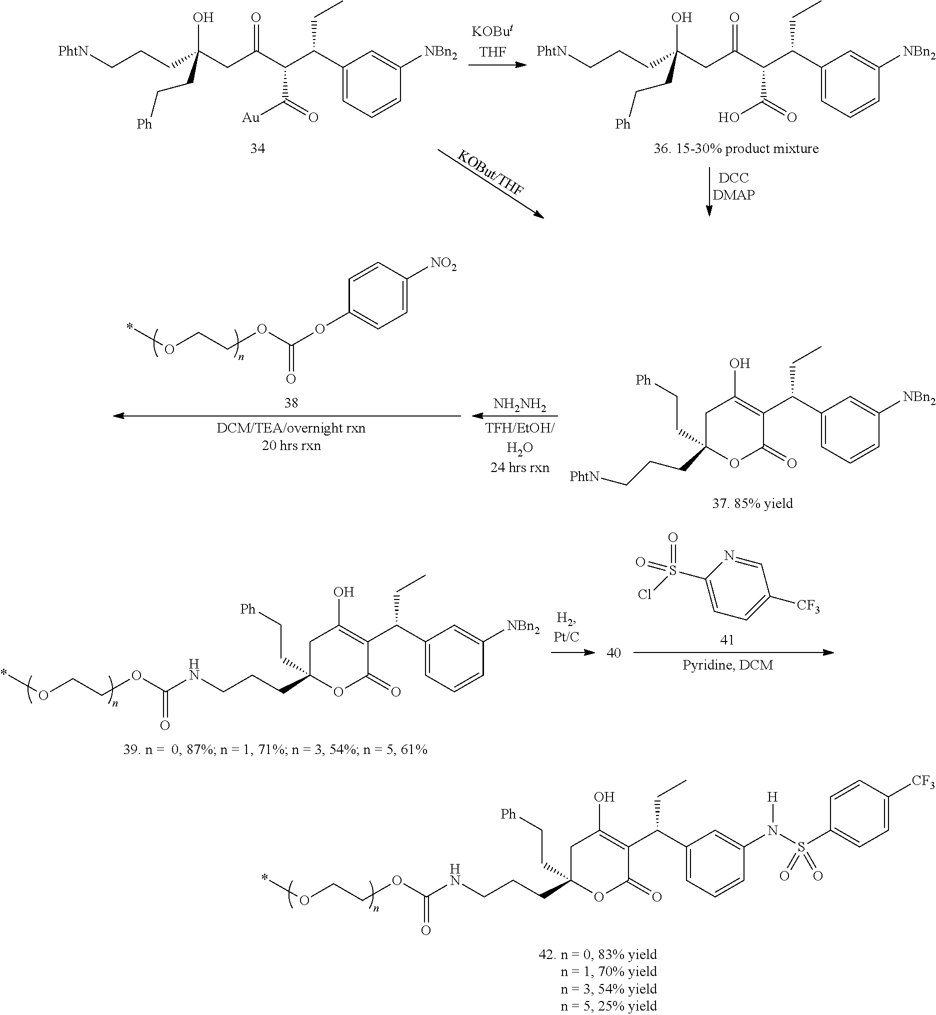

Basic Lactonization (2 Different Processes):

The starting material (34) (3.23 g, 3.66 mmol) was dissolved in THF (91 mL). The solution was cooled down to 0° C. in ice-water bath before KOBu^{t} (1M, 4.21 mL, 4.21 mmol) was added. The reaction was kept at this temperature for 25 minutes and quenched with NH₄Cl aqueous solution (200 mL). EtOAc (200 mL) was added and the separated aqueous phase was extracted with EtOAc (50 mL×2). The combined organic phase was washed with brine (100 mL×2) and dried over Na₂SO₄. It was concentrated and the product mixture was performed DCC/DMAP lactonization without purification.

Lactonization Via DCC/DMAP:

The DCC/DMAP lactonization was applied based on the amount of free acid in the product mixture (36). The design was based on the hplc-UV detector in diluted solution (0.02 M). The DCC (6 eq of remaining free acid) and DMAP (25% of DCC) was added at ambient temperature. In general, this lactonization was accomplished in one hour and DCM was evaporated. The product residue was loaded on the Biotage column (40M, 15-48% EtOAc/Hex in 16 CV). The collected product (37) (1.82 g with 94% purity) and product mixture (858 mg, 59% purity) was obtained after high vacuo (84% total yield). R_{f}=0.10 (Hex:EtOAc=3:1), RP-HPLC (betasil C18, 0.5 mL/min, 30-100% ACN in 10 minutes) 5.16 minutes; LC-MS (ESI, MH⁺) 718.3.

Phthalimide Deprotection:

The above lactone product (37) (507 mg, 0.705 mmol) was dissolved in THF (5.2 mL), EtOH (5.2 mL) and water (4 mL) was dropwise added until the solution start to be cloudy. NaHCO₃ (318 mg, 3 mmol) and NH₂NH₂ (342 μL, 7.05 mmol) was added. The reaction was kept at room temperature for six hours before another NH₂NH₂ (171 μL, 3.52 mmol) portion was added. After 23 hours, HPLC shows the starting material is less than 2%. The reaction was then stopped by adding NaHCO₃ (80 mL) and extracted with DCM (50 mL×3). The combined DCM solution was washed with brine and dried over Na₂SO₄. After filtration, it was concentrated and solidified during the high vacuo drying. The product residue was used in next step PEG conjugation without further purification. RP-HPLC (betasil C18, 0.5 mL/min, 30-100% ACN in 10 minutes) 6.50 minutes; LC-MS (ESI, MH⁺) 589.3.

An approach for preparing mPEG carbonate (38) activation useful in the preparation of mPEGn-OCONH-Tripranavir conjugates is provided below.

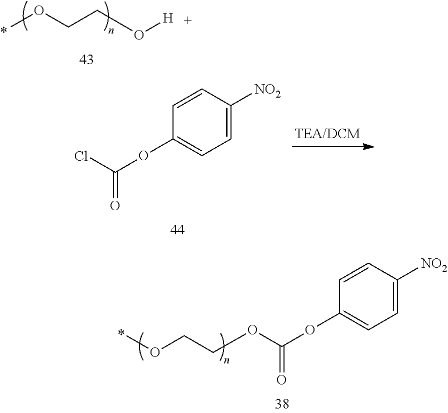

mPEG₁-4-nitrophenyl carbonate: In a 25-mL flask, 2-methoxyethanol (56 μL, 0.705 mmol) was added in DCM (5 mL). p-Nitrophenyl-chloroformate (44) (128 mg, 0.635 mmol) and TEA (147 μL, 1.06 mmol) was added. The reaction was kept at ambient temperature for 30 minutes. The DCM solution was concentrated to 3 mL in order to complete this reaction in next two hours. The reaction was stopped by addition of NH₄Cl (100 mL) and the product was extracted with DCM (30 mL×3). The combined DCM solution was dried over Na₂SO₄ and concentrated under the vacuo. The product (38) was used after high vacuo drying 10 minutes without further purification. RP-HPLC (betasil C18, 0.5 mL/min, 30-100% ACN in 10 minutes) 5.14 minutes.

mPEG₀-4-nitrophenyl carbonate: Methanol (10 eq), 4-nitropheyl chloroformate (1.1 eq), and TEA (1.5 eq). RP-HPLC (betasil C18, 0.5 mL/min, 30-100% ACN in 10 minutes) 4.84 minutes.

mPEG₃-4-nitrophenyl carbonate: Substrate (0.439 mmol), 4-nitrophenyl-chloroformate (1.8 eq), and TEA (1.6 eq). RP-HPLC (betasil C18, 0.5 mL/min, 30-100% ACN in 10 minutes) 5.01 minutes; LC-MS (ESI, MH⁺) 330.1.

mPEG₅-4-nitrophenyl carbonate: Substrate (0.439 mmol), 4-nitrophenyl-chloroformate (1.25 eq), and TEA (1.6 eq). RP-HPLC (betasil C18, 0.5 mL/min, 30-100% ACN in 10 minutes) 4.96 minutes; LC-MS (ESI, MH⁺) 418.1. ¹H NMR (500 MHz, CDCl₃) δ 3.38 (3H, s), 3.54-3.82 (18H, m), 4.43-4.45 (2H, m), 7.39 (2H, d, J=5.4 Hz), 8.28 (2H, d, J=5.4 Hz).

mPEG-Carbamate Conjugation

mPEG₁-OCONH-core (39a): The product after phthalimide deprotecton (0.352 mmol) was dissolved in DCM (3 mL). The vacuo dried mPEG₁-p-nitrophenyl-carbonate (0.635 mmol) was transferred to the above solution with DCM (6 mL in total). TEA (147 μL, 1.05 mmol) was added and the reaction was kept at room temperature for 20 hours. After the reaction was completed, it was quenched with NH₄Cl aqueous solution and extracted with DCM (30 mL×3). The combined organic phase was dried over Na₂SO₄. After filtration, it was concentrated and the residue to loaded on Biotage column (25S, 20-75% of EtOAc in Hex in 16 CV). The desired product 39a was collected as the last portion and almost colorless product (172 mg) and a mixture (˜16 mg, 71% yield in total) was collected. As a distereomeric mixture, ¹H NMR cannot be read. R_{f}=0.15 (Hex:EtOAc=1:1), RP-HPLC (betasil C18, 0.5 mL/min, 30-100% ACN in 10 minutes) 8.65 minutes; LC-MS (ESI, MH⁺) 691.2.

mPEG₀-OCONH-core (39b): Substrate (0.352 mmol), mPEG₀-p-nitrophenyl-carbonate (0.635 mmol), and TEA (147 μL). Biotage (25S, 20%-90% EtOAc in Hex in 16 CV). The colorless product (197 mg, 87% yield) and a mixture (30 mg) were collected. R_{f}=0.31 (Hex:EtOAc=1:1), RP-HPLC (betasil C18, 0.5 mL/min, 30-100% ACN in 10 min) 8.66 min; LC-MS (ESI, MH⁺) 647.2.

mPEG₃-OCONH-core (39c): Substrate (0.439 mmol), mPEG₃-p-nitrophenyl-carbonate (0.702 mmol), and TEA (122 μL). Biotage (25S, 15-68% EtOAc in Hex in 16 CV). The colorless product (183 mg, 54% yield) was collected. R_{f}=0.55 (EtOAc), RP-HPLC (betasil C18, 0.5 mL/min, 30-100% ACN in 10 minutes) 8.27 minutes; LC-MS (ESI, MH⁺) 779.5.

mPEG₅-OCONH-core (39d): Substrate (0.406 mmol), mPEG₀-p-nitrophenyl-carbonate (0.508 mmol), and TEA (113 μL). Biotage (25S, 15-68% EtOAc in Hex in 16 CV following by 2-7% MeOH in DCM in 16 CV). The colorless product (216 mg, 61% yield) and a mixture (29.5 mg) were collected. R_{f}=0.22 (EtOAc), RP-HPLC (betasil C18, 0.5 mL/min, 30-100% ACN in 10 minutes) 8.34 minutes; LC-MS (ESI, MH⁺) 867.5.

Hydrogenolysis:

mPEG₀-OCONH-core-NH₂ (40a): The substrate (39a) mPEG₀-OCONH-core-NBn₂ (197.2 mg, 0.305 mmol) was dissolved in EtOAc (6.0 mL) and MeOH (6.0 mL) mixture solution. The solution vial was bubbling N₂ for exchange at lease 15 minutes before catalyst addition. Stop stirring, and the Pd/C catalyst (39 mg, 10 wt %×2) was added slowly. The system was evacuated and recharged with hydrogen gas (˜50 psi) three times (stop stirring during vacuo). The hydrogenolysis was then kept at room temperature under 50 psi for 24 hrs to complete. After release the pressure, the reaction mixture was first checked with HPLC to see the completeness before a filtration was performed. The catalyst residue and filter paper was carefully washed with methanol. The solution was then evaporated and vacuo drying to give oil-like product (145.3 mg, >100% yield). No further purification is necessary. No proton NMR due to the diastereoisomer mixture and low solubility in CHCl₃. RP-HPLC (betasil C18, 0.5 mL/min, 20-60% ACN in 10 minutes) 7.03+7.44 minutes, LC-MS (ESI, MH⁺) 467.3.

mPEG₁-OCONH-core-NH₂ (40b): RP-HPLC (betasil C18, 0.5 mL/min, 20-600% ACN in 10 minutes) 6.89+7.18 minutes; LC-MS (ESI, MH⁺) 511.3.

mPEG₃-OCONH-core-NH₂ (40c): RP-HPLC (betasil C18, 0.5 mL/min, 20-60% ACN in 10 minutes) 7.20+7.43 minutes; LC-MS (ESI, MH⁺) 599.3.

mPEG₅-OCONH-core-NH₂ (40d): RP-HPLC (betasil C18, 0.5 mL/min, 30-100% ACN in 10 minutes) 4.05+4.29 minutes; LC-MS (ESI, MH⁺) 687.4.

Sulphonate Amide Conjugation:

mPEG₃-OCONH-Tipranavir-2 (42c): The free amine mPEG₃-OCONH-core-NH₂ (40c) (67.3 mg, 0.112 mmol) was dissolved in DCM (3.0 mL) under N₂ protection. After dissolving, the solution was cool down in an ice-water bath and sulphonyl chloride (27 mg, 0.112 mmol) was added. Pyridine (18 μL, 0.224 mmol) was then added and the reaction was kept at 0° C. for 30 minutes. Methyl amine (2M, 500 μL, 1.0 mmol) was added and the reaction was kept at this temperature for three hours. After HPLC show the reaction was completed, it was quenched with NH₄Cl (10 mL) and diluted with DCM and H₂O. The separated organic phase was washed with brine (10 mL×2). The organic phase was then dried over Na₂SO₄, filtrated, and concentrated. The crude product was purified on Biotage (40-90% EtOAc in Hex in 16 CV) provided a slightly yellowish solid product (42.6 mg) and a less pure product (6.6 mg) with the total yield about 54%. Proton NMR cannot read due to its diastereomer mixture. Isomer ratio=54/41. R_{f}=0.37 (EtOAc), RP-HPLC (betasil C18, 0.5 mL/min, 60-100% ACN in 8 minutes) 7.16+7.28 minutes; LC-MS (ESI, MH⁺) 808.3.

mPEG₀-OCONH-Tipranavir-2 (42a): The free amine mPEG₀-OCONH-core-NH₂ 40a (71.7 mg, 0.154 mmol), sulphonyl chloride (37.0 mg, 0.154 mmol), Pyridine (25 μL, 0.308 mmol). Biotage (20-80% EtOAc in Hex in 16 CV) provided a slightly yellowish solid product (51.5 mg). Isomer ratio=55/43. R_{f}=0.12 (Hex:EtOAc=1:1), RP-HPLC (betasil C18, 0.5 mL/min, 30-100% ACN in 10 minutes) 7.51+7.61 minutes; LC-MS (ESI, MH⁺) 676.2.

mPEG₁-OCONH-Tipranavir-2 (42c): The free amine mPEG₁-OCONH-core-NH₂ 40c (62.0 mg, 0.122 mmol), sulphonyl chloride (31.0 mg, 0.128 mmol), Pyridine (20 μL, 0.244 mmol). Biotage (25-90% EtOAc in Hex in 16 CV) provided a slightly yellowish solid product. Isomer ratio=58/40. R_{f}=0.06 (Hex:EtOAc=1:1); 0.72 (EtOAc), RP-HPLC (betasil C18, 0.5 mL/min, 30-100% ACN in 10 minutes) 7.49+7.60 minutes; LC-MS (ESI, MH⁺) 720.3.

mPEG₅-OCONH-Tipranavir-2 (42d): The free amine mPEG₅-OCONH-core-NH₂ 42d (156 mg, 0.227 mmol), sulphonyl chloride (64.0 mg, 0.254 mmol), Pyridine (42 μL, 0.254 mmol). Biotage (1-7% MeOH in DCM in 16 CV) provided a slightly yellowish solid product. Isomer ratio=56/41. R_{f}=0.06 (EtOAc), RP-HPLC (betasil C18, 0.5 mL/min, 30-100% ACN in 10 minutes) 7.26+7.37 minutes; LC-MS (ESI, MH⁺) 918.3.

### Example 1

### Preparation of mPEG₆-Atazanavir-NH-Ethyl Carbamate

mPEG₆-atazanavir-NH-ethyl carbamate was prepared in two steps. Schematically, the first step can be schematically represented as follows.

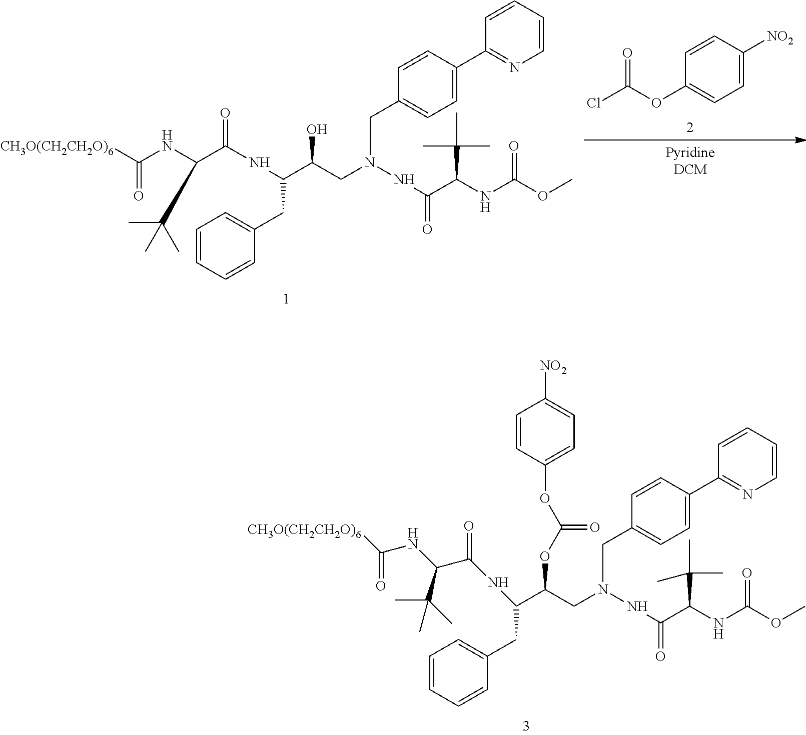

Thereafter, the second step to provide the desired mPEG₆-atazanavir-NH-ethyl carbamate can be represented as follows.

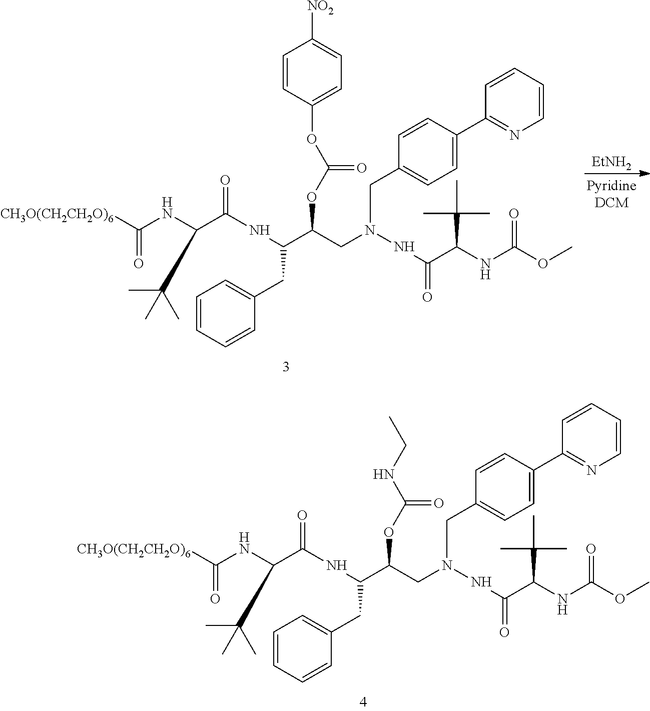

### mPEG₆-Atazanavir-nitrophenyl carbonate (3)

Into a 500 mL flask was added previously prepared mPEG₆-atazanavir (1) (5.0 gm, 5.16 mmol) and anhydrous dichloromethane (150 mL). To this solution was added anhydrous pyridine (4.18 mL, 51.6 mmol, 10.0 equivalents) and the yellow solution was stirred under nitrogen at room temperature for 45 minutes. To this solution was then added 4-nitrophenyl chloroformate (2) (5.47 gm, 25.8 mmol, 5.0 equivalents) and the cloudy suspension was stirred under nitrogen at room temperature. After approximately twenty hours, the reaction mixture was diluted with dichloromethane (175 mL) and divided into two portions, for subsequent treatment. Each portion was partitioned with deionized water (130 mL). The aqueous layer was extracted with dichloromethane (3×30 mL). The combined organics were washed successively with saturated sodium bicarbonate, deionized water, 1 N HCl, deionized water, and saturated sodium chloride (130 mL each). The combined organic layers were dried over sodium sulfate, filtered and concentrated under reduced pressure to give an off-white solid. Purification by biotage chromatography (gradient elution: 0 to 10% methanol-dichloromethane over 20 CV) gave 4.06 gm (70%) of mPEG₆-Atazanavir-nitrophenyl carbonate, compound (3), as a light-yellow foam solid.

### mPEG₆-Atazanavir-NH-ethyl carbamate (4)

Into a 250 mL flask was added compound (3) (4.35 gm, 3.83 mmol) and anhydrous dichloromethane (80 mL). To the yellow solution was added pyridine (0.77 mL, 9.59 mmol, 2.5 equivalents), followed by ethylamine (1.33 mL, 19.1 mmol, 5.0 equivalents). The yellow reaction mixture was stirred under nitrogen at room temperature. After approximately 18 hours the yellow mixture was diluted with dichlormethane (100 mL). The mixture was transferred to a reparatory funnel and partitioned with saturated sodium bicarbonate (100 mL). The aqueous layer was extracted with dichloromethane (3×30 mL). The combined organics were washed successively with deionized water, 1N HCl, deionized water and saturated sodium chloride (100 mL each). The organic layer was filtered, and concentrated under reduced pressure to give an off-white solid. Purification by biotage chromatography (gradient elution: 0 to 5% methanol/dichloromethane over 20 CV) gave 2.92 gm (75%) of 4 as a light-yellow solid.

### mPEG₃-atazanavir-NH-ethyl carbamate

Using an approach similar to that used to make mPEG₆-atazanavir-NH-ethyl carbamate, mPEG₃-atazanavir-NH-ethyl carbamate was prepared.

### mPEG₅-atazanavir-NH-ethyl carbamate

Using an approach similar to that used to make mPEG₆-atazanavir-NH-ethyl carbamate, mPEG₅-atazanavir-NH-ethyl carbamate was prepared.

### Example 2

### Preparation of mPEG₃-Atazanavir-L-Valine HCl

mPEG₃-Atazanavir-L-valine HCl was prepared in two steps. Schematically, the first step can be schematically represented as follows.

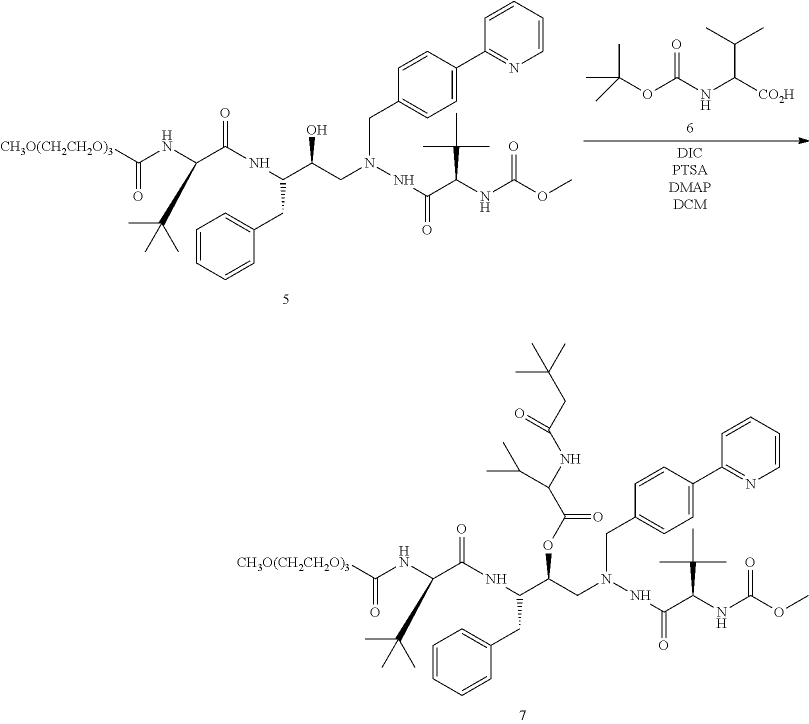

Thereafter, the second step to provide the desired mPEG₃-atazanavir-L-valine HCl can be schematically represented as follows.

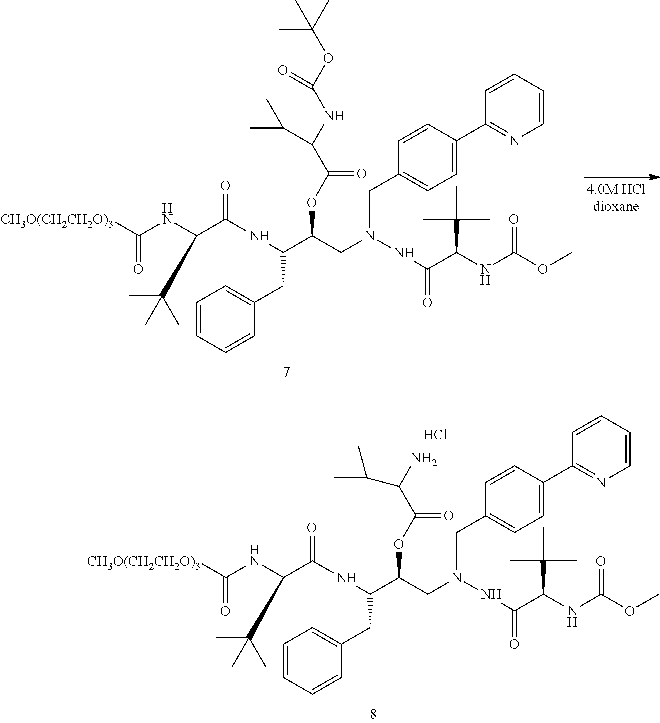

### mPEG₃-Atazanavir-Boc-L-valine (7)

Into a 500 mL flask was added previously prepared mPEG₃-atazanavir (5) (4.0 gm, 4.7 mmol) and anhydrous dichloromethane (140 mL). To the clear solution was added compound (6) (15.5 gm, 71.6 mmol, 15.0 equivalents) and DPTS (1:1 DMAP:PTSA; 1.48 gm, 4.7 mmol, 1.0 equivalents). To the light-yellow solution was added DIC (N,N′-diisopropylcarbodiimide) (14.8 mL, 95.5 mmol, 20.0 equivalents), and the reaction mixture turned cloudy yellow. The reaction mixture was stirred under nitrogen at room temperature. After approximately four hours the reaction mixture was diluted with dichloromethane (100 mL) and transferred to a separatory funnel. The mixture was partitioned with water (150 mL). A white insoluble solid (the excess Boc-L-valine) was filtered off. The aqueous layer was extracted with dichloromethane (3×25 mL). The combined organic layers were washed with water, saturated sodium bicarbonate, water, and saturated sodium chloride (150 mL each). After filtering and concentrating under reduced pressure, the residue was purified by biotage chromatography (gradient elution: 0 to 5% methanol/dichloromethane) to give 3.76 gm (77%) of mPEG₃-atazanavir-Boc-L-valine (7) as a white foam solid.

### mPEG₃-Atazanavir-L-valine HCl (8)

Into a 100 mL flask was added mPEG₃-atazanavir-Boc-L-valine (7) (1.9 gm, 1.8 mmol) and 1,4-dioxane (12 mL). To the clear solution was added 4.0 M HCl in dioxane (5 mL) and the reaction mixture was stirred under at room temperature. After approximately 18 hours the reaction mixture was concentrated under reduced pressure. The residue was taken up in dichloromethane (30 mL) and transferred to a separatory funnel. The organic layer was partitioned with saturated sodium chloride (10 mL), and the layers were separated. The organic layer was concentrated under reduced pressure to give 1.26 gm (71%) of mPEG₃-atazanavir-L-valine HCl (8) as a light-yellow solid.

### Example 3

### Preparation of mPEG₅-Atazanavir-L-Valine HCl

mPEG₅-Atazanavir-L-valine HCl was prepared in two steps. Schematically, the first step can be schematically represented as follows.

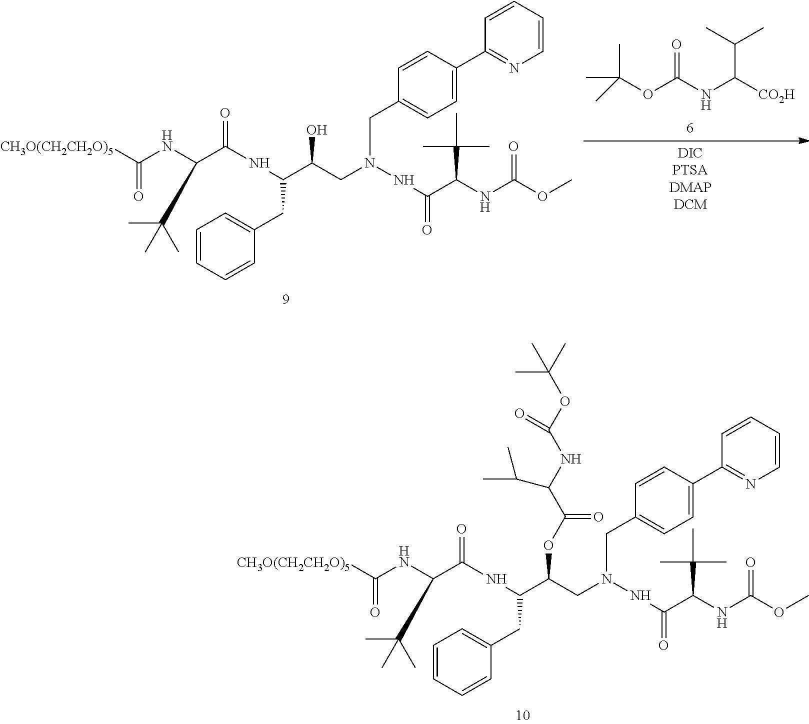

Thereafter, the second step to provide the desired mPEG₅-atazanavir-L-valine HCl can be schematically represented as follows.

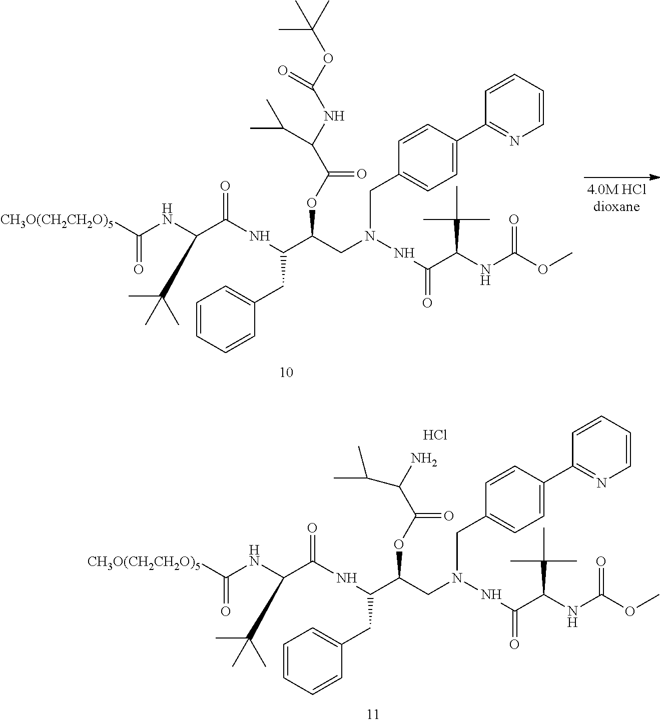

### mPEG₅-Atazanavir-Boc-L-valine (10)

Into a 500 mL flask was added previously prepared mPEG₅-atazanavir (9) (5.0 gm, 5.4 mmol) and anhydrous dichloromethane (160 mL). To the clear solution was added compound (6) (17.6 gm, 81.0 mmol, 15.0 equivalents) and DPTS (1:1 DMAP:PTSA; 1.67 gm, 5.4 mmol, 1.0 equivalents). To the light-yellow solution was added DIC (N,N′ diisopropylcarbodiimide (16.7 mL, 108.1 mmol, 20.0 equivalents), and the reaction mixture turned cloudy yellow. The reaction mixture was stirred under nitrogen at room temperature. After approximately one hour the reaction mixture was diluted with dichloromethane (100 mL) and transferred to a separatory funnel. The mixture was partitioned with water (150 mL). A white insoluble solid (the excess Boc-L-valine) was filtered off. The aqueous layer was extracted with dichloromethane (3×30 mL). The combined organic layers were washed with water, saturated sodium bicarbonate, water, and saturated sodium chloride (150 mL each). After filtering and concentrating under reduced pressure, the residue was purified by biotage chromatography (gradient elution: 0 to 5% methanol/dichloromethane) to give 5.04 gm (84%) of mPEG₅-atazanavir-Boc-L-valine (10) as a white foam solid.

### mPEG₅-Atazanavir-L-valine HCl (11)

Into a 100 mL flask was added mPEG₅-atazanavir-Boc-L-valine (10) (2.57 gm, 2.28 mmol) and 1,4-dioxane (20 mL). To the clear solution was added 4.0 M HCl in dioxane (6.4 mL) and the reaction mixture was stirred under at room temperature. After approximately 18 hours the reaction mixture was concentrated under reduced pressure. The residue was taken up in dichloromethane (30 mL) and transferred to a separatory funnel. The organic layer was partitioned with saturated sodium chloride (10 mL), and the layers were separated. The organic layer was concentrated under reduced pressure to give 1.43 gm (64%) of mPEG₅-atazanavir-L-valine HCl (11) as a light-yellow solid.

### Examples 4a-4-h

### Preparation of Lipid Esters of mPEGₙ-Atazanavir

The approach schematically provided below was generally followed to prepare lipid esters of mPEGₙ-atazanavir.

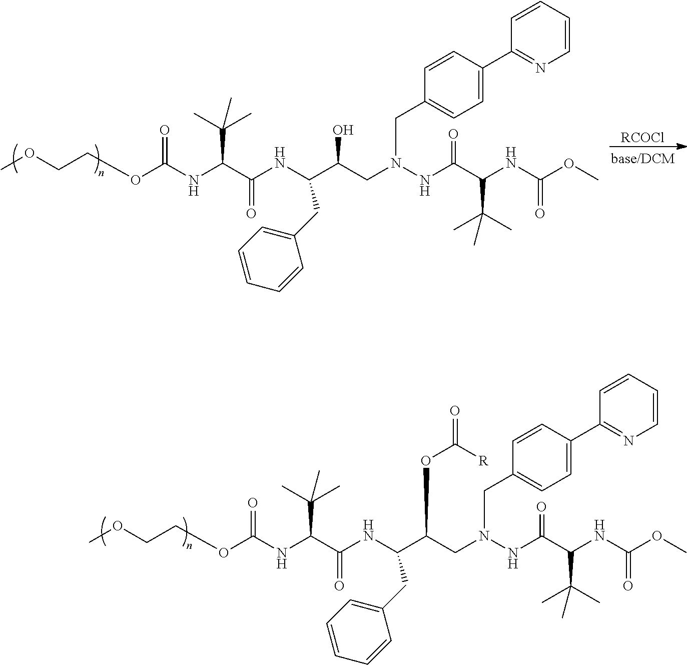

Materials. In preparing Examples 4a through 4h, the following materials were used: each of pyridine, valeroyl chloride (C₅H₉ClO), hexanolyl chloride (C₆H₁₁ClO), and lauroyl chloride (C₁₂H₂₃ClO) were purchased from Sigma-Aldrich (St Louis, Mo.) or other commercial source; each of mPEG₃-atazanavir, mPEG₃-atazanavir and mPEG₃-atazanavir was prepared previously; each of sodium bicarbonate (NaHCO₃), ammonium chloride (NH₄Cl), sodium sulfate (Na₂SO₄), sodium chloride (NaCl), sodium hydroxide (NaOH), and hydrochloride acid (HCl) was purchased from EM Science (Gibbstown, N.J.). DCM was prepared by freshly distilled from CaH₂ and other materials (e.g., methanol, EtOAc, and other organic solvents) were used as purchased.

Examples 4a through 4h were prepared following the same general approach. Briefly, in an N₂ protected dried 250-mL flask, mPEGₙ-atazanavir (3.0 g) was dissolved in freshly distilled DCM (48 mL). The solution was cooled down with an ice-water bath before pyridine (12 eq) was added three minutes later. The lipid acid chloride (2.8 eq) was then added dropwise. The ice-water bath was removed after addition and the reaction was kept at ambient temperature for six hours when the reaction was complete. The reaction was monitored by HPLC and additional quantities of acid chloride was added (1.5 eq) if starting material was remaining.

Thereafter, the reaction solution was diluted to aprroximately (80 mL) and was poured into a saturated NH₄Cl aqueous solution (100 mL). HCl (1N, 5 mL) was added to the aqueous phase as a wash, and another HCl (1N, 5 mL) aliquot was added into the same aqueous phase as a second wash. The double acidic wash was repeated three times until the aqueous solution shows a pH <3. The DCM solution was then washed with saturated NaHCO₃ (100 mL) and NaCl (100 mL) before it was dried over Na₂SO₄ and the solvent was evaporated under vacuo. The residue was loaded onto a Biotage column (40 M) and a MeOH-DCM (program based on the polarity of product) gradient elution was used to purify the product. The product fraction's quality was confirmed by HPLC (>95%) before fractions were combined. Then the resulting solution was concentrated under reduced pressure and the residue was redissolved in EtOAc (80 mL). This organic solution was washed three times with a mixture of NaHCO₃ (90 mL) and NaOH (1N, 5 mL) solution (pH=˜10). The EtOAc solution was then washed with aqueous NH₄Cl (90 mL×2) and then was adjusted to pH <7 before it was dried over Na₂SO₄. After filtration, it was concentrated and dried under vacuum to give a colorless oily-like solid product.

### Example 4a

### C₄H₉CO-mPEG₃-atazanavir

Biotage program 1-7% MeOH in DCM in 30 CV, RP-HPLC (betasil C18, 0.5 mL/min, 30-80% ACN in 10 min) 7.05 min, LC-MS (ESI, MH⁺) 921.5; ¹H NMR (500 MHz, CDCl₃) δ 0.76 (9H, s), 0.84 (9H, s), 0.99 (3H, t, J=7.0 Hz), 1.45 (2H, h, J=7.5 Hz), 1.68 (2H, p, J=7.5 Hz), 2.40 (2H, t, J=7.5 Hz), 2.62 (1H, dd, J=8.5, 13.0 Hz), 2.72-2.78 (2H, m), 3.25 (1H, d, J=9.5 Hz), 3.36 (3H, s), 3.53-3.68 (15H, m), 3.74 (1H, d, J=9.5 Hz), 4.15-4.24 (4H, m), 4.34 (1H, q, J=6.0 Hz), 5.04 (1H, d, J=5.5 Hz), 5.29 (1H, d, J=9.5 Hz), 5.44 (1H, d, J=9.0 Hz), 5.99 (1H, d, J=8.0 Hz), 7.12 (2H, d, J=7.5 Hz), 7.18-7.26 (6H, m), 7.70-7.75 (2H, m), 7.90 (2H, d, J=8.0 Hz), 8.68 (1H, d, J=4.5 Hz).

### Example 4b

### C₄H₉CO-mPEG₅-atazanavir

Biotage program 1-8% MeOH in DCM in 30 CV, RP-HPLC (betasil C18, 0.5 mL/min, 30-80% ACN in 10 min) 6.89 min, LC-MS (ESI, MH⁺) 1009.7; ¹H NMR (500 MHz, CDCl₃) δ 0.76 (9H, s), 0.84 (9H, s), 1.01 (3H, t, J=7.0 Hz), 1.45 (2H, h, J=7.5 Hz), 1.68 (2H, p, J=7.5 Hz), 2.40 (2H, t, J=7.5 Hz), 2.62 (1H, dd, J=8.5, 13.0 Hz), 2.71-2.77 (2H, m), 3.25 (1H, d, J=9.5 Hz), 3.36 (3H, s), 3.53-3.68 (23H, m), 3.74 (1H, d, J=9.5 Hz), 4.14-4.23 (4H, m), 4.34 (1H, q, J=5.5 Hz), 5.04 (1H, d, J=5.5 Hz), 5.28 (1H, d, J=9.5 Hz), 5.42 (1H, d, J=9.5 Hz), 5.95 (1H, d, J=8.5 Hz), 7.12 (2H, d, J=7.5 Hz), 7.18-7.26 (6H, m), 7.70-7.75 (2H, m), 7.90 (2H, d, J=8.0 Hz), 8.68 (1H, d, J=4.5 Hz).

### Example 4c

### C₄H₉CO-mPEG₆-atazanavir

Biotage program 1-9% MeOH in DCM in 30 CV, RP-HPLC (betasil C18, 0.5 mL/min, 30-80% ACN in 10 min) 6.70 min, LC-MS (ESI, MH⁺) 1053.7; ¹H NMR (500 MHz, CDCl₃) δ 0.76 (9H, s), 0.83 (9H, s), 1.00 (3H, t, J=7.0 Hz), 1.44 (2H, h, J=7.5 Hz), 1.68 (2H, p, J=7.5 Hz), 2.40 (2H, t, J=7.5 Hz), 2.62 (1H, dd, J=8.5, 13.0 Hz), 2.71-2.77 (2H, m), 3.25 (1H, d, J=9.5 Hz), 3.37 (3H, s), 3.53-3.68 (27H, m), 3.74 (1H, d, J=9.5 Hz), 4.16-4.24 (4H, m), 4.34 (1H, q, J=5.5 Hz), 5.04 (1H, d, J=5.5 Hz), 5.28 (1H, d, J=9.5 Hz), 5.42 (1H, d, J=9.5 Hz), 5.95 (1H, d, J=7.5 Hz), 7.12 (2H, d, J=7.5 Hz), 7.18-7.26 (6H, m), 7.70-7.75 (2H, m), 7.90 (2H, d, J=8.0 Hz), 8.68 (1H, d, J=4.5 Hz).

### Example 4d

### C₅H₁₁CO-mPEG₃-atazanavir

Biotage program 1-6% MeOH in DCM in 30 CV, RP-HPLC (betasil C18, 0.5 mL/min, 30-80% ACN in 10 min) 7.63 min, LC-MS (ESI, MH⁺) 935.6; ¹H NMR (500 MHz, CDCl₃) δ 0.76 (9H, s), 0.84 (9H, s), 0.95 (3H, t, J=7.0 Hz), 1.41 (4H, bs), 1.71 (2H, bs), 2.39 (2H, t, J=7.5 Hz), 2.62 (1H, dd, J=9.5, 13.0 Hz), 2.72-2.78 (2H, m), 3.25 (1H, d, J=11.0 Hz), 3.37 (3H, s), 3.53-3.68 (15H, m), 3.74 (1H, d, J=9.0 Hz), 4.15-4.23 (4H, m), 4.34 (1H, q, J=7.0 Hz), 5.04 (1H, d, J=5.5 Hz), 5.29 (1H, d, J=9.0 Hz), 5.44 (1H, d, J=9.0 Hz), 5.99 (1H, d, J=8.0 Hz), 7.12 (2H, d, J=7.0 Hz), 7.18-7.25 (6H, m), 7.70-7.75 (2H, m), 7.91 (2H, d, J=8.0 Hz), 8.68 (1H, d, J=4.0 Hz).

### Example 4e

### C₅H₁₁CO-mPEG₅-atazanavir

Biotage program 1-7% MeOH in DCM in 30 CV, RP-HPLC (betasil C18, 0.5 mL/min, 30-80% ACN in 10 min) 7.45 min, LC-MS (ESI, MH⁺) 1023.5; ¹H NMR (500 MHz, CDCl₃) δ 0.77 (9H, s), 0.84 (9H, s), 0.96 (3H, t, J=6.0 Hz), 1.40-1.41 (4H, m), 1.69-1.71 (2H, m), 2.39 (2H, t, J=7.5 Hz), 2.62 (1H, dd, J=8.5, 13.0 Hz), 2.72-2.78 (2H, m), 3.25 (1H, d, J=10.0 Hz), 3.36 (3H, s), 3.53-3.68 (23H, m), 3.74 (1H, d, J=9.5 Hz), 4.16-4.24 (4H, m), 4.34 (1H, q, J=6.5 Hz), 5.04 (1H, d, J=6.0 Hz), 5.29 (1H, d, J=9.0 Hz), 5.42 (1H, d, J=9.0 Hz), 5.96 (1H, d, J=8.0 Hz), 7.12 (2H, d, J=7.5 Hz), 7.19-7.26 (6H, m), 7.70-7.76 (2H, m), 7.91 (2H, d, J=7.5 Hz), 8.68 (1H, d, J=4.5 Hz).

### Example 4f

### C₅H₁₁CO-mPEG₆-atazanavir

Biotage program 1-8% MeOH in DCM in 30 CV, RP-HPLC (betasil C18, 0.5 mL/min, 30-80% ACN in 10 min) 7.40 min, LC-MS (ESI, MH⁺) 1067.7; ¹H NMR (500 MHz, CDCl₃) δ 0.77 (9H, s), 0.84 (9H, s), 0.96 (3H, t, J=6.0 Hz), 1.41 (4H, bs), 1.71 (2H, bs), 2.39 (2H, t, J=7.5 Hz), 2.62 (1H, dd, J=9.0, 13.0 Hz), 2.72-2.78 (2H, m), 3.25 (1H, d, J=10.5 Hz), 3.37 (3H, s), 3.54-3.68 (27H, m), 3.74 (1H, d, J=9.5 Hz), 4.16-4.24 (4H, m), 4.34 (1H, q, J=6.5 Hz), 5.04 (1H, d, J=6.0 Hz), 5.27 (1H, d, J=9.0 Hz), 5.42 (1H, d, J=9.0 Hz), 5.96 (1H, d, J=8.0 Hz), 7.12 (2H, d, J=7.5 Hz), 7.18-7.26 (6H, m), 7.70-7.76 (2H, m), 7.91 (2H, d, J=7.5 Hz), 8.68 (1H, d, J=4.5 Hz).

### Example 4g

### C₇H₁₅CO-mPEG₅-atazanavir

Biotage program 1-6% MeOH in DCM in 30 CV, RP-HPLC (betasil C18, 0.5 mL/min, 30-80% ACN in 10 min) 8.66 min, LC-MS (ESI, MH⁺) 1051.6; ¹H NMR (500 MHz, CDCl₃) δ 0.76 (9H, s), 0.84 (9H, s), 0.90 (3H, t, J=7.0 Hz), 1.33-1.39 (8H, m), 1.66-1.70 (2H, m), 2.39 (2H, t, J=7.5 Hz), 2.62 (1H, dd, J=9.0, 13.5 Hz), 2.71-2.78 (2H, m), 3.26 (1H, d, J=13.0 Hz), 3.37 (3H, s), 3.53-3.68 (23H, m), 3.73 (1H, d, J=9.5 Hz), 4.16-4.24 (4H, m), 4.34 (1H, q, J=6.5 Hz), 5.04 (1H, d, J=6.0 Hz), 5.27 (1H, d, J=9.0 Hz), 5.41 (1H, d, J=9.0 Hz), 5.92 (1H, d, J=8.0 Hz), 7.12 (2H, d, J=7.0 Hz), 7.19-7.25 (6H, m), 7.70-7.75 (2H, m), 7.91 (2H, d, J=8.0 Hz), 8.68 (1H, d, J=5.0 Hz).

### Example 4h

### C₁₁H₂₃CO-mPEG₆-atazanavir

R_{f}=0.46 (DCM:MeOH=15:1). Biotage program 1-6% MeOH in DCM in 30 CV, RP-HPLC (betasil C18, 0.5 mL/min, 30-80% ACN in 10 min) 7.60 min, LC-MS (ESI, MH⁺) 1053.7; ¹H NMR (500 MHz, CDCl₃) δ 0.77 (9H, s), 0.84 (9H, s), 0.88 (3H, t, J=7.5 Hz), 1.26-1.41 (20H, m), 2.39 (2H, t, J=7.5 Hz), 2.62 (1H, dd, J=8.5, 13.5 Hz), 2.72-2.77 (2H, m), 3.25 (1H, d, J=10.0 Hz), 3.37 (3H, s), 3.53-3.68 (27H, m), 3.74 (1H, d, J=9.5 Hz), 4.16-4.25 (4H, m), 4.34 (1H, q, J=6.5 Hz), 5.04 (1H, d, J=6.0 Hz), 5.29 (1H, d, J=9.5 Hz), 5.44 (1H, d, J=9.5 Hz), 5.98 (1H, d, J=7.5 Hz), 7.12 (2H, d, J=7.0 Hz), 7.19-7.25 (6H, m), 7.70-7.75 (2H, m), 7.89-7.91 (2H, d, J=8.0 Hz), 8.69 (1H, d, J=4.5 Hz).

### Example 5

### Synthesis of Butyl Carbamate of mPEG₆-Atazanavir

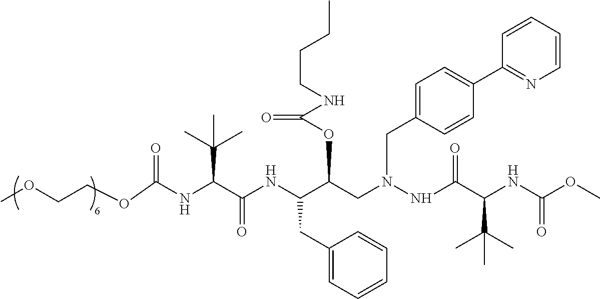

Butyl carbamate of mPEG₆-atazanavir was prepared in accordance with the schematic provided below.

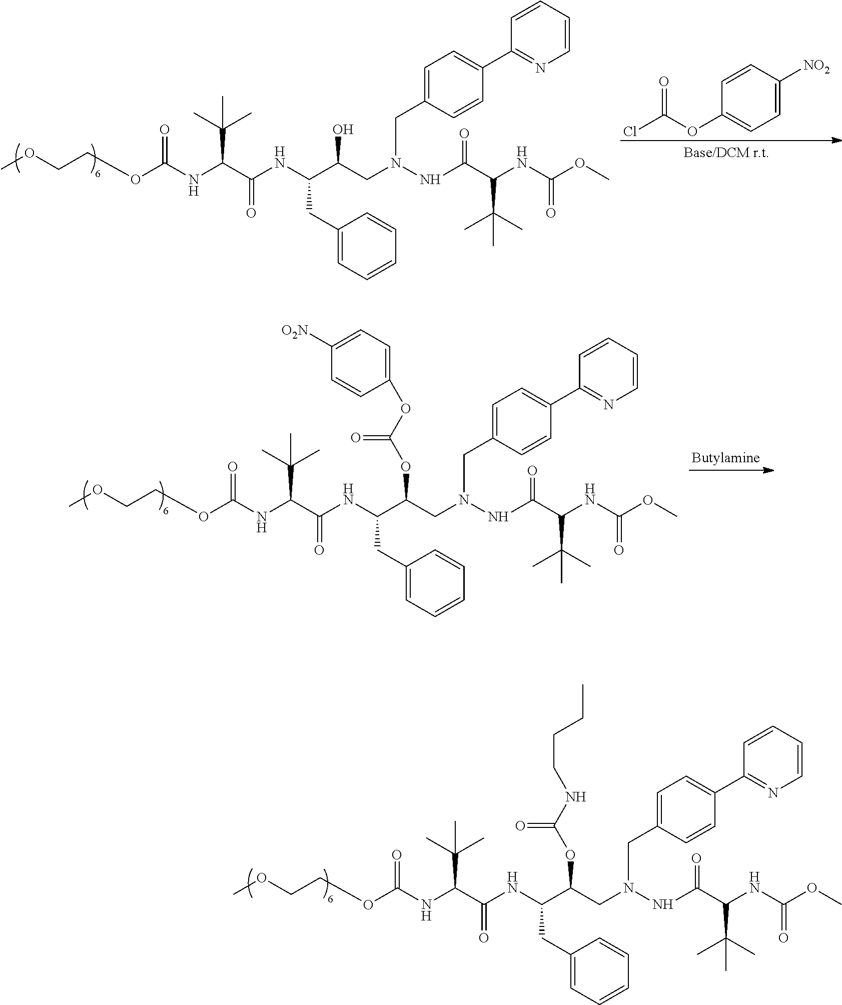

Previously prepared mPEG₆-atazanavir (1.086 g, 1.12 mmol) was dissolved in anhydrous dichloromethane (15 mL) at room temperature. Anhydrous pyridine (0.5 mL, 6.18 mmol) was added, followed by an addition of 4-nitrophenyl chloroformate (766 mg, 3.65 mmol). Additional solvent dichloromethane (9 mL) was added. The resulting mixture was stirred at room temperature for two hours and forty minutes, Butylamine (0.5 mL, 5.01 mmol) was added dropwise. The mixture was stirred at room temperature for nineteen hours, at which point more butylamine (1.0 mL, 10.08 mmol) was added. The mixture was stirred for anther six hours at room temperature. NaHCO₃ aqueous solution was added to quench the reaction. The organic phase was separated and the aqueous solution was extracted with dichloromethane (20 mL). The combined organic solution was washed with water, brine, dried over Na₂SO₄, concentrated to afford a residue. The residue was purified with flash column chromatography on silica gel using 0-5% MeOH/CH₂Cl₂ to afford 0.6728 g of butyl carbamate of mPEG₆-atazanavir in 56% yield. ¹H-NMR (CDCl₃): 8.676 (d, J=5.0 Hz, 1H, Ar—H), 7.877 (d, J=8.5 Hz, 2H, 2Ar—H), 7.745 (m, 1H, 1Ar—H), 7.696 (m, 1H, 1Ar—H), 7.304 (d, J=8.0 Hz, 2H, 2Ar—H), 7.236-7.143 (m, 6H, 6Ar—H), 5.913 (d, J=8.0 Hz, 1H, NH), 5.383 (d, J=8.5 Hz, 1H, NH), 5.303 (d, J=9.0 Hz, 1H, NH), 5.061 (br, 1H, NH), 4.916 (d, J=9.5 Hz, 1H, NH), 4.309-4.170 (m, 5H, 2CHBu^{t}, CH₂, and CHCH₂Ph), 3.666-3.525 (m, 28H, OCH₃, 12CH₂ and CHOCO), 3.365 (s, 3H, CH₃), 3.301 (m, 2H, CH₂), 3.178 (m, 1H, CH), 2.773-2.644 (m, 3H, CH and CH₂), 1.640-1.595 (m, 2H, CH₂), 1.502-1.429 (m, 2H, CH₂), 1.011 (t, J=7.0 Hz, 3H, CH₃), 0.844 (s, 9H, Bu^{t}), 0.795 (s, 9H, Bu^{t}). LC-MS: 1068.7 (MH⁺).

### Example 6

### Synthesis of mPEG₃-Atazanavir Butyrate

mPEG₃-Atazanavir butyrate was prepared in accordance with the schematic provided below.

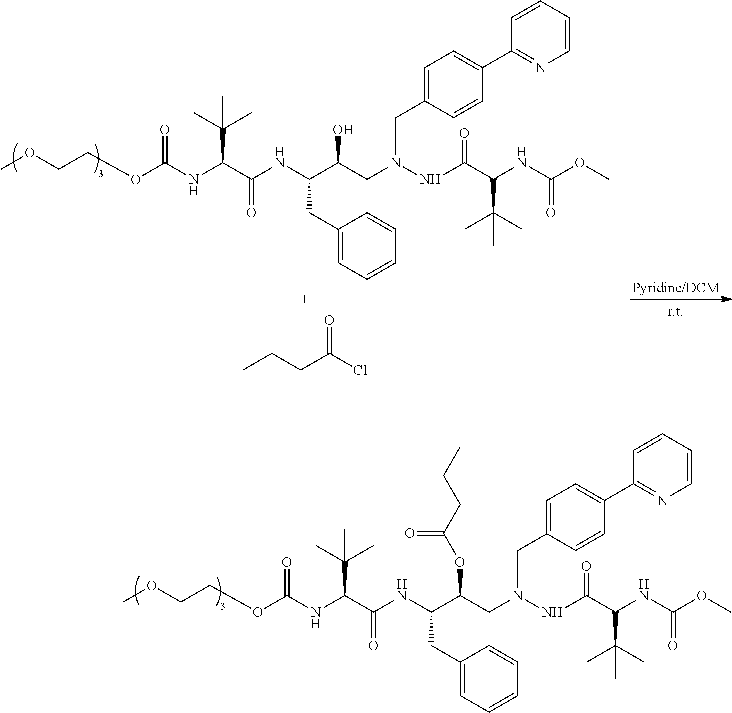

Previously prepared mPEG₃-atazanavir (3.045 g. 3.63 mmol) was dissolved in anhydrous dichloromethane (50 mL) at room temperature. Anhydrous pyridine (2.9 mL, 35.86 mmol) was added. The mixture was cooled to 0° C., butyryl chloride (1.1 mL, 10.41 mmol) was added dropwise. The resulting mixture was stirred at 0° C. for sixty minutes, at room temperature for nineteen hours. An additional amount of butyryl chloride (0.1 mL, 0.946 mmol) was added. The reaction mixture was stirred at room temperature for another four hours. 5% NaHCO₃ aqueous solution (100 mL) was added to quench the reaction. The mixture was concentrated to remove the organic solvent and the remaining mixture was extracted with ethyl acetate (2×100 mL). The ethyl acetate solution was washed with saturated NaCl solution (pH˜1.0 by addition of 1N HCl) (4×100 mL), 5% NaHCO₃ aqueous solution (2×100 mL) and saturated NH₄Cl solution, dried over Na₂SO₄, concentrated. The residue was purified with flash column chromatography on silica gel and reverse column chromatography to afford the product mPEG₃-atazanavir butyrate (2.504 g, yield: 76%). ¹H-NMR (CDCl₃): 8.679 (d, J=5.0 Hz, 1H, Ar—H), 7.896 (d, J=8.5 Hz, 2H, Ar—H), 7.764-7.700 (m, 2H, 2Ar—H), 7.263-7.213 (m, 4H, 4Ar—H), 7.196 (m, 2H, 2Ar—H), 7.116 (d, J=7.0 Hz, 2Ar—H), 5.955 (m, 1H, NH), 5.405 (d, J=10 Hz, 1H, NH), 5.263 (d, J=9.0 Hz, 1H, NH), 5.038 (m, 1H, NH), 4.380-4.334 (m, 1H, CHCH₂Ph), 4.231-4.144 (m, 4H, 2CHBu^{t}, and CH₂), 3.743-3.526 (m, 16H, OCH₃, 6CH₂ and CHOCO), 3.362 (s, 3H, CH₃), 3.244-3.237 (m, 1H, CH), 2.776-2.718 (m, 2H, CH₂), 2.642-2.598 (m, 1H, CH), 2.379 (t, J=7.0-7.5 Hz, CH₂), 1.747 (m, 2H, CH₂), 1.056 (t, J=7.0-7.5 Hz, 3H, CH₃), 0.838 (s, 9H, Bu^{t}), 0.760 (s, 9H, Bu^{t}). LC-MS: 907.5 (MH⁺).

### Example 7

### Synthesis of mPEG₅-Atazanavir Butyrate

mPEG₅-Atazanavir butyrate was prepared in accordance with the schematic provided below.

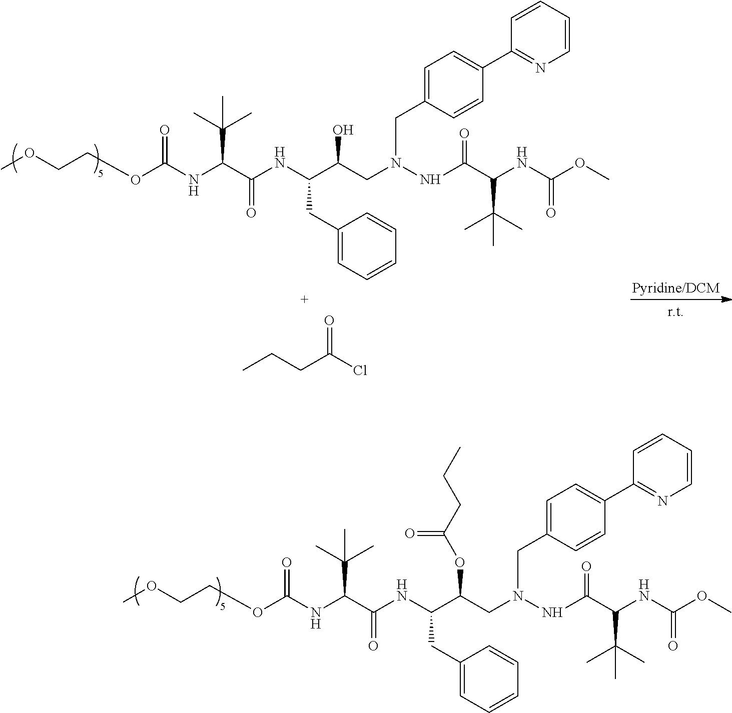

Previously prepared mPEG₅-atazanavir (1.0667 g. 1.15 mmol) was dissolved in anhydrous dichloromethane (30 mL) at room temperature. Anhydrous pyridine (0.9 mL, 11.13 mmol) was added. The mixture was cooled to 0° C., butyryl chloride (0.35 mL, 3.31 mmol) was added dropwise. The resulting mixture was stirred for sixteen hours, during which the temperature was warmed from 0° C. to room temperature. 5% NaHCO₃ aqueous solution was added to quench the reaction. The mixture was concentrated to remove the organic solvent and the remaining mixture was extracted with ethyl acetate (2×80 mL). The ethyl acetate solution was washed with saturated NaCl solution (pH 0.98 by addition of 1N HCl) (4×100 mL), 5% NaHCO₃ aqueous solution (2×100 mL) and saturated NH₄Cl solution, dried over Na₂SO₄, concentrated. The residue was purified with flash column chromatography on silica gel and reverse column chromatography to afford the product mPEG₅-atazanavir butyrate (1.059 g, yield: 92%). ¹H-NMR (CDCl₃): 8.678 (d, J=5.0 Hz, 1H, Ar—H), 7.894 (d, J=8.5 Hz, 2H, Ar—H), 7.761-7.697 (m, 2H, 2Ar—H), 7.257-7.207 (m, 4H, 4Ar—H), 7.179 (m, 2H, 2Ar—H), 7.113 (d, J=7.0 Hz, 2Ar—H), 5.946 (m, 1H, NH), 5.414 (d, J=9.0 Hz, 1H, NH), 5.268 (d, J=9.0 Hz, 1H, NH), 5.041 (m, 1H, NH), 4.359-4.312 (m, 1H, CHCH₂Ph), 4.238-4.140 (m, 4H, 2CHBu^{t}, and CH₂), 3.741-3.522 (m, 24H, OCH₃, 10CH₂ and CHOCO), 3.362 (s, 3H, CH₃), 3.269-3.242 (m, 1H, CH), 2.773-2.716 (m, 2H, CH₂), 2.639-2.594 (m, 1H, CH), 2.375 (t, J=7.5 Hz, CH₂), 1.715 (m, 2H, CH₂), 1.053 (t, J=7.5 Hz, 3H, CH₃), 0.836 (s, 9H, Bu^{t}), 0.757 (s, 9H, Bu^{t}). LC-MS: 995.6 (MH⁺).

### Example 8

### Synthesis of mPEG₆-Atazanavir Butyrate

mPEG₆-Atazanavir butyrate was prepared in accordance with the schematic provided below.

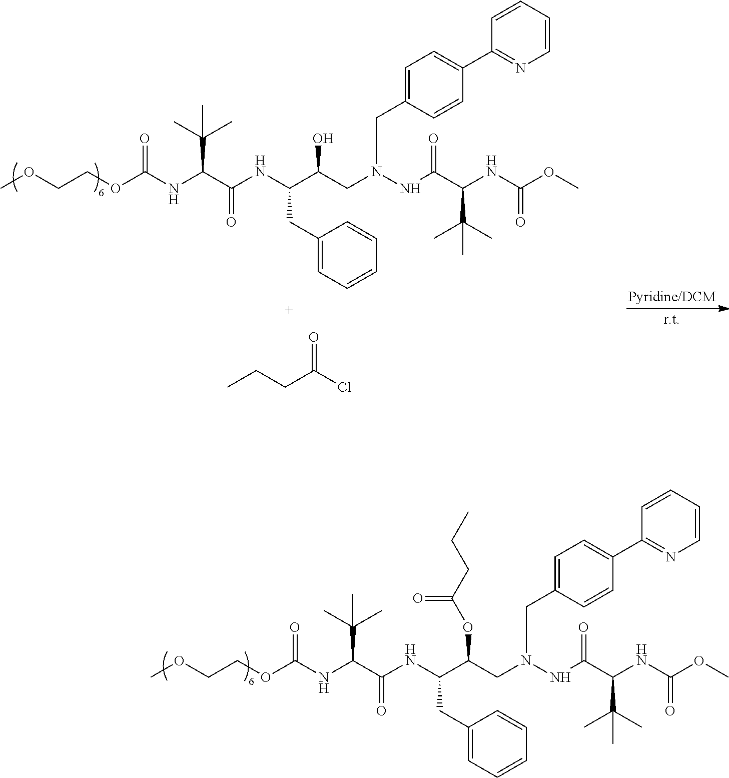

Previously prepared mPEG₆-atazanavir (2.6064 g. 2.69 mmol) was dissolved in anhydrous dichloromethane (80 mL) at room temperature. Anhydrous pyridine (2.2 mL, 27.20 mmol) was added. The mixture was cooled to 0° C., butyryl chloride (0.9 mL, 8.51 mmol) was added dropwise. The resulting mixture was stirred at 0° C. for fifty minutes, and then at room temperature for 22 hours. 5% NaHCO₃ aqueous solution was added to quench the reaction. The mixture was concentrated to remove the organic solvent and the remaining mixture was extracted with ethyl acetate (2×90 mL). The ethyl acetate solution was washed with saturated NaCl solution (pH˜1.24 by addition of 1N HCl) (3×150 mL), 5% NaHCO₃ aqueous solution (2×150 mL) and saturated NH₄Cl solution, dried over Na₂SO₄, concentrated. The residue was purified with flash column chromatography on silica gel and reverse column chromatography to afford 2.39 g of product mPEG₆-atazanavir butyrate in 86% yield. ¹H-NMR (CDCl₃): 8.679 (d, J=4.0 Hz, 1H, Ar—H), 7.895 (d, J=8.5 Hz, 2H, Ar—H), 7.764-7.699 (m, 2H, 2Ar—H), 7.254-7.213 (m, 4H, 4Ar—H), 7.195-7.180 (m, 2H, 2Ar—H), 7.113 (d, J=7.0 Hz, 2Ar—H), 5.925 (m, 1H, NH), 5.406 (d, J=9.0 Hz, 1H, NH), 5.254 (d, J=9.0 Hz, 1H, NH), 5.040 (m, 1H, NH), 4.335-4.308 (m, 1H, CHCH₂Ph), 4.229-4.151 (m, 4H, 2CHBu^{t}, and CH₂), 3.737-3.526 (m, 28H, OCH₃, 12 CH₂ and CHOCO), 3.366 (s, 3H, CH₃), 3.260-3.233 (m, 1H, CH), 2.773-2.736 (m, 2H, CH₂), 2.636-2.580 (m, 1H, CH), 2.387 (t, J=7.0-7.5 Hz, CH₂), 1.733 (m, 2H, CH₂), 1.055 (t, J=7.0-7.5 Hz, 3H, CH₃), 0.835 (s, 9H, Bu^{t}), 0.755 (s, 9H, Bu^{t}). LC-MS: 1039.6 (MH⁺).

### Example 9

### Synthesis of mPEG₃-Atazanavir Propionate

mPEG₃-Atazanavir propionate was prepared in accordance with the schematic provided below.

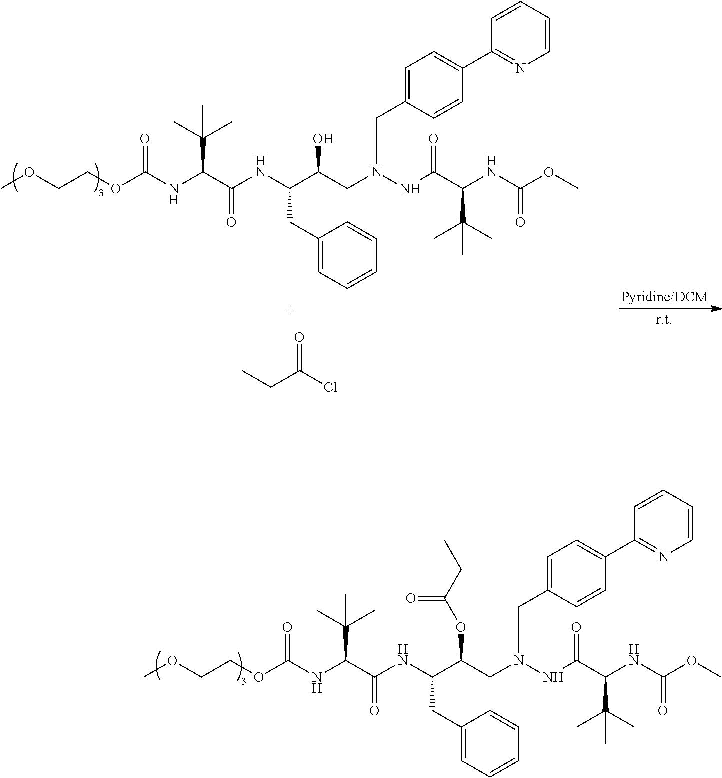

Propionyl chloride (1.2 mL, 13.46 mmol) was added dropwise to a stirred mixture of previously prepared mPEG₃-atazanavir (3.6235 g. 4.329 mmol) and anhydrous pyridine (3.5 mL, 43.27 mmol) in anhydrous dichloromethane (100 mL) at 0° C. The resulting mixture was stirred at 0° C. for about two hours and then at room temperature for twenty hours. More of propionyl chloride (0.06 mL, 0.67 mmol) was added. The reaction mixture was stirred at room temperature for another five hours. 5% NaHCO₃ aqueous solution was added to quench the reaction. The mixture was concentrated to remove the organic solvent, and the remaining mixture was extracted with ethyl acetate (2×100 mL). The ethyl acetate solution was washed with saturated NaCl solution (pH˜1.0 by addition of 1N HCl) (4×150 mL), 5% NaHCO₃ aqueous solution (2×150 mL) and saturated NH₄Cl solution (120 mL), dried over Na₂SO₄, concentrated. The residue was purified with flash column chromatography on silica gel and reverse column chromatography to afford the product mPEG₃-atazanavir propionate (2.5104 g, yield: 65%). ¹H-NMR (CDCl₃): 8.682 (m, 1H, Ar—H), 7.902 (d, J=8.5 Hz, 2H, Ar—H), 7.766-7.700 (m, 2H, 2Ar—H), 7.258-7.170 (m, 6H, 6Ar—H), 7.117 (d, J=8.0 Hz, 2Ar—H), 5.958 (m, 1H, NH), 5.416 (d, J=9.5 Hz, 1H, NH), 5.269 (d, J=9.0 Hz, 1H, NH), 5.043 (m, 1H, NH), 4.359-4.314 (m, 1H, CHCH₂Ph), 4.237-4.145 (m, 4H, 2CHBu^{t}, and CH₂), 3.739-3.529 (m, 16H, OCH₃, 6 CH₂ and CHOCO), 3.364 (s, 3H, CH₃), 3.268-3.240 (m, 1H, CH), 2.778-2.708 (m, 2H, CH₂), 2.650-2.607 (m, 1H, CH), 2.414 (q, J=7.5 Hz, CH₂), 1.232 (t, J=7.5 Hz, 3H, CH₃), 0.838 (s, 9H, Bu^{t}), 0.769 (s, 9H, Bu^{t}). LC-MS: 893.5 (MH⁺).

### Example 10

### Synthesis of mPEG₅-Atazanavir Propionate

mPEG₅-Atazanavir propionate was prepared in accordance with the schematic provided below.

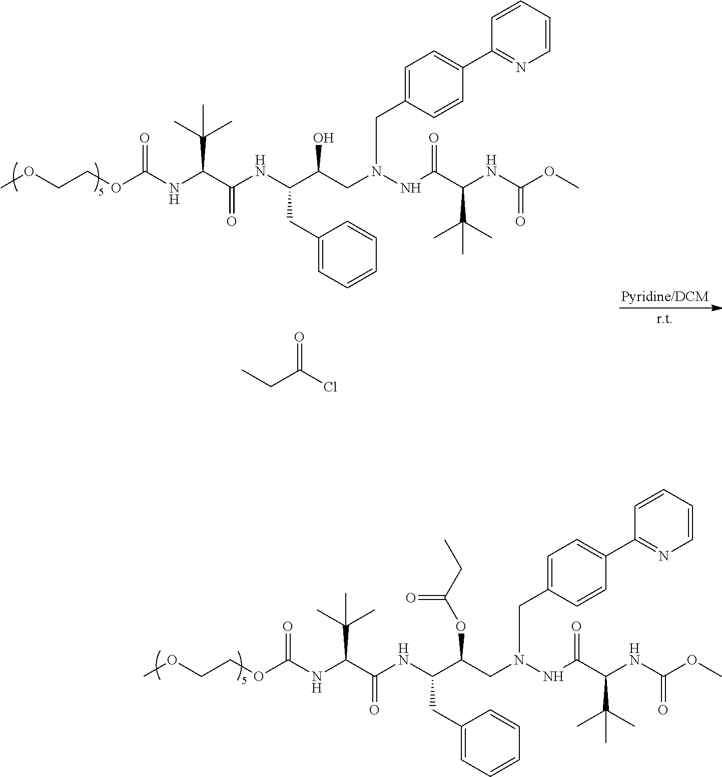

Propionyl chloride (1.04 mL, 11.67 mmol) was added dropwise to a stirred mixture of previously prepared mPEG₅-atazanavir (3.5970 g. 3.888 mmol) and anhydrous pyridine (3.2 mL, 11.67 mmol) in anhydrous dichloromethane (60 mL) at 0° C. The resulting mixture was stirred at 0° C. for about two hours then at room temperature for 21.5 hours. More propionyl chloride (0.05 mL, 0.561 mmol) was added. The reaction mixture was stirred at room temperature for another five hours 5% NaHCO₃ aqueous solution was added to quench the reaction. The mixture was concentrated to remove the organic solvent and the remaining mixture was extracted with ethyl acetate (2×120 mL). The ethyl acetate solution was washed with saturated NaCl solution (pH 0.98 by addition of 1N HCl) (3×150 mL), 5% NaHCO₃ aqueous solution (2×180 mL), dried over Na₂SO₄, concentrated. The residue was purified with flash column chromatography on silica gel and reverse column chromatography to afford the product mPEG₅-atazanavir propionate (2.0763 g, yield: 54%). ¹H-NMR (CDCl₃): 8.682 (m, 1H, Ar—H), 7.901 (d, J=8.5 Hz, 2H, Ar—H), 7.766-7.703 (m, 2H, 2Ar—H), 7.250-7.169 (m, 6H, 6Ar—H), 7.115 (d, J=7.0 Hz, 2Ar—H), 5.944 (m, 1H, NH), 5.416 (d, J=9.0 Hz, 1H, NH), 5.267 (d, J=9.0 Hz, 1H, NH), 5.047-5.031 (m, 1H, NH), 4.341-4.310 (m, 1H, CHCH₂Ph), 4.228-4.141 (m, 4H, 2CHBu^{t}, and CH₂), 3.737-3.527 (m, 24H, OCH₃, 10CH₂ and CHOCO), 3.367 (s, 3H, CH₃), 3.275-3.247 (m, 1H, CH), 2.788-2.707 (m, 2H, CH₂), 2.671-2.592 (m, 1H, CH), 2.412 (q, J=7.5 Hz, CH₂), 1.232 (t, J=7.5 Hz, 3H, CH₃), 0.838 (s, 9H, Bu^{t}), 0.767 (s, 9H, Bu^{t}). LC-MS: 981.5 (MH⁺).

### Example 11

### Synthesis of mPEG₆-Atazanavir Propionate

mPEG₆-Atazanavir propionate was prepared in accordance with the schematic provided below.

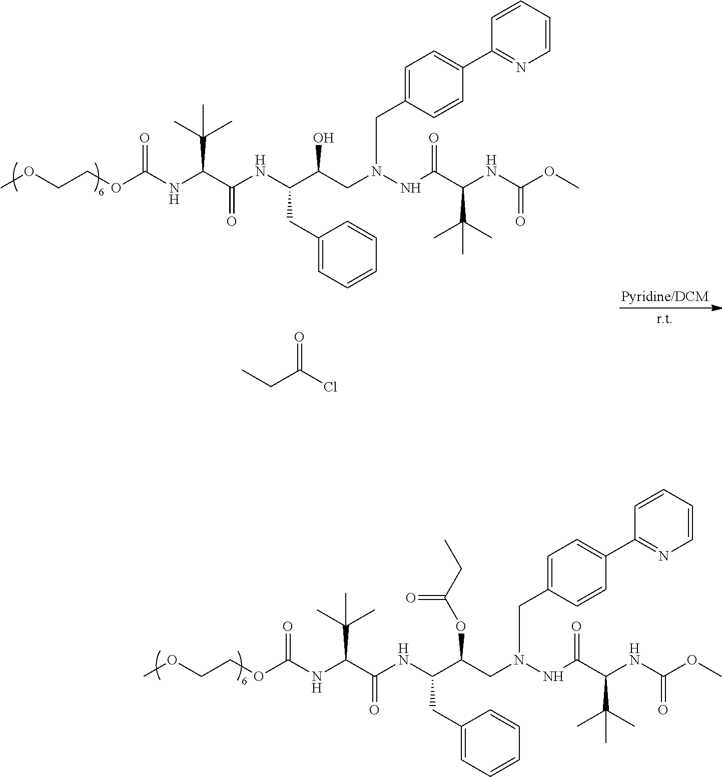

Propionyl chloride (1.03 mL, 11.55 mmol) was added dropwise to a stirred mixture of previously prepared mPEG₆-atazanavir (3.6864 g. 3.804 mmol) and anhydrous pyridine (3.09 mL, 38.20 mmol) in anhydrous dichloromethane (95 mL) at 0° C. The resulting mixture was stirred at 0° C. for about two hours, at room temperature for 16.5 hours. More of propionyl chloride (0.075 mL, 0.84 mmol) was added. The reaction mixture was stirred at room temperature for another four hours. 5% NaHCO₃ aqueous solution was added to quench the reaction. The mixture was concentrated to remove the organic solvent, and the remaining mixture was extracted with ethyl acetate (2×100 mL). The ethyl acetate solution was washed with saturated NaCl solution (pH 0.98 by addition of 1N HCl) (4×120 mL), 5% NaHCO₃ aqueous solution (3×120 mL) and saturated NH₄Cl solution, dried over Na₂SO₄, concentrated. The residue was purified with flash column chromatography on silica gel and reverse column chromatography to afford the product mPEG₆-atazanavir propionate (1.9375 g, yield: 50%). ¹H-NMR (CDCl₃): 8.682 (d, J=4.5 Hz, 1H, Ar—H), 7.903 (d, J=8.5 Hz, 2H, Ar—H), 7.768-7.704 (m, 2H, 2Ar—H), 7.258-7.226 (m, 4H, 4Ar—H), 7.199-7.170 (m, 2H, 2Ar—H), 7.115 (d, J=7.0 Hz, 2Ar—H), 5.947 (m, 1H, NH), 5.413 (d, J=9.5 Hz, 1H, NH), 5.265 (d, J=8.5 Hz, 1H, NH), 5.058-5.038 (m, 1H, NH), 4.361-4.312 (m, 1H, CHCH₂Ph), 4.207-4.140 (m, 4H, 2CHBu^{t}, and CH₂), 3.740-3.530 (m, 28H, OCH₃, 12CH₉ and CHOCO), 3.370 (s, 3H, CH₃), 3.270-3.227 (m, 1H, CH), 2.775-2.712 (m, 2H, CH₂), 2.652-2.607 (m, 1H, CH), 2.424 (q, J=7.5 Hz, CH₂), 1.232 (t, J=7.5 Hz, 3H, CH₃), 0.840 (s, 9 H, Bu^{t}), 0.770 (s, 9H, LC-MS: 1025.6 (MH⁺).

### Examples 12a-12c

### Preparation of O-Acetyl-mPEGₙ-Atazanavir Compounds

O-Acetyl-mPEGₙ-atazanavir compounds were prepared in accordance with the schematic provided below.

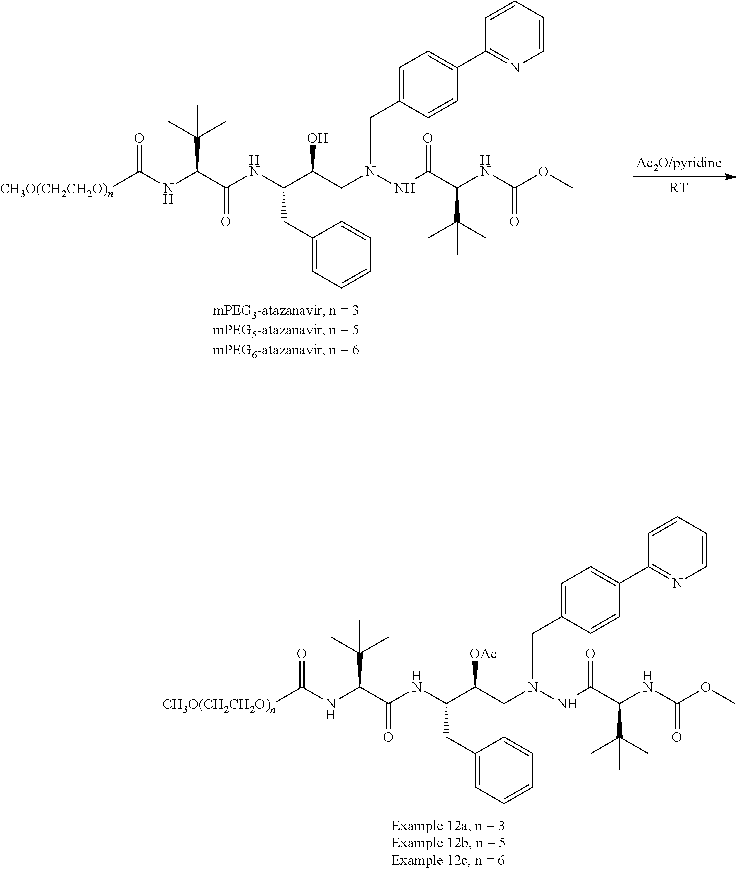

### Example 12a

### O-Acetyl-mPEG₃-Atazanavir

Previously prepared mPEG₃-atazanavir (3.5 g, 4.2 mmol) was added to anhydrous pyridine (3.5 ml, 32.9 mmol). Acetic anhydride (1.22 ml, 12.7 mmol) was added and stirred at room temperature for 21 hours. The reaction solution was diluted in DCM (150 ml) and washed with 0.1 N HCl solution (160 ml×3, additional 1.0 N HCl solution was added to the first extraction to adjust PH=2). The organic phase was separated and washed with sat. NaHCO₃ solution (100 ml×2). The organic phase was separated and dried with anhydrous Na₂SO₄. After removal of the solid by filtration, the solvent was evaporated. The residue was dissolved in ethyl acetate (10 ml). Hexanes (300 ml) were added to the solution to form a white precipitate. The white solid product, O-acetyl-mPEG₃-atazanavir, (3.20 g, yield 87%) was obtained after filtration and dried under vacuum overnight. ¹H NMR (CDCl₃) δ 8.68-8.67 (m, 1H), 7.91 (d, 2H), 7.75-7.71 (m 2H), 7.28-7.13 (m, 6H), 7.12 (d, 2H), 5.88-5.85 (m, 1H), 5.42-5.40 (m, 1H), 5.31-5.29 (m, 1H), 5.06-5.04 (m, 1H), 4.36-4.33 (m, 1H), 4.24-4.12 (m, 4H), 3.75-3.51 (m, 15H), 3.36 (s, 3H), 3.28-3.26 (m, 1H), 2.82-2.59 (m, 3H), 2.15 (s, 3H), 0.84 (s, 9H), 0.77 (s, 9H). LC/MS 879 [M+H]⁺, 901 [M+Na]⁺, 917 [M+K]⁺.

### Example 12b

### O-Acetyl-mPEG₅-Atazanavir

Previously prepared mPEG₅-Atazanavir (2.98 g, 3.23 mmol) was added to anhydrous pyridine (2.5 ml, 23.5 mmol). Acetic anhydride (0.87 ml, 9.1 mmol) was added and stirred at room temperature for 18 hours. The reaction solution was diluted in DCM (150 ml) and washed with 0.1 N HCl solution (160 ml×3, additional 1.0 N HCl solution was added to the first extraction to adjust PH=2). The organic phase was separated and washed with sat. NaHCO₃ solution (100 ml×2). The organic phase was separated and dried with anhydrous Na₂SO₄. After removal of the solid by filtration, the solvent was evaporated. The residue was dissolved in ethyl acetate (10 ml). Hexanes (300 ml) were added to the solution to form a white precipitate. The white solid product, O-acetyl-mPEG₅-atazanavir, (3.1 g, yield 99%) was obtained after filtration and dried under vacuum overnight. ¹H NMR (CDCl₃) δ 8.68-8.67 (m, 1H), 7.91 (d, 2H), 7.74-7.70 (m 2H), 7.28-7.17 (m, 6H), 7.12 (d, 2H), 5.95-5.93 (m, 1H), 5.41-5.39 (m, 1H), 5.30-5.28 (m, 1H), 5.06-5.04 (m, 1H), 4.37-4.34 (m, 1H), 4.25-4.17 (m, 4H), 3.73-3.52 (m, 23H), 3.36 (s, 3H), 3.27-3.25 (m, 1H), 2.81-2.60 (m, 3H), 2.15 (s, 3H), 0.84 (s, 9H), 0.77 (s, 9H). LC/MS 967 [M+H]⁺, 989 [M+Na]⁺, 1005 [M+K]⁺.

### Example 12c

### O-Acetyl-mPEG₆-Atazanavir

Previously prepared mPEG₆-Atazanavir (2.71 g, 2.80 mmol) was added to anhydrous pyridine (2.28 ml, 28 mmol). Acetic anhydride (0.81 ml, 8.4 mmol) was added and stirred at room temperature for eighteen hours. The reaction solution was diluted in DCM (150 ml) and washed with 0.1 N HCl solution (160 ml×3, additional 1.0 N HCl solution was added to the first extraction to adjust PH=2). The organic phase was separated and washed with saturated NaHCO₃ solution (100 ml×2). The organic phase was separated and dried with anhydrous Na₂SO₄. After removal of the solid by filtration, the solvent was evaporated. The residue was dissolved in ethyl acetate (10 ml). Hexanes (300 ml) were added to the solution to form a white precipitate. The white solid product, O-acetyl-mPEG₅-atazanavir, (2.45 g, yield 87%) was obtained after filtration and dried under vacuum overnight. ¹H NMR (CDCl₃) δ 8.6-8.66 (m, 1H), 7.89 (d, 2H), 7.73-7.68 (m 2H), 7.26-7.17 (m, 6H), 7.11 (d, 2H), 6.01-5.98 (m, 1H), 5.43-5.41 (m, 1H), 5.31-5.29 (m, 1H), 5.00-4.98 (m, 1H), 4.36-4.31 (m, 1H), 4.28-4.17 (m, 4H), 3.75-3.52 (m, 27H), 3.36 (s, 3H), 3.21-3.18 (m, 1H), 2.75-2.56 (m, 3H), 2.15 (s, 3H), 0.83 (s, 9H), 0.76 (s, 9H). LC/MS 1011 [M+H]⁺, 1033 [M+Na]⁺.

### Examples 13a-13c

### Preparation of O-Octanoyl-mPEGₙ-Atazanavir Compounds

O-Octanoyl-mPEGₙ-atazanavir compounds were prepared in accordance with the schematic provided below.

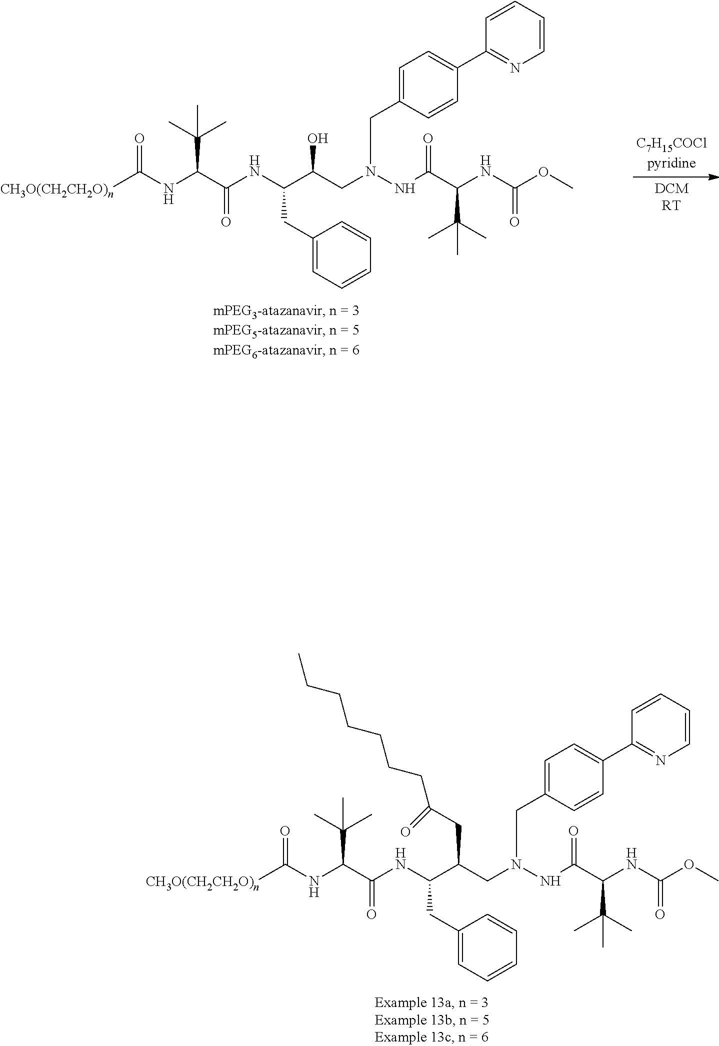

### Example 13a

### O-Octanoyl-mPEGₙ-Atazanavir

This compound can be prepared in accordance with the approach set forth for Example 13b, wherein mPEG₃-atazanavir is substituted for mPEG₅-atazanavir monophosphate.

### Example 13b

### O-Octanoyl-mPEG₆-Atazanavir

Previously prepared mPEG₅-atazanavir (3.30 g, 3.57 mmol) was dissolved in anhydrous DCM (40 ml) and anhydrous pyridine (2.9 ml, 35.7 mmol). At room temperature, octanoyl chloride (1.82 ml, 10.7 mmol) was added slowly into the stirred solution. The solution was stirred at room temperature for 4.5 hours. Saturated NaHCO₃ solution (10 ml) was added and stirred for five minutes. DCM was evaporated and the residue solution was extracted with ethyl acetate (200 ml) and 0.1 N HCl solution (100 ml×3, additional 1.0 N HCl solution was added to the first extraction to adjust PH=2). The organic phase was separated and washed with sat. NaHCO₃ solution (100 ml×2). The organic phase was separated and dried with anhydrous Na₂SO₄. After removal of the solid by filtration, the solvent was evaporated.

### Example 13c

### O-Octanoyl-mPEG₆-Atazanavir

Previously prepared mPEG₆-Atazanavir (2.90 g, 3.0 mmol) was dissolved in anhydrous DCM (30 ml) and anhydrous pyridine (2.44 ml, 30 mmol). At room temperature, octanoyl chloride (1.53 ml, 9.0 mmol) was added slowly into the stirred solution. The solution was stirred at room temperature for six hours. Saturated NaHCO₃ solution (10 ml) was added and stirred for five minutes. DCM was evaporated and the residue solution was extracted with ethyl acetate (200 ml) and 0.1 N HCl solution (100 ml×3, additional 1.0 N HCl solution was added to the first extraction to adjust pH=2). The organic phase was separated and washed with sat. NaHCO₃ solution (100 ml×2). The organic phase was separated and dried with anhydrous Na₂SO₄. After removal of the solid by filtration, the solvent was evaporated. The residue was subjected to flash chromatography (methanol in DCM 1%˜4%) to obtain O-octanoyl-mPEG₆-atazanavir (2.24 g, yield 68%) as clear glassy semi solid. ¹H NMR (CDCl₃) δ 8.69-8.67 (m, 1H), 7.90 (d, 2H), 7.75-7.70 (m 2H), 7.25-7.12 (m, 6H), 7.11 (d, 2H), 5.98-5.96 (m, 1H), 5.43-5.41 (m, 1H), 5.35-5.33 (m, 1H), 5.06-5.04 (m, 1H), 4.38-4.32 (m, 1H), 4.23-4.15 (m, 4H), 3.70-3.51 (m, 27H), 3.37 (s, 3H), 3.23-3.20 (m, 1H), 2.79-2.52 (m, 3H), 2.39 (t, 2H), 1.70-1.68 (m, 2H), 1.45-1.25 (m, 8H), 0.91 (t, 3H), 0.84 (s, 9H), 0.76 (s, 9H). LC/MS 1095 [M+H]⁺, 1117 [M+Na]⁺.

### Example 14

### Preparation of mPEG₆-Atazanavir-L-Valine HCl

mPEG₆-Atazanavir-L-valine HCl was prepared in two steps. Schematically, the first step can be schematically represented as follows.

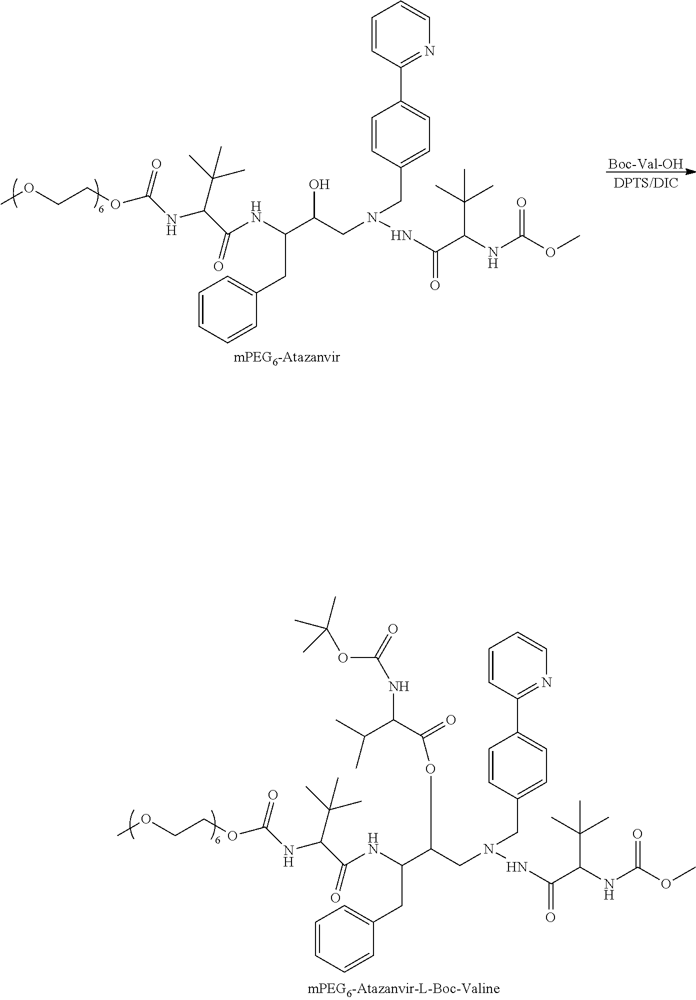

Thereafter, the second step to provide the desired mPEG₆-atazanavir-L-valine HCl can be schematically represented as follows.

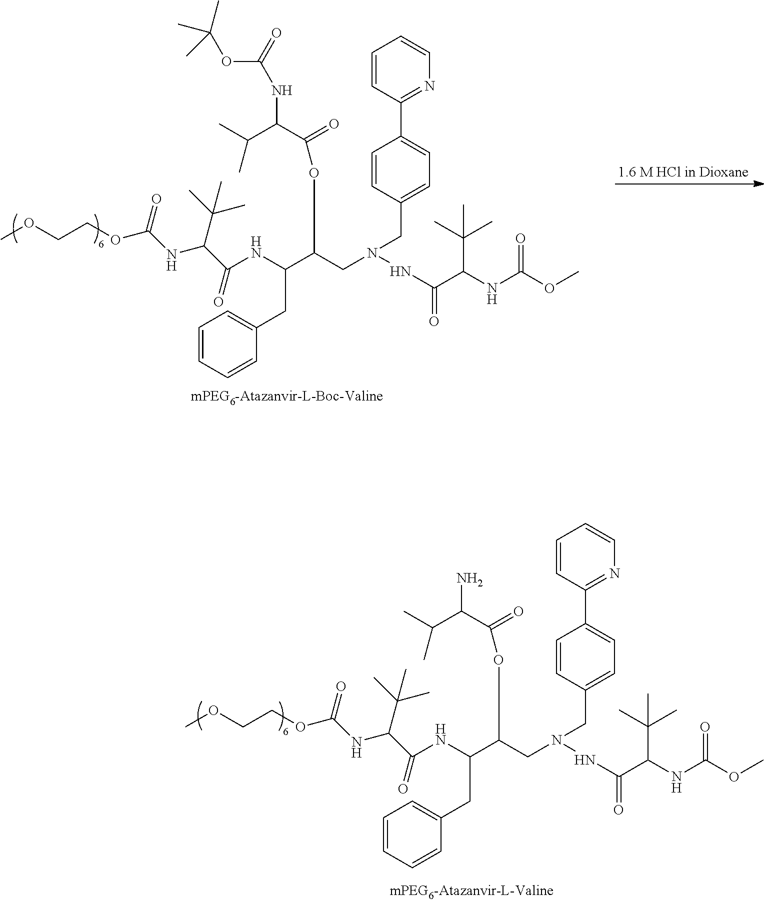

### mPEG₆-Atazanavir-L-Boc-valine

Previously prepared mPEG₆-Atazanavir (2.62 g; 2.7 mmol), Boc-L-valine (11.72 g, 54 mmol), and DPTS (1:1 salt of 4 (Dimethylamino)pyridine and p-toluensulfonic acid, 0.84 g, 2.7 mmol) were dissolved in 100 ml of DCM. To the solution, DIC (8.4 g, 67.5 mmol) was added dropwise under stirring. The resulting mixture was stirred at room temperature for four hours. After this period, the solid was filtered out and 150 ml of DCM was added into the filtration. The solution was washed with H₂O (100 ml), 5% NaHCO₃ (100 ml), and H₂O (100 ml). The organic phase was dried over Na₂SO₄ and concentrated under reduced pressure. The residue was purified by column chromatography (biotage: DCM/CH₃OH, CH₃OH: 3-6%, 15 CV). The product, mPEG₆-atazanavir-L-Boc-valine, was obtained as a white solid (2.77 g, yield: 88%). ¹H NMR (500 MHz, CDCl₃) δ 0.77 (s, 9H), 0.91 (s, 9H), 1.03 (d, 3H), 1.14 (d, 3H), 1.48 (s, 9H), 1.88 (br., 3H), 2.25 (m, 1H), 2.60 (m, 1H), 2.75 (m, 1H), 3.10 (br., 2H), 3.38 (s, 3H), 3.54 (m, 2H), 3.65 (m, 22H), 3.76 (m, 1H), 4.25 (br., 6H), 5.12 (d, 1H), 5.18 (m, 1H), 5.32 (m, 1H), 5.45 (m, 1H), 6.00 (m, 1H), 7.15 (m, 3H), 7.22 (m, 3H), 7.30 (d, 2H), 7.72 (m, 2H), 7.90 (d, 2H), 8.68 (d, 1H), LC-MS (m/z) calcd., 1167.7, found 1168.6 [M+H]+.

### mPEG₆-Atazanavir-L-Valine

mPEG₆-Atazanavir-L-Boc-valine (3.50 g, 3.0 mmol) was dissolved in 15 ml of dioxane. To the solution, 10 ml of 4.0 M HCl in dioxane was added. The mixture was stirred at room temperature for two hours. After this period, 200 ml of DCM was added into the reaction mixture. The resulting solution was washed with saturated NaCl (100 ml) and dried over Na₂SO₄. The reaction mixture was then concentrated under reduced pressure, and the product, mPEG₆-atazanavir-L-valine, was obtained as white solid (HCl salt, yield: 95%). ¹H NMR (500 MHz, DMSO) δ 0.72 (s, 9H), 0.82 (s, 9H), 1.04 (m, 6H), 2.72 (d, 2H), 3.00 (m, 2H), 3.22 (s, 3H), 3.41 (m, 5H), 3.50 (m, 20H), 3.75 (d, 1H), 3.98 (m, 4H), 4.06 (m, 2H), 4.65 (m, 2H), 5.09 (m, 1H), 6.75 (dd, 2H), 7.15 (m, 1H), 7.20 (m, 5H), 7.42 (m, 3H), 7.95 (d, 2H), 8.08 (m, 3H), 8.75 (m, 4H), 9.10 (s, 1H); LC-MS (m/z) calcd., 1067.7, found 1068.7 [M+H]+.

### Examples 15a and 15b

### Preparation of mPEGₙ-Atazanavir-L-Leucine Compounds

mPEGₙ-Atazanavir-L-leucine compounds were prepared in two steps. Schematically, the first step can be schematically represented as follows.

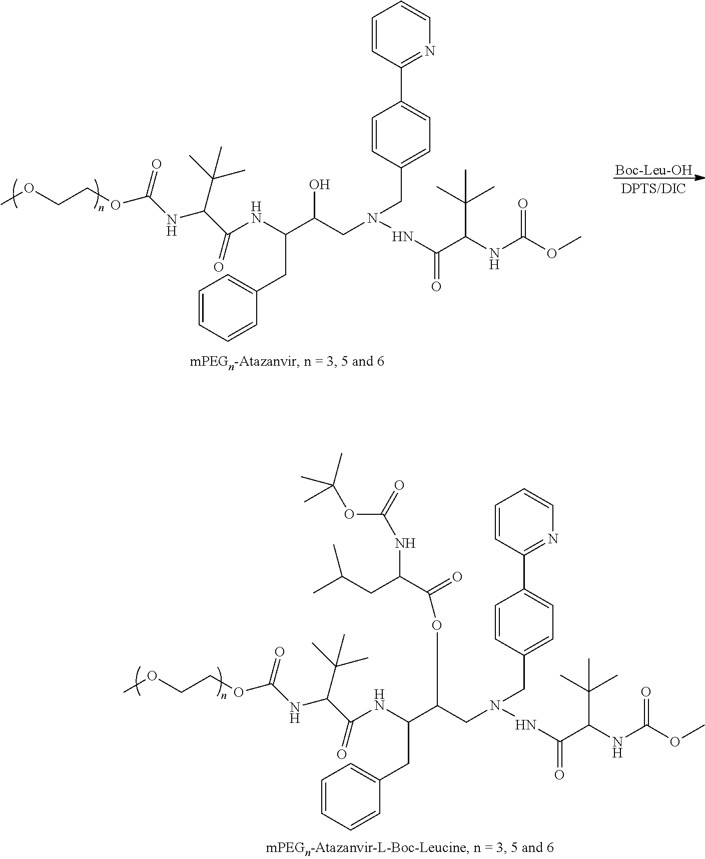

Thereafter, the second step to provide the desired mPEGₙ-atazanavir-L-leucine compounds can be schematically represented as follows.

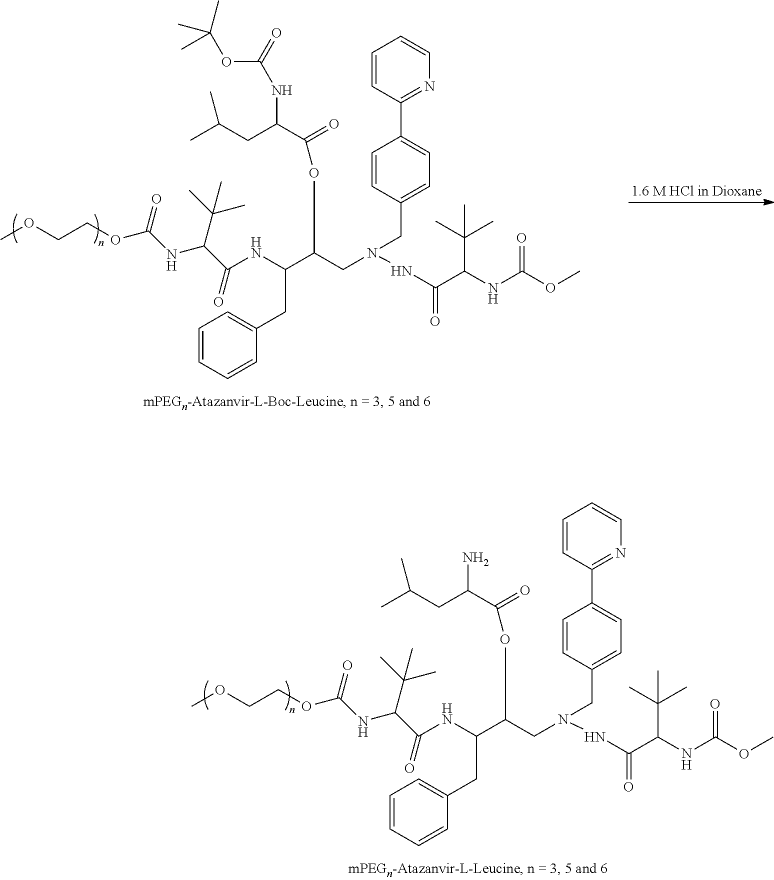

### mPEGn-Atazanavir-L-Sac-Leucine (n=3, 5 and 6)

Previously prepared mPEGₙ-atazanavir (n=3, 5 and 6; 4.1 mmol), Boc-Leu-OH (19.0 g, 82 mmol), and DPTS (1:1 salt of 4 (Dimethylamino)pyridine and p-toluensulfonic acid, 1.27 g, 4.1 mmol) were dissolved in 100 ml of DCM. To the solution, DIC (13.0 g, 103.2 mmol) was added dropwise under stirring. The resultant mixture was stirred at room temperature for four hours. After this period, the solid was filtered out and 200 ml of DCM was added into the filtration. The solution was washed with H₂O (200 ml), 5% NaHCO₃ (200 ml), and H₂O (200 ml). The organic phase was dried over Na₂SO₄ and concentrated under reduced pressure. The residue was purified by column chromatography (biotage: DCM/CH₃OH, CH₃OH: 3-6%, 15 CV). The product, mPEGn-atazanavir-L-Boc-leucine (n=3, 5 and 6), was obtained as a white solid (yield: 71-76%)

mPEG₃-Atazanavir-L-Boc-Leucine: ¹H NMR (500 MHz, DMSO) δ 0.72 (s, 9H), 0.82 (s, 9H), 0.87 (d, 3H), 0.92 (d, 3H), 1.36 (s, 9H), 1.50-1.70 (m, 4H), 2.65 (m, 2H), 3.00 (m, 2H), 3.22 (s, 3H), 3.40 (m, 5H), 3.50 (m, 7H), 3.56 (m, 2H), 3.65 (d, 1H), 3.98 (m, 3H), 4.06 (m, 2H), 4.12 (m, 1H), 4.57 (m, 1H), 4.92 (m, 1H), 6.68 (d, 1H), 6.80 (d, 1H), 7.16 (m, 5H), 7.32 (m, 4H), 7.70 (d, 1H), 7.86 (m, 2H), 7.92 (d, 2H), 8.62 (d, 1H), 8.88 (s, 1H); LC-MS (m/z) calcd., 1049.6, found 1050.6 [M+H]+.

mPEG₅-Atazanavir-L-Boc-Leucine: ¹H NMR (500 MHz, DMSO) δ 0.72 (s, 9H), 0.82 (s, 9H), 0.87 (d, 3H), 0.92 (d, 3H), 1.36 (s, 9H), 1.50-1.70 (m, 4H), 2.65 (m, 2H), 3.00 (m, 2H), 3.22 (s, 3H), 3.40 (m, 5H), 3.50 (m, 15H), 3.56 (m, 2H), 3.65 (d, 1H), 3.98 (m, 3H), 4.06 (m, 2H), 4.12 (m, 1H), 4.57 (m, 1H), 4.92 (m, 1H), 6.68 (d, 1H), 6.80 (d, 1H), 7.16 (m, 5H), 7.32 (m, 4H), 7.70 (d, 1H), 7.86 (m, 2H), 7.92 (d, 2H), 8.62 (d, 1H), 8.88 (s, 1H); LC-MS (m/z) calcd., 1137.7, found 1138.8 [M+H]+.

### Synthesis of mPEGₙ-Atazanavir-L-Leucine (Example 15a when n=3, Example 15b when n=5, and Example 15c when n=6)

mPEGₙ-Atazanavir-L-Boc-leucine (3.0 mmol) was dissolved in 15 ml of dioxane. To the solution, 10 ml of 4.0 M HCl in dioxane was added. The mixture was stirred at room temperature for two hours. After this period, 200 ml of DCM was added into the reaction mixture. The resulted solution was washed with saturated NaCl (100 ml) and dried over Na₂SO₄. The reaction mixture was then concentrated under reduced pressure. The product was obtained as white solid (HCl salt, yield: 90-95%).

mPEG₃-Atazanavir-L-Leucine: ¹H NMR (500 MHz, DMSO) δ 0.72 (s, 9H), 0.82 (s, 9H), 0.94 (m, 6H), 1.75 (m, 1H), 1.85 (m, 2H), 2.75 (m, 2H), 3.00 (m, 2H), 3.22 (s, 3H), 3.41 (m, 5H), 3.50 (m, 7H), 3.75 (d, 1H), 3.98 (m, 3H), 4.06 (m, 3H), 4.57 (m, 1H), 5.09 (m, 1H), 6.75 (m, 2H), 7.15 (m, 1H), 7.20 (m, 4H), 7.42 (d, 2H), 7.52 (m, 1H), 7.95 (d, 2H), 8.08 (m, 3H), 8.75 (m, 4H), 9.20 (s, 1H); LC-MS (m/z) calcd., 949.6, found 950.6 [M+H]+.

mPEG₅-Atazanavir-L-Leucine: ¹H NMR (500 MHz, DMSO) δ 0.72 (s, 9H), 0.82 (s, 9H), 0.94 (m, 6H), 1.75 (m, 1H), 1.85 (m, 2H), 2.75 (m, 2H), 3.00 (m, 2H), 3.22 (s, 3H), 3.41 (m, 5H), 3.50 (m, 15H), 3.75 (d, 1H), 3.98 (m, 3H), 4.06 (m, 3H), 4.57 (m, 1H), 5.09 (m, 1H), 6.75 (m, 2H), 7.15 (m, 1H), 7.20 (m, 4H), 7.42 (d, 2H), 7.52 (m, 1H), 7.95 (d, 2H), 8.08 (m, 3H), 8.75 (m, 4H), 9.20 (s, 1H); LC-MS (m/z) calcd., 1037.6, found 1038.6.6 [M+H]+.

### Example 16

### Synthesis of mPEGₙ-Atazanavir Phospholipids

mPEGₙ-atazanavir phospholipid compounds were prepared in accordance with the general scheme depicted below.

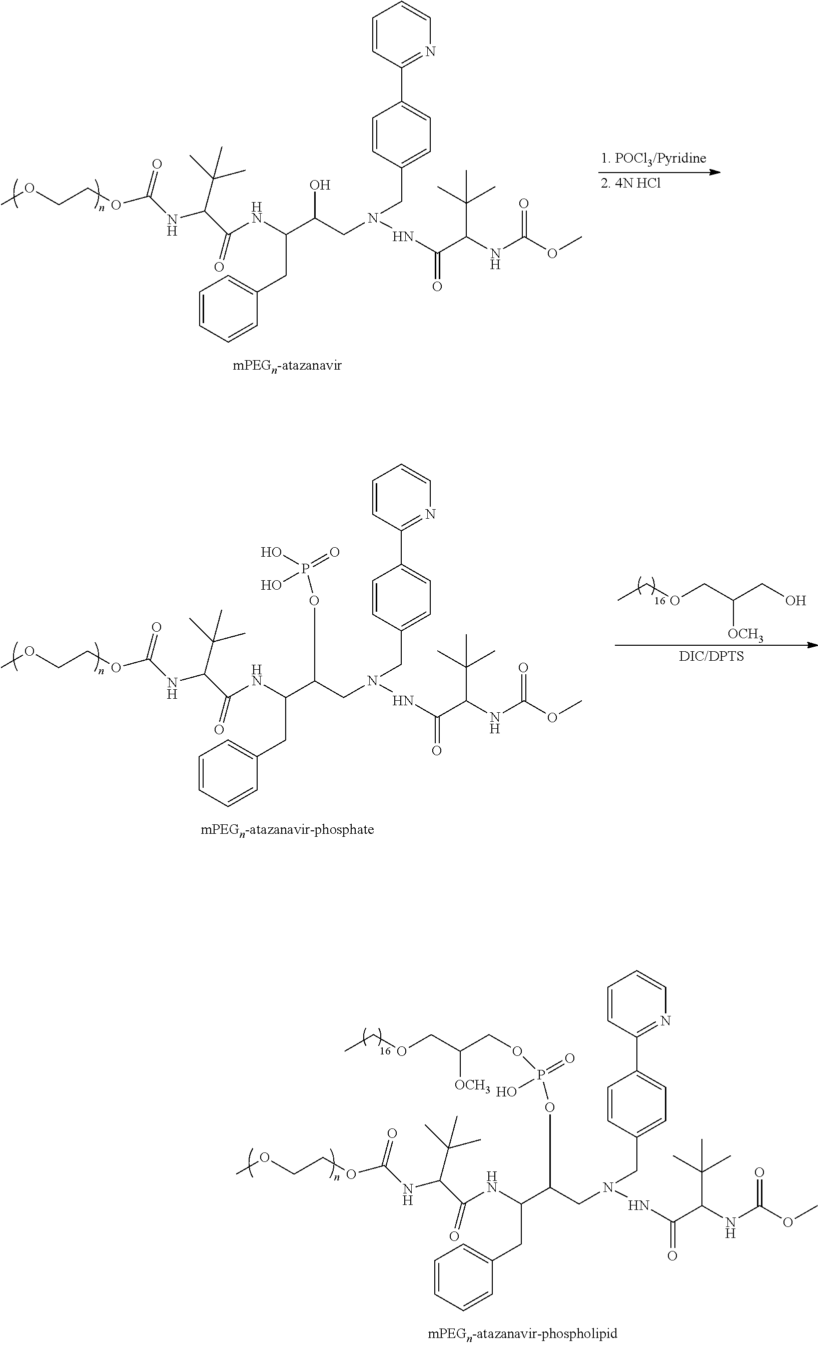

### Preparation of mPEG₃-atazanavir phosphate

Phosphorus oxychloride (8.91 g, 60.0 mmol) was dissolved in methylisobutyl ketone (50 mL). The resulting solution was cooled under stirring in an ice-bath, and then previously prepared mPEG₃-atazanavir (8.37 g, 10.0 mmol) and pyridine (15.33 g, 100 mmol) in 50 mL of methylisobutyl ketone was added dropwise over one hour. After the addition, the reaction was continued at room temperature for three hours before 4 N HCl (100 mL) was added. The mixture was stirred at 60° C. for 2.5 hours. After the reaction, two phases were separated. The methylisobutylketone phase contained complicated impurities along with trace amount of product. The acidic aqueous phase contained product and impurities at the same ratio as reaction mixture. The aqueous phase was first extracted with ethyl acetate (150 mL×3), and then with dichloromethane after saturated with sodium chloride (200 mL×5). The DCM phase was dried over sodium sulfate and solvent was removed by rotary evaporator. Crude product was dissolved in D.I. water (60 mL) and the water solution was extracted with ethyl acetate (50 mL) and then DCM (100 mL×4). The DCM phase was dried over sodium sulfate and solvent was removed by rotary evaporator. The product was obtained as white solid (6.97 g, 7.14 mmol) with a yield of 70%. ¹H NMR (500 MHz, DMSO) δ 0.72 (s, 9H), 0.76 (s, 9H), 2.74-2.95 (m, 4H), 3.22 (s, 3H), 3.35 (br., 3H), 3.41 (m, 2H), 3.50 (m, 6H), 3.55 (m, 2H), 3.65 (d, 1H), 3.95 (d, 1H), 4.05 (m, 2H), 4.10-4.30 (m, 2H), 4.65 (br., 1H), 6.81 (d, 2H), 7.12 (m, 1H), 7.18 (m, 2H), 7.25 (m, 2H), 7.46 (br., 1H), 7.56 (d, 2H), 7.82 (d, 1H), 7.90 (m, 2H), 8.02 (m, 2H), 8.70 (s, 1H), 9.84 (s, 1H); LC-MS (m/z) calcd., 916.5, found 917.5 [M+H]+.

### Preparation of mPEG₅-atazanavir phosphate

Phosphorus oxychloride (4.01 g, 27.0 mmol) was dissolved in methylisobutyl ketone (50 mL). The resulting solution was cooled under stirring in an ice-bath, and then previously prepared mPEG₅-atazanavir (4.16 g, 4.5 mmol) and pyridine (6.90 g, 45 mmol) in 50 mL of methylisobutyl ketone was added dropwise over one hour. After the addition, the reaction was continued at room temperature for three hours before 4 N HCl (100 mL) was added. The mixture was stirred at 60° C. for 2.5 hours. After the reaction, two phases were separated. The methylisobutylketone phase contained complicated impurities along with trace amount of product. The acidic aqueous phase contained product and impurities at the same ratio as reaction mixture. The aqueous phase was first extracted with ethyl acetate (150 mL×3), and then with dichloromethane after saturated with sodium chloride (200 mL×5). The DCM phase was dried over sodium sulfate and solvent was removed by rotary evaporator. Crude product was dissolved in D.I. water (60 mL) and the water solution was extracted with ethyl acetate (50 mL) and then DCM (100 mL×4). The DCM phase was dried over sodium sulfate and solvent was removed by rotary evaporator. The product was obtained as white solid (3.70 g, 3.68 mmol) with a yield of 76%. ¹H NMR (500 MHz, DMSO) δ 0.71 (s, 9H), 0.75 (s, 9H), 2.74-2.95 (m, 4H), 3.23 (s, 3H), 3.32 (br., 3H), 3.41 (m, 2H), 3.50 (m, 14H), 3.55 (m, 2H), 3.65 (d, 1H), 3.95 (d, 1H), 4.05 (m, 2H), 4.10-4.30 (m, 2H), 4.65 (br., 1H), 6.80 (d, 2H), 7.12 (m, 1H), 7.18 (m, 2H), 7.25 (m, 2H), 7.62 (br., 3H), 7.82 (d, 1H), 7.90 (m, 2H), 8.15 (m, 1H), 8.22 (br., 1H), 8.75 (s, 1H), 9.75 (s, 1H); LC-MS (m/z) calcd., 1004.5, found 1005.5 [M+H]+.

### Preparation of mPEG₆-atazanavir phosphate

Phosphorus oxychloride (4.60 g, 30 mmol) was dissolved in methylisobutyl ketone (50 mL). The resulted solution was cooled under stirring in an ice-bath, and then previously prepared mPEG₆-atazanavir (9.69 g, 10 mmol) and pyridine (7.91 g, 100 mmol) in 50 mL of methylisobutyl ketone was added dropwise over one hour. After the addition, the reaction was continued at room temperature for three hours before 4 N HCl (100 mL) was added. The mixture was stirred at 60° C. for 2.5 hours. After the reaction, two phases were separated. The methylisobutylketone phase contained complicated impurities along with trace amount of product. The acidic aqueous phase contained product and impurities at the same ratio as reaction mixture. The aqueous phase was first extracted with ethyl acetate (150 mL×3), and then with dichloromethane after saturated with sodium chloride (200 mL×5). The DCM phase was dried over sodium sulfate and solvent was removed by rotary evaporator. Crude product was dissolved in D.I. water (60 mL) and the water solution was extracted with ethyl acetate (50 mL) and then DCM (100 mL×4). The DCM phase was dried over sodium sulfate and solvent was removed by rotary evaporator. The product was obtained as white solid (6.57 g, 6.26 mmol) with a yield of 63%. ¹H NMR (500 MHz, DMSO) δ 0.71 (s, 9H), 0.75 (s, 9H), 2.72-2.95 (m, 4H), 3.22 (s, 3H), 3.32 (br., 3H), 3.41 (m, 2H), 3.50 (m, 20H), 3.65 (d, 1H), 3.95 (d, 1H), 4.05 (m, 2H), 4.10-4.30 (m, 2H), 4.65 (br., 1H), 6.82 (d, 2H), 7.12 (m, 1H), 7.18 (m, 2H), 7.25 (m, 2H), 7.62 (br., 3H), 7.82 (d, 1H), 7.94 (m, 2H), 8.15 (m, 1H), 8.22 (br., 1H), 8.75 (s, 1H), 9.78 (s, 1H); LC-MS (m/z) calcd., 1048.6, found 1049.6 [M H]+.

### Preparation of mPEG₆-atazanavir monophospholipid

### Example 16a

mPEG₆-Atazanavir-phosphate, C16-glycerol, and DPTS were dissolved in DCM (1 mL). The solution was stirred for ten minutes before the dropwise addition of DIC. The reaction mixture was stirred at room temperature for three hours. After the reaction, DCM (100 mL) was added into the mixture. The resulted solution was washed with water (100 mL×2) and dried over sodium sulfate. Crude product was obtained after removing solvent.

The major by-product was an intermediate of mPEG₆-atazanavir-phosphate with DIC, which was difficult to separate but could be completely converted to mPEG₆-atazanavir-phosphate methyl ester by simply dissolving the crude product in methanol and allowing the dissolved crude product to incubate for a couple of hours (methyl ester was confirmed by LC-MS). After the conversion, it was easily separated from product by silica column. The product was confirmed by HPLC, NMR, LC-MS and MALDI-TOF.

### Preparation of mPEG₃-atazanavir monophospholipid

### Example 16b

Using an approach similar to the one used to make mPEG₆-atazanavir monophospholipid (Example 16a), mPEG₃-atazanavir monophospholipid was prepared.

### Preparation of mPEG₅-atazanavir monophospholipid

### Example 16c

Using an approach similar to the one used to make mPEG₆-atazanavir monophospholipid (Example 16a), mPEG₅-atazanavir monophospholipid was prepared.

### Example 17

### Synthesis of mPEGₙ-Atazanavir-CME-Leucine Compounds

mPEGₙ-Atazanavir-CME-leucine compounds were prepared in two steps. Schematically, the first step can be schematically represented as follows.

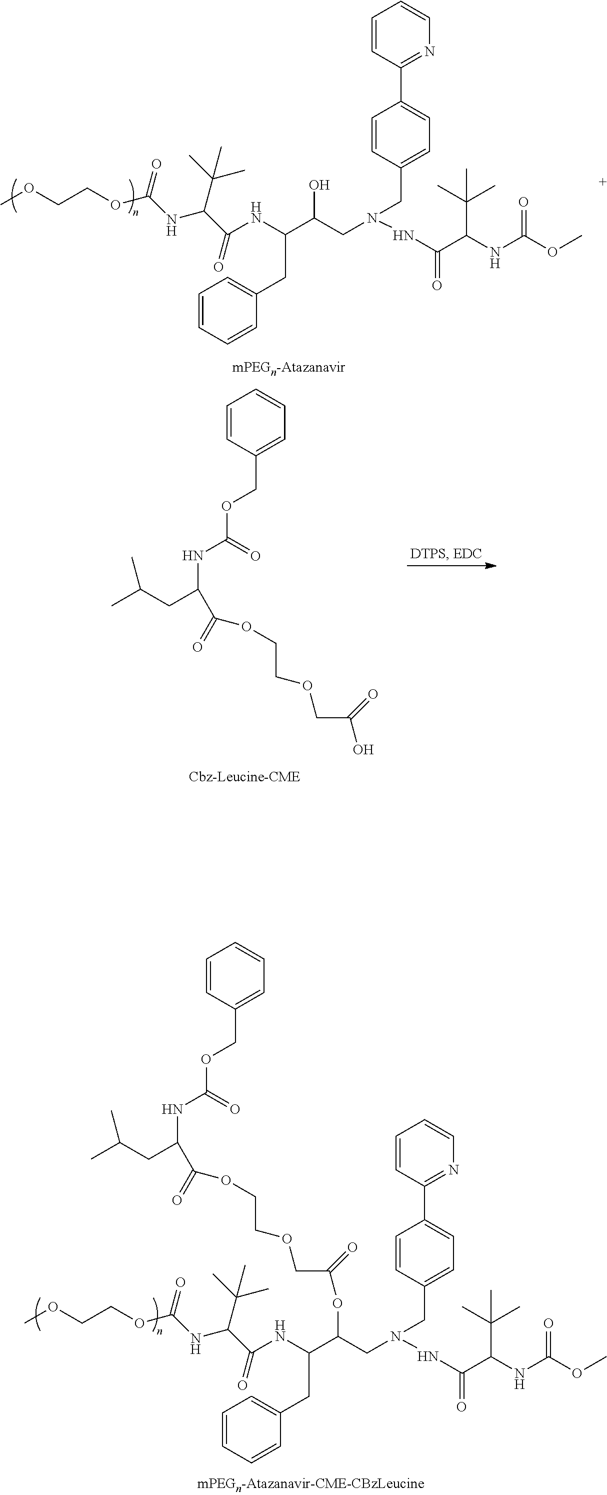

Thereafter, the second step to provide the desired mPEGₙ-Atazanavir-CME-leucine can be represented as follows.

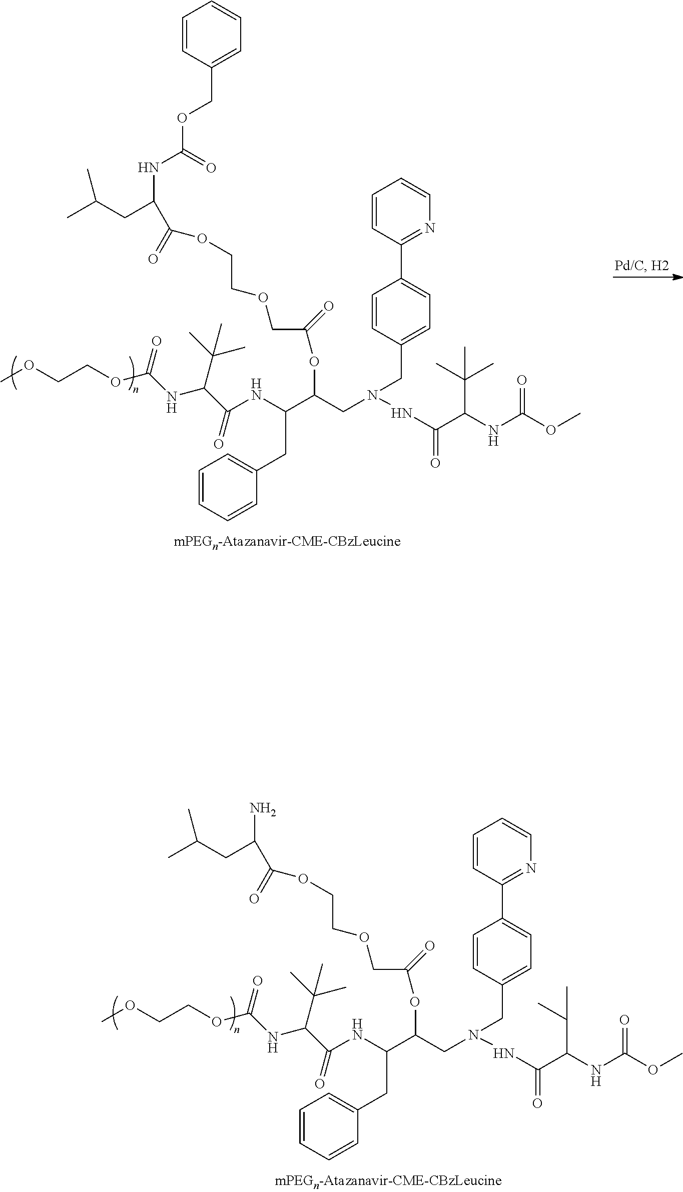

### Preparation of mPEG₃-Atazanavir-CME-CbzLeucine

Previously prepared mPEG₃-atazanavir (0.837 g, 1.0 mmol), Cbz-Leucine-CME (0.514 g, 1.4 mmol), and DPTS (0.155 g, 0.5 mmol) were dissolved in 10 mL of DCM. To the solution, EDC (0.465 g, 3.0 mmol) was added dropwise under stirring. The resulting mixture was stirred at room temperature for sixteen hours. After this period, the solid was filtered out and 200 mL of DCM was added into the filtration. The solution was washed with 0.5 N HCl (100 mL) and H₂O (100 ml). The organic phase was dried over Na₂SO₄ and concentrated under reduced pressure. The residue was purified by biotage (DCM/Methanol: 3% of methanol (equilibrium 3 CV); 3-6% of methanol, 17 CV, 6-8% of methanol, 5 CV). The product was obtained as white solid with the yield of 87%. ¹H NMR (500 MHz, CDCl₃) δ 0.75 (s, 9H), 0.85 (s, 9H), 0.95 (m, 6H), 1.55-1.80 (m, 3H), 2.62 (m, 1H), 2.75 (m, 2H), 3.32 (m, 1H), 3.34 (s, 3H), 3.50-3.70 (m, 15H), 3.75-3.95 (m, 3H), 4.05-4.25 (m, 6H), 4.32-4.50 (m, 4H), 5.00 (d, 1H), 5.05-5.15 (m, 2H), 5.30 (m, 1H), 5.42 (m, 1H), 5.92 (m, 1H), 6.26 (m, 1H), 7.10 (d, 2H), 7.15 (m, 1H), 7.20-7.45 (m, 10H), 7.75 (m, 2H), 7.93 (d, 2H), 8.70 (d, 1H).

### Preparation of mPEG₅-Atazanavir-CME-CbzLeucine

Previously prepared mPEG₅-atazanavir (0.925 g, 1.0 mmol), Cbz-Leucine-CME (0.551 g, 1.5 mmol), and DPTS (0.155 g, 0.5 mmol) were dissolved in 10 mL of DCM. To the solution, EDC (0.465 g, 3.0 mmol) was added dropwise under stirring. The resulted mixture was stirred at room temperature for sixteen hours. After this period, the solid was filtered out and 200 mL of DCM was added into the filtration. The solution was washed with 0.5 N HCl (100 mL) and H₂O (100 ml). The organic phase was dried over Na₂SO₄ and concentrated under reduced pressure. The residue was purified by biotage (DCM/Methanol: 3% of methanol (equilibrium. 3 CV); 3-6% of methanol, 17 CV, 6-8% of methanol, 5 CV). The product was obtained as a white solid with the yield of 85%. ¹H NMR (500 MHz, CDCl₃) δ 0.78 (s, 9H), 0.85 (s, 9H), 0.95 (m, 6H), 1.55-1.80 (m, 3H), 2.62 (m, 1H), 2.75 (m, 2H), 3.32 (m, 1H), 3.34 (s, 3H), 3.50-3.70 (m, 22H), 3.75-3.95 (m, 3H), 4.05-4.25 (m, 6H), 4.32-4.50 (m, 4H), 5.00 (d, 1H), 5.05-5.15 (m, 2H), 5.30 (m, 1H), 5.42 (m, 1H), 5.92 (m, 1H), 6.26 (m, 1H), 7.10 (d, 2H), 7.15 (m, 1H), 7.20-7.45 (m, 10H), 7.75 (m, 2H), 7.94 (d, 2H), 8.71 (d, 1H).

### Preparation of mPEG₆-Atazanavir-CME-CbzLeucine

Previously prepared mPEG₆-atazanavir (0.582 g, 0.6 mmol), Cbz-Leucine-CME (0.242 g, 0.66 mmol), and DPTS (0.093 g, 0.3 mmol) were dissolved in 10 mL of DCM. To the solution, EDC (0.279 g, 1.8 mmol) was added dropwise under stirring. The resulted mixture was stirred at room temperature for sixteen hours. After this period, the solid was filtered out and 200 mL of DCM was added into the filtration. The solution was washed with 0.5 N HCl (100 mL) and H₂O (100 ml). The organic phase was dried over Na₂SO₄ and concentrated under reduced pressure. The residue was purified by biotage (DCM/Methanol: 3% of methanol (equilibrium. 3 CV); 3-6% of methanol, 17 CV, 6-8% of methanol, 5 CV). The product was obtained as a white solid with the yield of 72%. ¹H NMR (500 MHz, CDCl₃) δ 0.78 (s, 9H), 0.85 (s, 9H), 0.95 (m, 6H), 1.55-1.80 (m, 3H), 2.62 (m, 1H), 2.75 (m, 2H), 3.32 (m, 1H), 3.34 (s, 3H), 3.50-3.70 (m, 26H), 3.75-3.95 (m, 3H), 4.05-4.25 (m, 6H), 4.32-4.50 (m, 4H), 5.00 (d, 1H), 5.05-5.15 (m, 2H), 5.30 (m, 1H), 5.42 (m, 1H), 5.92 (m, 1H), 6.26 (m, 1H), 7.10 (d, 2H), 7.15 (m, 1H), 7.20-7.45 (m, 10H), 7.75 (m, 2H), 7.94 (d, 2H), 8.71 (d, 1H); LC-MS (m/z) calcd., 1317.7, found 1318.7 [M+H]+.

### Preparation of mPEG₃-Atazanavir-CME-Leucine

In a 125 mL of hydrogenation reactor was charged 0.2 g of Pd/C (wet). mPEG₃-Atazanavir-CME-CbzLeucine (1.0 g, 0.84 mmol) in 10 mL THF was added into the reactor. The mixture was hydrogenated at 25 psi for four hours. HPLC showed that the reaction was not completed (20% SM remained). 0.1 g more of the Pd/C was added into the reaction mixture. Hydrogenated at the same condition for additional two hours and the reaction completed. The crude product was filtered through celite 545, but the catalyst could not be completely removed. An additional filtration was tried, but the solution was still in dark color. The Quadra Sil metal scavenger (Aldrich-07768HJ) was employed to remove the catalyst. The crude product was dissolved in 5 mL of ethyl acetate and 500 mg of the scavenger was added. The solution became colorless after shaking. The scavenger was filtered out and white solid was obtained after removing solvent. The product was dissolved in DCM (200 mL) and the solution was washed with 0.5 N HCl which was saturated with sodium chloride (50 mL). The DCM phase was dried over sodium sulfate and white solid was obtained as HCl salt after removing solvent. Yield: 44%. ¹H NMR (500 MHz, DMSO) δ 0.74 (s, 9H), 0.78 (s, 9H), 0.90 (m, 6H), 1.50-1.90 (m, 3H), 2.60 (m, 1H), 2.75 (m, 1H), 2.90 (m, 1H), 3.06 (m, 1H), 3.22 (s, 3H), 3.42 (m, 5H), 3.49-3.51 (m, 8H), 3.65 (d, 1H), 3.80 (m, 4H), 3.90 (d, 1H), 3.95 (m, 3H), 4.05 (m, 2H), 4.25 (s, 3H), 4.30-4.50 (m, 6H), 5.10 (m, 1H), 6.70 (m, 2H), 7.10-7.20 (m, 5H), 7.40 (m, 3H), 7.82 (m, 1H), 7.95 (d, 2H), 8.00 (s, 1H), 8.42 (br., 2H), 8.70 (s, 1H), 9.05 (s, 1H); LC-MS (m/z) calcd., 1051.8, found 1052.8 [M+H]+.

### Preparation of mPEG₅-Atazanavir-CME-Leucine

In a 250 mL of hydrogenation reactor was charged 0.4 g of Pd/C (wet). Previously prepared mPEG₅-atazanavir-CME-CbzLeucine (1.0 g, 0.785 mmol) in 10 mL THF was added into the reactor. The mixture was hydrogenated at 25 psi and the reaction was completed after six hours. The crude product was filtered through celite 545 and solvent was removed. The residue was dissolved in DCM (200 mL) and the solution was washed with 0.5 M HCl which was saturated with sodium chloride. The DCM was removed after dried over sodium sulfate. The solid was dissolved in a mixture of ethyl acetate and methanol (5:1, 5 mL). 1.0 g of Quadra Sil metal scavenger (Aldrich-07768HJ) was added. The solution became colorless after shaking. The scavenger was filtered out and white solid was obtained after removing solvent. Yield: 73.6%. ¹H NMR (500 MHz, DMSO) δ 0.74 (s, 9H), 0.77 (s, 9H), 0.88 (m, 6H), 1.45 (m, 1H), 1.54 (m, 1H), 1.75 (m, 1H), 2.60 (m, 1H), 2.75 (m, 1H), 2.90 (m, 1H), 3.06 (m, 1H), 3.22 (s, 3H), 3.35-3.70 (m, 22H), 3.69 (m, 2H), 3.90 (d, 1H), 3.95 (m, 2H), 4.05 (m, 2H), 4.20-4.35 (m, 4H), 4.45 (m. 1H), 5.10 (m, 1H), 6.70 (m, 2H), 7.10-7.20 (m, 5H), 7.30-7.40 (m, 3H), 7.85 (m, 2H), 7.95 (m, 3H), 8.65 (s, 1H), 9.02 (s, 1H); LC-MS (m/z) calcd., 1139.9, found 1140.9 [M+H]+.

### Preparation of mPEG₆-Atazanavir-CME-Leucine

In a 125 mL of hydrogenation reactor was charged 0.2 g of Pd/C (wet). Previously prepared mPEG₆-atazanavir-CME-CbzLeucine (0.85 g, 0.645 mmol) in 10 mL THF was added into the reactor. The mixture was hydrogenated at 25 psi for six hours. The reaction was completed. The crude product was filtered through celite 545 and solvent was removed. The residue was dissolved in DCM (200 mL) and the solution was washed with 0.5 M HCl in saturated sodium chloride. The DCM was removed after dried over sodium sulfate. The solid was dissolved in a mixture of ethyl acetate and methanol (5:1, 5 mL). 1.0 g of Quadra Sil metal scavenger (Aldrich-07768HJ) was added. The solution became colorless after shaking. The scavenger was filtered out and white solid was obtained after removing solvent. Yield: 72%. ¹H NMR (500 MHz, DMSO) δ 0.74 (s, 9H), 0.77 (s, 9H), 0.88 (m, 6H), 1.5-1.90 (m, 6H), 2.60 (m, 1H), 2.75 (m, 1H), 2.90 (m, 1H), 3.06 (m, 1H), 3.22 (s, 3H), 3.35-3.70 (m, 28H), 3.80 (m, 3H), 3.90 (d, 1H), 3.95 (m, 3H), 4.05 (m, 2H), 4.25 (s, 2H), 4.30-4.50 (m, 5H), 5.10 (m, 1H), 6.70 (m, 2H), 7.10-7.20 (m, 5H), 7.30-7.40 (m, 3H), 7.85 (m, 1H), 7.95 (m, 2H), 8.01 (m, 2H), 8.45 (br., 2H), 8.70 (s, 1H), 9.05 (s, 1H); LC-MS (m/z) calcd., 1183.7, found 1184.7 [M+H]+.

### Example 18

### Synthesis of mPEGₙ-Atazanavir-CME-PhePhenylalanine Compounds

mPEGₙ-Atazanavir-CME-PhePhenylalanine compounds were prepared in two steps. Schematically, the first step can be schematically represented as follows.

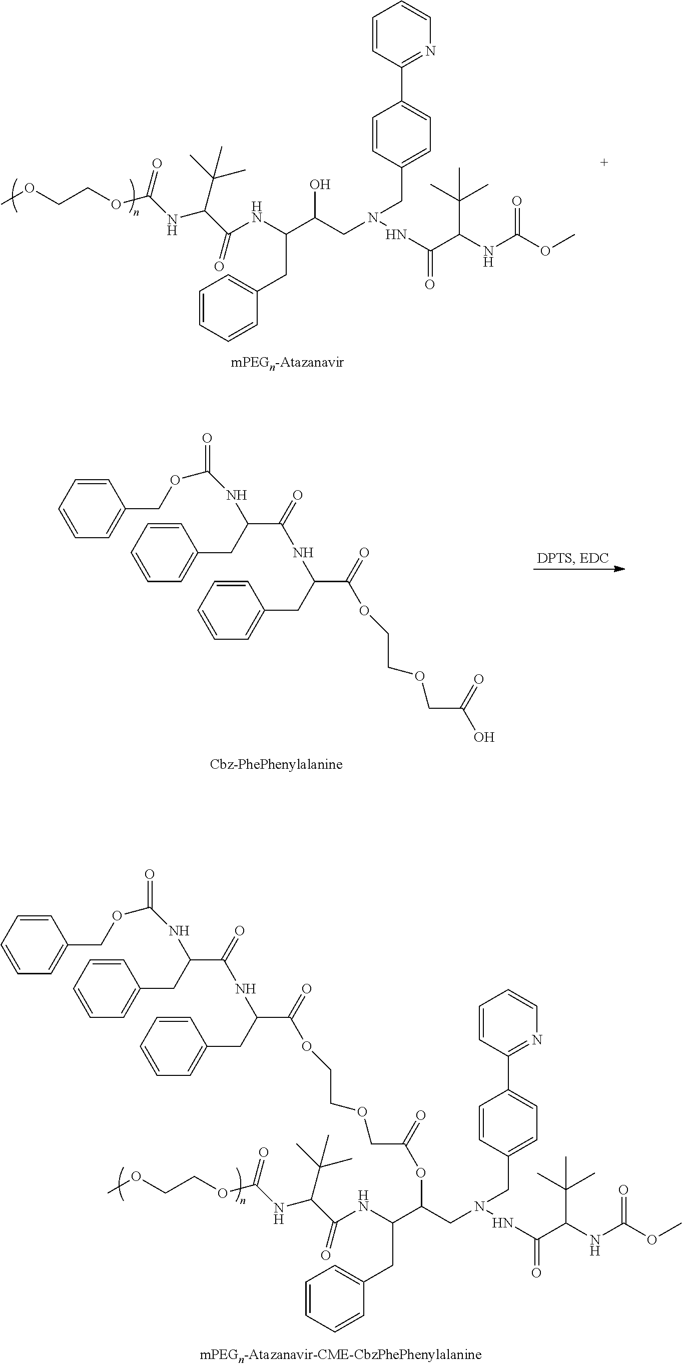

Thereafter, the second step to provide the desired mPEGₙ-atazanavir-CME-PhePhenylalanine can be represented as follows.

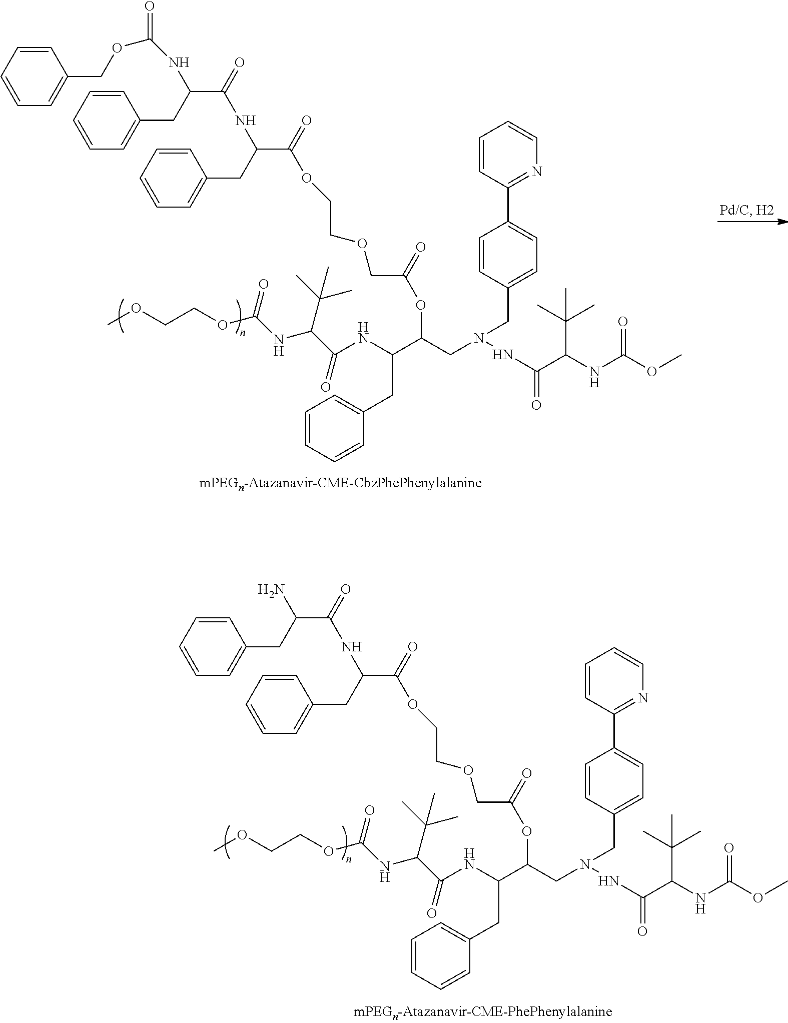

### Preparation of mPEG₃-Atazanavir-CME-CbzPhePhenylalanine

Previously prepared mPEG₃-atazanavir (0.837 g, 1.0 mmol), Cbz-PhePhenylanine-CME (0.768 g, 1.4 mmol), and DPTS (0.155 g, 0.5 mmol) were dissolved in 10 mL of DCM. To the solution, EDC (0.465 g, 3.0 mmol) was added dropwise under stirring. The resulting mixture was stirred at room temperature for sixteen hours. After this period, the solid was filtered out and 200 mL of DCM was added into the filtration. The solution was washed with 0.5 N HCl (100 mL) and H₂O (100 ml). The organic phase was dried over Na₂SO₄ and concentrated under reduced pressure. The residue was purified by biotage (DCM/methanol: 3% of methanol (equilibrium 3 CV); 3-6% of methanol, 17 CV, 6-8% of methanol, 5 CV). The product was obtained as a white solid with the yield of 80%. ¹H NMR (500 MHz, DMSO) δ 0.74 (s, 9H), 0.85 (s, 9H), 2.60 (m, 1H), 2.70 (m, 2H), 2.86 (m, 1H), 2.96 (m, 2H), 3.08 (m, 2H), 3.22 (s, 3H), 3.35-3.60 (m, 13H), 3.65 (d, 1H), 3.70 (m, 2H), 3.90 (d, 1H), 3.95 (s, 2H), 4.05 (m, 2H), 4.20 (m, 4H), 4.30 (m, 1H), 4.45 (m, 1H), 4.55 (m, 1H), 4.90 (s, 2H), 5.10 (m, 1H), 6.70 (m, 2H), 7.05-7.34 (m, 22H), 7.35 (d, 2H), 7.45 (d, 1H), 7.84 (m, 2H), 7.90 (d, 1H), 7.95 (d, 2H), 8.50 (d, 1H), 8.62 (d, 1H), 9.00 (d, 1H).

### Preparation of mPEG₅-Atazanavir-CME-CbzPhePhenylanine

Previously prepared mPEG₅-atazanavir (0.925 g, 1.0 mmol), Cbz-PhePhenylalanine-CME (0.823 g, 1.5 mmol), and DPTS (0.155 g, 0.5 mmol) were dissolved in 10 mL of DCM. To the solution, EDC (0.465 g, 3.0 mmol) was added dropwise under stirring. The resulted mixture was stirred at room temperature for sixteen hours. After this period, the solid was filtered out and 200 mL of DCM was added into the filtration. The solution was washed with 0.5 N HCl (100 mL) and H₂O (100 ml). The organic phase was dried over Na₂SO₄ and concentrated under reduced pressure. The residue was purified by biotage (DCM/methanol: 3% of methanol (equilibrium. 3 CV); 3-6% of methanol, 17 CV, 6-8% of methanol, 5 CV). The product was obtained as a white solid with the yield of 87%. ¹H NMR (500 MHz, DMSO) δ 0.74 (s, 9H), 0.85 (s, 9H), 2.60 (m, 1H), 2.70 (m, 2H), 2.86 (m, 1H), 2.96 (m, 2H), 3.08 (m, 2H), 3.22 (s, 3H), 3.35-3.60 (m, 21H), 3.65 (d, 1H), 3.70 (m, 2H), 3.90 (d, 1H), 3.95 (s, 2H), 4.05 (m, 2H), 4.20 (m, 4H), 4.30 (m, 1H), 4.45 (m, 1H), 4.55 (m, 1H), 4.90 (s, 2H), 5.10 (m, 1H), 6.70 (m, 2H), 7.05-7.34 (m, 22H), 7.35 (d, 2H), 7.45 (d, 1H), 7.84 (m, 2H), 7.90 (d, 1H), 7.95 (d, 2H), 8.50 (d, 1H), 8.62 (d, 1H), 9.00 (d, 1H).

### Preparation of mPEG₃-Atazanavir-CME-PhePhenylalanine

In a 125 mL of hydrogenation reactor was charged 0.4 g of Pd/C (wet). mPEG₃-Atazanavir-CME-CbzPhePhenylalanine (1.1 g, 0.804 mmol) in 15 mL THF was added into the reactor. The mixture was hydrogenated at 25 psi for sixteen hours. Some decomposed by-product (mPEG₃-atazanavir-CME-OH, 13%) was observed. The reaction mixture was filtered through celite 545 and solvent was removed. The crude product was purified by biotage (DCM/MeOH, 4% of MeOH (equilibrium. 3 CV); 4-8% of MeOH, 17 CV; 8-10% of MeOH, 5 CV). The product was transformed into an HCl salt by dissolving the product in DCM and adding an equal mole of HCl (4 N in dioxane). White solid was obtained as an HCl salt after removing solvent and dried (yield 65%). The product was unstable especially when it was impure. Some product was lost during hydrogenation, work-up, and column purification. ¹H NMR (500 MHz, DMSO) δ 0.74 (s, 9H), 0.85 (s, 9H), 2.60 (m, 1H), 2.75 (m, 1H), 2.86 (m, 1H), 2.95 (m, 1H), 3.02 (m, 1H), 3.06 (m, 2H), 3.19 (m, 1H), 3.22 (s, 3H), 3.40 (m, 5H), 3.50 (m, 9H), 3.65 (d, 1H), 3.75 (m, 2H), 3.90 (d, 1H), 4.00 (s, 2H), 4.05 (m, 3H), 4.20 (m, 4H), 4.50 (m, 1H), 4.56 (m, 1H), 5.10 (m, 1H), 6.70 (m, 2H), 7.05-7.34 (m, 16H), 7.45 (d, 2H), 7.60 (m, 1H), 7.84 (d, 1H), 7.96 (d, 2H), 8.10 (m, 1H), 8.20 (m, 2H), 8.72 (d, 1H), 9.08 (d, 1H), 9.24 (d, 1H); LC-MS (m/z) calcd., 1232.6, found 1233.6 [M+H]+.

### Preparation of mPEG₅-Atazanavir-CME-PhePhenylalanine

In a 125 mL of hydrogenation reactor was charged 0.4 g of Pd/C (wet). mPEG₅-Atazanavir-CME-CbzPhePhenylalanine (1.0 g, 0.687 mmol) in 15 mL THF was added into the reactor. The mixture was hydrogenated at 25 psi for sixteen hours. Because the reaction was not completed, 0.2 g of Pd/C was added and hydrogenated for additional six hours. Some decomposed by-product (mPEG₅-atazanavir-CME-OH, 20%) was observed. The reaction mixture was filtered through celite 545 and solvent was removed. The crude product was purified by biotage (DCM/MeOH, 4% of MeOH (equilibrium. 3 CV); 4-8% of MeOH, 17 CV; 8-10% of MeOH, 5 CV). The product was transformed into an HCl salt by dissolving the product in DCM and adding an equal mole of HCl (4 N in dioxane). White solid was obtained as an HCl salt after removing solvent and dried (yield, 43%). The product was unstable especially when it was impure. Some product was lost during hydrogenation, work-up, and column purification. ¹H NMR (500 MHz, DMSO) δ 0.74 (s, 9H), 0.85 (s, 9H), 2.60 (m, 1H), 2.75 (m, 1H), 2.86 (m, 1H), 2.95 (m, 1H), 3.02 (m, 1H), 3.06 (m, 2H), 3.19 (m, 1H), 3.22 (s, 3H), 3.40 (m, 6H), 3.50 (m, 15H), 3.65 (d, 1H), 3.75 (m, 2H), 3.90 (d, 1H), 4.00 (s, 2H), 4.05 (m, 3H), 4.20 (m, 4H), 4.50 (m, 1H), 4.56 (m, 1H), 5.10 (m, 1H), 6.70 (m, 2H), 7.05-7.34 (m, 16H), 7.45 (d, 2H), 7.60 (m, 1H), 7.84 (d, 1H), 7.96 (d, 2H), 8.10 (m, 1H), 8.20 (m, 2H), 8.72 (d, 1H), 9.08 (d, 1H), 9.24 (d, 1H); LC-MS (m/z) calcd., 1320.6, found 1321.7 [M+H]+.

### Preparation of mPEG₆-Atazanavir-CME-PhePhenylalanine

Using an approach similar to the one taken to prepare mPEG₅-atazanavir-CME-PhePhenylalanine, mPEG₆-atazanavir-CME-PhePhenylalanine was prepared.

### Preparation of mPEG₃-atazanavir-CME-phenylalanine, mPEG₃-atazanavir-CME-phenylalanine, and mPEG₃-atazanavir-CME-phenylalanine

Using an approach similar to that used to make mPEG₅-atazanavir-CME-PhePhenylalanine, each of mPEG₃-atazanavir-CME-phenylalanine, mPEG₃-atazanavir-CME-phenylalanine, and mPEG₃-atazanavir-CME-phenylalanine was prepared.

### Examples 19a-19c

### Preparation of mPEGₙ-Atazanavir-Ethyl Carbonate Compounds

mPEGₙ-Atazanavir-ethyl carbonate compounds were prepared in accordance with the schematic provided below.

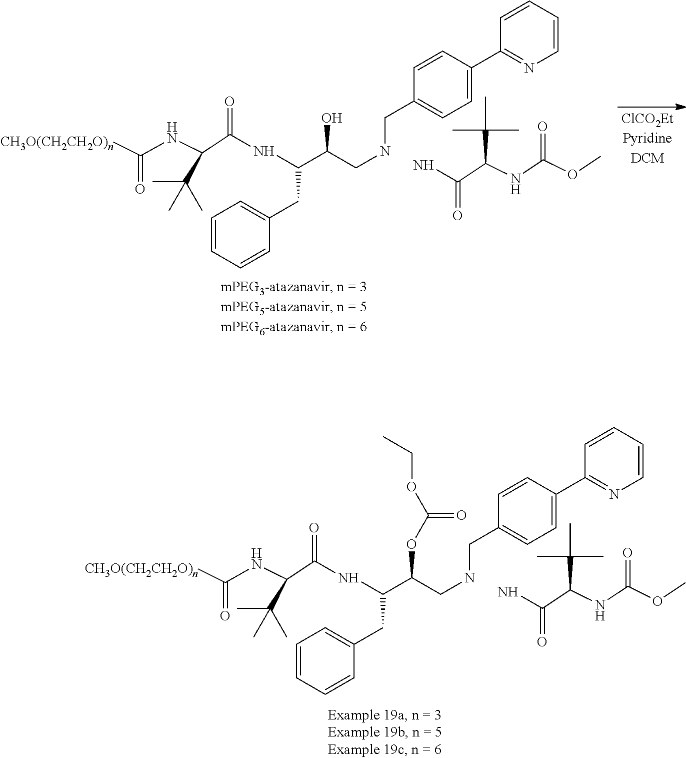

In preparing the compounds of Example 19, all reactions with air- or moisture-sensitive reactants and solvents were carried out under nitrogen atmosphere. In general, reagents and sovents were used as purchased without further purification. Analytical thin-layer chromatography was performed on silica F₂₅₄ glass plates (Biotage). Components were visualized by UV light of 254 nm or by spraying with phosphomolybdic acid. Flash chromatography was performed on a Biotage SP4 system. ¹H NMR spectra: Bruker 500 MHz; chemical shifts of signals are expressed in parts per million (ppm) and are referenced to the deuterated solvents used. MS spectra: rapid resolution Zorbax C18 column; 4.6×50 mm; 1.8 μm. HPLC method had the following parameters: column, Betasil C18, 5-μm (100×2.1 mm); flow, 0.5 mL/min; gradient, 0-23 min, 20% acetonitrile/0.1% TFA in water/0.1% TFA to 100% acetonitrile/0.1% TFA; detection, 230 nm. “t_{R}” refers to the retention time.

### Example 19a

### mPEG₃-Atazanavir Ethyl Carbonate

Into a 250 mL round bottom flask was added previously prepared mPEG₃-atazanavir (1.16 gm, 1.38 mmol) and anhydrous dichloromethane (30 mL). To the clear solution was added anhydrous pyridine (2.24 mL, 27.6 mmol), followed by ethyl chloroformate (1.4 mL, 14.5 mmol). The reaction progressed very slowly, and it was necessary to add additional equivalents of reagents to ensure nearly complete conversion. After a total of three days at room temperature, and a total of 80 equivalents of pyridine, and 42 equivalents of ethyl chloroformate the reaction was worked up. The reaction mixture was diluted with dichlormethane (100 mL) and transferred to a separatory funnel, where it was partitioned with deionized water (130 mL). The aqueous layer was extracted with dichloromethane (3×25 mL). The combined organic layers were washed successively with water, saturated sodium bicarbonate, water, 1N HCl, water and saturated sodium chloride (130 mL each). The organic layer was dried over anhydrous sodium sulfate, filtered, and concentrated under reduced pressure to give a light-yellow oil. Purification by Biotage chromatography (gradient elution: 0 to 5% methanol/dichloromethane over 20 column volumes) gave 1.0 gm (80%) of product as a white solid; R_{f} 0.60 (10% methanol-dichloromethane); ¹H NMR (DMSO-d6): δ 9.00 (bs, 1H), 8.64 (d, 1H), 7.94 (m, 3H), 7.86 (m, 2H), 7.36 (m, 3H), 7.19 (m, 5H), 6.74 (d, 2H), 4.89 (bs, 1H), 4.47 (bs, 1H), 4.18 (q, 2H), 4.04 (m, 4H), 3.92 (d, 1H), 3.58 (d, 1H), 3.33-3.51 (m, 12H), 3.22 (s, 3H), 2.95 (m, 1H), 2.78 (m, 1H), 2.65 (m, 1H), 1.27 (t, 3H), 0.76 (s, 9H), 0.72 (s, 9H). MS 909 (M+H)⁺.

### Example 19b

### mPEG₅-Atazanavir Ethyl Carbonate

Into a 250 mL round bottom flask was added previously prepared mPEG₅-atazanavir (1.0 gm, 1.08 mmol) and anhydrous dichloromethane (35 mL). To the clear solution was added anhydrous pyridine (1.75 mL, 21.6 mmol), followed by ethyl chloroformate (1.04 mL, 10.8 mmol). The reaction progressed very slowly, and it was necessary to add additional equivalents of reagents to ensure nearly complete conversion. After a total of five days at room temperature, and a total of 119 equivalents of pyridine, and 60 equivalents of ethyl chloroformate, the reaction was worked up. The reaction mixture was diluted with dichloromethane (100 mL) and transferred to a separatory funnel, where it was partitioned with deionized water (130 mL). The aqueous layer was extracted with dichloromethane (3×25 mL). The combined organic layers were washed successively with water, saturated sodium bicarbonate, water, 1N HCl, water and saturated sodium chloride (130 mL each). The organic layer was dried over anhydrous sodium sulfate, filtered, and concentrated under reduced pressure to give a light-yellow oil. Purification by Biotage chromatography (gradient elution: 0 to 5% methanol/dichloromethane over 20 column volumes) gave 1.0 gm (80%) of product as a white solid; R_{f} 0.56 (10% methanol-dichloromethane); NMR (DMSO-d6): δ 8.98 (bs, 1H), 8.64 (d, 1H), 7.93 (m, 3H), 7.86 (m, 2H), 7.33 (m, 2H), 7.32 (m, 1H), 7.20 (m, 5H), 6.76 (d, 2H), 4.89 (bs, 1H), 4.46 (bs, 1H), 4.17 (q, 2H), 4.05 (m, 4H), 3.92 (d, 1H), 3.68 (d, 1H), 3.40-3.57 (m, 20H), 3.22 (s, 3H), 2.92 (m, 1H), 2.80 (m, 1H), 2.66 (m, 1H), 1.27 (t, 3H), 0.76 (s, 9H), 0.72 (s, 9H). MS 997 (M+H)⁺.

### Example 19c

### mPEG₆-Atazanavir Ethyl Carbonate

Into a 250 mL round bottom flask was added previously prepared mPEG₆-atazanavir (1.0 gm, 1.03 mmol) and anhydrous dichloromethane (35 mL). To the clear solution was added anhydrous pyridine (1.67 mL, 20.6 mmol), followed by ethyl chloroformate (0.99 mL, 10.8 mmol). The reaction progressed very slowly, and it was necessary to add additional equivalents of reagents to ensure nearly complete conversion. After a total of five days at room temperature, and a total of 119 equivalents of pyridine, and 60 equivalents of ethyl chloroformate, the reaction was worked up. The reaction mixture was diluted with dichlormethane (100 mL) and transferred to a separatory funnel, where it was partitioned with deionized water (130 mL). The aqueous layer was extracted with dichloromethane (3×25 mL). The combined organic layers were washed successively with water, saturated sodium bicarbonate, water, 1N HCl, water and saturated sodium chloride (130 mL each). The organic layer was dried over anhydrous sodium sulfate, filtered, and concentrated under reduced pressure to give a light-yellow oil. Purification by Biotage chromatography (gradient elution: 0 to 5% methanol/dichloromethane over 20 column volumes) gave 0.77 gm (72%) of product as a white solid; R_{f} 0.54 (10% methanol-dichloromethane); ¹H NMR (DMSO-d6): δ 9.98 (bs, 1H), 8.64 (d, 1H), 7.94 (m, 3H), 7.86 (m, 2H), 7.36 (m, 2H), 7.33 (m, 1H), 7.12-7.19 (m, 5H), 6.76 (d, 2H), 4.89 (bs, 1H), 4.47 (bs, 1H), 4.17 (q, 2H), 4.02-4.06 (m, 4H), 3.92 (d, 1H), 3.67 (d, 1H), 3.57 (m, 2H), 3.49 (m, 18H), 3.45 (m, 2H), 3.42 (m, 2H), 3.22 (s, 3H), 2.92 (m, 1H), 2.79 (m, 1H), 2.66 (m, 1H), 1.27 (t, 3H), 0.76 (s, 9H), 0.73 (s, 9H). MS 1041 (M+H)⁺.

### Example 20

### mPEGₙ-Atazanavir Carbonate Compounds

mPEGₙ-Atazanavir carbonate compounds were prepared in accordance with the schematic provided below, wherein the organic radical-containing “R” groups can be attached via a releasable carbonate linkage from the intermediate 1-chloroethylcarbonate of mPEGₙ-atazanavir.

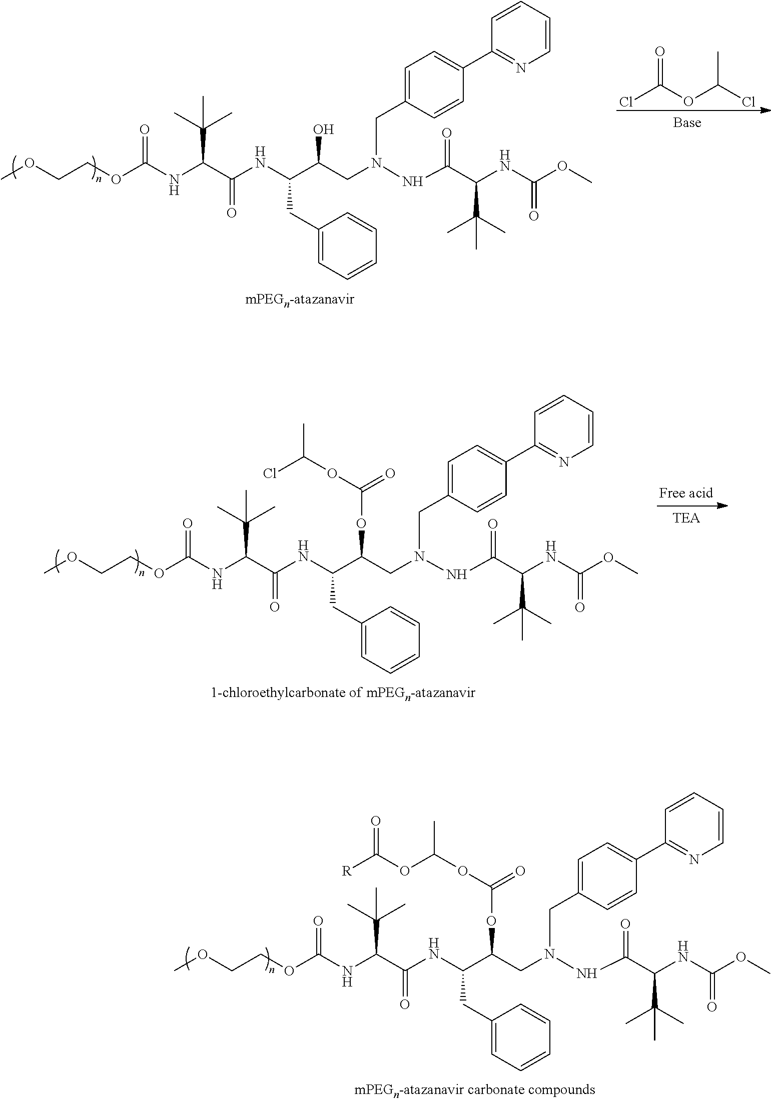

Exemplary compounds of Example 20 were prepared using 1-chloroethyl chloroformate, pyridine, methoxyacetic acid and triethylamine, which were purchased from Sigma-Aldrich (St Louis, Mo.). Sodium bicarbonate (NaHCO₃), ammonium chloride (NH₄Cl), sodium sulfate (Na₂SO₄), and sodium chloride (NaCl), hydrochloric acid (conc. HCl) were purchased from EM Science (Gibbstown, N.J.). DCM was freshly distilled from CaH₂. Acetone, hexane, and other organic solvents were used as they purchased.

### Preparation of 1-Chloroethylcarbonate of mPEG₅-Atazanavir

In a 50-mL flask, previously prepared mPEG₅-atazanavir (450 mg, 0.486 mmol) was dissolved in DCM (2 mL). Pyridine (708 μL, 8.76 mmol) and 1-chloroethyl chloroformate (318 μL, 2.92 mmol) were premixed in DCM (8 mL) under controlled temperature via a water bath (10° C.). The mPEG₅-atazanavir- and DCM-containing solution was added to this active solution and the reaction was kept at room temperature for two hours before the reaction was stopped with saturated NH₄Cl (50 mL). The mixture solution was extracted with DCM (20 mL×3). The organic phase was combined and washed with saturated NH₄Cl (100 mL+0.5 mL conc. HCl) and saturated NaCl (50 mL) solution and dried over Na₂SO₄. After filtration, it was then concentrated under the pressure and high vacuo before the next reaction.

From a 1-chloroethylcarbonate of mPEGₙ-atazanavir intermediate, any number of organic radical-containing “R” groups can be attached via a releasable carbonate linkage to form mPEGₙ-atazanavir carbonate compounds. Exemplary mPEGₙ-Atazanavir carbonate compounds are described herein.

### Preparation of Methoxyacetate Ester-mPEG₅-Atazanavir

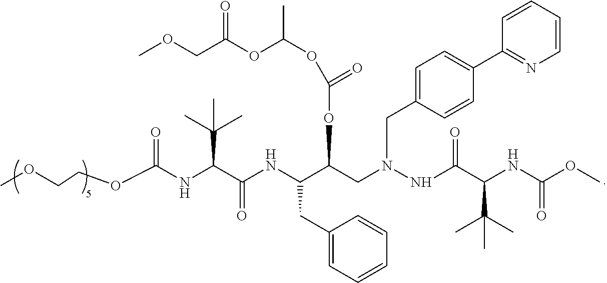

1-Chloroethylcarbonate of mPEGₙ-atazanavir was dissolved in DCM (2 mL). Then, 2-methoxyacetic acid (373 μL, 4.86 mmol) and triethylamine (TEA, 610 μL, 4.37 mmol) were premixed in DCM (5 mL) under controlled temperature via a water bath. After cooling to room temperature, this mixed solution was dropwise added to the 1-chloroethylcarbonate of mPEGₙ-atazanavir dissolved in DCM. The reaction was kept at ambient temperature and the solvent was dried off via a slow N₂ gas bubbling. The reaction was monitored via HPLC and was worked up after four days. It was quenched with NH₄Cl (50 mL) and extracted with DCM (20 mL×3). The combined organic phase was washed with NaCl (50 mL) and dried over Na₂SO₄. After filtration, it was concentrated under pressure and the product mixture was purified two times on Biotage silica gel column (32-65% acetone/hexane in 20 CV). The combined product solidified during the high vacuo drying. RP-HPLC (betasil C18, 0.5 mL/min, 10-100% ACN in 10 min) 7.18 min, LC-MS (ESI, MH⁺) 1085.6; ¹H NMR (500 MHz, CDCl₃) δ 0.72-0.82 (18H, m), 1.65-1.68 (3H, m), 2.63-2.88 (3H, m), 3.36-3.70 (29H, m), 4.08-4.35 (7H, m), 4.84-4.89 (1H, m), 5.28-5.44 (2H, m), 5.96 (1H, d, J=9.0 Hz), 6.88 (1H, d, J=4.5 Hz), 7.12-7.35 (10H, m), 7.70-7.76 (2H, m), 7.88-7.92 (2H, m), 8.67 (1H, d, J=3.5 Hz).

### Preparation of Acetate Ester-mPEG₅-Atazanavir

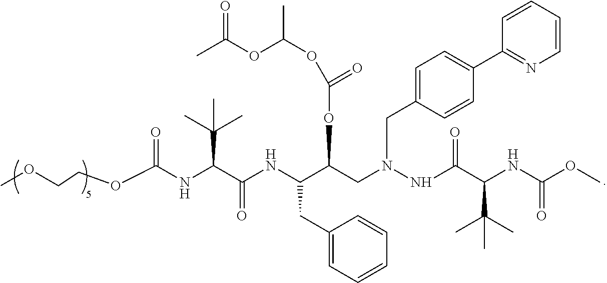

The reaction was performed in a manner similar to the approach described above for the preparation of methoxyacetate ester-mPEG₅-atazanavir. Briefly, acetic acid (20 eq) (rather than 2-methoxyacetic acid) and TEA (18 eq) were used, each in an amount that was effectively doubled. The reaction was monitored via HPLC. The reaction was worked up as before and purified on Biotage column (32-65% acetone/hexane in 20 CV) one time. The combined product was obtained after high vacuo drying. RP-HPLC (betasil C18, 0.5 mL/min, 10-100% ACN in 10 min) 7.28 min, LC-MS (ESI, MH⁺) 1055.6; ¹H NMR (500 MHz, CDCl₃) δ 0.71-0.81 (18H, m), 1.61-1.65 (3H, m), 2.13-2.15 (3H, m), 2.63-2.87 (3H, m), 3.36-3.71 (27H, m), 4.13-4.23 (4H, m), 4.83-4.89 (1H, m), 5.29-5.42 (2H, m), 5.93-5.98 (1H, m), 6.80 (1H, m), 7.12-7.38 (10H, m), 7.69-7.76 (2H, m), 7.88-7.92 (2H, m), 8.67 (1H, d, J=4.5 Hz).

### Examples 21a-21c

### Preparation of mPEGₙ-Atazanavir-Methyl Methyl Ether Compounds

mPEGₙ-Atazanvir compounds were prepared in accordance with the schematic provided below.

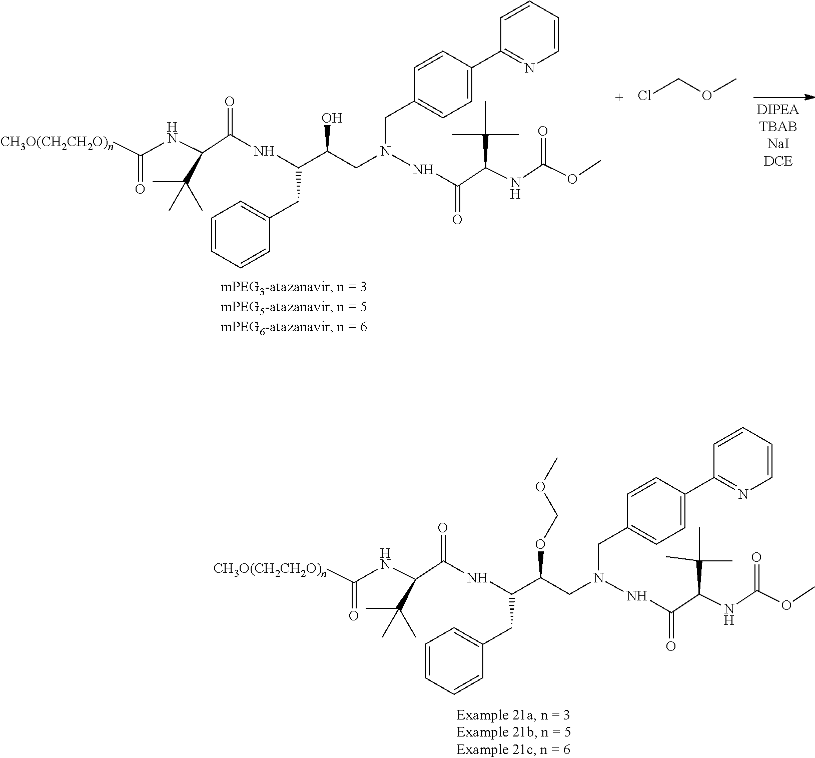

In preparing compounds associated with of Example 21 (as well as compounds associated with Examples 22, 23, 26 and 27), all reactions with air- or moisture-sensitive reactants and solvents were carried out under nitrogen atmosphere. In general, reagents and sovents were used as purchased without further purification. Analytical thin-layer chromatography was performed on silica F₂₅₄ glass plates (Biotage). Components were visualized by UV light of 254 nm or by spraying with phosphomolybdic acid. Flash chromatography was performed on a Biotage SP4 system. ¹H NMR spectra: Bruker 500 MHz; chemical shifts of signals are expressed in parts per million (ppm) and are referenced to the deuterated solvents used. MS spectra: rapid resolution Zorbax C18 column; 4.6×50 mm; 1.8 μm. HPLC method had the following parameters: column, Betasil C18, 5-μm (100×2.1 mm); flow, 0.5 mL/min; gradient, 0-23 min, 20% acetonitrile/0.1% TFA in water/0.1% TFA to 100% acetonitrile/0.1% TFA; detection, 230 nm. “t_{R}” refers to the retention time.

### Example 21a

### mPEG₃-Atazanavir Methyl Methyl Ether

Into a 100 mL round bottom flask was added previously prepared mPEG₃-atazanavir (0.857 gm, 1.02 mmol) and anhydrous 1,2-dichloroethane (25 mL). To the clear solution was added diisopropyl ethyl amine (0.91 mL, 5.12 mmol), followed by chloromethyl methyl ether (0.40 mL, 5.12 mmol), sodium iodide (0.077 gm, 0.51 mmol), and tetrabutylammonium bromide (0.066 gm, 0.20 mmol). The clear reaction mixture was heated to 70° C. under nitrogen. After approximately eighteen hours at 70° C., the dark amber reaction mixture was cooled to room temperature, and diluted with dichloromethane (100 mL). The organic mixture was transferred to a separatory funnel and partitioned with deionized water (100 mL). The aqueous layer was extracted with dichloromethane (3×30 mL). The combined organic layers were washed with saturated sodium bicarbonate, deionized water and saturated sodium chloride (100 mL each). The organic layer was dried over anhydrous sodium sulfate, filtered, and concentrated under reduced pressure to give 1.44 gm of a dark oil. Purification by Biotage chromatography (gradient elution: 0 to 5% methanol/dichloromethane over 20 column volumes) gave 0.74 gm (82%) of mPEG₃-atazanavir methyl methyl ether as a yellow oil; R_{f} 0.42 (5% methanol-dichloromethane); NMR (DMSO-d6): δ 8.97 (bs, 1H), 8.64 (d, 1H), 7.97 (d, 2H), 7.92 (d, 1H), 7.87 (t, 1H), 7.58 (d, 1H), 7.39 (d, 2H), 7.32 (m, 1H), 7.21 (m, 4H), 7.18 (m, 1H), 6.82 (m, 2H), 4.94 (d, 1H), 4.67 (d, 1H), 4.38 (bs, 1H), 4.04 (m, 2H), 3.92 (m, 3H), 3.67 (m, 2H), 3.54 (m, 2H), 3.49 (m, 9H), 3.41 (m, 2H), 3.35 (s, 3H), 2.87 (m, 2H), 2.79 (m, 1H), 2.75 (m, 1H), 0.75 (s, 3H), 0.73 (s, 3H). MS 881.5 (M+H)⁺.

### Example 21b

### mPEG₅-Atazanavir Methyl Methyl Ether

Into a 100 mL round bottom flask was added previously prepared mPEG₅-atazanavir (0.86 gm, 0.94 mmol) and anhydrous 1,2-dichloroethane (24 mL). To the clear solution was added diisopropyl ethyl amine (0.91 mL, 5.12 mmol), followed by chloromethyl methyl ether (0.40 mL, 5.12 mmol), sodium iodide (0.070 gm, 0.47 mmol), and tetrabutylammonium bromide (0.060 gm, 0.18 mmol). The clear reaction mixture was heated to 70° C. under nitrogen. After approximately eighteen hours at 70° C., the dark amber reaction mixture was cooled to room temperature, and diluted with dichloromethane (100 mL). The organic mixture was transferred to a separatory funnel and partitioned with deionized water (100 mL). The aqueous layer was extracted with dichloromethane (3×30 mL). The combined organic layers were washed with saturated sodium bicarbonate, deionized water and saturated sodium chloride (100 mL each). The organic layer was dried over anhydrous sodium sulfate, filtered, and concentrated under reduced pressure to give 1.71 gm of a dark oil. Purification by Biotage chromatography (gradient elution: 0 to 5% methanol/dichloromethane over 20 column volumes) gave 0.67 gm (74%) of mPEG₅-atazanavir methyl methyl ether as a yellow oil; R_{f} 0.42 (5% methanol-dichloromethane); NMR (DMSO-d6): δ 8.97 (bs, 1H), 8.64 (d, 1H), 7.97 (d, 2H), 7.91 (d, 1H), 7.85 (t, 1H), 7.57 (d, 1H), 7.39 (d, 2H), 7.32 (m, 1H), 7.22 (m, 4H), 7.18 (m, 1H), 6.82 (d, 2H), 4.95 (d, 1H), 4.67 (d, 1H), 4.38 (bs, 1H), 4.04 (m, 2H), 3.91 (m, 3H), 3.68 (m, 2H), 3.54 (m, 2H), 3.49 (m, 18H), 3.41 (m, 2H), 3.35 (s, 3H), 3.22 (s, 3H), 2.88 (m, 2H), 2.79 (m, 1H), 2.73 (m, 1H), 0.76 (s, 3H), 0.74 (s, 3H). MS 969.6 (M+H)⁺.

### Example 21c

### mPEG₆-Atazanavir Methyl Methyl Ether

Into a 100 mL round bottom flask was added mPEG₆-atazanavir (0.87 gm, 0.89 mmol) and anhydrous 1,2-dichloroethane (22 mL). To the clear solution was added diisopropyl ethyl amine (0.80 mL, 4.49 mmol), followed by chloromethyl methyl ether (0.35 mL, 4.49 mmol), sodium iodide (0.067 gm, 0.44 mmol), and tetrabutylammonium bromide (0.058 gm, 0.18 mmol). The clear reaction mixture was heated to 70° C. under nitrogen. After approximately eighteen hours at 70° C., the dark amber reaction mixture was cooled to room temperature, and diluted with dichloromethane (100 mL). The organic mixture was transferred to a separatory funnel and partitioned with deionized water (100 mL). The aqueous layer was extracted with dichloromethane (3×30 mL). The combined organic layers were washed with saturated sodium bicarbonate, deionized water and saturated sodium chloride (100 mL each). The organic layer was dried over anhydrous sodium sulfate, filtered, and concentrated under reduced pressure to give 1.74 gm of a dark oil. Purification by Biotage chromatography (gradient elution: 0 to 5% methanol/dichloromethane over 20 column volumes) gave 0.74 gm (81%) of mPEG₆-atazanavir methyl methyl ether as a yellow oil; R_{f} 0.37 (5% methanol-dichloromethane); ¹H NMR (DMSO-d6): δ 8.97 (bs, 1H), 8.64 (d, 1H), 7.96 (d, 2H), 7.92 (d, 1H), 7.86 (t, 1H), 7.58 (d, 1H), 7.39 (d, 2H), 7.32 (m, 1H), 7.22 (m, 4H), 7.18 (m, 1H), 6.82 (d, 2H), 4.94 (d, 1H), 4.66 (d, 1H), 4.37 (bs, 1H), 4.03 (m, 2H), 3.92 (m, 3H), 3.53 (m, 2H), 3.51 (m, 2H), 3.49 (m, 20H), 3.41 (m, 2H), 3.35 (s, 3H), 3.22 (s, 3H), 2.87 (m, 2H), 2.80 (m, 1H), 2.75 (m, 1H), 0.75 (s, 3H), 0.73 (s, 3H). MS 1013.6 (M+H)⁺.

### Examples 22a-22c

### Preparation of mPEGₙ-Atazanavir-Methyl Ethyl Ether Compounds

mPEGₙ-Atazanvir compounds were prepared in accordance with the schematic provided below.

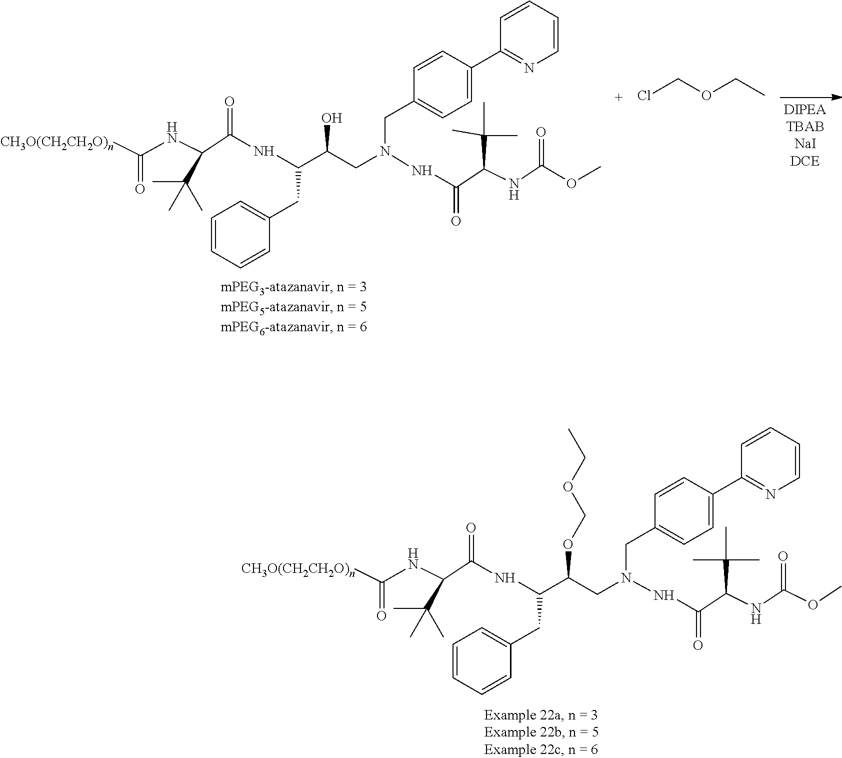

### Example 22a

### mPEG₃-Atazanavir Methyl Ethyl Ether

Into a 100 mL round bottom flask was added previously prepared mPEG₃-atazanavir (0.85 gm, 1.02 mmol) and anhydrous 1,2-dichloroethane (25 mL). To the clear solution was added diisopropyl ethyl amine (0.89 mL, 5.11 mmol), followed by chloromethyl ethyl ether (0.64 mL, 5.11 mmol), sodium iodide (0.077 gm, 0.51 mmol), and tetrabutylammonium bromide (0.066 gm, 0.20 mmol). The clear reaction mixture was heated to 70° C. under nitrogen. After approximately eighteen hours at 70° C., the dark amber reaction mixture was cooled to room temperature, and diluted with dichloromethane (100 mL). The organic mixture was transferred to a separatory funnel and partitioned with deionized water (100 mL). The aqueous layer was extracted with dichloromethane (3×30 mL). The combined organic layers were washed with saturated sodium bicarbonate, deionized water and saturated sodium chloride (100 mL each). The organic layer was dried over anhydrous sodium sulfate, filtered, and concentrated under reduced pressure to give 1.26 gm of a dark oil. Purification by Biotage chromatography (gradient elution: 0 to 5% methanol/dichloromethane over 20 column volumes) gave 0.72 gm (79%) of mPEG₃-atazanavir methyl ethyl ether as a white foam solid; R_{f} 0.43 (5% methanol-dichloromethane); ¹H NMR (DMSO-d6): δ 8.97 (bs, 1H), 8.64 (d, 1H), 7.96 (d, 2H), 7.92 (d, 1H), 7.86 (t, 1H), 7.56 (d, 1H), 7.39 (d, 2H), 7.32 (m, 1H), 7.22 (m, 4H), 7.18 (m, 1H), 6.82 (m, 2H), 4.94 (d, 1H), 4.72 (d, 1H), 4.38 (bs, 1H), 4.03 (m, 2H), 3.90 (m, 3H), 3.69 (On, 3H), 3.55 (On, 3H), 3.50 (m, 9H), 3.42 (m, 2H), 3.33 (s, 3H), 3.22 (s, 3H), 2.87 (m, 2H), 2.79 (m, 1H), 2.73 (m, 1H), 1.13 (t, 3H), 0.75 (s, 3H), 0.73 (s, 3H). MS 895.5 (M+H)⁺.

### Example 22b

### mPEG₅-Atazanavir Methyl Ethyl Ether

Into a 100 mL round bottom flask was added mPEG₅-atazanavir (0.85 gm, 0.91 mmol) and anhydrous 1,2-dichloroethane (22 mL). To the clear solution was added diisopropyl ethyl amine (0.80 mL, 4.59 mmol), followed by chloromethyl ethyl ether (0.57 mL, 4.59 mmol), sodium iodide (0.069 gm, 0.45 mmol), and tetrabutylammonium bromide (0.059 gm, 0.18 mmol). The clear reaction mixture was heated to 70° C. under nitrogen. After approximately eighteen hours at 70° C., the dark amber reaction mixture was cooled to room temperature, and diluted with dichloromethane (100 mL). The organic mixture was transferred to a separatory funnel and partitioned with deionized water (100 mL). The aqueous layer was extracted with dichloromethane (3×30 mL). The combined organic layers were washed with saturated sodium bicarbonate, deionized water and saturated sodium chloride (100 mL each). The organic layer was dried over anhydrous sodium sulfate, filtered, and concentrated under reduced pressure to give 1.10 gm of a dark oil. Purification by Biotage chromatography (gradient elution: 0 to 5% methanol/dichloromethane over 20 column volumes) gave 0.67 gm (74%) of mPEG₅-atazanavir methyl ethyl ether as a light-yellow foam solid; R_{f} 0.37 (5% methanol-dichloromethane); ¹H NMR (DMSO-d6): δ 8.97 (bs, 1H), 8.64 (d, 1H), 7.96 (d, 2H), 7.92 (d, 1H), 7.86 (t, 1H), 7.56 (d, 1H), 7.39 (d, 2H), 7.32 (m, 1H), 7.22 (m, 4H), 7.18 (m, 1H), 6.82 (m, 2H), 4.94 (d, 1H), 4.72 (d, 1H), 4.38 (bs, 1H), 4.03 (m, 2H), 3.90 (m, 3H), 3.69 (m, 3H), 3.55 (m, 3H), 3.50 (m, 17H), 3.42 (m, 2H), 3.33 (s, 3H), 3.22 (s, 3H), 2.87 (m, 2H), 2.81 (m, 1H), 2.72 (m, 1H), 1.13 (t, 3H), 0.75 (s, 3H), 0.73 (s, 3H). MS 983.6 (M+H)⁺.

### Example 22c

### mPEG₆-Atazanavir Methyl Ethyl Ether

Into a 100 mL round bottom flask was added mPEG₆-atazanavir (0.88 gm, 0.90 mmol) and anhydrous 1,2-dichloroethane (22 mL). To the clear solution was added diisopropyl ethyl amine (0.79 mL, 4.54 mmol), followed by chloromethyl ethyl ether (0.57 mL, 4.54 mmol), sodium iodide (0.068 gm, 0.45 mmol), and tetrabutylammonium bromide (0.058 gm, 0.18 mmol). The clear reaction mixture was heated to 70° C. under nitrogen. After approximately eighteen hours at 70° C., the dark amber reaction mixture was cooled to room temperature, and diluted with dichloromethane (100 mL). The organic mixture was transferred to a separatory funnel and partitioned with deionized water (100 mL). The aqueous layer was extracted with dichloromethane (3×30 mL). The combined organic layers were washed with saturated sodium bicarbonate, deionized water and saturated sodium chloride (100 mL each). The organic layer was dried over anhydrous sodium sulfate, filtered, and concentrated under reduced pressure to give 1.90 gm of a dark oil. Purification by Biotage chromatography (gradient elution: 0 to 5% methanol/dichloromethane over 20 column volumes) gave 0.75 gm (80%) of mPEG₆-atazanavir methyl ethyl ether as a light-yellow oil; R_{f} 0.42 (5% methanol-dichloromethane); ¹H NMR (DMSO-d6): δ 8.97 (bs, 1H), 8.64 (d, 1H), 7.96 (d, 2H), 7.92 (d, 1H), 7.86 (t, 1H), 7.56 (d, 1H), 7.39 (d, 2H), 7.32 (m, 1H), 7.22 (m, 4H), 7.18 (m, 1H), 6.82 (m, 2H), 4.94 (d, 1H), 4.72 (d, 1H), 4.38 (bs, 1H), 4.03 (m, 2H), 3.90 (m, 3H), 3.69 (m, 3H), 3.55 (m, 3H), 3.50 (m, 17H), 3.42 (m, 2H), 3.33 (s, 3H), 3.22 (s, 3H), 2.87 (m, 2H), 2.81 (m, 1H), 2.72 (m, 1H), 1.13 (t, 3H), 0.75 (s, 3H), 0.73 (s, 3H). MS 1027.6 (M+H)⁺.

### Examples 23a-23c

### Preparation of mPEG₃-Atazanavir-Methyl Ethyl Methyl Ether

mPEGₙ-Atazanvir-methyl, ethyl, methyl ether was prepared in accordance with the schematic provided below.

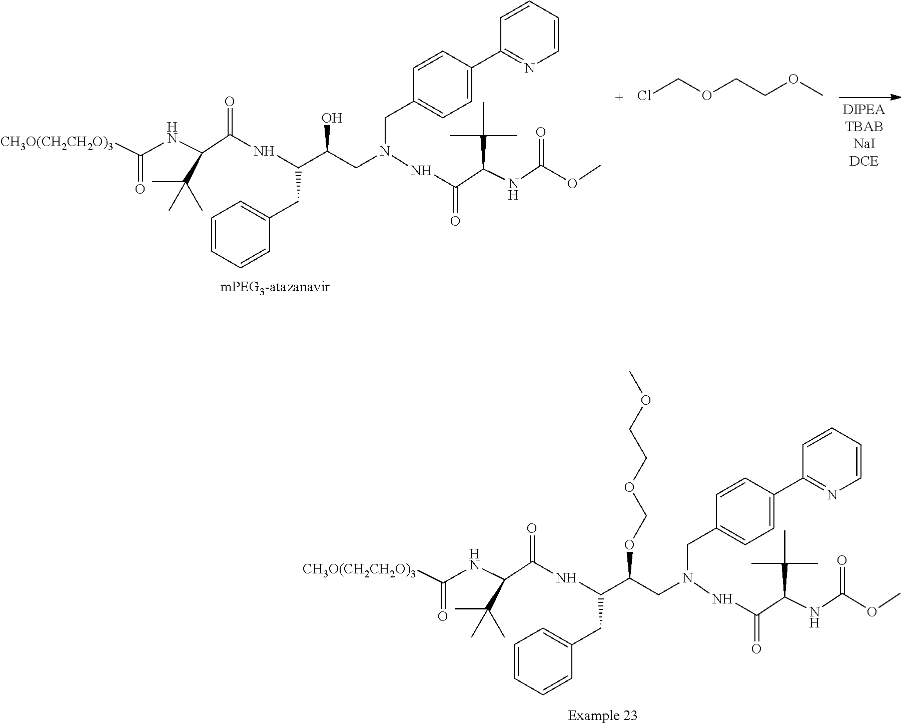

### Example 23a

### mPEG₃-Atazanavir Methyl Ethyl Methyl Ether

Into a 50 mL round bottom flask was added previously prepared mPEG₃-atazanavir (0.15 gm, 0.19 mmol) and anhydrous 1,2-dichloroethane (6 mL). To the clear solution was added diisopropyl ethyl amine (0.16 mL, 0.93 mmol), followed by 2-methoxyethoxymethyl chloride (0.10 mL, 0.93 mmol), and tetrabutylammonium bromide (0.012 gm, 0.03 mmol). The clear reaction mixture was heated to 70° C. under nitrogen. After approximately eighteen hours at 70° C., the dark amber reaction mixture was cooled to room temperature, and diluted with dichloromethane (50 mL). The organic mixture was transferred to a separatory funnel and partitioned with deionized water (50 mL). The aqueous layer was extracted with dichloromethane (3×10 mL). The combined organic layers were washed with saturated sodium bicarbonate, deionized water and saturated sodium chloride (50 mL each). The organic layer was dried over anhydrous sodium sulfate, filtered, and concentrated under reduced pressure to give a dark oil. Purification by Biotage chromatography (gradient elution: 0 to 5% methanol/dichloromethane over 20 column volumes) gave 0.12 gm (70%) of mPEG₃-atazanavir methyl ethyl methyl ether as a clear oil; R_{f} 0.42 (5% methanol-dichloromethane); ¹H NMR (DMSO-d6): δ 8.96 (bs, 1H), 8.64 (d, 1H), 7.96 (d, 2H), 7.92 (d, 1H), 7.86 (t, 1H), 7.57 (d, 1H), 7.39 (d, 2H), 7.32 (m, 1H), 7.22 (m, 4H), 7.18 (m, 1H), 6.82 (m, 2H), 4.99 (d, 1H), 4.76 (d, 1H), 4.35 (bs, 1H), 4.03 (m, 2H), 3.90 (m, 3H), 3.76 (m, 1H), 3.74 (m, 3H), 3.55 (m, 2H), 3.49 (m, 10H), 3.42 (m, 2H), 3.33 (s, 3H), 3.22 (s, 3H), 2.86 (m, 2H), 2.79 (m, 1H), 2.73 (m, 1H), 0.75 (s, 3H), 0.73 (s, 3H). MS 925.5 (M+H)⁺.

### Example 23b

### mPEG₅-Atazanavir Methyl Ethyl Methyl Ether

Using an approach similar to the one used to prepare mPEG₃-atazanavir methyl ethyl methyl ether, mPEG₅-atazanavir methyl ethyl methyl ether was prepared.

### Example 23c

### mPEG₆-Atazanavir Methyl Ethyl Methyl Ether

Using an approach similar to the one used to prepare mPEG₃-atazanavir methyl ethyl methyl ether, mPEG₆-atazanavir methyl ethyl methyl ether was prepared.

### Examples 24a-24b

### Monophospholipid Compounds of mPEGₙ-Atazanavir

Monophospholipids of mPEGₙ-atazanavir were prepared. One approach corresponds to the schematic provided below.

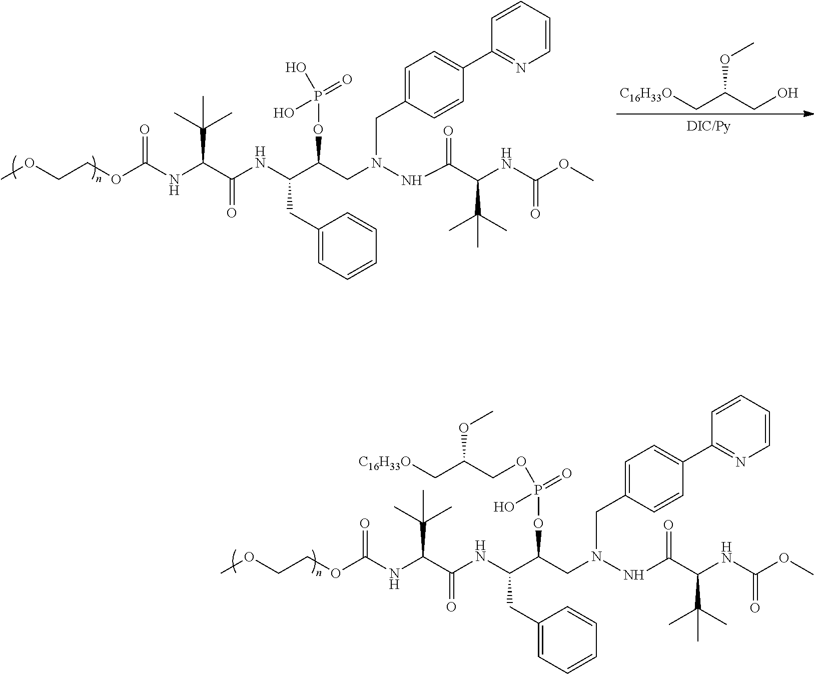

### Example 24a

mPEG₃-Atazanavir monophosphate was prepared in accordance with the approach set forth for Example 24b, wherein mPEG₃-atazanavir monophosphate (preparation set forth in Example 16) is substituted for mPEG₅-atazanavir monophosphate.

### Example 24b

mPEG₅-Atazanavir monophosphate (2.6717 g, 2.66 mmol) and 1-O-hexadecyl-2-O-methyl-sn-glycerol (>98% TLC) (1.3321 g, 3.95 mmol) were mixed with toluene (150 mL). After sonication for about three minutes, a suspension was observed. The toluene was removed under reduced pressure. The residue was dried under high vacuum for about ten minutes. Anhydrous pyridine (52 mL) was added. Thereafter, N,N-diisopropylcarbodiimide (DIC) (1.7 mL, 10.98 mmol) was added. The resulting mixture was stirred at room temperature for five minutes, at 50° C. for 100 minutes. EtOAc (300 mL) was added to the cooled reaction mixture, washed with 1 N HCl solution (2×100 mL) and 1 N HCl solution saturated with NaCl (2×100 mL), dried over Na₂SO₄, and concentrated. The residue was dissolved in small amount of EtOAc (˜15 mL), and then filtered to remove the white solid (Note: Based on the ¹H-NMR in DMSO, it is the byproduct diisopropyl urea (DIU). The EtOAc solution was added dropwise to hexane (˜100 mL), and centrifuge at 3000 rps for ten minutes. The precipitation was collected (purity ˜70% based on HPLC). The crude product was dissolved in EtOAc (100 mL), washed with 5% NaHCO₃ aqueous solution (2×120 mL), brine (120 mL), dried over Na₂SO₄, and concentrated (with purity improved to ˜84%). The similar precipitation process was repeated twice with Et₂O/hexane and EtOAc/Hexane. The purity was about 90% based on the HPLC analysis.

Part A: 14.6671-13.5884=1.0787 g (After treatment with aq. NaHCO₃ solution in EtOAc, purity: about 90%). Part B was mixed with all of hexane solutions generated from the precipitation process, concentrated. The residue was purified by flash column chromatography on silica gel and eluted with 5-10% MeOH/dichloromethane (40 CV, 15 CV, Biotage) to afford pure product (purity: >97%). After the purification with FCC, the product was dissolved in EtOAc (˜150 mL), washed with 5% aqueous NaHCO₃ solution (˜300 mL), dried over Na₂SO₄, concentrated and dried under high vacuum overnight to afford the final product, Example 24b, (774.9 mg) as sodium salt (white foam). Total: 1.0787×90%+0.7749=1.7457 g. Yield: 49%. ¹H-NMR (DMSO-d₆): 10.323 (s, 1H, NH), 8.638 (d, J=5.0 Hz, 1H, Ar—H), 7.912 (t, J=8.0 Hz, 4H, 4Ar—H), 7.847 (m, 1H), 7.342 (d, J=8.0 Hz, 2H, 2Ar—H), 7.323-7.299 (m, 1H), 7.249-7.234 (m, 2H, 2Ar—H), 7.171 (d, J=7.0 Hz, 2Ar—H), 7.130-7.102 (m, 1H), 6.925 (d, J=10 Hz, 1H, NH), 6.810 (d, J=9.0 Hz, 1H, NH), 4.369 (br, 1H), 4.136-4.006 (m, 5H, 2CHBu^{t}, CH and CH₂), 3.892-3.823 (m, 2H), 3.796-3.764 (m, 1H), 3.639 (d, J=10.0 Hz, 1H), 3.359 (t. J=5.0 Hz, 2H), 3.487 (m, 18H, 9CH₂), 3.413 (m, 2H, CH₂), 3.374-3.330 (m, 7H), 3.223 (s, 3H, CH₃), 2.966-2.927 (m, 3H), 2.686-2.641 (m, 1H), 1.430 (m, 2H, CH₂), 1.273-1.156 (m, 26H, 13CH₂), 0.843 (t, J=7.0 Hz, CH₂), 0.779 (s, 9H, Bu^{t}), 0.721 (s, 9H, Bu^{t}). LC-MS (ESI): 1317.8 (MH⁺).

### Example 25

### Amino Acid- and Peptide-Conjugates of Atazanavir

The following series of syntheses provide various approaches for adding a single amino acid to a protease inhibitor. Using the orthogonal approach of sequential attachment of amino acids provided herein, it is possible to attach an additional amino acid to the one immediately attached immediately before to thereby provide a peptide. Although the syntheses of Example 25 were carried out with specific amino acids, the approach can be carried out with all amino acids (including non-naturally occurring amino acids).

### Synthesis of Boc-Gly-mPEG₅-Atazanavir

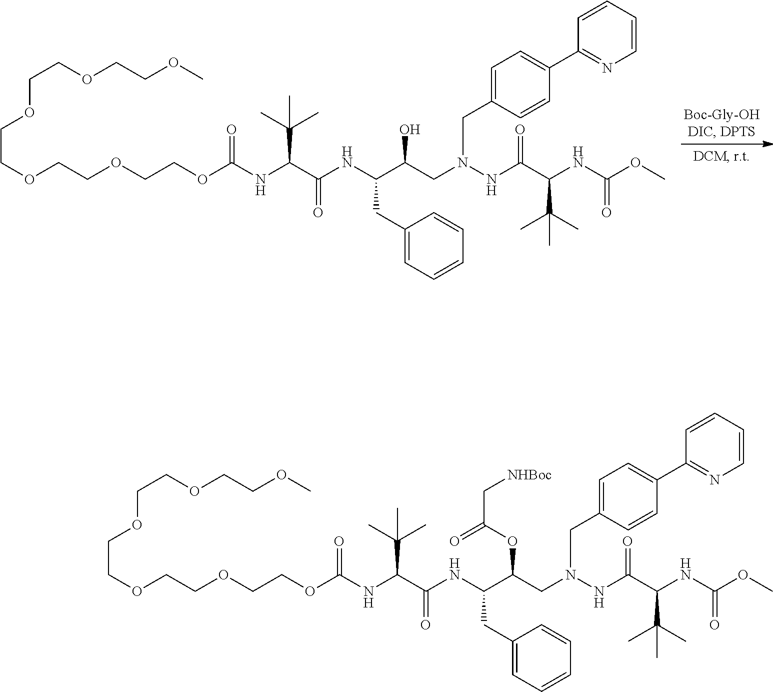

mPEG₅-Atazanavir (3.3002 g, 3.567 mmol), Boc-Gly-OH (12.0957 g, 68.357 mmol) and DPTS (1:1 mixture of DMAP and p-toluenesulfonic acid) (1.1116 g, 1776 mmol) were dissolved in anhydrous DCM (90 mL) at room temperature. DIC (11 mL, 70.328 mmol) was added. The resulting mixture was stirred at room temperature for four hours. The mixture was filtered through a celite funnel to remove the white solid and the solid was washed with DCM. The solution was collected and washed with 5% aq. NaHCO₃ solution (20 mL), dried over Na₂SO₄, and concentrated. The residue was purified with flash column chromatography on silica gel and eluted with 0-3% MeOH/DCM (15 CV, 40 CM) to afford 3.5443 g product as white foam. The yield was 92%. ¹H-NMR (CDCl₃): 8.685-8.670 (m, 1H), 7.901 (d, J=8.5 Hz, 1H, Ar—H), 7.767-7.733 (m, 1H, Ar—H), 7.713-7.697 (m, 1H, 1Ar—H), 7.311-7.295 (m, 2H, 2Ar—H), 7.238-7.211 (m, 3H, 3Ar—H), 7.184-7.140 (m, 3H, 3Ar—H), 6.162-6.152 (m, 1H, NH), 5.425-5.408 (m, 1H, NH), 5.298-5.286 (m, 2H, 2NH), 5.133-5.119 (br, 1H, NH), 4.352-4.340 (m, 1H, CH), 4.197-4.140 (m, 4H, 2CHBu^{t}, CH₂), 4.018-3.894 (m, 2H), 3.773-3.754 (m, 1H), 3.682-3.530 (m, 23H), 3369 (s, 3H, CH₃), 3.189-3.163 (m, 1H), 2.992-2.950 (m, 1H), 2.769-2.664 (m, 2H, CH₂), 1.484 (s, 9H, Bu^{t}), 0.866 (s, 9H, Bu^{t}), 0.770 (s, 9H, Bu^{t}). LC-MS (ESI): 1082.6 (MH⁺).

### Synthesis of Gly-mPEG₅-Atazanavir Hydrochloride

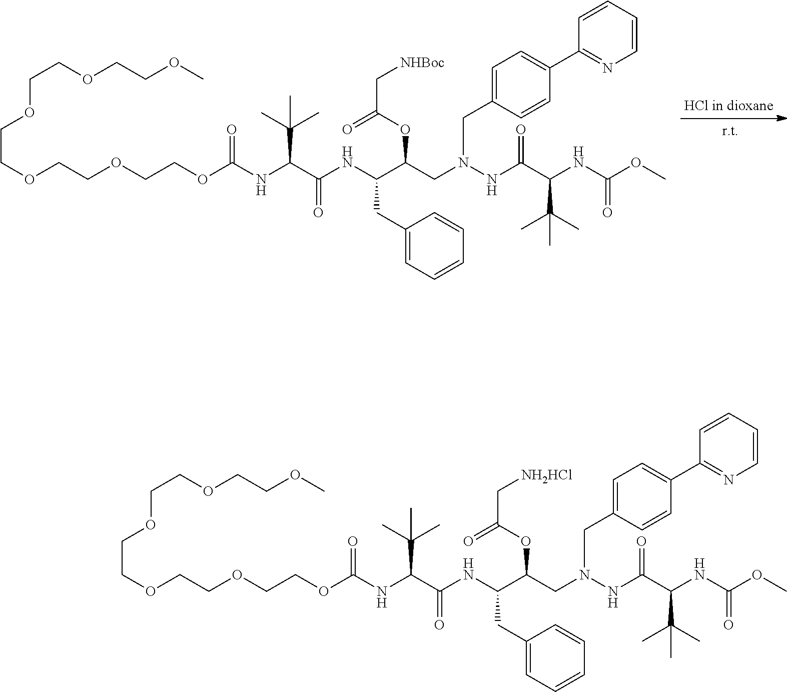

Boc-Gly-mPEG₅-Atazanavir (3.5443 g, 3.27 mmol) was dissolved in anhydrous dioxane (15 mL) at room temperature. Thereafter, 4N HCl solution dioxane (15 mL) was added. The resulting mixture was stirred at room temperature for 1.5 hours. DCM (200 mL) was added to dilute the reaction mixture. Saturated NaCl solution was added. A small amount of precipitation was observed. Thereafter, a small amount of water was added. The organic solution was separated and the aqueous solution was extracted with DCM. The combined organic solution was dried over anhydrous sodium sulfate, concentrated and dried under high vacuum to afford 3.3775 g product as white foam. The purity was 96% based on HPLC and the yield was 97%. ¹H-NMR (DMSO-d₆): 9.131 (m, 1H), 8.689 (d, J=5.0 Hz, 1H, 1Ar—H), 8.473 (m, 3H, 3 NH), 8.00 (br, 2H, 2NH), 7.956 (d, J=8.0 Hz, 2H, 2Ar—H), 7.907 (d, J=9.5 Hz, 1H, 1Ar—H), 7.453 (br, 1H, 1NH), 7.411 (d, J=8.5 Hz, 2H, 2Ar—H), 7.218-7.124 (m, 5H, 5 Ar—H), 6.791 (d, J=9.5 Hz, 1H, 1Ar—H), 6.739 (d, J=9.5 Hz, 1H, 1Ar—H), 5.152 (m, 1H), 4.60 (br, 1H), 4.079-3.820 (m, 4H), 3.721-3.641 (m, 2H), 3.592-3.406 (m, 23H), 3.227 (s, 3H, CH₃), 3.002-2.941 (m, 2H), 2.746-2.635 (m, 2H), 0.809 (s, 9 H, Bu^{t}), 0.739 (s, 9H, Bu^{t}). LC-MS (ESI): 982.6 (MH⁺).

### Synthesis of Gly-mPEG₅-Atazanavir Hydrochloride

Using an approach similar to that used to prepare Gly-mPEG₅-atazanavir hydrochloride, Gly-mPEG₃-atazanavir hydrochloride can be prepared.

### Synthesis of Gly-mPEG₆-Atazanavir Hydrochloride

Using an approach similar to that used to prepare Gly-mPEG₅-atazanavir hydrochloride, Gly-mPEG₆-atazanavir hydrochloride was prepared.

### Synthesis of Boc-Phe-Gly-mPEG₅-Atazanavir

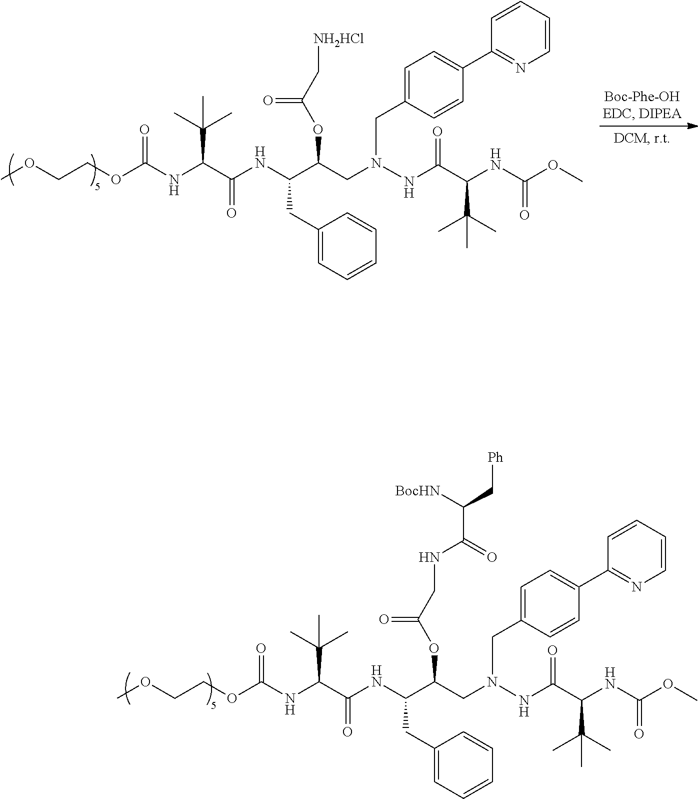

Gly-mPEG₅-atazanavir hydrochloride (709 mg, 0.668 mmol) and Boc-Phe-OH (556.3 mg, 2.097 mmol) were dissolved in anhydrous DCM (10 mL) at room temperature. DIPEA (0.65 mL, 3.713 mmol) was added, and then EDC.HCl (471.5 mg, 2.41 mmol) was added. The resulting mixture was stirred at room temperature for two hours. Aqueous NaHCO₃ solution (5%) (50 mL) was added to quench the reaction. DCM (50 mL) was added to dilute the mixture. The organic solution was separated and washed with 5% aqueous NaHCO₃ solution (100 mL), saturated NH₄Cl solution (100 mL), dried over sodium sulfate and concentrated. The residue was purified by flash column chromatography on silica gel and eluted with 1-5% MeOH in DCM (25M, 15 CV) to afford 721 mg of product as white foam. The yield was 85%. ¹H-NMR (CDCl₃): 8.642 (m, 1H, Ar—H), 8.008 (br, 1H, NH), 7.869 (d, J=8.5 Hz, 2H, Ar—H), 7.730-7.699 (m, 1H, Ar—H), 7.655-7.639 (m, 1H, 1Ar—H), 7.317 (d, J=7.0 Hz, 2H, 2Ar—H), 7.270-7.172 (m, 11H, 11 Ar—H), 6.515 (m, 1H, NH), 5.179-6.148 (m, 1H, NH), 5.394 (m, 1H, 1NH), 5.018 (br, 1H, NH), 4.669-4.558 (m, 3H, CH), 4.457-4.427 (m, 1H), 4.066-4.004 (m, 3H), 3.912-3.862 (m, 2H), 3.690-3.454 (m, 25H, CH₂Ph, 5CH₂CH₂, and OCH₃), 3.370 (s, 3H, CH₃), 3.044-2.999 (m, 1H), 2.937-2.775 (m, 3H), 1.297 (s, 9H, Bu^{t}), 0.804 (s, 9H, Bu^{t}), 0.769 (s, 9H, Bu^{t}). LC-MS (ESI): 1229.6 (MH⁺).

### Synthesis of Phe-Gly-mPEG₆-Atazanavir Hydrochloride

Using an approach similar to the one make Phe-Gly-mPEG₅-atazanavir hydrochloride, Phe-Gly-mPEG₆-atazanavir hydrochloride was prepared.

### Synthesis of Phe-Gly-mPEG₅-Atazanavir Hydrochloride

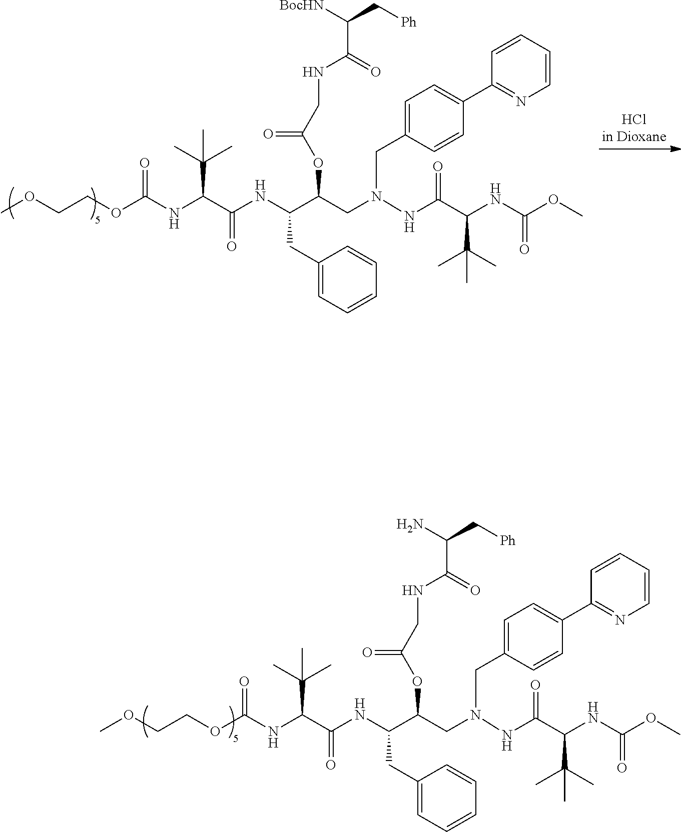

Boc-Phe-Gly-mPEG₅-Atazanavir (721 mg, 0.586 mmol) was dissolved in anhydrous dioxane (5 mL) at room temperature. Thereafter, 4N HCl solution dioxane (5 mL) was added. The resulting mixture was stirred at room temperature for 1.5 hours. Saturated NaCl solution was added to quench the reaction. The mixture was extracted with DCM (3×40 mL). The combined organic solution was dried over anhydrous sodium sulfate, concentrated and dried under high vacuum to afford 876.6 mg of crude product The product was dissolved in DCM (100 mL), washed with water, and saturated ammonium chloride solution, dried over sodium sulfate, concentrated and dried under high vacuum to afford 663.9 mg final product as white foam. Purity: >96% (based on HPLC). The yield was 93%. ¹H-NMR (DMSO): 9.197 (m, 1H), 9.090 (br, 1H), 8.709 (d, J=4.5 Hz, 1H), 8.339 (m, 3H), 8.062 (m, 2H), 7.975 (d, J=8.0 Hz, 3H), 7.524 (br, 1H), 7.427 (d, J=7.5 Hz, 2H), 7.331-7.263 (m, 5H), 7.207-7.123 (m, 5H), 6.719 (m, 2H), 5.088 (m, 1H), 4.561 (m, 1H), 4.204-3.959 (m, 4H), 3.719-3.646 (m, 2H), 3.563-3.397 (m, 23H), 3.224 (s, 3H), 3.194-3.184 (m, 1H), 3.086-3.043 (m, 2H), 2.919-2.610 (m, 2H), 0.810 (s, 9H, Bu^{t}), 0.742 (s, 9H, Bu^{t}). LC-MS (ESI): 1129.7 (MH⁺).

### Synthesis of Phe-Gly-mPEG₃-Atazanavir Hydrochloride

Using an approach similar to the one used to prepared Phe-Gly-mPEG₅-atazanavir hydrochloride, Phe-Gly-mPEG₃-atazanavir hydrochloride was prepared.

### Synthesis of Boc-Leu-Gly-mPEG₅-Atazanavir

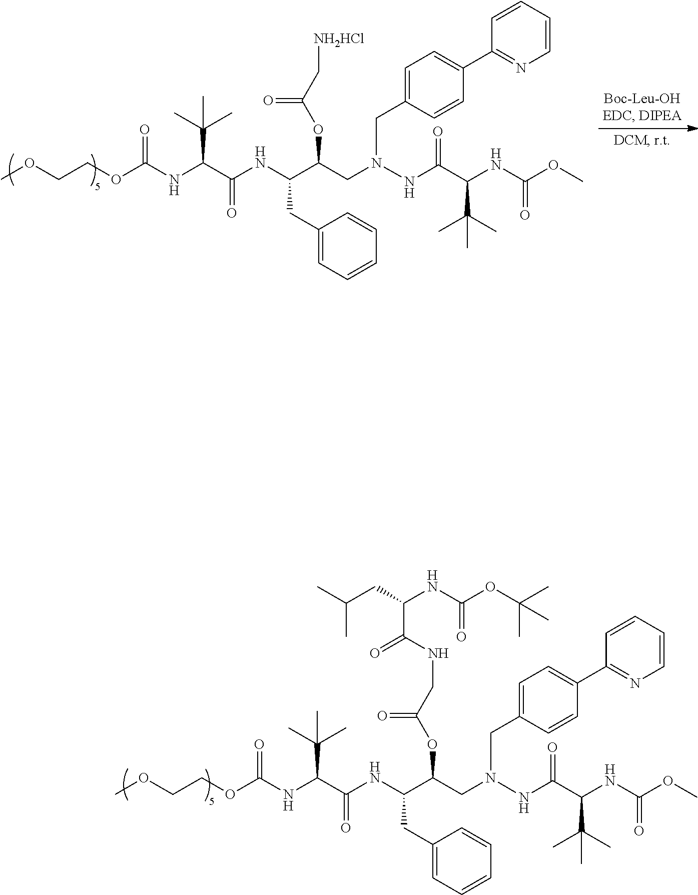

Gly-mPEG₅-atazanavir hydrochloride (775 mg, 0.730 mmol) and Boc-Leu-OH (532 mg, 2.277 mmol) were dissolved in anhydrous DCM (10 mL) at room temperature. DIPEA (0.65 mL, 3.713 mmol) was added, and then EDC.HCl (499 mg, 2.55 mmol) was added. The resulting mixture was stirred at room temperature for 2.5 hours. More of DCM (˜20 mL) was added to dilute the reaction mixture. Aqueous NaHCO₃ solution (5%) (100 mL) was added. The organic solution was separated and washed with saturated NH₄Cl solution (100 mL), dried over sodium sulfate, and concentrated. The residue was purified by flash column chromatography on silica gel and eluted with 1-10% MeOH in DCM (25M, 25 CV) to afford 819.5 mg of product as white foam. Purity: about 96%. Yield was 90%. ¹H-NMR in CDCl₃: 8.662 (m, 1H), 8.084 (br, 1H), 7.885 (d, J=8.0 Hz, 2H, Ar—H), 7.757-7.692 (m, 2H), 7.312 (d, J=8.0 Hz, 2H), 7.232-7.202 (m, 5H), 7.160-7.143 (m, 1H), 6.649 (m, 1H), 5.130 (m, 1H), 5.034 (m, 1H), 5.344 (m, 1H), 5.034 (m, 1H), 4.630-4.570 (m, 1H), 4.445-4.419 (m, 1H), 4.367-4.333 (m, 1H), 4.080-3.983 (m, 3H), 3.893-3.847 (m, 2H), 3.753-3.486 (s, 24H), 3.378 (s, 3H), 3.347 (m, 1H), 3.028-3.982 (m, 1H), 2.834-2.747 (m, 2H), 2.059-1.997 (m, 1H), 1.690 (m, 2H), 1.455 (s, 9H, Bu^{t}), 0.975-0.960 (m, 6 H), 0.825 (s, 9H, Bu^{t}), 0.792 (s, 9H, Bu^{t}). LC-MS (ESI): 1195.7 (MH⁺).

### Synthesis of Leu-Gly-mPEG₅-Atazanavir Hydrochloride

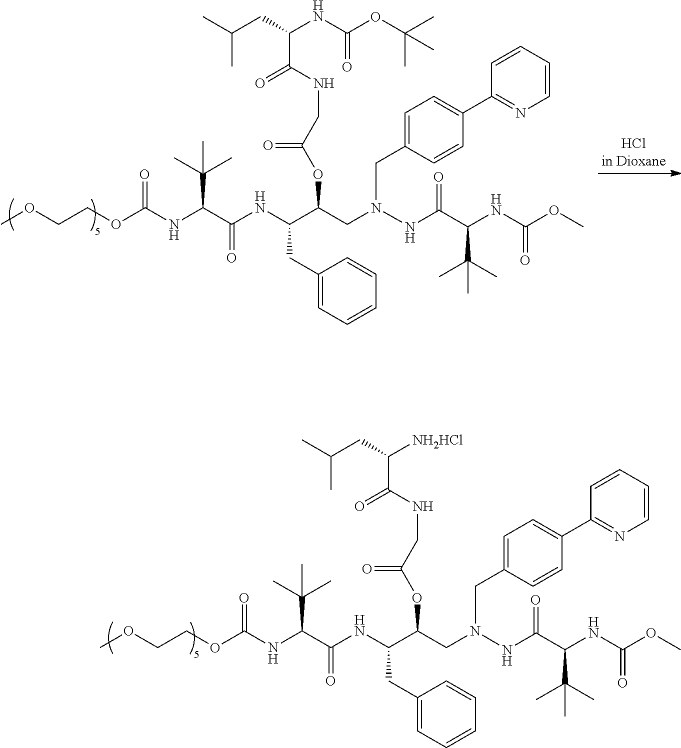

Boc-Leu-Gly-mPEG₅-Atazanavir (819 mg, 0.658 mmol) was dissolved in anhydrous dioxane (5 mL) at room temperature. Thereafter, 4N HCl solution dioxane (5 mL) was added. The resulting mixture was stirred at room temperature for two hours. DCM (100 mL) was added to dilute the reaction mixture. Sat. NaCl solution (120 mL) was added. The organic phase was separated and the aqueous phase was extracted with DCM (20 mL). The combined organic solution was washed with saturated NH₄Cl solution (100 mL), dried over anhydrous sodium sulfate, concentrated and dried under high vacuum to afford 765.4 mg of product. Purity: >95% (based on HPLC). The yield was 94%. ¹H-NMR in DMSO: 9.059-9.038 (m, 2H), 8.655 (d, J=4.5 Hz, 1H), 8.291 (m, 3H), 7.958-7.900 (m, 5H), 7.383-7.352 (m, 3H), 7.207-7.131 (m, 5H), 6.759-6.712 (m, 2H), 5.101 (m, 1H), 4.452 (m, 1H), 4.128-3.653 (m, 7H), 3.588-3.445 (m, 18H), 3.423-3.404 (m, 5H), 3.225 (s, 3H), 3.075-3.065 (m, 1H), 2.940-2.880 (m, 1H), 2.729-2.632 (m, 2H), 1.780-1.697 (m, 1H), 1.658-1.568 (m, 1 H), 0.912 (t, J=6.5 Hz, 6H), 0.810 (s, 9H, 0.741 (s, 9H, Bu^{t}). LC-MS (ESI): 1095.7 (MH⁺).

### Synthesis of Leu-Gly-mPEG₃-Atazanavir Hydrochloride

Using an approach similar to the one make Leu-Gly-mPEG₅-atazanavir hydrochloride, Leu-Gly-mPEG₃-atazanavir hydrochloride was prepared.

### Synthesis of Leu-Gly-mPEG₆-Atazanavir Hydrochloride

Using an approach similar to the one make Leu-Gly-mPEG₅-atazanavir hydrochloride, Leu-Gly-mPEG_{G}-atazanavir hydrochloride was prepared.

### Synthesis of Boc-Val-Gly-mPEG₅-Atazanavir

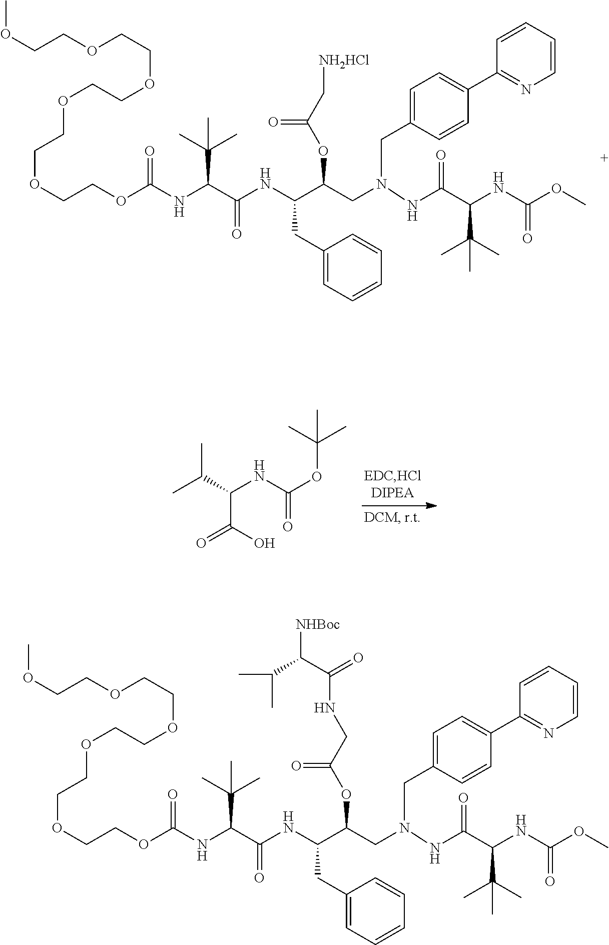

mPEG₅-Atazanavir-Gly-NH₂.HCl (886 mg, 0.870 mmol) and Boc-Val-OH (560 mg, 2.55 mmol) were dissolved in anhydrous dichloromethane (10 mL) at room temperature. DIPEA (0.75 mL, 4.31 mmol) was added. After a few minutes, the solid was completely dissolved. EDC.HCl (564 mg, 2.94 mmol) was added. The resulting mixture was stirred at room temperature for 4.5 hours. NaHCO₃ aqueous solution (5%) (50 mL) was added to quench the reaction. The organic solution was separated and the aqueous component was extracted with DCM (50 mL). The combined organic solution was washed with saturated NaCl (2×100 mL), dried over Na₂SO₄, and concentrated. The residue was purified by flash column chromatography on silica gel and eluted with 1-10% MeOH in DCM (biotage SP⁴ system, 25M column, 25 CV) to afford 0.9038 g product as white foam. The yield was 88%. ¹H-NMR in CDCl₃: 8.665 (d, J=4.5 Hz, 1H), 7.888 (d, J=8.0 Hz, 3 H), 7.757-7.695 (m, 2H), 7.297 (d, J=8.0 Hz, 2H), 7.237-7.201 (m, 5H), 7.168-7.143 (m, 1H), 6.516 (br, 1H), 6.113-6.094 (m, 1H), 5.922-5.904 (m, 1H), 5.333-5.315 (m, 1H), 5.056-5.045 (m, 1H), 4.564-4.501 (m, 1H), 4.416-4389 (m, 1H), 4.266-4.255 (m, 1H), 4.083-4.047 (m, 3H), 3.900-3.866 (m, 2H), 3.722-3.481 (s, 24H), 3.377 (s, 3H), 3.346-3.305 (m, 1H), 3.042-2.998 (m, 1H), 2.794 (d, J=8.0 Hz, 2H), 2.563 (m, 1H), 1.462 (s, 9 H, Bu^{t}), 0.975 (d, J=6.5-7.0 Hz, 3H), 0.875 (d, J=6.5-7.0 Hz, 3H), 0.853 (s, 9H, Bu^{t}), 0.783 (s, 9H, Bu^{t}). LC-MS (ESI): 1181.7 (MH⁺).

### Synthesis of Val-Gly-mPEG₅-Atazanavir Hydrochloride

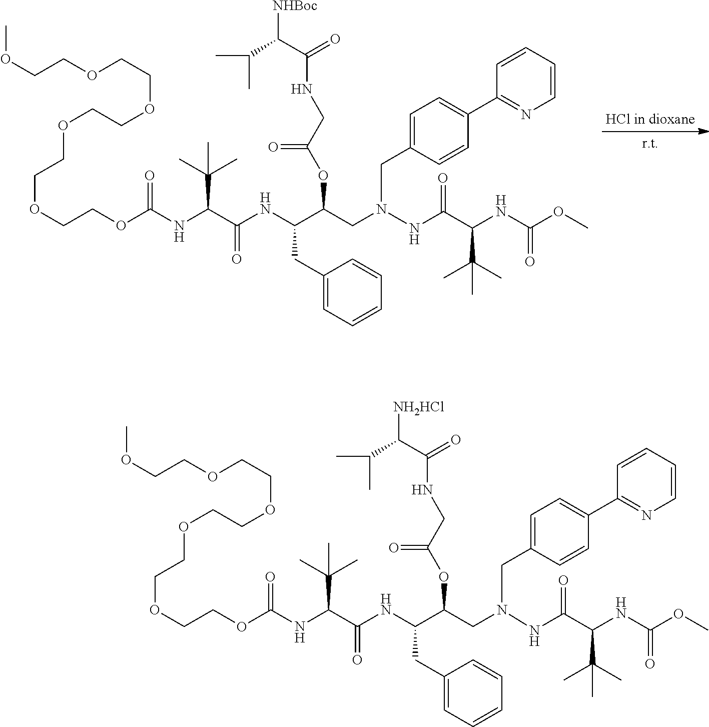

Boc-Val-Gly-mPEG₅-atazanavir (903 mg, 0.764 mmol) was dissolved in dioxane (5 mL) at room temperature, 4N HCl solution in dioxane was added. The resulting solution was stirred at room temperature for one hour, thirty-five minutes. DCM (˜100 mL) was added to dilute the reaction mixture. Sat. NaCl solution was added to quench the reaction. Small amount of water was added to dissolve the white precipitation. The organic solution was separated and the aqueous solution was extracted with DCM (20 mL). The combined organic solution was washed with sat. NH₄Cl solution (2×100 mL), dried over anhydrous sodium sulfate, and concentrated. The residue was dried under high vacuum to afford 810.3 mg final product as white foam (purity: >96% based on HPLC). The yield was 91%. ¹H-NMR in DMSO: 9.050 (m, 1H), 8.926 (t, J=5.0-5.5 Hz, 1H), 8.652 (d, J=5.0 Hz, 1H), 8.215 (m, 3H), 7.954-7.873 (m, 5H), 7.380-7.319 (m, 3H), 7.217-7.118 (m, 5H), 6.747-6.693 (m, 2H), 5.105 (t, J=6.0 Hz, 1H), 4.530 (m, 1H), 4.129-3.654 (m, 7H), 3.589-3.458 (m, 18H), 3.424-3.405 (m, 5H), 3.227 (s, 3H), 3.106-3.070 (m, 1H), 2.940-2.902 (m, 1H), 2.742-2.708 (m, 1H), 2.652-2.604 (m, 1H), 2.182-2.116 (m, 1H), 0.986 (t, J=6.0-6.5 Hz, 6H), 0.813 (s, 9H, Bu^{t}), 0.741 (s, 9H, Bu^{t}). LC-MS (ESI): 1081.6 (MH⁺).

### Synthesis of Val-GIy-mPEG₆-Atazanavir Hydrochloride

Using an approach similar to the one make Val-Gly-mPEG₅-atazanavir hydrochloride, Val-Gly-mPEG₆-atazanavir hydrochloride was prepared.

### Synthesis of Boc-Phe-mPEG₃-Atazanavir

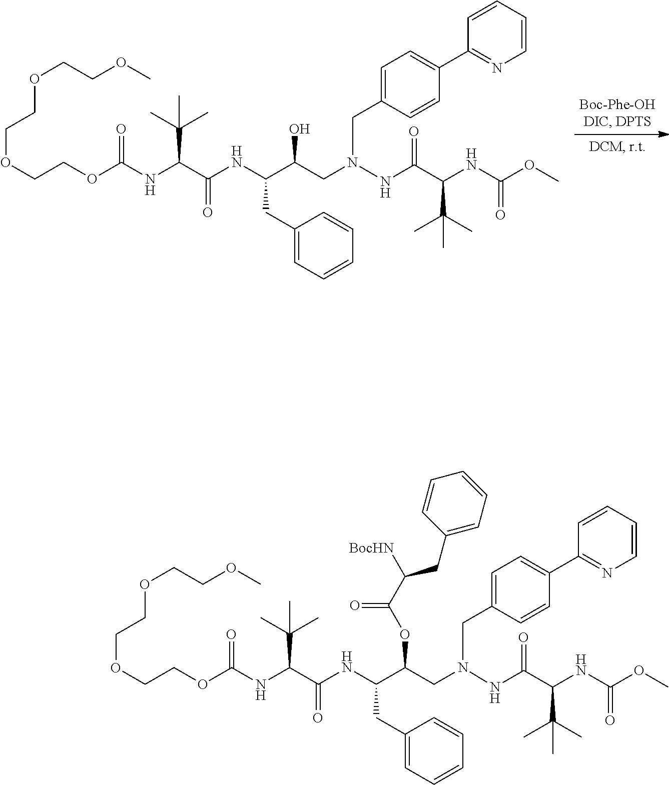

Boc-Phe-OH (7.1890 g, 27.098 mmol) was dissolved in DCM (70 mL). mPEG₃-atazanavir (2.3027 g, 2.75 mmol), DPTS (1:1 mixture of DMAP and p-toluenesulfonic acid) (843.2 mg, 2.864 mmol) were added and then DIC (5.2 mL, 33.2 mmol) was added to the stirred solution. After a few minutes, precipitation was observed. The resulting mixture was stirred at room temperature for 4.5 hours. The mixture was filtered through a celite funnel and the solid was washed with DCM. The solution was collected and washed with 5% NaHCO₃ aq. solution (150 mL), dried over sodium sulfate, and concentrated. The residue was purified by flash column chromatography on silica gel and eluted with 1-6% MeOH in DCM (25 CV) to afford 3.1797 g of product. Purity: 95% (based on HPLC). ¹H-NMR (CDCl₃): 8.676 (d, J=5.0 Hz, 1H), 7.844 (d, J=8.0 Hz, 2H), 7.745 (dt, J=8.0 Hz and 1.5 Hz, 1H), 7.678 (d, J=8.0 Hz, 1H), 7.734 (br, 1H), 7.400 (m, 3H), 7.298 (d, J=8.0 Hz, 2H), 7.257-7.179 (m, 4H), 7.109 (t, J=7.0-7.5 Hz, 1H), 7.062 (d, J=7.5 Hz, 2H), 5.420 (d, J=9.0 Hz, 2H), 5.305 (br, 1H), 5.140 (m, 2H), 4.723 (m, 1H), 4.264-4.085 (m, 5H), 3.798-3.752 (m, 1H), 3.700-3.626 (m, 13H), 3.548-3.529 (m, 2H), 3.361 (s, 3H, CH₃), 3.246-2.89 (m, 4H), 2.391-2.300 (m, 2H), 1.432 (s, 9H, Bu^{t}), 0.863 (s, 9H, Bu^{t}), 0.755 (s, 9H, Bu^{t}). LC-MS (ESI): 1084.6 (MH⁺)

Based on NMR in CDCl₃, the product contained 9.6% DIU (per mole). The product was direct used for the next steps.

### Synthesis of Phe-mPEG₃-Atazanavir Hydrochloride

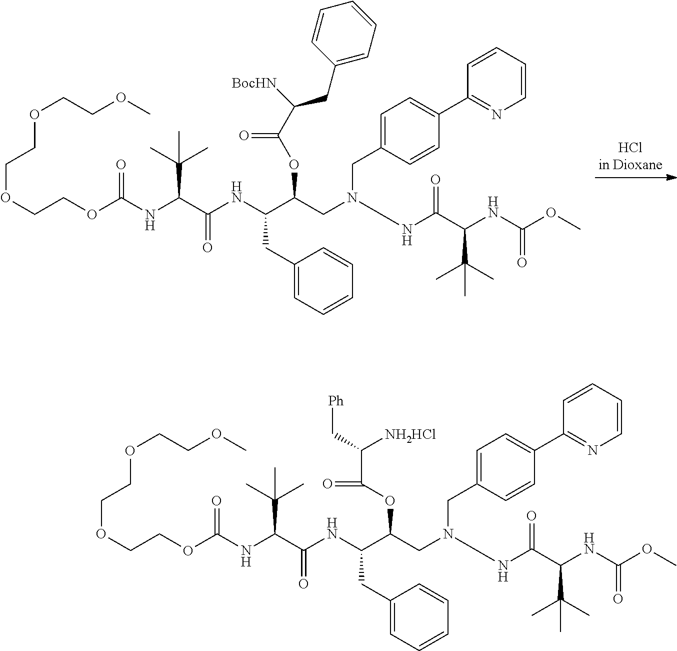

Boc-Phe-mPEG₃-Atazanavir (3.1797 g, 2.79 mmol) was dissolved in anhydrous dioxane 20 mL) at room temperature. Thereafter, 4N HCl solution dioxane (20 mL) was added. The resulting mixture was stirred at room temperature for one hour. DCM (150 mL) was added to dilute the reaction mixture. Saturated NaCl solution (120 mL) was added. The organic phase was separated and washed with saturated NH₄Cl solution (100 mL), dried over anhydrous sodium sulfate, concentrated and dried under high vacuum to afford 2.1009 g of product. Purity: >95% (based on HPLC). ¹H-NMR in DMSO: 9.173 (m, 1H), 8.660 (d, J=4.5 Hz, 1H), 8.585 (m, 2H), 7.962-7.901 (m, 4H), 7.800 (d, J=9.0 Hz, 1 H), 7.399-7.290 (m, 8H), 7.210-7.132 (m, 5H), 6.849 (d, J=9.5 Hz, 1H), 6.757 (d, J=9.5 Hz, 1H), 5.102 (m, 1H), 4.580 (m, 1H), 4.412 (m, 1H), 4.060 (m, 2H), 3.973-3.890 (m, 3H), 3.715-3.388 (m, 16H), 3.227 (s, 3H), 3.208-3.151 (m, 1H), 2.998-2.914 (m, 2H), 2.686-2.575 (m, 2H), 0.823 (s, 9H, Bu^{t}), 0.749 (s, 9H, Bu^{t}). LC-MS (ESI): 984.5 (MH⁺).

### Synthesis of Boc-Leu-Phe-mPEG₃-Atazanavir

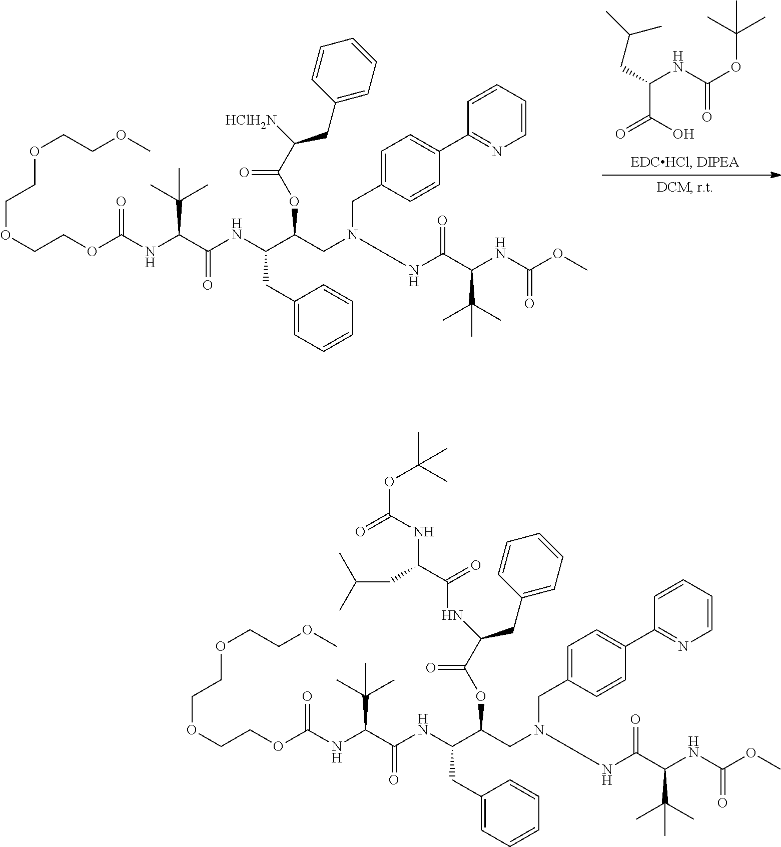

Phe-mPEG₃-atazanavir hydrochloride (855 mg, 0.830 mmol) was dissolved in anhydrous dichloromethane (12 mL) at room temperature. DIPEA (0.7 mL, 4.02 mmol) was added, followed by addition of Boc-Leu-OH (579.8 mg, 2.482 mmol). After a few minutes, the solid was completed dissolved. EDC.HCl (555.3 mg, 2.90 mmol) was added. The resulting mixture was stirred at room temperature for 2.5 hours. NaHCO₃ aqueous solution (5%) (50 mL) was added to quench the reaction. The organic solution was separated, the aqueous was extracted with DCM (50 mL). The combined organic solution was washed with sat. NaCl (2×100 mL), dried over Na₂SO₄, and concentrated

The residue was purified by flash column chromatography on silica gel and eluted with 1-10% MeOH in DCM (biotage SP⁴ system, 25M column, 25 CV) to afford 887.1 mg product contained small amount of DIU. LC-MS: 1197.11 (MH+). Due to the DIU, the product was purified again by flash column chromatography on silica gel and eluted with 10-50% acetone in hexane (biotage SP⁴ system, 25M column, 25 CV) to afford 730.1 mg pure product. The yield was 75%. ¹H-NMR (500 MHz, CDCl₃) δ 8.677 (d, J=4.5 Hz, 1H), 8.013 (br, 1H), 7.882 (d, J=8.0 Hz, 2H), 7.745 (dt, J=2.0 and 8.0 Hz, 1H), 7.711 (d, J=8.0 Hz, 1H), 7.383-7.295 (m, 7H), 7.234-7.201 (m, 3H), 7.149-7.099 (m, 2H), 5.562 (br, 1H), 5.476 (br, 1H), 5.250 (br, 1H), 5.098 (br, 1H), 4.738 (br, 1H), 4.277-4.066 (m, 6H), 3.730-3.625 (m, 14H), 3.548-3.529 (m, 3H), 3.369 (s, 3H), 3.272-3.180 (m, 1H), 3.119-3.073 (m, 1H), 3.000 (br, 2H), 2.458 (br, 2H), 1.675 (m, 2H), 1.528 (m, 1H), 1.463 (s, 9H), 0.930 (d, J=6.0 Hz, 3H), 0.899 (d, J=6.0 Hz, 3H), 0.857 (s, 9H), 0.776 (s, 9H). LC-MS (ESI): 1197.7 (MH⁺).

### Synthesis of Leu-Phe-mPEG₃-Atazanavir Hydrochloride

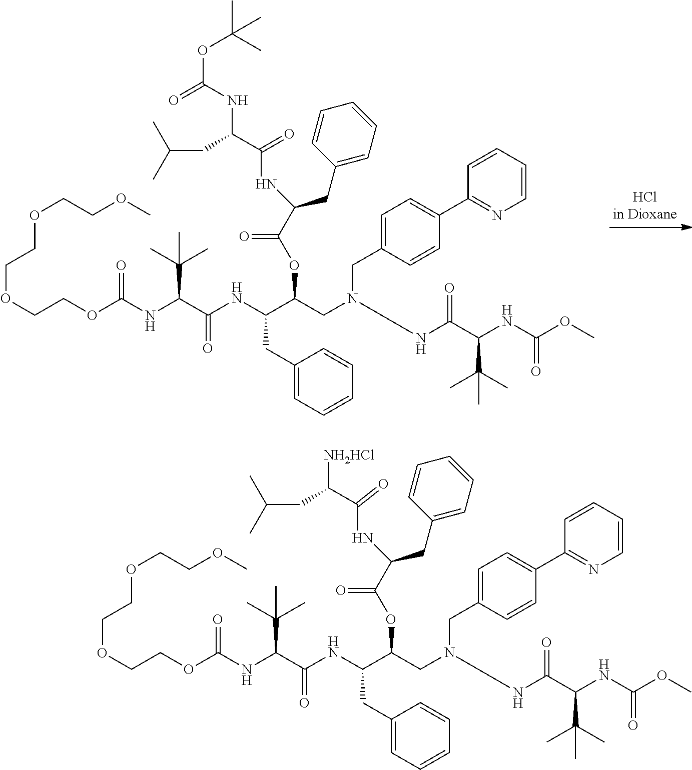

Boc-Leu-Phe-mPEG₃-atazanavir (730 mg, 0.610 mmol) was dissolved in anhydrous dioxane (5 mL) at room temperature. Thereafter, 4N HCl solution dioxane (5 mL) was added. The resulting mixture was stirred at room temperature for 1.5 hours. Saturated NaCl solution was added. The organic solution was separated and the aqueous solution was extracted with DCM (25 mL). The combined organic solution was washed with saturated NH₄Cl solution (100 mL), dried over anhydrous sodium sulfate, concentrated and dried under high vacuum to afford 747.5 mg product as white foam. The purity was >96% based on HPLC. ¹H-NMR in DMSO: 9.182-9.114 (br, 1H), 8.943 (br, 1H), 8.666 (d, J=3.5 Hz, 1H), 8.179 (m, 3H), 7.949-7.874 (m, 5H), 7.390 (d, J=8.0 Hz, 3H), 7.329-7.285 (m, 4H), 7.241-7.144 (m, 6H), 6.779 (d, J=10 Hz, 1H), 6.669 (d, J=10 Hz, 1H), 5.023 (m, 1H), 4.758 (m, 1H), 4.630 (m, 1H), 4.075-3.950 (m, 5H), 3.808 (m, 2H), 3.721-3.404 (m, 14H), 3.221 (s, 3H), 3.064-3.017 (m, 2H), 2.902 (m, 2H), 2.632 (m, 2H), 1.697-1.535 (m, 3H), 0.876-0.841 (s, 15H, 2 Me and Bu^{t}), 0.753 (s, 9H, Bu^{t}). LC-MS (ESI): 1097.6 (MH⁺).

### Synthesis of Leu-Phe-mPEG₅-Atazanavir Hydrochloride

Using an approach similar to the one used to make Leu-Phe-mPEG₃-atazanavir hydrochloride, Leu-Phe-mPEG₅-atazanavir hydrochloride was prepared.

### Synthesis of Leu-Phe-mPEG₆-Atazanavir Hydrochloride

Using an approach similar to the one used to make Leu-Phe-mPEG₃-atazanavir hydrochloride, Leu-Phe-mPEG₆-atazanavir hydrochloride was prepared.

### Synthesis of Boc-Phe-Phe-mPEG₃-Atazanavir

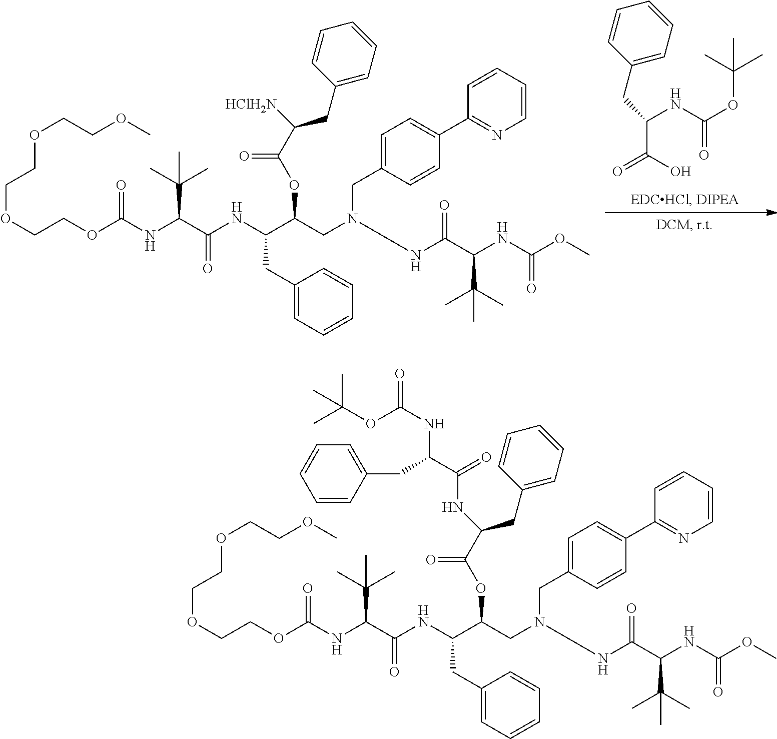

Phe-mPEG₃-atazanavir hydrochloride (881.6 mg, 0.864 mmol) was dissolved in anhydrous dichloromethane (10 mL) at room temperature. DIPEA (0.73 mL, 4.19 mmol) was added, followed by addition of Boc-Phe-OH (684.5 mg, 2.58 mmol). After a few minutes, the solid was completed dissolved. EDC.HCl (570.6 mg, 2.98 mmol) was added. The resulting mixture was stirred at room temperature for three hours. (The reaction was finished in one hour). 5% NaHCO₃ aqueous solution (50 mL) was added to quench the reaction. The organic solution was separated, the aqueous was extracted with DCM (50 mL). The combined organic solution was washed with sat. NaCl (2×100 mL), dried over Na₂SO₄, concentrated.

The residue was purified by flash column chromatography on silica gel and eluted with 10-50% acetone in hexane (biotage SP⁴ system, 25M column, 25 CV). Due to the DIU, the product was purified again by flash column chromatography on silica gel and eluted with 1-6% MeOH in DCM (25 CV, 25M) to afford 906.3 mg of product as white foam. The yield was 85%. ¹H-NMR (CDCl₃): 8.546 (br, 1H), 7.900 (br, 1H), 7.854 (d, J=8.5 Hz, 2H), 7.728-7.665 (m, 2H), 7.349 (d, J=4.5 Hz, 3H), 7.270-7.107 (m, 15H), 7.046 (br, 1H), 5.098-5.487 (m, 3H), 5.110 (br, 1H), 4.773 (m, 1H), 4.307-4.129 (m, 5H), 4.035 (m, 1H), 3.738-3.500 (m, 16H), 3.361 (s, 3H, CH₃), 3.218-3.019 (m, 6H), 2.465 (m, 2H), 1.399 (s, 9 H, Bu^{t}), 0.854 (s, 9H, Bu^{t}), 0.776 (s, 9H, Bu^{t}). LC-MS (ESI): 1231.6 (MH⁺).

### Synthesis of Phe-Phe-mPEG₃-Atazanavir Hydrochloride

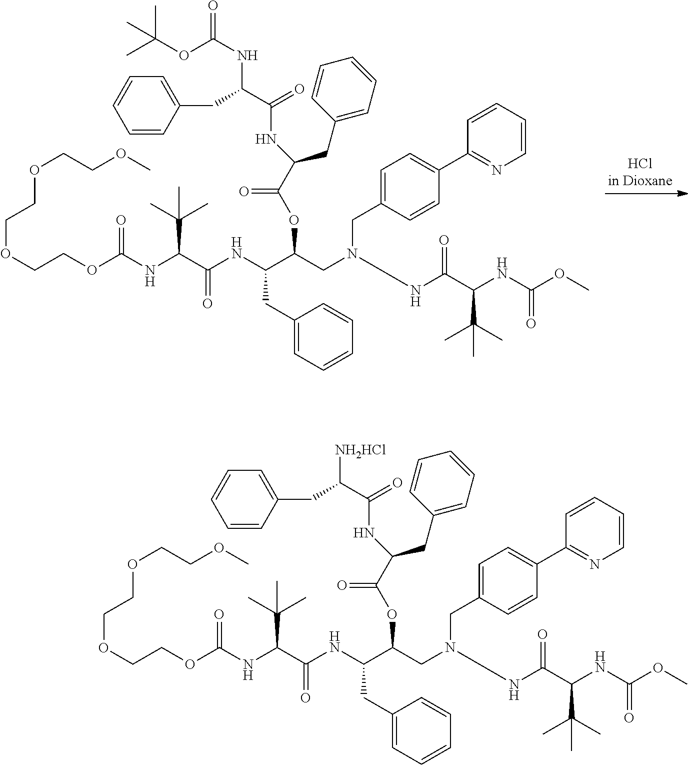

Boc-Phe-Phe-mPEG₃-atazanavir (906 mg, 0.736 mmol) was dissolved in anhydrous dioxane (5 mL) at room temperature. Thereafter, 4N HCl solution dioxane (5 mL) was added. The resulting mixture was stirred at room temperature for one hour, twenty minutes. Saturated NaCl solution was added. The organic solution was separated and the aqueous solution was extracted with DCM (25 mL). The combined organic solution was washed with saturated NaCl solution (2×100 mL), dried over anhydrous sodium sulfate, concentrated and dried under high vacuum to afford 835 mg product as white foam. The yield was 97%. ¹H-NMR in DMSO: 9.352 (d, J=8.5 Hz, 1H), 8.934 (br, 1H), 8.637 (d, J=4.5 Hz, 1H), 8.132 (m, 3H), 7.940-7.867 (m, 3H), 7.854-7.827 (m, 2H), 7.389 (d, J=8.5 Hz, 2H), 7.341-7.122 (m, 16H), 6.792 (d, J=9.5 Hz, 1H), 6.700 (d, J=9.5 Hz, 1H), 5.038 (m, 1 H), 4.811-4.767 (m, 1H), 4.640 (m, 1H), 4.122-3.993 (m, 6H), 3.724-3.673 (m, 2H), 3.565-3.339 (m, 13H), 3.268-3.225 (m, 1H), 3.223 (s, 3H), 3.095-3.048 (m, 2H), 2.968-2.890 (m, 2H), 2.706-2.622 (m, 2H), 0.838 (s, 9H, BO, 0.747 (s, 9H, Bu^{t}). LC-MS (ESI): 1131.6 (MH⁺).

### Synthesis of Phe-Phe-mPEG₃-Atazanavir Hydrochloride

Using an approach similar to the one used to make Phe-Phe-mPEG₃-atazanavir hydrochloride, Phe-Phe-mPEG₅-atazanavir hydrochloride was prepared.

### Synthesis of Phe-Phe-mPEG₆-Atazanavir Hydrochloride

Using an approach similar to the one used to make Phe-Phe-mPEG₃-atazanavir hydrochloride, Phe-Phe-mPEG₆-atazanavir hydrochloride was prepared.

### Synthesis of Boc-Val-Phe-mPEG₃-Atazanavir

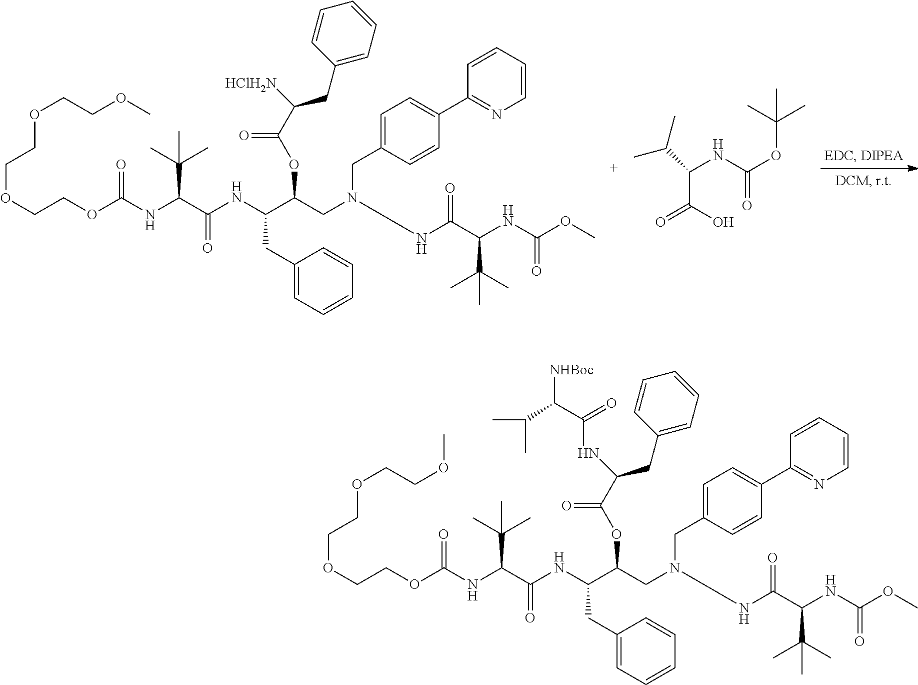

Phe-mPEG₃-atazanavir hydrochloride (95%) (833.6 mg, 0.776 mmol) was dissolved in anhydrous DCM (10 mL) at room temperature, DIPEA (0.7 mL, 4.02 mmol) was added. Thereafter, Boc-Val-OH (534.9 mg, 2.437 mmol) was added, followed by an addition of EDC.HCl (581.5 mg, 3.03 mmol). The resulting solution was stirred at room temperature for three hours. DCM (˜100 mL) was added to dilute the reaction mixture. NaHCO₃ aqueous solution (5%) (35 mL) was added to quench the reaction. The organic solution was separated, the aqueous was extracted with DCM (30 mL). The combined organic solution was washed with saturated NaCl (100 mL), dried over Na₂SO₄, and concentrated. The residue was purified by flash column chromatography (FCC) on silica gel and eluted with 1-6% MeOH in DCM (biotage SP⁴ system, 25M column, 25 CV). Due to DIU, the product was purified again by FCC on silica gel and eluted with 10-50% acetone in hexane (Biotage SP⁴ system, 25M column, 25 CV) and 50% acetone/hexane (3 CV) to afford 704.8 mg final product as white solid foam. ¹H-NMR (500 MHz, CDCl₃) δ 8.680 (m, 1H), 8.097 (br, 1H), 7.888 (d, J=8.5 Hz, 2H), 7.745 (dt, J=1.5 and 8.0 Hz, 1H), 7.712 (d. J=7.5 Hz, 1H), 7.384-7.353 (m, 5H), 7.300 (d, J=7.5 Hz, 2H), 7.232-7.189 (m, 3H), 7.135-7.075 (m, 3H), 6.910 (br, 1H), 5.505 (br, 2H), 5.250 (br, 1H), 5.100 (br, 1H), 4.850 (br, 1H), 4.274-4.234 (m, 1H), 4.200-4.053 (m, 5H), 3.725-3.525 (m, 16H), 3.362 (s, 3H), 3.267-3.209 (m, 1H), 3.132-3.087 (m, 1H), 3.036 (br, 1H), 2.468 (br, 2H), 2.173 (m, 1H), 1.007 (d, J=7.0 Hz, 3 H), 0.926 (d, J=7.0 Hz, 3H), 0.857 (s, 9H), 0.777 (s, 9H). LC-MS (ESI): 1183.9 (MH⁺).

### Synthesis of Val-Phe-mPEG₃-Atazanavir Hydrochloride

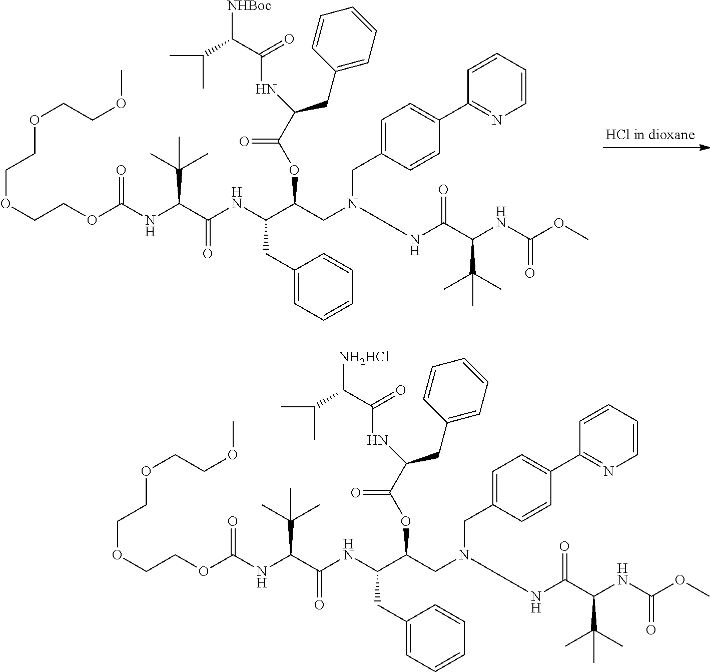

Boc-Val-Phe-mPEG₃-Atazanavir (704.8 mg, 0.596 mmol) was dissolved in anhydrous dioxane (5 mL) at room temperature. Therafter, 4N HCl solution dioxane (5 mL) was added. The resulting mixture was stirred at room temperature for one hour, fifteen minutes. DCM (100 mL) was added to diluted the reaction mixture. Saturated NaCl solution (100 mL) was added. The organic solution was separated and the aqueous solution was extracted with DCM (2×40 mL). The combined organic solution was washed with saturated NH₄Cl solution (100 mL), dried over anhydrous sodium sulfate, concentrated and dried. The product was dissolved in DCM (˜150 mL), washed with NaCl solution and NH₄Cl aqueous solution. The combined aqueous solution was extracted with DCM (20 mL). The combined organic solution was dried over sodium sulfate, and concentrated to afford 674.3 mg final product. The purity was ˜96% based on HPLC. The yield was 97%. ¹H-NMR in DMSO: 9.071 (br, 1H), 8.916 (br, 1H), 8.666 (dt, J=1.5 Hz and 5.0 Hz, 1H), 8.115 (m, 3H), 7.937 (d, J=8.5 Hz, 2H), 7.905-7.837 (m, 3H), 7.381 (d, J=8.5 Hz, 1H), 7.242-7.112 (m, 6H), 6.778 (d, J=9.5 Hz, 1H), 6.661 (d, J=9.5 Hz, 1H), 5.041 (t, J=6.5 Hz, 1H), 4.792-4.748 (m, 1H), 4.628 (m, 1H), 4.064-3.977 (m, 5H), 3.712-3.651 (m, 2H), 3.565-3.343 (m, 14H), 3.223 (s, 3H), 3.080-3.004 (m, 2H), 2.900 (m, 2H), 2.634 (d, J=7.0 Hz, 2H), 2.232-2.194 (m, 1H), 0.970 (d, J=7.0 Hz, 3H), 0.917 (d, J=7.0 Hz, 3H), 0.842 (s, 9H, Bu^{t}), 0.751 (s, 9H, Bu^{t}). LC-MS (ESI): 1083.8 MH⁺).

### Synthesis of Boc-Leu-mPEG₅-Atazanavir

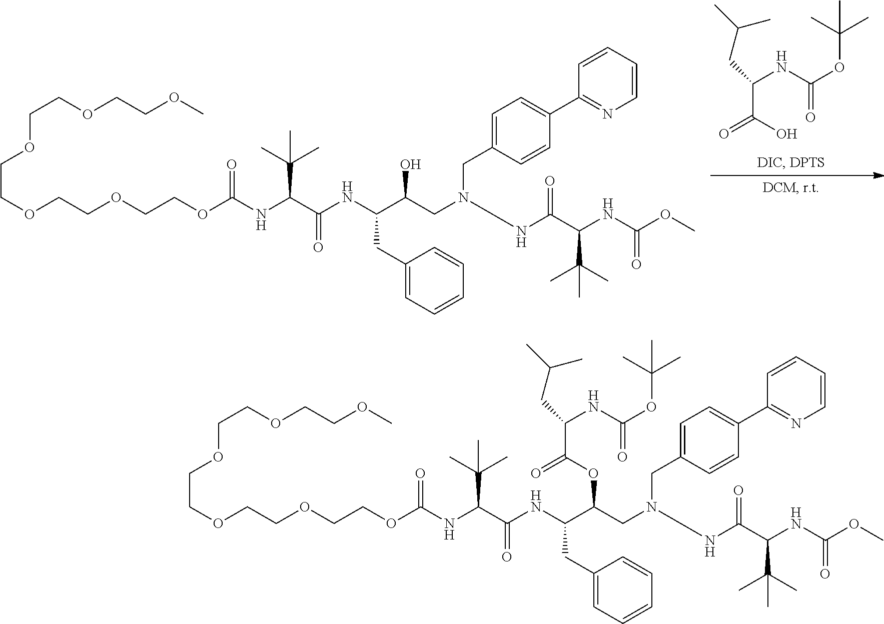

mPEG₅-Atazanavir (2.3679 g, 2.56 mmol), Boc-Leu-OH (12.296 g, 52.6 mmol) and DPTS (1:1 mixture of DMAP and p-toluenesulfonic acid) (772 mg, 2.62 mmol) were dissolved in anhydrous dichloromethane (90 mL) at room temperature. After about fifteen minutes, DIC (9 mL, 57.5 mmol) was added. The resulting mixture was stirred at room temperature for four hours. After three hours, HPLC analysis showed only about 40% conversion. Therefore, the reaction mixture was filtered to remove the solid and the solid was washed with fresh distillated DCM. The combined organic solution was concentrated to about 50 mL. Boc-Leu-OH (12.162 g, 52.06 mol) and DPTS (834.9 mg, 2.84 mmol) were added. Thereafter, DIC (9.5 mL, 61.35 mmol) was added. The mixture was stirred at room temperature for nineteen hours. The mixture was filtered to remove the white solid. The solid was washed with DCM. The combined organic solution was concentrated. The residue was separated with flash column chromatography on silica gel and eluted with 1-6% MeOH/DCM (40M, 25 CV). Due to DIU, the product was purified again with flash column chromatography on silica gel and diluted with 10-50% acetone/hexanes (25 m, 25 CV) to afford 1.4492 g final pure product, as well as 821.8 mg of starting material (in 35% recovery ¹H-NMR (CDCl₃): 8.675 (m, 1H), 7.889 (d, J=8.5 Hz, 2H), 7.743 (dt, J=1.5 and 8.0 Hz, 1H), 7.694 (d, J=8.0 Hz, 1H), 7.325 (d, J=8.0 Hz, 2H), 7.246-7.203 (m, 3H), 7.162-7.120 (m, 3H), 6.000 (m, 1H), 5.425 (m, 1H), 5.298 (m, 1H), 5.180 (br, 1H), 5.026 (br, 1H), 4.350-4.143 (m, 7H), 3.779-3.529 (m, 23H), 3.370 (s, 3H, CH₃), 3.110 (m, 2H), 2.745-2.607 (m, 2H), 1.843-1.789 (m, 2H), 1.452 (s, 9H, Bu^{t}), 1.436 (m, 1H), 0.896 (s, 9 H, Bu^{t}), 0.770 (s, 9H, Bu^{t}). LC-MS (ESI): 1138.7 (MH⁺).

### Synthesis of Leu-mPEG₅-Atazanavir Hydrochloride

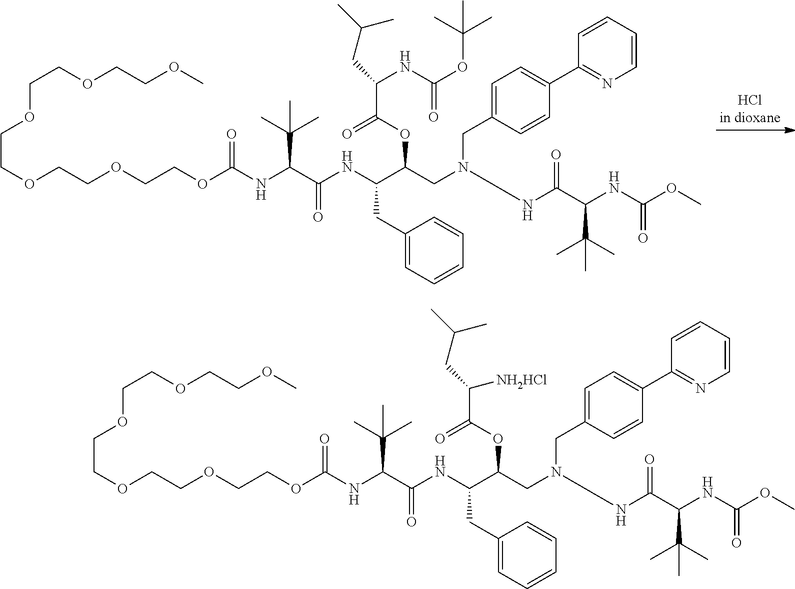

Boc-Leu-mPEG₅-Atazanavir (1.4492 g, 1.273 mmol) was dissolved in anhydrous dioxane (13 mL) at room temperature. Thereafter, 4N HCl solution dioxane (13 mL) was added. The resulting mixture was stirred at room temperature for one hour, twenty minutes. Saturated NaCl solution was added. The organic solution was separated and the aqueous solution was extracted with DCM (25 mL). The combined organic solution was washed with saturated NH₄Cl solution (2×100 mL), dried over anhydrous sodium sulfate, concentrated and dried under high vacuum to afford 1.3684 g product as white form. The purity was 96% based on HPLC. The yield was 96%. ¹H-NMR (DMSO-d₆): 9.154 (m, 1H), 8.646 (dd, J=4.5 and 1.5 Hz, 1H), 8.444 (br, 3H), 7.948-7.852 (m, 5H), 7.372 (d, J=8.0 Hz, 2H), 7.331 (dt, J=5.0 and 1.5 Hz, 1H), 7.211-7.146 (m, 5H), 6.831 (d, J=9.5 Hz, 1H), 6.796 (d, J=9.5 Hz, 1H), 5.124 (m, 1H), 4.493 (br, 1H), 4.095-4.053 (m, 3H), 3.973-3.871 (m, 3H), 3.722-3.405 (m, 23H), 3.227 (s, 3H, CH₃), 2.977-2.876 (m, 2H), 2.769-2.631 (m, 2H), 1.907-1.814 (m, 2H), 1.704-1.672 (m, 1H), 0.971 (d, J=6.5 Hz, 3H), 0.952 (d, J=6.5 Hz, 3H), 0.802 (s, 9H, Bu^{t}), 0.745 (s, 9H, Bu^{t}). LC-MS (ESI): 1038.8 (MH⁺).

### Synthesis of Sac-Leu-Leu-mPEG₅-Atazanavir

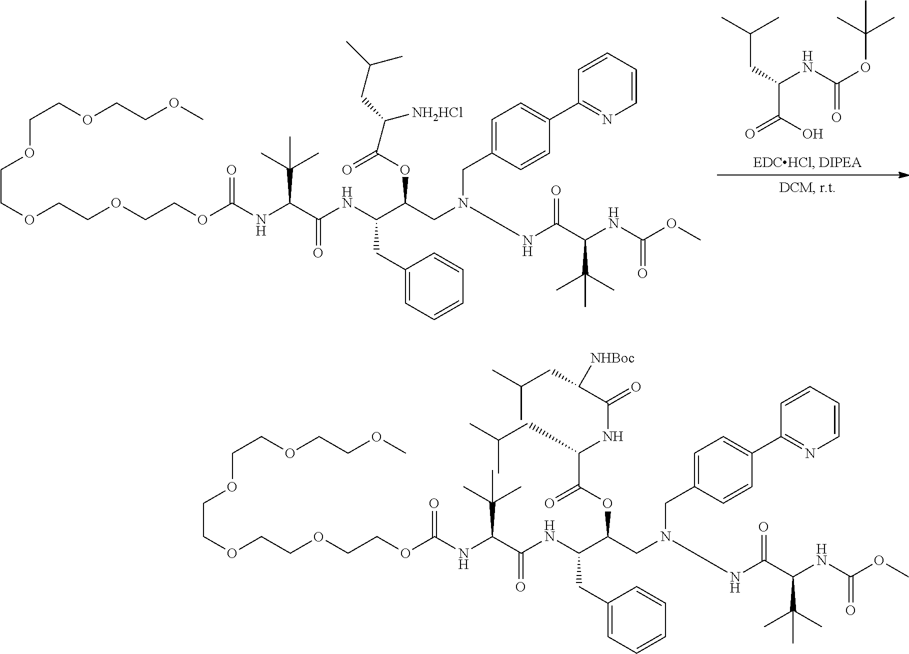

Leu-mPEG₅-Atazanavir hydrochloride (96%) (695.4 mg, 0.621 mmol) was dissolved in anhydrous dichloromethane (10 mL) at room temperature. DIPEA (0.55 mL, 3.16 mmol) was added, followed by addition of Boc-Leu-OH (446 mg, 1.909 mmol). EDC.HCl (496 mg, 2.59 mmol) was added. The resulting mixture was stirred at room temperature for 3.5 hours. NaHCO₃ aqueous solution (5%) (35 mL) was added to quench the reaction. The organic solution was separated, the aqueous was extracted with DCM (30 mL). The combined organic solution was washed with saturated NaCl (100 mL), dried over Na₂SO₄, and concentrated. The residue was purified by flash column chromatography on silica gel and eluted with 1-6% MeOH in DCM (biotage SP⁴ system, 25 M column, 25 CV) to afford 727.9 mg of product as white foam. The purity was 98% based on HPLC. The yield was 92%. ¹H-NMR in CDCl₃: 8.667 (d, J=4.5 Hz, 1H), 7.890 (d, J=8.5 Hz, 1H), 7.752-7.666 (m, 3H), 7.314 (d, J=7.5 Hz, 2H), 7.247-7.196 (m, 3H), 7.174-7.135 (m, 3H), 7.000 (m, 1H), 6.231 (m, 1H), 5.750 (m, 1H), 5.404 (m, 2H), 5.116 (m, 1H), 4.475 (m, 1H), 4.366 (m, 1H), 4.192-4.101 (m, 5H), 3.790 (d, J=8.5 Hz, 1H), 3.673-3.530 (s, 22H), 3.371 (s, 3H), 3.228-3.194 (m, 1H), 3.061-3.013 (m, 1H), 2.803-2.761 (m, 1H), 2.720-2.677 (m, 1H), 1.798-1.686 (m, 5H), 1.559-1.502 (m, 1H), 1.462 (s, 9H, Bu^{t}), 1.034 (d, J=6.5 Hz, 3H), 1.002 (d, J=6.5 Hz, 3H), 0.936 (d, J=6.5 Hz, 3H), 1.906 (d, J=6.5 Hz, 3H), 0.887 (s, 9H, Bu^{t}), 0.787 (s, 9H, Bu^{t}). LC-MS (ESI): 1252.0 (MH⁺).

### Synthesis of Leu-Leu-mPEG₅-Atazanavir Hydrochloride

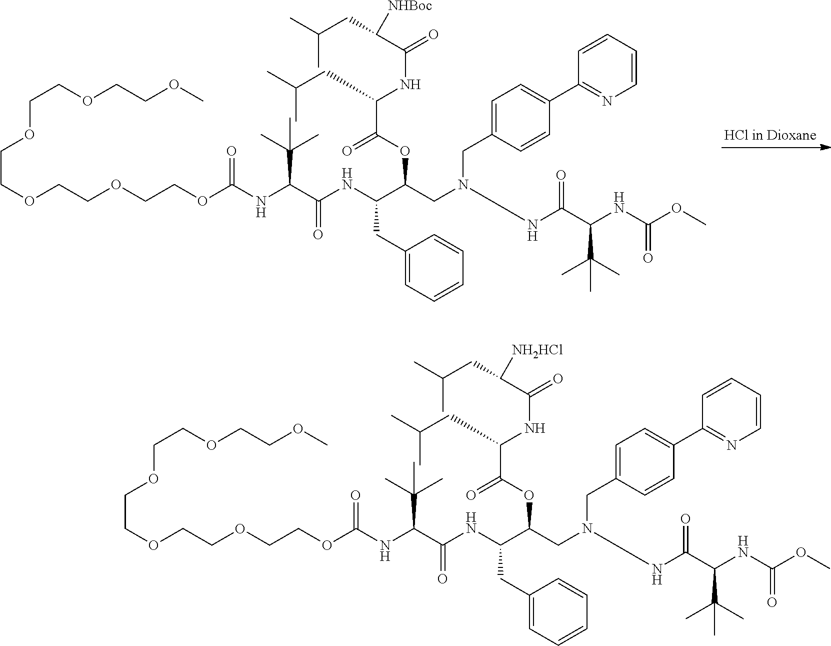

Boc-Leu-Leu-mPEG₅-Atazanavir (98%) (0.7279 g, 0.582 mmol) was dissolved in anhydrous dioxane (5 mL) at room temperature. Thereafter, 4N HCl solution dioxane (5 mL) was added. The resulting mixture was stirred at room temperature for one hour, twenty minutes. DCM (100 mL) was added to dilute the reaction mixture. Saturated NaCl solution (100 mL) was added. The organic solution was separated and the aqueous solution was extracted with DCM (40 mL). The combined organic solution was washed with saturated NH₄Cl solution (2×100 mL), dried over anhydrous sodium sulfate, concentrated and dried under high vacuum to afford 726 mg product as white foam. The purity was about 95% based on HPLC. ¹H-NMR (DMSO-d₆): 8.944 (d, J=8.0 Hz, 1H), 8.900 (br, 1H), 8.638 (m, 1H), 8.148 (m, 3H), 7.942 (d, J=8.0 Hz, 2H), 7.908-7.842 (m, 3H), 7.358 (d, J=8.0 Hz, 2H), 7.336-7.310 (m, 1H), 7.208-7.199 (m, 4H), 7.167-7.142 (m, 1H), 6.747 (d, J=9.5 Hz, 1H), 6.669 (d, J=10.0 Hz, 1H), 4.999 (m, 1H), 4.555 (m, 2H), 4.065-3.988 (m, 5H), 3.856 (m, 1H), 3.674 (m, 1H), 3.582-3.4002 (m, 21H), 3.225 (s, 3H, CH₃), 3.111-3.073 (m, 1H), 2.902 (m, 1H), 2.739-2.630 (m, 2H), 1.803-1.665 (m, 3H), 1.645-1.529 (m, 3H), 0.961 (d, J=6.0 Hz, 3H), 0.910 (d, J=6.0 Hz, 3H), 0.873 (d, J=6.0 Hz, 3H), 0.869 (d, J=6.0 Hz, 3H), 0.831 (s, 9H, Bu^{t}), 0.743 (s, 9H, Bu^{t}). LC-MS (ESI): 1151.9 (MH⁺).

### Synthesis of Leu-Leu-mPEG₆-Atazanavir Hydrochloride

Using an approach similar to the one make Leu-Leu-mPEG₅-atazanavir hydrochloride, Leu-Leu-mPEG₆-atazanavir hydrochloride was prepared.

### Synthesis of Boc-Phe-Leu-mPEG₅-Atazanavir

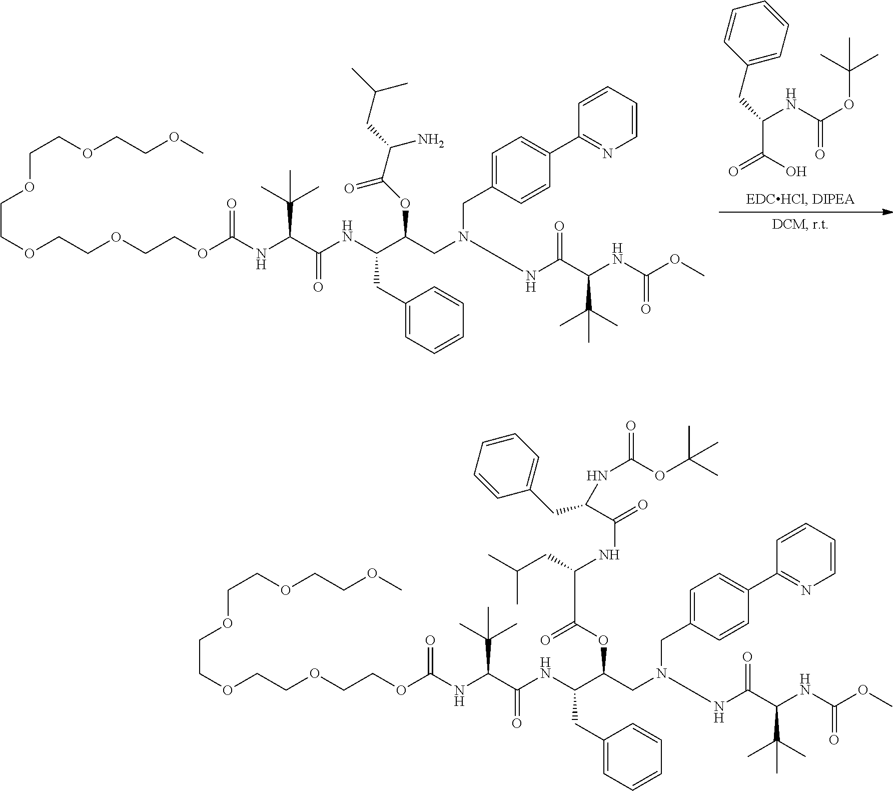

Leu-mPEG₅-Atazanavir hydrochloride (96%) (675.8 mg, 0.604 mmol) was dissolved in anhydrous dichloromethane (10 mL) at room temperature. DIPEA (0.55 mL, 3.16 mmol) was added, followed by addition of Boc-Phe-OH (482 mg, 1.817 mmol). EDC.HCl (409 mg, 2.134 mmol) was added. The resulting mixture was stirred at room temperature for 3.5 hours. NaHCO₃ aqueous solution (5%) (35 mL) was added to quench the reaction. The organic solution was separated, the aqueous was extracted with DCM (30 mL). The combined organic solution was washed with saturated NaCl (100 mL), dried over Na₂SO₄, and concentrated.

The residue was purified by flash column chromatography on silica gel and eluted with 1-6% MeOH in DCM (biotage SP⁴ system, 25M column, 25 CV) to afford 0.7264 g product as white foam. The purity was 96% based on HPLC. The yield was 90%. ¹H-NMR in CDCl₃: 8.571 (m, 1H), 7.875 (d, J=8.0 Hz, 1H), 7.714 (dt, J=1.5 and 8.0 Hz, 1H), 7.674 (d, J=8.0 Hz, 1H), 7.300 (d, J=8.0 Hz, 2H), 7.257-7.228 (m, 4H), 7.210-7.157 (m, 7H), 6.853 (m, 1H), 6.323 (m, 1H), 5.706 (m, 1H), 5.591 (m, 2H), 5.415-5.398 (m, 1H), 5.129 (m, 1H), 4.519 (m, 1H), 4.363 (m, 2H), 4.202-4.101 (m, 4H), 3.815 (d, J=8.5 Hz, 1H), 3.700-3.530 (s, 21H), 3.371 (s, 3H), 3.267-3.103 (m, 3H), 3.050-3.006 (m, 1H), 2.822-2.783 (m, 1H), 2.724-2.680 (m, 1H), 1.720-1.634 (m, 3H), 1.395 (s, 9H, Bu^{t}), 1.003 (d, J=6.0 Hz, 3H), 0.987 (d, J=6.0 Hz, 3H), 0.894 (s, 9H, Bu^{t}), 0.788 (s, 9H, Bu^{t}). LC-MS (ESI): 1285.9 (MH⁺).

### Synthesis of Phe-Leu-mPEG₅-Atazanavir Hydrochloride

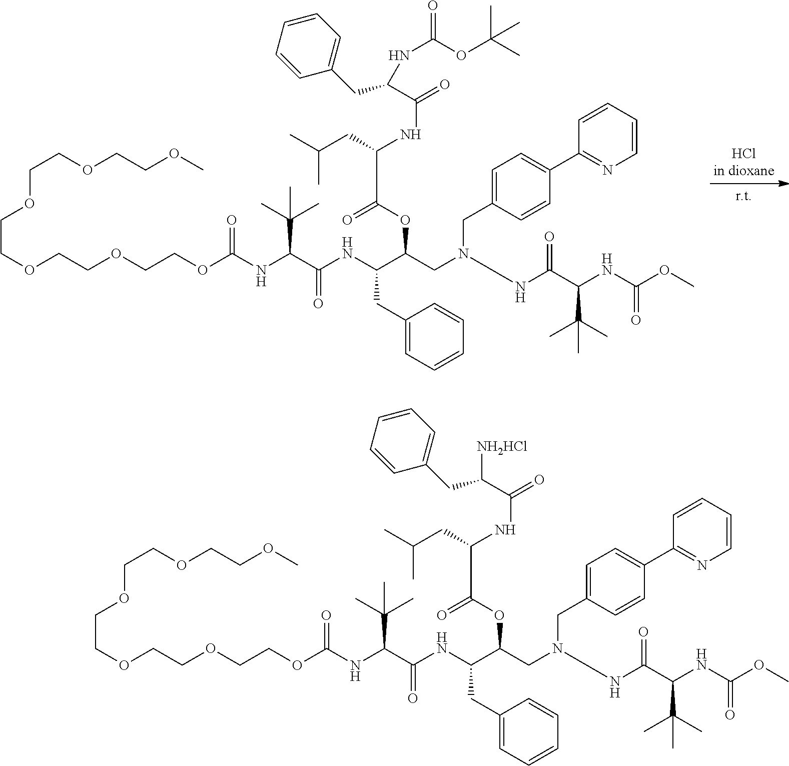

Boc-Phe-Leu-mPEG₅-Atazanavir (0.726.4 g, 0.565 mmol) was dissolved in anhydrous dioxane (5 mL) at room temperature. Therafter, 4N HCl solution dioxane (5 mL) was added. The resulting mixture was stirred at room temperature for one hour, twenty minutes. DCM (100 mL) was added to dilute the reaction mixture. Saturated NaCl solution (100 mL) was added. The organic solution was separated and the aqueous solution was extracted with DCM (40 mL). The combined organic solution was washed with saturated NH₄Cl solution (2×100 mL), dried over anhydrous sodium sulfate, concentrated and dried under high vacuum to afford 754.2 mg product as white foam. The purity was about 95% based on HPLC. ¹H-NMR (DMSO-d₆): 9.115 (m, 1H), 8.907 (br, 1H), 8.643-8.629 (m, 1H), 8.085 (br, 1H), 7.978-7.930 (m, 3H), 7.854 (m, 2H), 7.372 (d, J=8.5 Hz, 2H), 7.334-7.299 (m, 5H), 7.277-7.243 (m, 1H), 7.201-7.141 (m, 5H), 6.746 (d, J=9.5 Hz, 1H), 6.689 (d, J=9.0 Hz, 1H), 5.005 (t, J=6.0 Hz, 1H), 4.588 (m, 2H), 4.150 (m, 1H), 4.063-4.005 (m, 4H), 3.677 (m, 1H), 3.579-3.404 (m, 21H), 3.225 (s, 3H, CH₃), 3.199 (m, 1H), 3.107-3.070 (m, 1H), 2.937 (m, 2H), 2.757-2.635 (m, 2H), 1.788-1.618 (m, 3H), 0.964 (d, J=6.5 Hz, 3H), 0.913 (d, J=6.0 Hz, 3H), 0.825 (s, 9H, Bu^{t}), 0.743 (s, 9H, Bu^{t}). LC-MS (ESI): 1185.9 (MH⁺).

### Synthesis of Phe-Leu-mPEG₆-Atazanavir Hydrochloride

Using an approach similar to the one make Phe-Leu-mPEG₅-atazanavir hydrochloride, Phe-Leu-mPEG₆-atazanavir hydrochloride was prepared.

### Examples 26a-26c

### Preparation of mPEGₙ-Atazanavir-Succinic-D-Glucofuranose Compounds

mPEGₙ-Atazanavir-succinic-D-glucofuranose compounds were prepared in accordance with the schematic provided below.

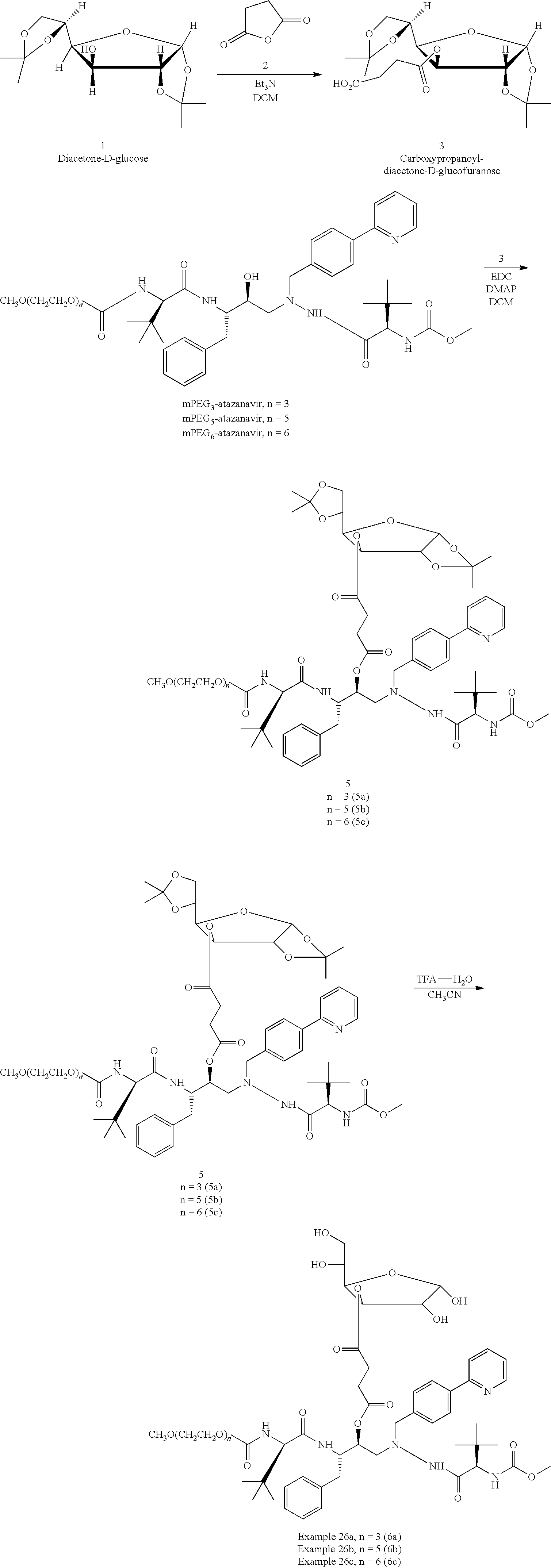

### Preparation of Compound 3, 5-((3aR,5R,6S,6aR)-5-(2,2-dimethyl-1,3-dioxolan-4-yl)-2,2-dimethyltetrahydrofuro[2,3-d][1,3]dioxol-6-yloxy)-5-oxopentanoic acid

Into a 500 mL flask was placed diacetone D-glucose (Compound 1) (5.0 gm, 19.2 mmol) and anhydrous dichloromethane (100 mL). To the clear solution was added succinic anhydride (Compound 2) (5.76 gm, 57.6 mmol, 3.0 equivalents) and triethylamine (6.7 mL, 48.0 mmol, 2.5 equivalents). The clear reaction mixture was stirred at room temperature under nitrogen. The reaction mixture gradually turned progressively darker. After approximately eighteen hours at room temperature the reaction mixture was diluted with dichloromethane (125 mL) and transferred to a separatory funnel. The organic layer was washed with 5% potassium hydrogen sulfate, deionized water and saturated sodium chloride (150 mL each). The organic layer was dried over sodium sulfate, filtered and concentrated under reduced pressure to give a dark oil. Purification by Biotage chromatography (gradient elution: 0 to 10% methanol/dichloromethane over 20 column volumes) gave 6.01 gm (87%) of Compound 3 as a yellow oil; R_{f} 0.30 (5% methanol-dichloromethane); ¹H NMR (DMSO-d6): δ 12.29 (bs, 1H), 5.87 (d, 1H), 5.02 (d, 1H), 4.48 (d, 1H), 4.18 (m, 2H), 4.01 (m, 1H), 3.84 (m, 1H), 2.55 (m, 2H), 1.42 (s, 3H), 1.31 (s, 3H), 1.24 (d, 6H). MS 360 (M+H)⁺.

### Preparation of Compound 5a, (5R,10S,11S,14R)-11-benzyl-5,14-di-tert-butyl-3,6,13,16-tetraoxo-8-(4-(pyridin-2-yl)benzyl)-2,17,20,23,26-pentaoxa-4,7,8,12,15-pentaazaheptacosan-10-yl (3aR,5R,6S,6aR)-5-(2,2-dimethyl-1,3-dioxolan-4-yl)-2,2-dimethyltetrahydrofuro[3,2-d][1,3]dioxol-6-yl succinate

Into a 500 mL flask was placed previously prepared mPEG₃-atazanavir (5.32 gm, 6.36 mmol) and anhydrous dichloromethane (100 mL). Then, Compound 3 (5.73 gm, 15.9 mmol, 2.5 equivalents) was added, followed by the addition of 4-dimethylaminopyridine (1.94 gm, 15.9 mmol, 2.5 equivalents). The yellow solution was cooled to 0° C. and then EDC-HCl (N-(3-dimethylaminopropyl)-N′-ethylcarbodiimide hydrochloride; 3.05 gm, 15.9 mmol, 2.5 equivalents) was added. The yellow reaction mixture was allowed to equilibrate to room temperature. After approximately eighteen hours at room temperature, the brown reaction mixture was diluted with dichloromethane (150 mL). The organic layer was transferred to a separatory funnel and partitioned with deionized water (200 mL). The aqueous layer was extracted with dichloromethane (3×50 mL). The combined organic layers were washed with deionized water and saturated sodium chloride (200 mL each). The organic layer was dried over sodium sulfate, filtered and concentrated under reduced pressure to give a brown oil. Purification by Biotage chromatography (gradient elution: 0 to 5% methanol/dichloromethane over 20 column volumes) gave 6.76 gm (91%) of Compound 5a as a light-beige solid; R_{f} 0.60 (10% methanol-dichloromethane); ¹H NMR (DMSO-d6): δ 8.98 (bs, 1H), 8.64 (d, 1H), 7.94 (m, 3H), 7.87 (m, 2H), 7.36 (d, 2H), 7.32 (m, 1H), 7.18 (m, 4H), 7.12 (m, 1H), 6.72 (d, 1H), 6.68 (d, 1H), 4.52-4.48 (m, 2H), 4.22 (m, 1H), 4.18 (m, 1H), 4.07 (m, 2H), 4.05 (m, 1H), 3.96 (m, 2H), 3.88 (m, 1H), 3.67 (d, 1H), 3.58 (m, 2H), 3.54 (m, 6H), 3.492 (m, 5H), 3.34 (m, 8H), 3.22 (s, 3H), 3.05 (m, 1H), 2.90 (m, 1H), 2.71 (m, 4H), 1.42 (s, 3H), 1.31 (s, 3H), 1.24 (s, 3H), 1.21 (s, 3H), 0.79 (s, 9H), 0.74 (s, 9H). MS 1180 (M+H)⁺

### Preparation of Example 26a, (Compound 6a), (5R,10S,11S,14R)-11-benzyl-5,14-di-tert-butyl-3,6,13,16-tetraoxo-8-(4-(pyridin-2-yl)benzyl)-2,17,20,23,26-pentaoxa-4,7,8,12,15-pentaazaheptacosan-10-yl (2R,3R,4R,5S)-2-((R)-1,2-dihydroxyethyl)-4,5-dihydroxytetrahydrofuran-3-yl succinate (6a) (NKT-10749-A-001)

Compound 5a (6.69 gm, 5.67 mmol) was taken up in anhydrous acetonitrile (200 mL). To the yellow solution was added trifluoroacetic acid:water (9:1; 100 mL). The yellow reaction mixture was stirred at room temperature. After approximately eighteen hours at room temperature, the solvents were removed under reduced pressure. Purification by Biotage chromatography (gradient elution: 0 to 25% methanol/dichloromethane over 20 column volumes) gave 2.62 gm (42%) of Example 26a (Compound 6a) as a white solid. ¹H NMR (DMSO-d6): δ 8.99 (s, 1H), 8.68 (d, 1H), 7.95 (m, 4H), 7.85 (d, 1H), 7.39 (m, 3H), 7.17-7.39 (m, 5H), 6.72 (d, 2H), 5.07 (m, 2H), 4.98 (d, 1H), 4.80 (m, 1H), 4.49 (bs, 1H), 4.06 (m, 2H), 3.98 (m, 3H), 3.68 (m, 2H), 3.56 (m, 2H), 3.51 (m, 7H), 3.42 (m, 5H), 3.30 (m, 2H). 3.22 (s, 3H), 3.08 (m, 2H), 2.88 (m, 1H), 2.70 (m, 6H), 0.78 (s, 9H), 0.73 (s, 9H). MS 1100 (M+H)⁺.

### Preparation of Compound 5b, (5R,10S,11S,14R)-11-benzyl-5,14-di-tert-butyl-3,6,13,16-tetraoxo-8-(4-(pyridin-2-yl)benzyl)-2,17,20,23,26,29,32-heptaoxa-4,7,8,12,15-pentaazatritriacontan-10-yl (3aR,5R,6S,6aR)-5-(2,2-dimethyl-1,3-dioxolan-4-yl)-2,2-dimethyltetrahydrofuro[3,2-d][1,3]dioxol-6-yl succinate

Into a 500 mL flask was placed previously prepared mPEG₅-atazanavir (5.60 gm, 6.05 mmol) and anhydrous dichloromethane (100 mL). Then Compound 3 (5.45 gm, 15.1 mmol, 2.5 equivalents) was added, followed by addition of 4-dimethylaminopyridine (1.85 gm, 15.1 mmol, 2.5 equivalents). The yellow solution was cooled to 0° C. and then EDC-HCl (N-(3-dimethylaminopropyl)-N′-ethylcarbodiimide hydrochloride; 2.90 gm, 15.1 mmol, 2.5 equivalents) was added. The yellow reaction mixture was allowed to equilibrate to room temperature. After approximately eighteen hours at room temperature, the brown reaction mixture was diluted with dichloromethane (150 mL). The organic layer was transferred to a separatory funnel and partitioned with deionized water (200 mL). The aqueous layer was extracted with dichloromethane (3×50 mL). The combined organic layers were washed with deionized water and saturated sodium chloride (200 mL each). The organic layer was dried over sodium sulfate, filtered and concentrated under reduced pressure to give a brown oil. Purification by Biotage chromatography (gradient elution: 0 to 5% methanol/dichloromethane over 20 column volumes) gave 6.21 gm (81%) of Compound 5b as a light-beige solid; R_{f} 0.60 (10% methanol-dichloromethane); NMR (DMSO-d6): δ 8.98 (bs, 1H), 8.64 (d, 1H), 7.92 (m, 3H), 7.85 (m, 2H), 7.34 (d, 2H), 7.17 (m, 4H), 7.13 (m, 1H), 6.71 (dd, 2H), 5.90 (d, 1H), 5.07 (m, 2H), 4.51 (d, 1H), 4.48 (bs, 1H), 4.20 (m, 1H), 4.18 (m, 1H), 4.07 (m, 2H), 4.02 (m, 1H), 3.95 (m, 2H), 3.86 (m, 1H), 3.65 (d, 1H), 3.58 (m, 2H), 3.49 (m, 20H), 3.42 (m, 4H), 3.33 (s, 6H), 3.04 (dd, 1H), 2.88 (m, 1H), 2.70 (m, 4H), 2.62 (m, 1H), 1.41 (s, 3H), 1.31 (s, 3H), 1.24 (d, 6H), 0.79 (s, 9H), 0.74 (s, 9H). MS 1267 (M+H)⁺.

### Preparation of Example 26b, (Compound 6b), (5R,10S,11S,14R)-11-benzyl-5,14-di-tert-butyl-3,6,13,16-tetraoxo-8-(4-(pyridin-2-yl)benzyl)-2,17,20,23,26,29,32-heptaoxa-4,7,8,12,15-pentaazatritriacontan-10-yl (2R,3R,4R,5S)-2-((R)-1,2-dihydroxyethyl)-4,5-dihydroxytetrahydrofuran-3-yl succinate

Compound 5b (6.15 gm, 4.85 mmol) was taken up in anhydrous acetonitrile (155 mL). To the yellow solution was added trifluoroacetic acid:water (9:1; 75 mL). The yellow reaction mixture was stirred at room temperature. After approximately eighteen hours at room temperature, the solvents were removed under reduced pressure. Purification by Biotage chromatography (gradient elution: 0 to 25% methanol/dichloromethane over 20 column volumes) gave 3.5 gm (52%) of Example 26b (Compound 6b) as a white solid; R_{f} 0.29 (10% methanol-dichloromethane); ¹H NMR (DMSO-d6): δ 8.99 (bs, 1H), 8.68 (d, 1H), 7.95 (m, 4H), 7.85 (d, 1H), 7.39 (m, 3H), 7.18 (m, 4H), 7.14 (m, 1H), 6.71 (d, 2H), 5.06 (m, 2H), 4.98 (d, 1H), 4.80 (m, 1H), 4.40 (d, 1H), 4.06 (m, 2H), 3.98 (m, 3H), 3.68 (m, 2H), 3.59 (m, 2H), 3.50 (m, 14H), 1H), 3.40 (m, 5H), 3.32 (m, 2H), 3.22 (s, 3H), 2.88 (m, 1H), 2.70 (m, 6H), 0.78 (s, 9H), 0.74 (s, 9H). MS 1187 (M+H)⁺.

### Preparation of Compound 5c, (5R,10S,11S,14R)-11-benzyl-5,14-di-tert-butyl-3,6,13,16-tetraoxo-8-(4-(pyridin-2-yl)benzyl)-2,17,20,23,26,29,32,35-octaoxa-4,7,8,12,15-pentaazahexatriacontan-10-yl (3aR,5R,6S,6aR)-5-(2,2-dimethyl-1,3-dioxolan-4-yl)-2,2-dimethyltetrahydrofuro[3,2-d][1,3]dioxol-6-yl succinate

Into a 500 mL flask was placed previously prepared mPEG₆-atazanavir (6.45 gm, 6.60 mmol) and anhydrous dichloromethane (100 mL). Then Compound 3 (6.0 gm, 16.6 mmol, equivalents) was added, followed by addition of 4-dimethylaminopyridine (2.03 gm, 16.6 mmol, 2.5 equivalents). The yellow solution was cooled to 0° C. and then EDC-HCl (N-(3-dimethylaminopropyl)-N′-ethylcarbodiimide hydrochloride; 3.19 gm, 16.6 mmol, 2.5 equivalents) was added. The yellow reaction mixture was allowed to equilibrate to room temperature. After approximately eighteen hours at room temperature, the brown reaction mixture was diluted with dichloromethane (150 mL). The organic layer was transferred to a separatory funnel and partitioned with deionized water (200 mL). The aqueous layer was extracted with dichloromethane (3×50 mL). The combined organic layers were washed with deionized water and saturated sodium chloride (200 mL each). The organic layer was dried over sodium sulfate, filtered and concentrated under reduced pressure to give a brown oil. Purification by Biotage chromatography (gradient elution: 0 to 5% methanol/dichloromethane over 20 column volumes) gave 8.19 gm (94%) of Compound 5c as a light-yellow solid; R_{I}-0.57 (10% methanol-dichloromethane); ¹H NMR (DMSO-d6): δ 8.89 (bs, 1H), 8.64 (d, 1H), 7.79 (m, 3H), 7.84 (m, 2H), 7.35 (d, 2H), 7.17 (m, 4H), 7.12 (m, 1H), 6.71 (dd, 2H), 5.90 (d, 1H), 5.05 (m, 2H), 4.51 (d, 1H), 4.48 (bs, 1H), 4.20 (m, 1H), 4.18 (m, 1H), 4.07 (m, 2H), 4.02 (m, 1H), 3.95 (m, 2H), 3.86 (m, 1H), 3.65 (d, 1H), 3.58 (m, 2H), 3.49 (m, 20H), 3.42 (m, 4H), 3.33 (s, 6H), 3.04 (dd, 1H), 2.88 (m, 1H), 2.70 (m, 4H), 2.62 (m, 1H), 1.41 (s, 3H), 1.31 (s, 3H), 1.22 (d, 6H), 0.80 (s, 9H), 0.73 (s, 9H). MS 1267 (M+H)⁺.

### Example 26c

### (Compound 6c), (5R,10S,11S,14R)-11-benzyl-5,14-di-tert-butyl-3,6,13,16-tetraoxo-8-(4-(pyridin-2-yl)benzyl)-2,17,20,23,26,29,32,35-octaoxa-4,7,8,12,15-pentaazahexatriacontan-10-yl (2R,3R,4R,5S)-2-((R)-1,2-dihydroxyethyl)-4,5-dihydroxytetrahydrofuran-3-yl succinate

Compound 5c (8.19 gm, 6.24 mmol) was taken up in anhydrous acetonitrile (200 mL). To the yellow solution was added trifluoroacetic acid:water (9:1; 100 mL). The yellow reaction mixture was stirred at room temperature. After approximately eighteen hours at room temperature, the solvents were removed under reduced pressure. Purification by Biotage chromatography (gradient elution: 0 to 25% methanol/dichloromethane over 20 column volumes) gave 3.5 gm (46%) of Example 26c (Compound 6c) as a white solid; R_{f} 0.30 (10% methanol-dichloromethane); ¹H NMR (DMSO-d6): δ 8.27 (bs, 1H), 7.94 (d, 1H), 7.23 (m, 4H), 7.21 (d, 1H), 6.65 (m, 3H), 6.46 (m, 5H), 5.99 (d, 2H), 4.34 (m, 2H), 4.25 (m, 2H), 3.00-4.00 (m, 10H), 2.86-2.69 (m, 24H), 1.77 (s, 3H), 0.01 (d, 18H). MS 1231 (M+H)⁺

### Examples 27a-27c

### Preparation of mPEGₙ-Atazanavir-Glutaric-D-Glucofuranose Compounds

mPEGₙ-Atazanavir-glutaric-D-glucofuranose compounds were prepared in accordance with the schematic provided below.

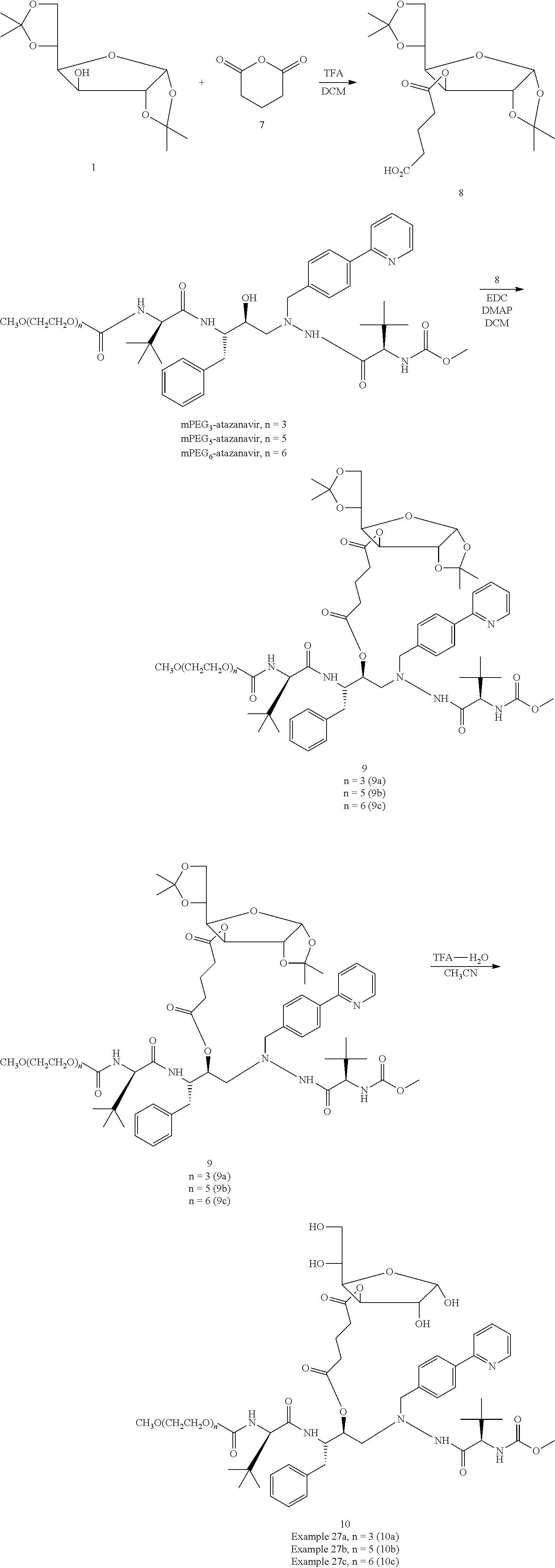

### Preparation of Compound 8, 5-((3aR,5R,6S,6aR)-5-(2,2-dimethyl-1,3-dioxolan-4-yl)-2,2-dimethyltetrahydrofuro[2,3-d][1,3]dioxol-6-yloxy)-5-oxopentanoic acid

Into a 250 mL flask was placed diacetone D-glucose (Compound 1) (2.0 gm, 7.68 mmol) and anhydrous dichloromethane (40 mL). To the clear solution was added glutaric anhydride (Compound 7) (2.92 gm, 23.0 mmol, 3.0 equivalents) and triethylamine (2.68 mL, 19.2 mmol, 2.5 equivalents). The clear reaction mixture was stirred at room temperature under nitrogen. After approximately eighteen hours at room temperature the clear reaction mixture was diluted with dichloromethane (60 mL) and transferred to a separatory funnel. The organic layer was washed with 5% potassium hydrogen sulfate (2×100 mL), deionized water (2×100 mL) and saturated sodium chloride (100 mL). The organic layer was dried over sodium sulfate, filtered and concentrated under reduced pressure to give a clear oil. Purification by Biotage chromatography (gradient elution: 0 to 10% methanol/dichloromethane over 20 column volumes) gave 2.51 gm (87%) of Compound 8 as a clear oil; R_{f} 0.26 (5% methanol-dichloromethane); ¹H NMR (DMSO-d6): δ 12.12 (bs, 1H), 5.89 (d, 1H), 5.75 (s, 1H), 5.03 (d, 1H), 4.54 (d, 1H), 4.15 (m, 2H), 4.02 (m, 1H), 3.83 (m, 1H), 2.70 (t, 3H), 2.38 (m, 2H), 2.26 (t, 2H), 1.87 (t, 2H), 1.75 (m, 2H), 1.42 (s, 3H), 1.31 (s, 3H), 1.24 (s, 6H).

### Example 27a

### Compound 10a

Using an approach similar to the approach used for Examples 27b and 27c, Example 27a (Compound 10a) can be prepared via intermediate Compound 9a (which itself can be prepared in an approach similar to that used for the preparation of Compounds 9b and 9c).

### Preparation of Compound 9b, (5R,10S,11S,14R)-11-benzyl-5,14-di-tert-butyl-3,6,13,16-tetraoxo-8-(4-(pyridin-2-yl)-benzyl)-2,17,20,23,26,29,32-heptaoxa-4,7,8,12,15-pentaazatritriacontan-10-yl (3aR,5R,6S,6aR)-5-(2,2-dimethyl-1,3-dioxolan-4-yl)-2,2-dimethyltetrahydrofuro[2,3-d][1,3]dioxol-6-yl glutarate

Into a 250 mL flask was placed previously prepared mPEG₅-atazanavir (2.48 gm, 2.68 mmol) and anhydrous dichloromethane (44 mL). Then, Compound 8 (2.51 gm, 6.71 mmol, 2.5 equivalents) was added, followed by the addition of 4-dimethylaminopyridine (0.82 gm, 6.71 mmol, 2.5 equivalents). The yellow solution was cooled to 0° C. and then EDC-HCl (N-(3-dimethylaminopropyl)-N′-ethylcarbodiimide hydrochloride; 1.28 gm, 6.71 mmol, 2.5 equivalents) was added. The yellow reaction mixture was allowed to equilibrate to room temperature. After approximately eighteen hours at room temperature, the brown reaction mixture was diluted with dichloromethane (80 mL). The organic layer was transferred to a separatory funnel and partitioned with deionized water (135 mL). The aqueous layer was extracted with dichloromethane (3×25 mL). The combined organic layers were washed with deionized water and saturated sodium chloride (130 mL each). The organic layer was dried over sodium sulfate, filtered and concentrated under reduced pressure to give a yellow oil. Purification by Biotage chromatography (gradient elution: 0 to 5% methanol/dichloromethane over 20 column volumes) gave 2.99 gm (87%) of Compound 9b as a white solid; R_{f} 0.50 (10% methanol-dichloromethane); ¹H NMR (DMSO-d6): δ 8.96 (bs, 1H), 8.64 (d, 1H), 7.94 (m, 3H), 7.85 (m, 2H), 7.33 (m, 2H), 7.32 (m, 1H), 7.18 (m, 4H), 7.12 (m, 1H), 6.71 (d, 2H), 5.85 (d, 1H), 5.06 (d, 2H), 4.55 (d, 1H), 4.48 (bs, 1H), 4.16 (m, 2H), 4.05 (m, 2H), 3.99 (m, 3H), 3.85 (m, 1H), 3.68 (d, 1H), 3.58 (m, 2H), 3.50 (m, 13H), 3.44 (m, 5H), 3.22 (s, 3H), 3.08 (m, 1H), 2.88 (m, 1H), 2.74 (m, 1H), 2.60 (m, 1H), 2.45 (m, 4H), 1.86 (m, 2H), 1.42 (s, 3H), 1.30 (s, 3H), 1.22 (s, 6H), 1.21, 0.78 (s, 9H), 0.74 (s, 9H). MS 1179 (M+H)⁺

### Example 27b

### Compound 10b, (5R,10S,11S,14R)-11-benzyl-5,14-di-tert-butyl-3,6,13,16-tetraoxo-8-(4-(pyridin-2-yl)benzyl)-2,17,20,23,26,29,32-heptaoxa-4,7,8,12,15-pentaazatritriacontan-10-yl (2R,3R,4R,5S)-2-((R)-1,2-dihydroxyethyl)-4,5-dihydroxytetrahydrofuran-3-yl glutarate

Compound 9b (2.97 gm, 2.31 mmol) was taken up in anhydrous acetonitrile (80 mL). To the yellow solution was added trifluoroacetic acid:water (9:1; 40 mL). The yellow reaction mixture was stirred at room temperature. After approximately eighteen hours at room temperature, the solvents were removed under reduced pressure. Purification by Biotage chromatography (gradient elution: 0 to 25% methanol/dichloromethane over 20 column volumes) gave 2.22 gm (80%) of Example 27b, (Compound 10b) as a white solid; R_{f} 0.50 (10% methanol-dichloromethane); NMR (DMSO-d6): δ 8.98 (bs, 1H), 8.66 (d, 1H), 7.95 (m, 4H), 7.82 (d, 1H), 7.37 (m, 3H), 7.18 (m, 5H), 6.71 (d, 1H), 5.05 (m, 2H), 4.48 (bs, 2H), 4.30 (m, 1H), 3.95-4.06 (m, 17H), 3.68 (d, 2H), 3.49 (m, 22H), 3.22 (s, 3H), 3.08 (m, 1H), 2.90 (m, 1H), 2.75 (m, 1H), 2.60 (m, 1H), 2.45 (m, 3H), 0.78 (s, 9H), 0.74 (s, 9H). MS 1201 (M+H)⁺.

### Preparation of Compound 9c, (5R,10S,11S,14R)-11-benzyl-5,14-di-tert-butyl-3,6,13,16-tetraoxo-8-(4-(pyridin-2-yl)benzyl)-2,17,20,23,26,29,32,35-octaoxa-4,7,8,12,15-pentaazahexatriacontan-10-yl (3aR,5R,6S,6aR)-5-(2,2-dimethyl-1,3-dioxolan-4-yl)-2,2-dimethyltetrahydrofuro[2,3-d][1,3]dioxol-6-yl glutarate

Into a 250 mL flask was placed previously prepared mPEG₆-atazanavir 4c (2.58 gm, 2.66 mmol) and anhydrous dichloromethane (40 mL). Then Compound 8 (2.49 gm, 6.65 mmol, 2.5 equivalents) was added, followed by the addition of 4-dimethylaminopyridine (0.81 gm, 6.71 mmol, 2.5 equivalents). The yellow solution was cooled to 0° C. and then EDC-HCl (N-(3-dimethylaminopropyl)-N′-ethylcarbodiimide hydrochloride; 1.28 gm, 6.65 mmol, 2.5 equivalents) was added. The light-yellow reaction mixture was allowed to equilibrate to room temperature. After approximately eighteen hours at room temperature the brown reaction mixture was diluted with dichloromethane (80 mL). The organic layer was transferred to a separatory funnel and partitioned with deionized water (135 mL). The aqueous layer was extracted with dichloromethane (3×30 mL). The combined organic layers were washed with deionized water and saturated sodium chloride (130 mL each). The organic layer was dried over sodium sulfate, filtered and concentrated under reduced pressure to give a yellow oil. Purification by Biotage chromatography (gradient elution: 0 to 5% methanol/dichloromethane over 20 column volumes) gave 3.0 gm (85%) of Compound 9c as a white solid; R_{f} 0.57 (10% methanol-dichloromethane); ¹H NMR (DMSO-d6): δ 8.96 (bs, 1H), 8.64 (d, 1H), 7.94 (m, 3H), 7.85 (m, 2H), 7.36 (d, 2H), 7.32 (m, 1H), 7.15 (m, 4H), 7.13 (m, 1H), 6.72 (d, 1H), 6.69 (d, 1H), 5.86 (d, 1H), 5.06 (m, 2H), 4.55 (d, 1H), 4.46 (bs, 1H), 4.16 (m, 2H), 4.05 (m, 2H), 3.97 (m, 4H), 3.86 (m, 1H), 3.67 (d, 1H), 3.49 (m, 19H), 3.42 (m, 5H), 3.22 (s, 3H), 3.05 (m, 1H), 2.88 (m, 1H), 2.75 (d, 1H), 2.61 (m, 1H), 2.46 (m, 4H), 1.86 (m, 2H), 1.42 (s, 3H), 1.30 (s, 3H), 1.22 (s, 3H), 1.22 (s, 3H), 0.78 (s, 9H), 0.74 (s, 9H). MS 1326 (M+H)⁺.

### Example 27c

### Compound 10c, (5R,10S,11S,14R)-11-benzyl-5,14-di-tert-butyl-3,6,13,16-tetraoxo-8-(4-(pyridin-2-yl)benzyl)-2,17,20,23,26,29,32,35-octaoxa-4,7,8,12,15-pentaazahexatriacontan-10-yl (2R,3R,4R,5S)-2-((R)-1,2-dihydroxyethyl)-4,5-dihydroxytetrahydrofuran-3-yl glutarate

Compound 9c (2.97 gm, 2.24 mmol) was taken up in anhydrous acetonitrile (50 mL). To the yellow solution was added trifluoroacetic acid:water (9:1; 40 mL). The yellow reaction mixture was stirred at room temperature. After approximately eighteen hours at room temperature, the solvents were removed under reduced pressure. Purification by Biotage chromatography (gradient elution: 0 to 25% methanol/dichloromethane over 20 column volumes) gave 1.85 gm (66%) of Example 27 (Compound 10e) as a white solid; R_{f} 0.26 (10% methanol-dichloromethane); ¹H NMR (DMSO-d6): δ 8.99 (bs, 1H), 8.68 (d, 1H), 7.96 (m, 3H), 7.90 (m, 1H), 7.82 (m, 1H), 7.35 (m, 3H), 7.20 (m, 4H), 7.14 (m, 1H), 6.72 (m, 2H), 5.08 (m, 2H), 4.45 (bs, 1H), 3.40-4.05 (m, 24H), 3.22 (s, 3H), 3.10 (m, 1H), 2.90 (m, 1H), 2.72 (m, 1H), 2.61 (m, 1H), 2.46 (m, 3H), 1.45 (m, 2H), 0.77 (s, 9H), 0.75 (s, 9H). MS 1245 (M+H)⁺.

### Example 28

### Other Compounds

Using the techniques described herein, other compounds having different water-soluble, non-peptidic oligomers, different sizes of water-soluble, non-peptidic oligomers, different lipophilic moiety-containing residues, different sizes of lipophilic moiety-containing residues, different protease inhibitors, and so forth can be prepared. For example, using the techniques above, the following compounds were made (nomenclature consistent with the generally accepted abbreviations and the usage adopted herein): mPEG₃-atazanavir-ethoxy-CME-Leu-Leu, mPEG₅-atazanavir-ethoxy-CME-Leu-Leu, mPEG₆-atazanavir-ethoxy-CME-Leu-Leu, mPEG₃-atazanavir-ethoxy-CME-Phe-Phe, mPEG₅-atazanavir-ethoxy-CME-Phe-Phe, mPEG₆-atazanavir-ethoxy-CME-Phe-Phe, ethoxy ethoxy mPEG₃-atazanavir, ethoxy ethoxy mPEG₅-atazanavir, ethoxy ethoxy mPEG₆-atazanavir, atazanavir-1-ethoxypropoxy-mPEG₃, atazanavir-1-ethoxypropoxy-mPEG₅ and atazanavir-1-ethoxypropoxy-mPEG₆.

### Example 29

### Cytoprotection in CEM-SS Cells Infected with HIV-1_{RF}

To evaluate whether the protease inhibitors described herein retain the ability to protect against HIV-1 infection, CEM-SS cells infected with the RF strain of HIV-1 were treated with test compounds for six days, and then cell viability was monitored using the tetrazolium dye XTT. Infection studies were performed and EC₅₀ values were calculated as the protease inhibitor concentration leading to 50% reduction in cell death compared to virus-infected cells without protease inhibitor. TC₅₀ values were calculated as the protease inhibitor concentration leading to 50% cell death in the absence of viral infection. A value for Therapeutic Index (TI) was calculated as TC₅₀/EC₅₀. The results are summarized in Table 1.

The mPEG₃-atazanavir-monophospholipid and mPEG₆-atazanavir monophospholipid conjugates were protective against HIV-1_{RF} infection, yielding EC₅₀ values of 0.35 and 14.2 μM, respectively. The mPEG₅-atazanavir-monophospholipid conjugate started to demonstrate anti-HIV activity concurrent with onset on intrinsic activity, and therefore no EC₅₀ value could be determined.

TABLE 1
Activity of Compounds in Anti-HIV Cytoprotection Assay

| | CEM-SS/HIV- | CEM-SS TC₅₀ | |
|---|---|---|---|
| Compound | 1_{RF} EC₅₀ (μM) | (μM) | TI |
| mPEG₃-Atazanavir- | 0.35 | 14.2 | 40.8 |
| monophospholipid | | | |
| (sodium salt) | | | |
| (Example 24a) | | | |
| mPEG₅-Atazanavir- | >200.0* | 12.0 | — |
| monophospholipid | | | |
| (sodium salt) | | | |
| (Example 24ba) | | | |
| mPEG₆-Atazanavir- | 14.2 | 12.6 | 0.9 |
| monophospholipid | | | |
| (sodium salt) | | | |
| (Example 16a) | | | |

*Start to see low level of anti-HIV activity coinciding with onset of cytotoxicity, thus 50% protection against HIV is never reached

### Example 30

### Effect of PEG-PI Compounds on Viral Replication in Human Peripheral Blood Mononuclear Cells and Monocytes

Fresh human peripheral blood mononuclear cells (PBMCs) obtained from a commercial source were purified following centrifugation using a Ficoll-Hypaque density gradient. Viable cells were induced to proliferate in the presence of PHA-P and recombinant human IL-2 for 72 hours. PBMCs pooled from three donors were cultured with test compounds in the presence of the HT/92/599 clinical subtype B HIV-1 for seven days, followed by measurement of reverse transcriptase activity in cell supernatants using a radioactive incorporation polymerization assay. Cell supernatant containing HIV-1 viral was incubated for 90 minutes at 37° C. with a reaction mixture containing EGTA, Triton X-100, Tris (pH 7.4), DTT, MgCl₂, poly rA, oligo dT, and tritiated thymidine triphosphate (TTP). Incorporation of TTP into DNA was monitored by spotting the reaction mixture onto a DEAE filter mat, followed by washes with 5% phosphate buffer, distilled water, and 70% ethanol, and subsequent drying of the mat. Radioactivity was measured using a Wallac 1450 Microbeta Trilux liquid scintillation counter using Opti-Fluor O. Test compound-associated toxicity was measured in PBMCs cultured for seven days in the absence of virus using XTT reagent. EC₅₀ and EC₉₀ values were calculated as the protease inhibitor concentration leading to 50% and 90% reduction in cell death, respectively, compared to virus-infected cells without protease inhibitor. TC₅₀ values were calculated as the protease inhibitor concentration leading to 50% cell death in the absence of viral infection. A value for Therapeutic Index (TI) was calculated as TC₅₀/EC₅₀. The data are summarized in Table 2.

mPEG₃-Atazanavir-monophospholipid, mPEG₅-atazanavir-monophospholipid, and mPEG6-atazanavir-monophospholipid conjugates were protective against HIV-1, yielding EC₅₀ values of 0.97, 12.3, and 12.4 μM, respectively (Table 2), due to intrinsic cytotoxicity potentials resulting in TI values of 45.05, 1.39, and 3.03 for mPEG₃-, mPEG₅-, and mPEG₆-atazanavir-monophospholipid conjugates, respectively.

TABLE 2
Effect of Test Compounds on HIV-1 Reverse Transcriptase
Activity Following Infection in Fresh Human PBMCs

| | PBMC/HIV- | PBMC/HIV- | PBMC | |
|---|---|---|---|---|
| | 1_{HT/92/599} EC₉₀ | 1_{HT/92/599} EC₅₀ | TC₅₀ | |
| Compound | (μM) | (μM) | (μM) | TI |
| mPEG₃-Atazanavir- | 2.52 | 0.97 | 43.7 | 45.05 |
| monophospholipid | | | | |
| (sodium salt) | | | | |
| (Example 24a) | | | | |
| mPEG₅-Atazanavir- | 26.8 | 12.3 | 17.1 | 1.39 |
| monophospholipid | | | | |
| (sodium salt) | | | | |
| (Example 24b) | | | | |
| mPEG₆-Atazanavir- | 26.3 | 12.4 | 37.6 | 3.03 |
| monophospholipid | | | | |
| (sodium salt) | | | | |
| (Example 16a) | | | | |

### Example 31

### Pharmacokinetic Analysis

Pharmacokinetic analysis of protease inhibitors described herein was conducted. To evaluate the pharmacokinetics of PEGylated protease inhibitor conjugates with releasable lipophilic attachments, test compounds were administered to male Sprague-Dawley rats.

At various times following test article administration, serial blood samples were collected from indwelling jugular vein catheters and transferred to micro-tubes containing 7.5 μL of 20% w/v K2EDTA as an anticoagulant. Following processing of the blood samples to plasma (10 minutes at 10000 rpm, Centrifuge Model: Kubota 3500), plasma concentrations of the test article or corresponding PEGylated molecule released from the test article was measured using LC-MS/MS methods. Pharmacokinetic parameters were estimated using noncompartmental methods. The data are summarized below.

Table 3 presents a summary of pharmacokinetic parameters of PEGylated protease inhibitor prodrugs, wherein Cmax is the maximum (peak) concentration, AUCall represents the area under the concentration-time curve from zero to time of last concentration value, T½ is half life, and MRTlast is the mean residence time to last observable concentration

Tables 4, 5 and 6 present a summary of pharmacokinetic parameters of released PEGylated protease inhibitors (for the PEG₃, PEG₅ and PEG₆ “series” of compounds, respectively) following P.O. administration of respective prodrugs, wherein C24 represents the concentration at 24 hours, Cmax is the maximum (peak) concentration, AUCall represents the area under the concentration-time curve from zero to time of last concentration value, Tmax is the time to reach maximum or peak concentration following administration, and MRTlast is the mean residence time to last observable concentration.

TABLE 3
Summary of Pharmacokinetic Parameters of PEGylated Protease Inhibitor Prodrugs

| | | AUCall | Cmax | Cmin(24 hr) | t½(z) | MRTlast |
|---|---|---|---|---|---|---|
| Compound | Example | (hr · ng/mL) | (ng/mL) | (ng/mL) | (hr) | (hr) |
| mono-mPEG₃-Atazanavir- | 15a | 1930 ± 1170 | 470 ± 179 | | 3.33 ± 0.958 | 2.73 ± 0.912 |
| L-Leucine | | | | | | |
| mPEG₅-Atazanavir-L- | 15b | 487 ± 348 | 191 ± 85.6 | | 2.37 ± 2.19 | 1.71 ± 0.327 |
| Leucine | | | | | | |
| mPEG₆-Atazanavir-L- | 15c | 107 ± 62.4 | 55.5 ± 41.6 | | 2.02 ± 1.07 | 1.72 ± 0.237 |
| Leucine | | | | | | |
| Leu-Phe-mPEG₃- | 25 | 157 ± 30.3 | 41.9 ± 7.92 | 0.154 ± 0.307 | 3.20 ± 0.981 | 3.42 ± 0.916 |
| Atazanavir | | | | | | |
| Phe-Phe-mPEG₃- | 25 | 609 ± 157 | 112 ± 31.6 | 3.15 ± 0.946 | 8.31 ± 1.21 | 6.27 ± 1.31 |
| Atazanavir | | | | | | |
| Val-Phe-mPEG₃- | 25 | 700 ± 361 | 334 ± 133 | | 2.25 ± 1.28 | 1.68 ± 0.297 |
| Atazanavir | | | | | | |
| mPEG₃-atazanavir ethyl | 19a | 4960 ± 442 | 1190 ± 294 | 2.42 ± 1.20 | 6.56 ± 5.13 | 3.12 ± 0.285 |
| carbonate | | | | | | |
| mPEG₃-atazanavir | 27a | 221 ± 57.1 | 167 ± 89.6 | 0.177 ± 0.353 | 3.24 ± 3.26 | 1.86 ± 1.49 |
| glutaric-D-glucofuranose | | | | | | |
| mPEG-₃-Atazanavir- | 28 | 8.27 ± 3.06 | 2.95 ± 1.30 | | 1.21 ± NC | 1.01 ± 0.095 |
| Ethoxy-CM-Leu-Leu | | | | | | |
| mPEG₃-Atazanavir- | 28 | 351 ± 50.2 | 61.3 ± 52.7 | 2.68 ± 1.51 | 7.69 ± 2.54 | 8.23 ± 3.48 |
| glutaric acid | | | | | | |
| Phe-Gly-mPEG₃- | 25 | 6.07 ± 3.82 | 7.38 ± 6.67 | | 1.24 ± 0.237 | 0.602 ± 0.164 |
| Atazanavir | | | | | | |
| Leu-Gly-mPEG₃- | 25 | 0.955 ± 1.02 | 0.833 ± 0.676 | | | 0.597 ± 0.376 |
| Atazanavir | | | | | | |
| mPEG₃-atazanavir-CME- | 18 | 0.919 ± 0.968 | 0.772 ± 0.253 | | | 0.696 ± 0.381 |
| phe-phenylalanine | | | | | | |
| Phe-Phe-mPEG₅- | 25 | 31.8 ± 13.7 | 17.9 ± 9.09 | | 2.01 | 1.49 ± 0.412 |
| Atazanavir HCl | | | | | (n = 1) | |
| Leu-Phe-mPEG₅- | 25 | 53.8 ± 34.1 | 16.5 ± 14.9 | 1.16 ± 2.31 | 0.825 | 7.57 ± 7.76 |
| Atazanavir HCl | | | | | (n = 1) | |
| Phe-Gly-mPEG₅- | 25 | 0.341 ± 0.313 | 0.895 ± 0.808 | | | 0.357 ± 0.129 |
| Atazanavir HCl | | | | | | |
| Leu-Gly-mPEG₅- | 25 | 1.53 ± 2.93 | 0.479 ± 0.572 | | | 2.85 ± 3.68 |
| Atazanavir HCl | | | | | | |
| Val-Gly-mPEG₅- | 25 | 14.9 ± 18.7 | 33.7 ± 53.9 | | 2.95 | 0.779 ± 0.513 |
| Atazanavir HCl | | | | | (n = 1) | |
| Leu-Leu-mPEG₅- | 25 | 24.9 ± 6.24 | 7.21 ± 0.417 | 0.201 ± 0.401 | 3.6 ± 2.59 | 3.59 ± 3.27 |
| Atazanavir HCl | | | | | | |
| Phe-Leu-mPEG₅- | 25 | 42.4 ± 11.6 | 17 ± 4.2 | BLQ | 1.52 ± NC | 1.46 ± 0.396 |
| Atazanavir HCl | | | | | | |
| mPEG₅-Atazanavir | 27b | 78.5 ± 78.2 | 13.1 ± 12.4 | 2.64 ± 2.87 | | 11.4 ± 7.37 |
| Glutaric-D-glucofuranose | | | | | | |
| HCl | | | | | | |
| mPEG₅-Atazanavir - | 19b | 2250 ± 389 | 740 ± 253 | 2.19 ± 0.700 | 7.46 ± 4.14 | 2.6 ± 0.514 |
| Ethyl Carbonate HCl | | | | | | |
| CME Phe-Phe-mPEG₅- | 28 | 1.22 | 2.36 | BLQ | NC | 1.26 |
| Atazanavir HCl | | (n = 1) | (n = 1) | | | (n = 1) |
| 1-ethoxyethoxy-mPEG₃- | 28 | 3950 ± 2740 | 1370 ± 549 | 0.370 ± 0.455 | 2.01 ± 0.88 | 1.62 ± 0.350 |
| Atazanivir | | | | | | |
| 1-ethoxyethoxy-mPEG₅- | 28 | 4960 ± 1730 | 1420 ± 595 | 0.208 ± 0.416 | 2.14 ± 1.85 | 1.80 ± 0.060 |
| Atazanivir | | | | | | |
| 1-ethoxyethoxy-mPEG₆- | 28 | 2200 ± 2000 | 630 ± 550 | 0.354 ± 0.410 | 2.45 ± 0.630 | 2.17 ± 0.340 |
| Atazanivir | | | | | | |
| 1-ethoxypropoxy-mPEG₃- | 28 | 8480 ± 1700 | 2060 ± 260 | BLQ | 1.39 ± 0.210 | 2.29 ± 0.280 |
| Atazanivir | | | | | | |
| mPEG₃-Atazanavir- | 21a | 7740 ± 935 | 1680 ± 150 | 6.52 ± 3.57 | 4.03 ± 1.33 | 3.00 ± 0.350 |
| methyl methyl ether | | | | | | |
| mPEG₅-Atazanavir- | 21b | 3530 ± 1960 | 773 ± 481 | 6.61 ± 8.39 | 4.54 ± 1.68 | 3.93 ± 1.41 |
| methyl methyl ether | | | | | | |
| mPEG₆-Atazanavir- | 21c | 1140 ± 312 | 222 ± 73 | 3.47 ± 2.81 | 11.2 ± 7.72 | 5.12 ± 1.74 |
| methyl methyl ether | | | | | | |
| mPEG₃-Atazanavir- | 22a | 6590 ± 2150 | 1600 ± 442 | 0.797 ± 0.260 | 9.41 ± 3.35 | 2.44 ± 0.920 |
| methyl ethyl ether | | | | | | |
| Methoxyacetate-OCHMe— | 20 | 298 ± 121 | 38.7 ± 13.8 | | 6.30 ± 2.06 | 5.81 ± 2.95 |
| OCO₂-mPEG5-Atazanavir | | | | | | |
| Acetate-OCHMe—OCO₂- | 20 | 616 ± 156 | 115 ± 29.0 | | 4.82 ± 3.10 | 6.21 ± 3.38 |
| mPEG5-Atazanavir | | | | | | |
| mPEG₃-Atazanavir-CME- | 17, n = 3 | 13.1 ± 17.2 | 11.5 ± 12.8 | | | 3.74 ± 6.09 |
| Leucine HCl | | | | | | |
| mPEG₅-Atazanavir-CME- | 17, n = 5 | 0.119 ± 0.0826 | 0.45 ± 0.330 | | | 0.250 ± 0.000 |
| Leucine HCl | | | | | | |
| mPEG₆-Atazanavir-CME- | 17, n = 5 | 2.56 ± 2.89 | 0.851 ± 0.730 | | | 2.24 ± 2.58 |
| Leucine HCl | | | | | | |
| mPEG₃-Atazanavir-CME- | 18 | 10.2 ± 8.7 | 7.98 ± 6.06 | | | 2.36 ± 3.57 |
| phenylalanine HCl | | | | | | |
| mPEG₅-Atazanavir-CME- | 18 | 0.0554 ± 0.096 | 0.222 ± 0.384 | | | 0.25 |
| phenylalanine HCl | | | | | | |
| mPEG₆-Atazanavir-CME- | 18 | 6.41 ± 9.53 | 8.21 ± 12.1 | | | 0.948 ± 0.0741 |
| phenylalanine HCl | | | | | | |

TABLE 4
Mean (+SD) PK parameters of Atazanavir, mPEG₃-Atazanavir Following PO Administration
and Released mPEG₃-Atazanavir Following PO Administration of mPEG₃-Atazanavir Having
a Lipophilic Moiety-Containing Residue Releasably Attached Thererto in Rats (n = 4-5)

| | | AUCall | | Cmax | | MRT last | | Tmax* | C24 | |
|---|---|---|---|---|---|---|---|---|---|---|
| | | (ng × hr/mL) | | (ng/mL) | | (hr) | | (hr) | (hr) | |
| Example | Compound | Mean | SD | Mean | SD | Mean | SD | Median | Mean | SD |
| | mPEG3-ATV | 445 | 125 | 111 | 48.7 | 5.64 | 3.31 | 0.250 | 1.40 | 2.42 |
| 4a | C₄H₉CO-mPEG3- | 40.1 | 17.7 | 7.42 | 0.710 | 3.00 | 1.30 | 1.25 | 0.00 | 0.00 |
| | ATV | | | | | | | | | |
| 12a | Acetyl-mPEG₃-ATV | 5.63 | 1.12 | 2.43 | 0.859 | 1.01 | 0.0892 | 0.750 | 0.00 | 0.00 |
| 2 | mPEG₃-ATV-L- | 70.4 | 13.2 | 11.8 | 1.53 | 4.45 | 1.08 | 0.500 | 0.213 | 0.369 |
| | valine | | | | | | | | | |
| 15a | mPEG₃-ATV-L- | 347 | 106 | 66.0 | 14.1 | 4.14 | 0.621 | 0.750 | 1.33 | 0.674 |
| | leucine | | | | | | | | | |
| 6 | C₃H₇CO-mPEG₃- | 11.4 | 2.80 | 3.08 | 0.441 | 1.56 | 0.689 | 1.00 | 0.00 | 0.00 |
| | ATV | | | | | | | | | |
| 9 | C₂H₅CO-mPEG₃- | 59.6 | 25.5 | 17.8 | 4.62 | 2.44 | 0.723 | 0.500 | 0.00 | 0.00 |
| | ATV | | | | | | | | | |
| 4d | C₅H₁₁CO-mPEG₃- | 33.9 | 19.4 | 7.51 | 3.76 | 2.78 | 1.97 | 2.00 | 0.120 | 0.269 |
| | ATV | | | | | | | | | |
| 16 | mPEG₃-ATV- | 16.8 | 8.13 | 1.66 | 0.518 | 7.37 | 4.28 | 1.25 | 0.219 | 0.439 |
| | phosphate | | | | | | | | | |
| 16b | PEG₃-Atazanavir | 7.72 | 9.20 | 18.7 | 28.7 | 0.640 | 0.359 | 0.250 | 0.00 | 0.00 |
| | Monophospholipid | | | | | | | | | |
| 25 | Leu-Phe-mPEG₃- | 448 | 101 | 84.9 | 21.4 | 7.56 | 2.38 | 2.00 | 2.35 | 1.08 |
| | ATV | | | | | | | | | |
| 25 | Phe-Phe-mPEG₃- | 652 | 263 | 199 | 91.5 | 5.32 | 1.62 | 0.500 | 1.96 | 0.554 |
| | ATV | | | | | | | | | |
| 25 | Val-Phe-mPEG₃- | 502 | 220 | 109 | 53.4 | 5.57 | 1.22 | 1.50 | 1.87 | 0.574 |
| | ATV | | | | | | | | | |
| 17, n = 3 | mPEG₃-ATV-CME- | 85.9 | 49.7 | 4.39 | 1.16 | 17.4 | 5.54 | 9.00 | 2.67 | 2.15 |
| | Leucine | | | | | | | | | |
| 1 | mPEG₃-ATV ethyl | 199 | 64.4 | 17.9 | 3.39 | 15.5 | 8.88 | 1.00 | 1.42 | 0.255 |
| | carbonate | | | | | | | | | |
| 27a | mPEG₃-ATV- | 84.4 | 8.98 | 3.62 | 0.714 | 22.5 | 0.438 | 0.250 | 1.55 | 0.118 |
| | glutaric-D- | | | | | | | | | |
| | glucofuranose | | | | | | | | | |
| 28 | mPEG₃-ATV- | 148 | 44.7 | 6.42 | 2.05 | 16.5 | 1.23 | 9.00 | 3.96 | 2.75 |
| | Ethoxy-CM-Leu-Leu | | | | | | | | | |
| 18 | mPEG₃-ATV- | 99.5 | 8.11 | 4.48 | 1.43 | 18.5 | 1.90 | 6.00 | 1.74 | 0.546 |
| | Phenylalanine | | | | | | | | | |
| 28 | mPEG₃-ATV- | 53.8 | 33.7 | 2.41 | 1.54 | 19.5 | 3.14 | 24.0 | 2.41 | 1.54 |
| | glutaric acid | | | | | | | | | |
| 25 | Phe-Gly-mPEG₃- | 69.6 | 19.2 | 5.02 | 0.994 | 12.6 | 4.59 | 1.00 | 0.807 | 0.307 |
| | ATV | | | | | | | | | |
| 25 | Leu-Gly-mPEG₃- | 44.9 | 15.1 | 2.10 | 0.765 | 15.9 | 6.72 | 4.00 | 1.09 | 0.235 |
| | ATV | | | | | | | | | |
| 18 | mPEG₃-ATV-CME- | 120 | 24.3 | 6.26 | 2.83 | 16.2 | 2.91 | 1.25 | 2.14 | 1.12 |
| | Phe-Phe | | | | | | | | | |
| 28 | Ethoxy ethoxy | 500 | 177 | 154 | 57.3 | 2.35 | 0.255 | 0.500 | 0.140 | 0.280 |
| | mPEG₃-ATV | | | | | | | | | |
| 28 | Ethoxy propoxy | 849 | 411 | 153 | 64.4 | 3.83 | 0.764 | 1.00 | 0.00 | 0.00 |
| | mPEG₃-ATV | | | | | | | | | |
| 22a | Methyl ethyl ether | 822 | 136 | 190 | 55.0 | 3.29 | 0.685 | 0.750 | 1.20 | 1.10 |
| | mPEG₃-ATV | | | | | | | | | |
| 21a | Methyl methyl ether | 110 | 28.6 | 23.0 | 4.35 | 3.26 | 1.75 | 1.00 | 0.218 | 0.435 |
| | mPEG₃-ATV | | | | | | | | | |
| 23a | mPEG₃ ATV-Methyl | 4.10 | 2.15 | 0.951 | 0.235 | 1.93 | 1.17 | 2.00 | 0.00 | 0.00 |
| | ethyl methyl ether | | | | | | | | | |

NC: Not calculated; ATV: atazanavir

TABLE 5
Mean (+SD) PK parameters of Atazanavir, mPEG₅-Atazanavir Following PO Administration
and Released mPEG₅-Atazanavir Following PO Administration of mPEG₅-Atazanavir Having
a Lipophilic Moiety-Containing Residue Releasably Attached Thererto in Rats (n = 4-5)

| | | AUCall | | Cmax | | MRT last | | Tmax | C24 | |
|---|---|---|---|---|---|---|---|---|---|---|
| | | (ng × hr/mL) | | (ng/mL) | | (hr) | | (hr) | (hr) | |
| Example | Compound | Mean | SD | Mean | SD | Mean | SD | Median | Mean | SD |
| | PEG5-ATV | 389 | 200 | 57.6 | 55.9 | 9.61 | 7.17 | 0.750 | 3.81 | 5.63 |
| 4b | C₄H₉CO-PEG₅- | 39.3 | 14.6 | 9.46 | 2.92 | 2.43 | 0.559 | 0.750 | 0.00 | 0.00 |
| | ATV | | | | | | | | | |
| 12b | Acetyl-mPEG₅-ATV | 0.00 | 0.00 | 0.00 | 0.00 | NC | NC | NC | 0.00 | 0.00 |
| 3 | mPEG₅-ATV-L- | 9.14 | 14.1 | 1.09 | 0.794 | 6.89 | 10.1 | 1.00 | 0.378 | 0.757 |
| | valine | | | | | | | | | |
| 15b | PEG₅-ATV-L- | 217 | 81.5 | 34.4 | 16.7 | 7.83 | 2.01 | 1.00 | 2.10 | 1.07 |
| | leucine | | | | | | | | | |
| 7 | C₃H₇CO-mPEG₅-ATV | 3.92 | 0.764 | 1.44 | 0.243 | 0.984 | 0.0638 | 1.00 | 0.00 | 0.00 |
| 10 | C₂H₅CO-mPEG₅-ATV | 39.1 | 15.0 | 6.90 | 3.96 | 4.04 | 1.54 | 0.750 | 0.00 | 0.00 |
| 4e | C₅H₁₁CO-mPE₅- | 23.7 | 9.15 | 7.38 | 4.22 | 2.42 | 0.800 | 0.500 | 0.00 | 0.00 |
| | ATV | | | | | | | | | |
| 16 | mPEG₅-ATV- | 23.1 | 19.1 | 1.52 | 1.03 | 10.9 | 4.16 | 7.00 | 0.602 | 0.784 |
| | phosphate | | | | | | | | | |
| 16a | PEG₅-Atazanavir | 0.00 | 0.00 | 0.00 | 0.00 | NC | NC | NC | 0.00 | 0.00 |
| | Monophospholipid | | | | | | | | | |
| 25 | Phe-Phe-mPEG₅- | 94.3 | 42.0 | 12.9 | 5.08 | 9.22 | 7.03 | 0.750 | 1.09 | 1.27 |
| | ATV | | | | | | | | | |
| 25 | Leu-Phe-mPEG₅- | 147 | 21.3 | 25.6 | 17.8 | 9.33 | 5.90 | 1.50 | 1.51 | 0.902 |
| | ATV | | | | | | | | | |
| 25 | Phe-Gly-mPEG₅- | 33.7 | 31.8 | 3.10 | 2.70 | 12.0 | 8.54 | 12.0 | 0.169 | 0.292 |
| | ATV | | | | | | | | | |
| 25 | Leu-Gly-mPEG₅- | 344 | 646 | 19.5 | 34.7 | 11.3 | 8.88 | 12.0 | 18.1 | 35.7 |
| | ATV | | | | | | | | | |
| 25 | Val-Gly-mPEG₅- | 58.4 | 44.4 | 4.09 | 3.64 | 23.0 | 12.3 | 12.3 | 0.597 | 0.408 |
| | ATV | | | | | | | | | |
| 25 | Leu-Leu-mPEG₅- | 118 | 19.6 | 14.1 | 5.05 | 10.7 | 4.86 | 0.750 | 2.98 | 0.195 |
| | ATV | | | | | | | | | |
| 25 | Phe-Leu-mPEG₅- | 245 | 149 | 16.9 | 4.14 | 10.9 | 3.44 | 1.25 | 8.77 | 7.94 |
| | ATV | | | | | | | | | |
| 17, n = 5 | mPEG₅-ATV-CME- | 65.6 | 35.5 | 2.88 | 1.23 | 16.5 | 6.72 | 9.00 | 1.71 | 1.57 |
| | Leucine | | | | | | | | | |
| 27b | mPEG₅-ATV | 50.2 | 18.8 | 2.74 | 1.06 | 27.1 | 8.69 | 24.0 | 2.05 | 0.630 |
| | glutaric-D- | | | | | | | | | |
| | glucofuranose | | | | | | | | | |
| 28 | mPEG₅ATV- | 50.3 | 24.7 | 1.86 | 1.07 | 26.7 | 3.17 | 24.0 | 1.82 | 1.12 |
| | Ethoxy-CM-Leu- | | | | | | | | | |
| | Leu | | | | | | | | | |
| 18 | mPEG₅-ATV- | 54.1 | 10.2 | 2.84 | 1.31 | 14.0 | 3.07 | 12.0 | 0.591 | 0.439 |
| | Phenylalanine | | | | | | | | | |
| 19b | mPEG₅-ATV-Ethyl | 93.2 | 8.99 | 16.7 | 4.73 | 8.51 | 3.02 | 2.00 | 2.76 | 1.45 |
| | Carbonate | | | | | | | | | |
| 28 | CME Phe-Phe- | 299 | 213 | 14.7 | 11.8 | 23.9 | 1.42 | 24.0 | 14.7 | 11.8 |
| | mPEG₅-ATV | | | | | | | | | |
| 20 | Acetate-OCHMeO— | 616 | 156 | 115 | 29.0 | 6.21 | 3.38 | 0.750 | 4.88 | 5.6 |
| | CO₂-mPEG₅ ATV | | | | | | | | | |
| 28 | ATV-1-Ethoxy | 235 | 37.3 | 55.3 | 24.6 | 3.53 | 1.56 | 1.00 | 0.281 | 0.327 |
| | propoxy-mPEG₅ | | | | | | | | | |
| 28 | Ethoxy Ethoxy | 329 | 116 | 109 | 64.2 | 1.95 | 0.341 | 1.50 | 0.00 | 0.00 |
| | mPEG₅-ATV | | | | | | | | | |
| 20 | Methoxyacetate- | 298 | 121 | 38.7 | 13.8 | 5.81 | 2.95 | 1.50 | 0.885 | 0.861 |
| | OCHMeO—CO2- | | | | | | | | | |
| | mPEG₅ ATV | | | | | | | | | |
| 22b | Methyl ethyl ether | 489 | 117 | 95.1 | 30.0 | 6.19 | 6.55 | 1.00 | 2.7 | 3.53 |
| | mPEG₅-ATV | | | | | | | | | |
| 21b | Methyl methyl ether | 137 | 41.0 | 23.3 | 8.93 | 4.76 | 2.26 | 2.00 | 0.281 | 0.324 |
| | mPEG₅-ATV | | | | | | | | | |
| 23b | mPEG₅-ATV- | 32.7 | 16.9 | 3.74 | 0.759 | 4.92 | 2.31 | 2.00 | 0.00 | 0.00 |
| | Methyl ethyl methyl | | | | | | | | | |
| | ether | | | | | | | | | |

NC: Not calculated; ATV: atazanavir

TABLE 6
Mean (+SD) PK parameters of Atazanavir, mPEG₆-Atazanavir Following PO Administration
and Released mPEG₆-Atazanavir Following PO Administration of mPEG₆-Atazanavir Having
a Lipophilic Moiety-Containing Residue Releasably Attached Thererto in Rats (n = 4-5)

| | | AUCall | | Cmax | | MRT last | | Tmax | C24 | |
|---|---|---|---|---|---|---|---|---|---|---|
| | | (ng × hr/mL) | | (ng/mL) | | (hr) | | (hr) | (hr) | |
| Example | Compound | Mean | SD | Mean | SD | Mean | SD | Median | Mean | SD |
| | PEG₆-ATV | 97.6 | 31.7 | 26.8 | 28.6 | 4.93 | 1.40 | 0.250 | 0.477 | 0.596 |
| 4c | C₄H₉CO-mPEG₆- | 36.5 | 33.5 | 13.8 | 8.37 | 1.14 | 0.416 | 0.750 | 0.00 | 0.00 |
| | ATV | | | | | | | | | |
| 12c | Acetyl-mPEG₆- | 150 | NC | 10.3 | NC | 13.8 | NC | 6.00 | 0.688 | 0.760 |
| | Atazanavir | | | | | | | | | |
| 5 | mPEG₆-ATV- | 0.00 | 0.00 | 0.00 | 0.00 | NC | NC | NC | 0.00 | 0.00 |
| | butylcarbamate | | | | | | | | | |
| 1 | mPEG₆-ATV- | 3.07 | 3.44 | 0.634 | 0.834 | 15.1 | 20.3 | 1.00 | 0.00 | 0.00 |
| | ethylcarbamate | | | | | | | | | |
| 15c | mPEG₆-ATV-L- | 148 | 82.6 | 9.49 | 3.14 | 10.2 | 6.15 | 0.500 | 3.76 | 2.92 |
| | leucine | | | | | | | | | |
| 14 | mPEG₆-ATV-L- | 73.1 | 15.4 | 4.05 | 0.860 | 18.9 | 2.27 | 12.0 | 0.778 | 0.152 |
| | valine | | | | | | | | | |
| 25 | mPEG₆-ATV-glycine | 12.8 | 14.3 | 0.778 | 0.146 | 13.3 | 11.0 | 6.00 | 0.189 | 0.423 |
| 13c | C₇H₁₅CO-mPEG₆- | 66.0 | 21.0 | 11.1 | 7.00 | 4.47 | 2.21 | 2.00 | 0.00 | 0.00 |
| | ATV | | | | | | | | | |
| 4f | C₅H₁₁CO-mPEG₆- | 78.1 | 30.2 | 21.4 | 15.7 | 5.01 | 6.11 | 0.500 | 0.00 | 0.00 |
| | ATV | | | | | | | | | |
| 8 | C₃H₇CO-mPEG₆- | 6.16 | 2.09 | 2.22 | 1.06 | 1.36 | 0.445 | 2.00 | 0.00 | 0.00 |
| | ATV | | | | | | | | | |
| 11 | C₂H₅CO-mPEG₆- | 13.2 | 14.6 | 1.86 | 0.938 | 8.28 | 14.4 | 2.00 | 0.00 | 0.00 |
| | ATV | | | | | | | | | |
| 16 | mPEG₆-ATV- | 12.1 | 14.4 | 1.23 | 1.66 | 23.4 | 16.1 | 6.13 | 0.00 | 0.00 |
| | phosphate | | | | | | | | | |
| 16a | PEG₆-Atazanavir | 0.108 | 0.216 | 0.432 | 0.865 | 0.250 | NC | 0.250 | 0.144 | 0.287 |
| | Monophospholipid | | | | | | | | | |
| 25 | Leu-Leu-mPEG₆- | 152 | 47.9 | 14.2 | 5.33 | 13.1 | 2.19 | 1.50 | 2.37 | 2.46 |
| | ATV | | | | | | | | | |
| 25 | Phe-Leu-mPEG₆-ATV | 216 | 23.1 | 27.4 | 6.88 | 11.0 | 5.84 | 1.50 | 2.48 | 1.61 |
| 17, n = 6 | mPEG₆-ATV-CME- | 287 | 41.5 | 8.65 | 0.436 | 21.8 | 0.723 | 1.50 | 6.66 | 1.67 |
| | Leucine | | | | | | | | | |
| 27c | mPEG₆-ATV | 33.2 | 30.0 | 2.53 | 1.43 | 22.1 | 11.6 | 6.00 | 0.389 | 0.487 |
| | glutaric-D- | | | | | | | | | |
| | glucofuranose | | | | | | | | | |
| 28 | mPEG₆-ATV- | 40.6 | 20.9 | 2.07 | 0.870 | 13.8 | 7.51 | 4.00 | 1.10 | 1.47 |
| | Ethoxy-CM-Leu-Leu | | | | | | | | | |
| 18 | mPEG₆-ATV- | 351 | 25.8 | 12.5 | 2.54 | 22.1 | 1.77 | 1.00 | 6.34 | 1.40 |
| | Phenylalanine | | | | | | | | | |
| 19c | mPEG₆-ATV-Ethyl | 207 | 138 | 19.9 | 5.51 | 10.3 | 5.83 | 1.50 | 4.39 | 7.01 |
| | Carbonate | | | | | | | | | |
| 25 | Phe-Gly-mPEG₆- | 217 | 68.8 | 8.86 | 4.10 | 26.1 | 5.94 | 24.0 | 6.62 | 4.11 |
| | ATV | | | | | | | | | |
| 25 | Leu-Gly-mPEG₆- | 294 | 126 | 13.7 | 8.53 | 27.2 | 7.93 | 30.0 | 3.50 | 0.881 |
| | ATV | | | | | | | | | |
| 25 | Phe-Phe-mPEG₆- | 279 | 70.0 | 27.0 | 14.5 | 16.2 | 3.29 | 2.00 | 3.63 | 0.960 |
| | ATV | | | | | | | | | |
| 25 | Leu-Phe-mPEG₆- | 418 | 49.5 | 37.2 | 12.3 | 13.8 | 3.57 | 1.50 | 8.68 | 4.38 |
| | ATV | | | | | | | | | |
| 25 | Val-Gly-mPEG₆- | 194 | 20.9 | 5.11 | 0.931 | 24.3 | 0.609 | 7.00 | 4.07 | 0.756 |
| | ATV | | | | | | | | | |
| 28 | mPEG₆-ATV-CME- | 102 | 31.9 | 7.06 | 3.26 | 21.4 | 3.36 | 3.25 | 2.31 | 1.17 |
| | Phe-Phe | | | | | | | | | |
| 28 | ATV-1-Ethoxy | 185 | 121 | 83.0 | 48.9 | 5.16 | 8.19 | 0.750 | 0.00 | 0.00 |
| | propoxy-mPEG₆ | | | | | | | | | |
| 28 | Ethoxy Ethoxy | 137 | 36.8 | 28.8 | 17.5 | 4.33 | 3.27 | 1.50 | 0.388 | 0.775 |
| | mPEG₆-ATV | | | | | | | | | |
| 22c | Methyl ethyl ether | 202 | 118 | 38.3 | 9.37 | 10.1 | 17.0 | 2.00 | 0.00 | 0.00 |
| | mPEG₆-ATV | | | | | | | | | |
| 21c | Methyl methyl ether | 55.8 | 13.9 | 11.3 | 3.40 | 3.11 | 1.03 | 1.50 | 0.186 | 0.372 |
| | mPEG₆-ATV | | | | | | | | | |
| 23c | mPEG₆-ATV-Methyl | NC | NC | NC | NC | NC | NC | NC | 0.00 | 0.00 |
| | ethyl methyl ether | | | | | | | | | |

NC: Not calculated; ATV: atazanavir

1. A compound comprising a protease inhibitor covalently attached to both (i) a water-soluble, non-peptidic oligomer, and (ii) a lipophilic moiety-containing residue, wherein the lipophilic moiety-containing residue is attached to the protease inhibitor via a releasable linkage-containing spacer moiety.

2. The compound of claim 1, wherein the water-soluble, non-peptidic oligomer is attached to the protease inhibitor via a stable spacer moiety.

3. The compound of claim 1, wherein the water-soluble, non-peptidic oligomer is attached to the protease inhibitor via a releasable linkage-containing spacer moiety.

4. The compound of claim 1, wherein the protease inhibitor is selected from the group consisting of amprenavir, atazanavir, fosamprenavir, indinavir, lopinavir, saquinavir, nelfinavir, ritonavir, tipranovir and darunavir.

5. The compound of claim 1, wherein the protease inhibitor is atazanavir.

6. The compound of claim 1, wherein the water-soluble, non-peptidic oligomer is a poly(alkylene oxide).

7. The compound of claim 6, wherein the poly(alkylene oxide) is a poly(ethylene oxide).

8. The compound of claim 7, wherein the poly(ethylene oxide) has between 1 and 30 monomers.

9. The compound of claim 8, wherein the poly(ethylene oxide) has between 1 and 10 monomers.

10. The compound of claim 1, wherein the water-soluble, non-peptidic oligomer is attached to the protease inhibitor via a carbamate linkage.

11. The compound of claim 1, wherein the lipophilic moiety-containing residue is a residue selected from the group consisting of an organic radical, naturally occurring amino acids, non-naturally occurring amino acids, lipids, carbohydrates, phosphoholipids and vitamins.

12. The compound of claim 1, wherein the lipophilic moiety-containing residue is a residue of an amino acid.

13. The compound of claim 12, wherein the amino acid is valine.

14. The compound of claim 12, wherein the amino acid is phenylalanine.

15. The compound of claim 12, wherein the amino acid is leucine.

16. The compound of claim 1, wherein the lipophilic moiety-containing residue is a residue of a dipeptide.

17. The compound of claim 16, wherein the dipeptide is selected from the group consisting of Leu-Leu, Phe-Phe, Val-Val, Leu-Val, Val-Leu, Phe-Leu, Leu-Phe, Phe-Val, and Val-Phe.

18. The compound of claim 1, wherein the lipophilic moiety-containing residue is a residue of a phospholipid.

19. The compound of claim 1, wherein the lipophilic moiety-containing residue is a residue of a C₂₋₂₀ alkyl carbonate.

20. The compound of claim 1, wherein the lipophilic moiety-containing residue is a residue of a carbohydrate.

21. A composition comprising a compound of claim 1 and a pharmaceutically acceptable excipient.

22. A method comprising covalently attaching a lipophilic moiety-containing molecule to a protease inhibitor having a water-soluble, non-peptidic oligomer attached thereto.

23. A method comprising administering a compound of claim 1 to a patient.
